SEQUENCE LISTING (55 sequences)

SEQ ID NO: 1 - DNA, 26 bp, Artificial Sequence
  Synthetic
gccaccatggcaggtggaaaagcagg

SEQ ID NO: 2 - DNA, 28 bp, Artificial Sequence
  Synthetic
gatacattgatgagtttggacaaaccac

SEQ ID NO: 3 - DNA, 16 bp, Artificial Sequence
  Synthetic
tcagtcagaccgacta

SEQ ID NO: 4 - DNA, 19 bp, Artificial Sequence
  Synthetic
tttctttgggatagtccaa

SEQ ID NO: 5 - DNA, 97 bp, Artificial Sequence
  Synthetic
ccttttctttgggatagtccaagacacccccaactgagagaactcaaaggttaccccagttggggtctgactgaacctccctgcctccagcgccgtc

SEQ ID NO: 6 - DNA, 23 bp, Artificial Sequence
  Synthetic
tggccacgttcatatttaagcgg

SEQ ID NO: 7 - DNA, 23 bp, Artificial Sequence
  Synthetic
agcccttgctgtggtagctgtgg

SEQ ID NO: 8 - DNA, 23 bp, Artificial Sequence
  Synthetic
ccaccaaacccaaacatccgtcg

SEQ ID NO: 9 - DNA, 23 bp, Artificial Sequence
  Synthetic
ccacagcgctggcggtcatagca

SEQ ID NO: 10 - DNA, 27 bp, Artificial Sequence
  Synthetic
gcaggtaaccataccagggataaaagg

SEQ ID NO: 11 - DNA, 26 bp, Artificial Sequence
  Synthetic
ggtaaagacgaaaagacgcaagactg

SEQ ID NO: 12 - DNA, 20 bp, Artificial Sequence
  Synthetic
caggaggaggccaatgatgg

SEQ ID NO: 13 - DNA, 24 bp, Artificial Sequence
  Synthetic
gcactgacatcactcttgctcaac

SEQ ID NO: 14 - DNA, 30 bp, Artificial Sequence
  Synthetic
ccatgaatgcagaaatctttgtattcctcc

SEQ ID NO: 15 - DNA, 24 bp, Artificial Sequence
  Synthetic
gcctcgagtttgaccttttcttcg

SEQ ID NO: 16 - DNA, 20 bp, Artificial Sequence
  Synthetic
atgaactttttaacagtctt

SEQ ID NO: 17 - DNA, 20 bp, Artificial Sequence
  Synthetic
cagagagaaatcgtgacatc

SEQ ID NO: 18 - DNA, 21 bp, Artificial Sequence
  Synthetic
acacgtttcctctagtcccag

SEQ ID NO: 19 - DNA, 23 bp, Artificial Sequence
  Synthetic
gggttttactgtgctcaaattgc

SEQ ID NO: 20 - DNA, 18 bp, Artificial Sequence
  Synthetic
atggtgagcaagggcgag

SEQ ID NO: 21 - DNA, 20 bp, Artificial Sequence
  Synthetic
cttgtacagctcgtccatgc

SEQ ID NO: 22 - DNA, 20 bp, Artificial Sequence
  Synthetic
gggccctcatgcatatgtga

SEQ ID NO: 23 - DNA, 20 bp, Artificial Sequence
  Synthetic
agaatttccacacggtcgcc

SEQ ID NO: 24 - DNA, 20 bp, Artificial Sequence
  Synthetic
agcgggatttacgcactcat

SEQ ID NO: 25 - DNA, 23 bp, Artificial Sequence
  Synthetic
gcatgcagaatatcttaatgcca

SEQ ID NO: 26 - DNA, 20 bp, Artificial Sequence
  Synthetic
gggcacggggaacaccaact

SEQ ID NO: 27 - DNA, 20 bp, Artificial Sequence
  Synthetic
tggtcgcttctcgctctcgg

SEQ ID NO: 28 - DNA, 18 bp, Artificial Sequence
  Synthetic
gacaacggctccggtatg

SEQ ID NO: 29 - DNA, 18 bp, Artificial Sequence
  Synthetic
catgccaaccatcactcc

SEQ ID NO: 30 - DNA, 25 bp, Artificial Sequence
  Synthetic
aacatgctacgcagagagggagtgg

SEQ ID NO: 31 - DNA, 29 bp, Artificial Sequence
  Synthetic
catgagacaaggaacccctagtgatggag

SEQ ID NO: 32 - DNA, 29 bp, Artificial Sequence
  Synthetic
catgagacaaggaacccctagtgatggag

SEQ ID NO: 33 - DNA, 12 bp, Artificial Sequence
  Synthetic
gaagcaattaag

SEQ ID NO: 34 - DNA, 310 bp, Danio rerio
agggttgattactcagccatttgccccgaatgtctaagcgtgagtcagagcacccagcctttgtgtctccatctgaaaccttttcctcccctgttgtgtttttttggggctgatgagcagatgaatgggccctggggacaaagagatgatgtaggtttctacgcatctggcccttcatcaatggactcggatcaaacactcaccacaatatatgcaaccaaggcaaatgttcagctgatgatctgcattctgctgtgagccagttgatgaaatcgctgaattacacatctgaatcagaagcaattaagct

SEQ ID NO: 35 - protein, 230 aa, Danio rerio
MNFLTVLRLFVFTIVAVRGASIPLVFESERPAQTAVIDLLEALSEKRSTVDNKLVEYEEEDEEYYYDDDVEEDEEDEFSGDYGLPIVAFSSQPKDPSAVLNAEKLEGSKRKGLGRKKGKGKGKGKKRNPCLKKYKDFCIHGTCQYFRDLKGPSCICDQGYSGERCHDFSLPVKTNDGGYDRTTALAVVAVVLSSLCLTIIGLLLALRFHKQGAYNVENEEKIKLGAAPHH

SEQ ID NO: 36 - protein, 208 aa, Mus musculus
MKLLPSVMLKLFLAAVLSALVTGESLERLRRGLAAATSNPDPPTGSTNQLLPTGGDRAQGVQDLEGTDLNLFKVAFSSKPQGLATPSKERNGKKKKKGKGLGKKRDPCLRKYKDYCIHGECRYLQEFRTPSCKCLPGYHGHRCHGLTLPVENPLYTYDHTTVLAVVAVVLSSVCLLVIVGLLMFRYHRRGGYDLESEEKVKLGVASSH

SEQ ID NO: 37 - protein, 208 aa, Homo sapiens
MKLLPSVVLKLFLAAVLSALVTGESLERLRRGLAAGTSNPDPPTVSTDQLLPLGGGRDRKVRDLQEADLDLLRVTLSSKPQALATPNKEEHGKRKKKGKGLGKKRDPCLRKYKDFCIHGECKYVKELRAPSCICHPGYHGERCHGLSLPVENRLYTYDHTTILAVVAVVLSSVCLLVIVGLLMFRYHRRGGYDVENEEKVKLGMTNSH

SEQ ID NO: 38 - protein, 643 aa, Danio rerio
MSSPKGIAIGIDLGTTYSCVGVFQHGKVEIIANDQGNRTTPSYVAFTDTERLIGDAAKNQVAMNPNNTVFDAKRLIGRRFDDPVVQSDMKHWSFKVVSDGGKPKVAVEHKGENKTFNPEEISSMVLVKMKEIAEAYLGQKVTNAVITVPAYFNDSQRRATKDAGVIAGLNVLRIINEPTAAAIAYGLDKGKSSERNVLIFDLGGGTFDVSILTIEDGIFEVKATAGDTHLGGEDFDNRMVNHFVEEFKRKHKKDISQNKRALRRLRTACERAKRTLSPSSQASIEIDSLYEGIDFYTSITRARFEELCSDLFRGTLDPVEKALRDAKMDKAQIHDIVLVGGSTRIPKIQKLLQDFFNGRELNKSINPDEAVAYGAAVQAAILMGDTSGNVQDLLLLDVAPLSLGIETAGGVMTALIKRNTTIPTKQTQTFTTYSDNQPGVLIQVFEGERAMTKDNNLLGKFELTGIPPAPRGVPQIEVTFDIDANGILNVSAADKSTGKQNRITITNDKGRLSKEEIERMVQEADKYKAEDDLQREKISAKNSLESYAFNMKNSVEDDNLKGKISEEDKKRVVEKCNEAVSWLENNQLADKEEYEHQLKELEKVCNPVISKLYQGGMPAGGCGAQARAASGASAQGPTIEEVD

SEQ ID NO: 39 - DNA, 2754 bp, Danio rerio
aaactcaaccgaagagaagcgacttgacaaagaagaaaagagcagcctgacaggacttttccccgacgaggtgtttattcgctctatttaagaatctactgtaaggtaagtctcaatatattgtactctagtggctaatcaaaattttatagagattatatgtacttaatgtcaaaaaatctactttgtatatgtaatctttttacatgtggactgcctatgttcatcttattttaggtctactagaaaattatatttcccgttttcacaataaggatttaaaaaaaaagcaatgaacagactggcatttactttatgttgctgacattattatatatgagcataataaccataaatactagcaaatgtcctaaatgaatttgtgttaatgttgtctacaaaagaaaagaaaattagcgttttacttgtacaactaataataacttagttattaagagaatttcacttgttgactagaaaaatcctttcataatgaaacaattgcaccataaattgtataaatataaaattaattctaattgttttttttattgcagtttcttgaaatcaatcatgtcttctccaaaagaaatcgctattggcattgacctgggcaccacctactcctgtgtgggggtgtttcagcatggaaaagtggagatcatcgccaacgaccagggcaacagaacaacacccagctatgttgccttcacagacacagagaggctgattggagacgcagctaaaaaccaggtggccatgaatcccaacaacacggtgtttgatgccaagaggctgatcggcaggaggtttgatgaccctgtagtgcagtctgacatgaagcactggtccttcaaagtcgtcagtgatggaggaaagccaaaagttgcagtggaacacaaaggagaaaacaagacctttaatcctgaagagatttcctccatggtcctggtgaagatgaaggagattgcagaagcttatctggggcagaaggtgaccaacgcagttatcacagttccagcctatttcaacgactcccagagacaggccactaaagatgctggagtcatcgctggactgaatgtgctccgcatcatcaacgagcccacggctgcagccatcgcctacggcctggacaaaggcaaatcctcagagcgcaacgtgctgatctttgacctgggcggaggcaccttcgacgtgtccatcctgaccattgaagacggcatctttgaggtgaaggccaccgctggagacactcatctgggcggtgaggactttgacaaccgcatggtgaaccactttgtggaagagttcaagaggaagcacaagaaggacatcagtcagaacaagagggccctaaggaggctgcgaaccgcatgtgaacgagccaagaggacgctctcgtccagctctcaggccagcattgagatcgactctctgtacgagggcatcgacttctacacgtccatcaccagagctcgcttcgaggagctctgctccgacctcttcaggggaacactagatcctgtggagaaagcactgagggacgccaagatggacaaggctcagatccacgacatcgtgctggttgggggatcaacaagaatcccaaagatccagaagcttctgcaggatttcttcaacggcagagaactgaacaagagcattaaccctgatgaagcggtggcttacggtgccgcggtgcaggccgctatcctcatgggcgacacctctggaaacgtgcaggacctgctgctgctggacgtggccccgctgtctctgggtattgagaccgcaggtggagtcatgacggccctcatcaagcgcaacacaaccatccccaccaaacagacccagaccttcaccacctactccgacaaccagcccggcgtcctgatccaggtgttcgagggagaaagagccatgaccaaagacaacaacctgctgggcaaatttgagctgacgggaattccacctgcgccacgtggcgtcccgcagatcgaagtgaccttcgacatcgacgccaacgggatcctaaatgtatcggcggcggacaaaagcaccggaaaacagaacaagatcaccatcaccaacgacaagggcaggctgagcaaagaggagatcgagagaatggtgcaggaggctgacaagtacaaagctgaagacgatctgcagagagagaagatttctgccaaaaactccctggagtcttacgccttcaacatgaagaacagtgtggaagacgacaacctgaaaggcaagatcagcgaggaggacaagaagagggttgttgagaagtgcaatgaagccgtgagctggctggagaacaaccagctggcggataaagaggagtacgaacatcagctgaaggagctggagaaagtctgcaatccagtcatctccaaactctaccagggagggatgccagctggaggatgtggagctcaggcacgtgccgcatcaggggccagcgctcaggggcccaccattgaagaggtggactaaagttatgaactcgcatgaacgtcgtctgtcagtcagaatccacgtaatgttttgaccaaacatatttttatacaacactgctgctcttatatttctgttgctatttagcaaccattcaccactgtaatttatttaaagtgtacattgtgatgtaaataaaagtgtaaatattttcatgaattcaaaaaacttttatgtaaaaaaaatatatatatgaaaatgattaataaagtgtttgtacatgagaac

SEQ ID NO: 40 - DNA, 2017 bp, Mus musculus
gagcagctacacgaagcctgtgtttctatatcgtgttgttctctttccctcccccctcttccgagtgagaaaaaaaaggggggcctaaattcccaccaacataaactaatgacatacaatgatgaaattctgttttcacctctgcctgtgacagggaatgcaaaaatagcaagtggcctatttccacgaatccccgcctccctgccctccccgctcccgctgggtttggatctctaagaatggaggctaacgcagaggcaggagccagccgggatcagccccccgaacccgagggtgatgtcagtcggcgggagcggacgcagcctggccagggaggcagaggttcctagggagcagggagggcgggtggcctggatgggcggaccgtgcgcccggccgctccgtcgcctggagacggccctggcggcgctgtgttgctgtaaacagccgcttcgctgttactatctatagaaagatcgcctagctcccgggtgcggcggcccgaggcaggtttgtggttctgcaccccttgcgcccctaaagatccatccgctgtcctaatcctctctagtccgtcttggaatgcccaccgtttggcttgacccttgggaccctcagaatggagacgagggggacacatgagttctgcgaggatctgcggtttcctatcccagaggtgaccggcccagtcagtctaacccggcttgtcctctgcggaaggacaggaggccgagggcaagtaggggtgtgtttgttctacactgaagcacctgacctcttcaaagttccatcttccaagactcaaagctgttctcaggtcccagacgccaaaatctcggcacagctgggaacctttcttcccgtcccctctgcgcccccaccccccttcccaagtccgatctggaaaatcacccgctgcaggcgggttccttgtaagcgcagtttccaggctgcacgtattcagacccccatctccccagcaccgacttgctttctcctccccccccccccccgagctcacctcactttgtaattctgagctccccccctgccccgactcgccctctggtctcagctcaaaactaaacatacgaccccttcaggcatacttgtagggtggttttgcacaatgtttatccgtcagtgtcaacggggactgtcgccttgatagctctaagtggctaagggtcggggagtaggtgctgccgtcctttaaaacacgaatttatgaatgaacccagtactgtagttaaatcaggttattgtacacttatttacaatccttcacttgctgcttccaacctcagtcctaaagtttctccaggcaaggagctggagagaggggctgagaagctgaccccccctttttcttctctgcactgatttgggatggggggctgatgtgggcaagctttcctttaggaacagaggcttcgagcctttaaggctgcgtacttgcttctcctaataccagagactcaaaaaaaaaaaaaaagttccagattgctggacaatgacccgggtctcatcccttgaccctgggaaccgggtccacattgaatcaggtgcgaatgttcgctcgccttctctgcctttcccgcctcccctcccccggccgcggccccggttccccccctgcgctgcaccctcagagttggctgcagccggcgagctgttcccgtcaatccctccctcctttacacaggatgtccatattaggacatctgcgtcagcaggtttccacggccggtccctgttgttctgggggggggaccatctccgaaatcctacacgcggaaggtctaggagaccccctaagatcccaaatgtgaacactcataggtgaaagatgtatgccaagacgggggttgaaagcctggggcgtagagttgacgacagagcgcccgcagagggccttggggcgcgcttccccccccttccagttccgcccagtgacgtaggaagtccatccattcacagcgcttctataaaggcgccagctgaggcgcctactact

SEQ ID NO: 41 - DNA, 6640 bp, adeno-associated virus 2
ctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgctagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgttaacgctagcatggatgttttcccagtcacgacgttgtaaaacgacggccagtcttaagctcgggccccaaataatgattttattttgactgatagtgacctgttcgttgcaacaaattgatgagcaatgcttttttataatgccaactttgtacaaaaaagcaggctccgataccgctcgccgcagccgaacgaccgagcgcagcgagtcagtgagcgaggaagcggaagagcgcctgatgcggtattttctccttacgcatctgtgcggtatttcacaccgcatagttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttatcgatatgaagctacagtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgagaagaccttgacatgattttgaaaatggattctttacaggatataaaagcattgttaacaggattatttgtacaagataatgtgaataaagatgccgtcacagatagattggcttcagtggagactgatatgcctctaacattgagacagcatagaataagtgcgacatcatcatcggaagagagtagtaacaaaggtcaaagacagttgactgtagagttcctggatatggccgacttcgagtttgagcagatgtttaccgatgcccttggaattgacgagtacggtgggctgcagatgaggcctgaatgtgtcatacaggagcccagtaaaaataaagacaggcaaagacaaaagaaagacaaaggaatattattacctgttagtacgaccacagtcgaagaccacatgcccccgatcatgcaatgtgatccacctccgcccgaggccgccaggattcacgaagtcgtcccgaggtatctttcggagaagctgatggagcagaacaggcagaagaacataccaccattgtcggcgaatcagaagtctctgatcgcgaggctcgtgtggtaccaggagggatatgagcagccctccgacgaggatctcaaaagagtaacgcagacttggcagtcggatgaagaggacgaggaatccgatctacccttccgccagatcacggagatgacgatcttaacggtccagttgatcgtcgagttcgccaagggtctaccgggcttttcgaagatatcacagtctgatcaaatcaccttattaaaagcctcgtccagcgaggtgatgatgctgcgggtggcgaggcgatacgacgccgcgtccgacagcattctgttcgccaacaacaaggcgtacacgcgcgacaactaccgcaaggcgggcatggccgaggtcatcgaagacctcctacacttctgccggtgcatgttcgcgatgggcatggacaatgtgcactttgcactgctcacggccatcgttatattctcagatcggcccgggctcgagcagccgtcgctggtagaagagatccagagatactacctgaacacgttgcgaatttacatcatcaaccagaacagcgcgtcgtcgcgctgcgccgtgatctacggcaggatcctgagcgtgctgaccgagctacgcacgctcggcacgcaaaactccaacatgtgcatctcgctgaagctgaagaacaggaagctgccgccgttcctcgaggagatctgggacgtggcggaggtggccacgacgcatcccacggtgctgccgcccaccaacccggtggtgctatagtctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgccggggatgcggtgggctctatggcttctgaggcggaaagttgagaggacattccaatcataggctgcccatccaccctctgtgtcctcctgttaattaggtcacttaacaaaaaggaaattgggtaggggtttttcacagaccgctttctaagggtaattttaaaatatctgggaagtcccttccactgctgtgttccagaagtgttggtaaacagcccacaaatgtcaacagcagaaacatacaagctgtcagctttgcacaagggcccaacaccctgctcatcaagaagcactgtggttgctgtgttagtaatgtgcaaaacaggaggcacattttccccacctgtgtaggttccaaaatatctagtgttttcatttttacttggatcaggaacccagcactccactggataagcattatccttatccaaaacagccttgtggtcagtgttcatctgctgactgtcaactgtagcattttttggggttacagtttgagcaggatatttggtcctgtagtttgctaacacaccctgcagctccaaaggttccccaccaacagcaaaaaaatgaaaatttgacccttgaatgggttttccagcaccattttcatgagttttttgtgtccctgaatgcaagtttaacatagcagttaccccaataacctcagttttaacagtaacagcttcccacatcaaaatatttccacaggttaagtcctcatttaaattaggcaaaggaattgctcaactttgtatagaaaagttggtaatacgactcactatagggcgaattgggtaccgggccccccctcgaggtcgacggtatcgataagcttaggcctccaaggcggagtactgtcctccgggctggcggagtactgtcctccggcaaggtcggagtactgtcctccgacactagaggtcggagtactgtcctccgacgcaaggcggagtactgtcctccgggctgcggagtactgtcctccggcaaggtcggagtactgtcctccgacactagaggtcggagtactgtcctccgacgcaaggcggagtactgtcctccgggctggcggagtactgtcctccggcaagggtcgactctagagggtatataatggatcccatcgcgtctcagcctcactttgagctcctccacacggagcttgatttaggtgacactatagaatacaagctacttgttctttttgcaggatcccatcgattcgaattcacgtgctagcgcttaagaaattcaaggcctctcgagcctctagaactatagtgagtcgtattacgtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcagggccgacccagctttcttgtacaaagttggcattataagaaagcattgcttatcaatttgttgcaacgaacaggtcactatcagtcaaaataaaatcattatttgccatccagctgatatcccctatagtgagtcgtattacatggtcatagctgtttcctggcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtt

SEQ ID NO: 42 - DNA, 11686 bp, Artificial Sequence
  Synthetic
actggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggagtacttcaagaactgctgacatcgagcttgctacaagggactttccgctggggactttccagggaggcgtggcctgggcgggactggggagtggcgagccctcagatgctgcatataagcagctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatctagaattaattccgtgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacacaagacaggcttgcgagatatgtttgagaataccactttatcccgcgtcagggagaggcagtgcgtaaaaagacgcggactcatgtgaaatactggtttttagtgcgccagatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctgtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctagtggtttcatttactctaaacctgtgattcctctgaattattttcattttaaagaaattgtatttgttaaatatgtactacaaacttagtagttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgataagcttgggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgactctagctagaggatccaggcctaagcttacgcgtgttaacagatccatgctagatcccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttaccatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataataataaccgggcaggggggatctgcatggatctctcgagggaattcgagctcggtacctttaagaccaatgacttacaaggcagctgtagatcttagccactttttaaaagaaaagggggg

SEQ ID NO: 43 - DNA, 38031 bp, Mus musculus
aggcaagcaccttactgactaagccatccctccagccccctcatccgcatgcaaatagcttgataccaggtaaactgtcccttcaagtttagccaaatcctagagctggaaacagctctctactgcaagcaattcagggcctacctcctttgtggggttaccacatcctgggtaacaattgccagacataaccaccacaggacccattatcagtcaactaggaacagcgctacaccccagagcagaggccgccgtggttcttcatatcagccaaaccaaaggctgctgatttagcctcattcgtcctacttcagaaacagtgaggctccagcccacaatcttccctcatcagccagacctaattctttccagtgtccccgcccctggcccccccagtgtgatgtgcaggactctacctagagatgaagcagtctgacaaacttccaaaggtggctagttaaggaaacttggcttgctgtacctaaagaactgtactttttacattgatttattcatgcattcgttcattatatatctgtgtgtgtgtgagtgcgcgcgcacacgcgcacgcacgcgcgcgtgagcgctcacatatatatctgcttgtacatgtggaggtcagaggacaacttatagaagttcgttctctcctatcacgtgggtcctgaggatcgaacttgggtcatcaggtttaacagcaagcacctctacccactgagccatctcaccagcctaatactaacacatttattaaaggcgcaataatttgtcacctattacagtgctgtggcataatatttatactagtttatactaaataataaacaacatgttgactatagtaaattgttaccgttttgaagcaacagcttttccactctaccctcttctggaatttccctcaacctattttccccaacctctttcttgccaaatccttaccacataccacccgtcatttcctcactggaacatattatttttgcaggtcactctgtctgggttcagtaccactggctctcgattcttctgctgttgctgcccccataagtagggtccagagctctgtacatttccagaactaactctggtgccaattgccacctatttgcaatttccgctagacaccttgctgggactctaacctgctccttggtccatgcacatgtctccaggcacctccagctccaccatgaacacatagcctttgccccagcactgtcttctccctcttatttcttcagagaagctctctagaaacctgggagccccttagccctcaccaaaaccctctcaggagtgcctgggcagtgtatatcctctggtcagcttctctgagcctgcagtcattacccacccccatgacagccccaccccctcagctggatttcagcctcttgtggacccctttgtcactcctgtgcaccccccccatgtacccctttcccacttatccatccctgacgcttcatcagacctctccccagatttaggaccttccatagttccctaccactcacaggaaagtctaaaacctctgtctttttggaatggcttcactttcctcctcccaccccatgcccttccccccccccccactcccccactccaccccgggcagcactcactccagaatgcagtgcagcgttcagacacacttctgttacttcactccttctcttgcttccgatgctcccttgagaatgcctacccataagtcactgctctctcccacaccttctccaaatgctctccttgtggcctcgtgtgatccttgtagtattagattgctcttgtttctacccgtgtttattcaaaggtctgtcttcccaggcatggtggtatacacctgcgatcccaacactcaggaatgccaggggaaggggaggaataggaggaagggatgacaaggctagccccatctacatagtaaattccaagccaggctagacctgaagacagctgaaaaccaggacctggtgagataattcagtggataaaatacaagcccggtgatctgagttcaaatccctgcaacctatgctggaatgagagccaactcctaagagttagtgcatgcttatcatgcacaccacacaataataataatacgttttaaatgtcctttcagcaatcagaaacattgagaaccagtgtcttagatacttttctgtttgctctgataaaacaccatgaccaaggcaacttatagaagaatttattggcacttacagtctcagagggtgagggcatgaccgtcatggcagggaacacggccgcagacaggcaggcaggcaggcaggcgtggtgctagagcagtacctgagagctcacatcttgatccatacgcaaaaaaaacagggcatagtggcacttgactttagtctcagcacttgtgtggcagagacaggtggatctctgtgagttcaaggccagcctgctctacatatggagttacagggcagcaagggctgcataaagagaccctgtctcaacaaacaaacaaagcagaagacagaggggaagggagaaggagggtgcactgggaatggtgtaggctttttaaacctcaaagcccacccccagtaacgcccctcccccaacaacattacctcctcattcttcccaaatagttcctccagccaagcattcaaaaatacatgagcttataggagccataggtatcattcaaaccctcacgtaggcatcatagcgcatgcctagcatgtcggcagtgaaaaggctgaagcagaaagatttctatgcgtttgtggccagcctgagctacgaagtcagtagtcagttccaatccacactgtgtgtgttaagaatctactttgtgccgggacctagagccctaaaaacatagcagtaaagaagacaaacacggactgtaatggacctcaaaggcaccgtttttcacgtattcctcctcctcagaaaccacttcctgcctcctagctcagtgctgacagagaacgagcataaagaaccaccagggcaggacaggatctctgtgggcattcagccagccccagccccagcccccatgcctccccaggggactcttactaaagaatacacctacacccccagcccaaccaaggtggaagcaagagaaggagttcaaggctagcctgtgctacagagtgatgccctgtctcaaaaaacgaacaacaaaacatctcccccactgtgtcccagcctctccctaaaccttccgcagctcccagctgccaagtccagacaggaccccatttctccagtctccattacgcatttgctgcccttccacttcgctctgctttgatcctaggcctttgctcatgctgccacctcggtcttaaccgcctcttctgcctcctgcttatttattgcagagtaagaactgcttccgctagcaaccaaaggcaggagtcctatgggtgtaactggatggtgttgtgtcaacttgacacaagctggagttatcacagagaaaggagcctcccttgaggaaatgcctccatgagatccagctgtgaggcattttctcaattagtgatcaagggtgggagggtccatagtgggtggtgccacccctggtagtattgggctctataagaaagcaagctgagcaagcctggggaagcaagccagtaagtaacatccctccatgacttctgcatcagctcctgctttctgacctgcttgagttcccgtcctaacttcctttggtgatgaacagcaatgtggaaagtgtaagctgaataaaccctttcctccccaacttgcttcttggtcatgatatttatgcagaaatagaaaccctgactaagacagtgggtcagcagagtagccccggagcagctgcaaaactcatctctgccctcgtccagttggctgaacgcatcaccaacggatatgtgcaccgaccggttgattgtggactggttcacttcacgtctggccattatcaggcaggtgtgtcagagcacagcatggcaggtcttcctcccatggcagccaggtgagacttgcacactgtgtttaggggaaaccatcagccaaagcatccatggtcaaacccatagccaatgtgaagggtgcacggatctggagcatcatccacatagggatcagctgtactctgcccctgctaatgtgtcccaagccttcacagtagacctgtccttcacgaaccacccataatactgtcagtccaacaagtccatagcataacctctaatatgcatctaacctccaggagggtggggttcctccaggacactccttggagggggatcctcttgattcagaggtccatgaactaaaaccgtaagtaatctacccccccatatgaagcaaggatgggatctgctcgatagagggggagaaataagcacagagaattgcccaaggcagttctgaacttgagccaggaacatggagtcagaaccatgctccagaggcggtattgcagctaaagagatccccgggtttcttagctcttggccacaccctctggctcttggtcctacctcctaagcatcacttcctcctattccttttcttcccctcttcttgtgtggtatatgtgagttatgtacagatacactcccctgggcactcacacacgtagaggccaaaggctgggtccagatgtcttcctcaattaccctcaactttattgttctcaaaaacttgtatgcatgtgtgtgcgtgtgtgggcatcagttcttctggagcagggattatgggtaattgtaagcagcctgacatgggtgctggggactaactctaatcttttttaagaatagccagcagctcttaactgataagccacctccaccttaggttttgagacagagtctctcactaaactttgtgctcatcattcaggctagactgagctcccaagatctacctctttctgtctccaccaccaccaccacccaccccttcagtgtcggagttctaggcatatgctgccacacccaactttttaagtgggtgctgtcaatcaaactcgggccctcccttttctcccactttcatctcccagacccttctcttcttttccctgagaaaaagctcatgtgtgcttagacctgcttcctgtccaacgaaagctgggttccactttcaccctgaacgtttcccagtcctttggcatccagctttctgcagctttgtctaatataatattcttttaaaaccttatgggctttctgtgactgcatcttgaacccatgtgtgcactaaatgccacacccatgattaatttcagaaagattctacgtaggctgctgaatctttaatattctcggaagcccctctgttgagctcaaagagtctgcaaaacaccatgaaatacttcctggggtcttcccaaaggccctgcagctacgctcttggtttaatctttcctttaggccattttcttactttgagaatccagtccctcacaggctgagcatgagggacagttttattttccaaagcacaaactgggcaggacctacttagctagtcccaccccaccccccaccccacactgccacaccctttcaatagtcacacagctagaagcatccaaatgtcactttcagcactctttctagattcctttagaactcacaacttcattaaagcactgcctgaccacgtggtggtaatacacacctttaagcactcgggatcccctgcagaggcaaatggatctctgagttggagaccagcctggtctatagagtaagacaggacagccaggaccatacagagagaccctgtctaaaaaaaagcggggaggggggagaaggaagaggagggagcaaaggaggaagaggaagaagaggaggaggggtatagtaatttgaataaaaatggcctccacaggcccatagggagtggcactattatgaggtgccctgttagaggaagtgtgtctctggggatgggcttttggggtttcagaagctcaagtcaggcctggtggctctctcttcctgctgcctgtagatccagatgcagaactctcagctccttctccagcaccacggctgcttcctgctgccatgctcccgccacgatgataatggaccaaaccttaaaacctcaagccagtcccaattaaatgttttcctttataagagttgccgtggtcatggtgtctcttcacagcaatattgttgcggtcatgaaagaccttaaccaaaagcaatctggggaggaaagggtgtggtgtgctttgttttctcatagttccatgtatctgttcatcttcaaaagcagtgagggcaggaactcaagcagagtaggaacctgcaggcaggagctgatgcagaagccatggaggggtgctgcttaaaggcttgcgccccatggcttgcccagtgtgctttcttatggaacctgggaccaccagcttaggggtgacaccacccataatggactgggcccttccacatcaatcactaattaagaaaatgtccttcaggcctaactacagcctgcttttatagaagcatttcctcaattgagtttccgtcttctctgatgactctagcttgtggcaaactgacttaaaactacccagggcagccagcctgagctgcacagttcagaaaaaagaaaagagggttcttgaggtgggtctttggctcaaggtgcttgccacaaaacttgatgacccgagtttaacccctgggtgccacatggtaggaaagaaccaacttctgcaaattgtcctctgatcaccacacatgggccatggcatgcaaaaggacatacacgaacatacacaataaagtgtaaagaaaaaaattatcttagcctgtgttctattgctgtgaagagacatcatgaccaaatcaactcttacaacagaaaacatttcgttggggctggtttgccatttcagaggtttagtccattatcatcatgacagggagcaaggcagcacactgccagatatgatgctggagaaggagctgagagttctgcatctggatctacaggcagcaggaagagagagacatcaggcctgacttgggcttttgaaacctcaaagcccacccccagtgacacactccttccaacaaggccacacctcctagtccttctcaagtagtgccactccaatgaccaagcattcaaatatatgagtcgatgggaccattcctatccacaccaccatatacttaaattcaagggggtttttggttggttggttggttagttttggtttagcaaaaaagaaaaactagaagaaaagagatgtgtttatctcactgatttggaatatgaagcccaaggttgtagacccatctgtcagtatctggtaggacccttccttccagctgcttctggggagtttgtagaagactcacaaggaagaaagatcacacggagtggaagcaaaaatgagatgagactaatccagtccttcattaacccactcatgaagatgcaacccttgtggccaaattaccttccaccttttaagagtaccaccatattcatacaggtgtactggggaccgagcttctagagtggatgtctgggagagtcacacacatctaaaccacagcaggtctcctgtcccctaggtcaccacacccaatgtttttccaactgtgtaaccactacatagcacaagcactcttgttccagacaccctgtaatttccttagtaagaaagcttgggaccaatcaggtagctacagaacaggcaggctttttccttatgggccccaggagagaatgttccagcaaatcagagacaaaatgcatggctctctaatagtcatatgcaggaatctcttggcatcacctctgcatgacctgttgactgaagtaatcagaagcccctagatagcccagaagggaacacacagggctgtggtggcacacactgtatgctcagcacttgggaggtacaaacaggacagtcagaattcaaagtcatcctcagctctatagcaagttagagacagagagattctctctctctctctctctctctctctctctctctctctcactcacacacacacacacacacacacacacacacacacacacacacacacacaccagggtctccctatgtctagtcttcacttctttttggttggttggttggctgggtttttttttttattttatttatttatttatttatttatttatttatttatttatttatttatttatttaatgtattatgagtgttctatctgcatgtacacctgcataccagaagaggcatcaggtcccattacagatggttgtgagccaccatgtggttgctgggacttgaactcaggacctctggtagagcaagcagtcaatgctctaaaccactgaaccttcatccctccagcaccccagacttcatttcttagtgaagaaatatacaataatttgcatctatgttttagaatcaacatgggctgacctggggtataccgtttgttgtttgttttgagaaattcgttcattcattcattcattcattcattgttggttttgggggataaggtctcatatatgccaggctggtctcaagctctctatgtagccaaacattgccttgaactcctgaccctcctgcctctaattcctgggttctgggatttcagacatgtagcaccatacctggtttagcccctaggattcatgcatactgggtaaacattctacccactgggctacatccccagcctcccttctattgtacattgaatcacaattacacatacttttatggtcatataatacccaaaatgtcacctgggagctgtctgagctaggcaatcccaaatggtgactcacagtcagttctgagctaaacaatcccaaatggtgactcacagtcagttctccacatatgtagctcagacttcttcacactgacactaatttccaagaaagcaactgccaacacaaaaacagtgcttaggcatgtgtttgcatcacaccaagtttgtcacctggccacacctagaactggtatagatgctgaaggcttaattctttgggggctacaaatgtggcaacctaccacccatgagcaactctggttcatctttaaagacacagtacacacctgctgcgaccacttcaggaagaagaaaaggaatggatgattctgccaacactctaatagcatttatgaaatgtggtggtttgaatgaaaatggccccccgacagacgcattaggaattacattattggaggcatggccttgttggaggacgttggaggtgtgatctttgaggtttcagaagctcaagccaggcccagtagcttgctgcctgtagatccagatgtagactgtcagctaactctccagcaccatgtctgtgtgccaccatgcttcctgccatgataaaggcctagacctctgaactatatgccaggtgccaggcccaattaaatgttttcctttctaagagttgttgtggtcatggtgtctcagaacaacaataaaaccctaactaagacaaaaatcctacatggttatatttatatgactatacagggtgactgccctctgggttgttctaaagaaaagtatttttgctgttgttttaatcttgagttttcctacccatgtgtcccaagctagagctactggagacaaaggagagggacccacctgggaccaacaagaggccatcttaccttgattcaattctttccagctttcagcttccgacttgtccattcggaaatgggagacttttagtctcctctgtctccatccatcatccctttccgaatccttggttgtcttctttgagcctttgattctaaccgactggagaggttgctcaagcagatacaaagttgcaagctctcaacaggccaggctcccctccccttcctgaaggcagaatgcctggggacccagaaagtaggccagggcctggaaaccttggcaaaatagcacagctaggcctggtatgggcatgcagagcagcatccagggcctcaggctgcctgagccctgacatatctaacagccctctgagtagtccgggcctgccccaaaataaaagaaccaaccagctcccaggctgtgggaggagaaagtgtcaaactaaagcaatagtaaagatccctgaagctgattcaaaagccactaagcaggaaggaacacacagcagttcggggacagaagactacagcatacatggagtgagaaagacaagaggctgagtgaatgagaaagaatggggttctgggtgagcagctgaggccagggcacctggggactttagaaattcccctatgcatttctaagaacccgcccagtaaagctgtaaaggggattcatctgtgaagggggtggtctttagggcccctgttccttagaacccaagccccggggaactttgcataaagtggcttaccagaaagatagcccccactcctgttagaatgtcccattgtctccagcactggggaagcacatagctctccaagggaggggactgaaattgcatctgcaggcccagggatggcttccaacagtgccatttactgtattgaaagcttcagtgaccgtgccagggccaacttctatgaagaaacaggagactgggcaggcagatggctgagaggcagagccaggccacaagactctccacccagaattcagagaccgggaaccaaactagcctttaaaaaacataagtacaggagccagcaagatggctcagtgggtaaaggtgcctaccagcaagcctgacagcctgagttcagtccccacgaactacgtggtaggagaggaccaaccaactctggaaatctgttctgcaaacacatgctcacacacacacacacaaatagtataaacaattttaaatttcatttaaaaataatttgtaaacaaaatcattagcacaggttttagaaagagcctcttggtgacatcaagttgatgctgtagatggggtatcattcctgaggacccaaaaccgggtctcagcctttccccattctgagagttctctcttttctcagccactagctgaagagtagagtggctcagcactgggctcttgagttcccaagtcctacaactggtcagcctgactactaaccagccatgaagaaacaaggagtggatgggctgagtctgctgggatgggagtggagttagtaagtggccatggatgtaatgaccccagcaatgctggctagaaggcatgcctcctttccttgtctggagacggaacgggatcatcttgtactcacagaagggagaacattctagctggttgggccaaaatgtgcaagttcacctggaggtggtggtgcatgcttttaactccagtactcaggaggcagggccaggtggatctctgtgagttcaagaccagcctgcactatggagagagttttgggacagccagagttacacagaaaaatcctggtggaaaatctgaaagaaagagagaaagaaagaaagaaagaaagaaagaaagaaagaaagaaagaaagaaagaaagaaagaaagaaaggaagaaagaaagaaagagtggcaggcaggcaggcaggaggaaggaaggaaggaaggaaggaaggaaggaaggaaggaaggaaggaaggaaggaaggaaggaaaataggtgcgacttcaagatccggagttacaagcagaatgcactgtttccctaacagggccaagtgttttgagtaactgaaggtgggcatgatgcctgggaagcagaaacaagccaggcagatgcaccccttgccttgcttccgaagggctgcagtagcatggaaaacatggaaaacaaccaatccattccctttgctgatataacaggctccaaagccaaaacctgtcactggaggctcaagagcagatctccagccaagaggcaaaggaatgggggaagctggagggcctccctctggttatccaggcttctgaaggttcaagcaaagaaagggttacaaccttaaaaggagagcgtcccggggtatgggtagaagactgctccaccccgacccccagggtccctaaccgtcttttccctgggcgagtcagcccaatcacaggactgagagtgcctctttagtagcagcaagccacttcggacacccaaatggaacacctccagtcagccctcgccgaccaccccaccccctccatccttttccctcagcctccgattggctgaatctagagtccctccctgctcccccctctctccccacccctggtgaaaactgcgggcttcagcgctgggtgcagcaactggaggcgttggcgcaccaggaggaggctgcagctaggggagtccaggtgagagcaggccgacgggagggacccgcacatgcaaggaccgccgcagggcgaggatgcaagccttccccagctacagttttgggaaaggataccagggcgctcctatatgggggcgcgggaactggggaaagaaggtgctcccaggtcgaggtgggagaggaaggcagtgcggggtcacgggctttctccctgctaacggacgctttcgaagagtgggtgccggaggagaaccatgaggaaggacatcaaggacagcctttggtccccaagctcaaatcgctttagtggtgcgaatagagggaggaggtgggtggcaaactggagggagtccccagcgggtgacctcgtggctggctgggtgcggggcaccgcaggtaagaaaaccgcaatgttgcgggaggggactgggtggcaggcgcgggggaggggaaagctagaaaggatgcgagggagcggaggggggagggagcgggagaatctcaactggtagaggaagattaaaatgaggaaatagcatcagggtggggttagccaagccgggcctcagggaaagggcgcaaagtttgtctgggtgtgggcttaggtgggctgggtatgagattcggggcgccgaaaacactgctgcgcctctgccaaatcacgctacccctgtatctagttctgccaggcttctccagccccagccccaattcttttctctagtgttcccccttccctcccctgaatctcaagcccacactccctcctccataacccactgttatcaaatccaagtcatttgccacccaacaaccatcaggaggcggaagcagacgggaggagtttgagatcaacttgggctacatcacgagttccaggctcaccaaggcttcttaaggagaccttgtctctaaaattaattaattaattaattaatagtcccctttctctgccacagaaccttgggatctggctcctggtcgcagctccccccaccccaggctgacattcactgccatagcccatccggaaatcctagtctatttccccatggatcttgaactgcagagagaatggcagagtggcccgccctgtgcaaaggatgttcctagcctaggtggagctcgcgaactcgcagactgtgcctctcttgggcaaggacaggctagacagcctgccggtgtgttgagctagggcactgtggggaaggcagagaacctgtgcagggcagcaatgaacacaggaccagaaaactgcagccctaggaacactcaagagctggccatttgcaagcatctctggcctccgtgcttctcactcatgtcccatgtcttatacaggcctctgtggcacctcgcttgcctgatctcatccctagccgttaagctttctgcatgacttatcacttggggcataatgctggatacctaccattttcttagaccccatcaaaatcctatttgagtgtacggttcggagaacctcatttatccggtaaatgtcttttactctgctctcagggagctgaggcaggacatcctgagatacattgggagaggagatacagtttcaataaaataataggttgggtggaggtacatgcctataatgccaccactcaggaaatggtggcagcttcgtgagtttgaggccaacccaagaaacatagtgaaaccctgtcagtaaataagtaagcaagtatttgagtatctactatatgctagggctgacctggacattaggggtcatcttctgaacaaactagtgcttgagggaggtatttggggtttttgtttgtttaatggatctgaatgagttccagagactggctacacagcgatatgactgagcttaacacccctaaagcatacagtcagaccaattagacaataaaaggtatgtatagcttaccaaataaaaaaattgtattttcaagagagtgtctgtctgtgtagccctggctgttcttgaactcactctgtagaccaggctggcctggaaatccatctgcctgcctctgcctctctgcctctctgcctctctgcctctctctctgcctctctctgcctctctctgcccctctctgcccctctctgcccctctctgcccctctctgccgccctctgccttctgccctctgccctctggcctctggcctctgccctctgccctctggcctctggcctctgcctctgcctcttgagtgctggaatcaaaggtgtgagctctgtaggtcttaagttccagaagaaagtaatgaagtcacccagcagggaggtgctcagggacagcacagacacacacccaggacataggctcccacttccttggctttctctgagtggcaaaggaccttaggcagtgtcactccctaagagaaggggataaagagaggggctgaggtattcatcatgtgctccgtggatctcaagccctcaaggtaaatggggacccacctgtcctaccagctggctgacctgtagctttccccaccacagaatccaagtcggaactcttggcaccatgaacccagccatcagcgtcgctctcctgctctcaggtactgggcaagggtcagggctggcattctaaggaatctggcttcctcccatcccgggaagtagcctctttgccatagtctcaggggcacaggtggttgggaggtgcgggggtggggagtggggaggagcctcaacctcaccagtggtggtctttgacatattagaaactccataatggatctaggaactcctcttgctgaaggcgcataacgataccacgatatcaacaagtttgtacaaaaaagcaggctggcgccggaaccgaagttcctattccgaagttcctattctctagaaagtataggaacttcgaaccctttcccacaccaccctccacacttgccccaaacactgccaactatgtaggaggaaggggttgggactaacagaagaacccgttgtggggaagctgttgggagggtcactttatgttcttgcccaaggtcagttgggtggcctgcttctgatgaggtggtcccaaggtctggggtagaaggtgagagggacaggccaccaaggtcagccccccccccctatcccataggagccaggtccctctcctggacaggaagactgaaggggagatgccagagactcagtgaagcctggggtaccctattggagtccttcaaggaaacaaacttggcctcaccaggcctcagccttggctcctcctgggaactctactgcccttgggatccccttgtagttgtgggttacataggaagggggacgggattccccttgactggctagcctactcttttcttcagtcttctccatctcctctcacctgtctctcgaccctttccctaggatagacttggaaaaagataaggggagaaaacaaatgcaaacgaggccagaaagattttggctgggcattccttccgctagcttttattgggatcccctagtttgtgataggccttttagctacatctgccaatccatctcattttcacacacacacacaccactttccttctggtcagtgggcacatgtccagcctcaagtttatatcaccacccccaatgcccaacacttgtatggccttgggcgggtcatccccccccccacccccagtatctgcaacctcaagctagcttgggtgcgttggttgtggataagtagctagactccagcaaccagtaacctctgccctttctcctccatgacaaccaggtcccaggtcccgaaaaccaaagaagaagaaccctaacaaagaggacaagcggcctcgcacagccttcactgctgagcagctccagaggctcaaggctgagtttcagaccaacaggtacctgacagagcagcggcgccagagtctggcacaggagctcggtacccggaagatctggactctagagaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataagcttgccacaaccatggaagatcccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgctggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataataataaccgggcaggggggatctaagctctagataagtaatgatcataatcagccatatcacatctgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatccggaataacttcgtatagcatacattatacgaagttatgtttaaacggcgcgccccggaattcgccttctgcaggagcgtacagaacccagggccctggcacccgtgcagaccctggcccaccccacctgggcgctcagtgcccaagagatgtccacacctaggatgtcccgcggtgggtggggggcccgagagacgggcaggccgggggcaggcctggccatgcggggccgaaccgggcactgcccagcgtgggcgcgggggccacggcgcgcgcccccagcccccgggcccagcaccccaaggcggccaacgccaaaactctccctcctcctcttcctcaatctcgctctcgctctttttttttttcgcaaaaggaggggagagggggtaaaaaaatgctgcactgtgcggcgaagccggtgagtgagcggcgcggggccaatcagcgtgcgccgttccgaaagttgccttttatggctcgagcggccgcggcggcgccctataaaacccagcggcgcgacgcgccaccaccgccgagaccgcgtccgccccgcgagcacagagcctcgcctttgccgatcctctagagtcgagatccgccgccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccccggatctaagctctagataagtaatgatcataatcagccatatcacatctgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatccgggggtaccgcgtcgagaagttcctattccgaagttcctattctctagaaagtataggaacttcgtcgagataacttcgtatagcatacattatacgaagttatgtcgagatatctagacccagctttcttgtacaaagtggttgatatctctatagtcgcagtaggcggcaaattccaggacagccagagctattctcaagatagagaatccctttcttgaaaaaaccatttaaaaacaaaaacaaaagcaacacactcctcttgatctcctgttcttgaaacacattgttgggacccagaacttcagtagattgatggaagttggagtctgcaagtggtggaacatcccaccaatacctcaagggcgagtgcaaaccccacatccccccagctcaagtctacttttcctgcaggtgggaggccccgggtctgtgtctccccaaattcagagaaggcactgctgtgccagtcttgcaggtgtcccgagggcagaaggtgaccagcctgacagcctgcctggtgaaccaaaaccttcgcctggactgccgccatgagaataacaccaaggataactccatccagcatgagttcagcctgacccgagagaagaggaagcacgtgctctcaggcacccttgggatacccgagcacacgtaccgctcccgcgtcaccctctccaaccagccctatatcaaggtccttaccctagccaacttcaccaccaaggatgagggcgactacttttgtgagcttcaagtctcgggcgcgaatcccatgagctccaataaaagtatcagtgtgtatagaggtgagactggttcccagaaagataaaatgtccaggttagccaggctggggtagccaataaaaaaaaaaaaaaaaaaaaaaaaaaaaacaggcacctccattacccttcccctaactgctggtctcctgggaaactgctgctgtctatgtgagtggggcaagattaggggccagaaagggggagcttgtagtaaaagcacagttgaggaaactaaatgggaaaggcagtacagtggtgattcttgtggtgtggaggttctgttacagcatccggtggagccgctaagatgagaaagcgccagctagctgccttgaacagctgacacctgtctttgcccgcctgagtcctgatctcccctcctcccggcaccccttctctatccacagacaagctggtcaagtgtggcggcataagcctgctggttcagaacacatcctggatgctgctgctgctgctttccctctccctcctccaagccctggacttcatttctctgtgactggttgggcccaaggagaaacaggagccctcgaggtccttcctctgcagaggtcttgcttctcccggtcagctgactccctccccaagtccttcaaatatctcagaacatggggagaaacggggaccttgtccctcctaaggaaccccagtgctgcatgccatcatcccccccaccctcgcccccacccccgccacttctccctccatgcataccactagctgtcattttgtactctgtatttattccagggctgcttctgattatttagtttgttctttccctggagacctgttagaacataagggcgtatggtgggtaggggaggcaggatatcagtccctggggcgagttcctccctgccaaccaagccagatgcctgaaagagatatggatgagggaagttggactgtgcctgtacctggtacagtcatactctgttgaaagaatcatcggggaggggggggggggctcaagatgggagagctctgctgagcctttgtggaccatccaatgaggatgagggcttagattctaccaggtcattctcagccaccacacacaagcgctctgccatcactgaagaagccccctagggctcttgggccagggcacactcagtaaagatgcaggttcagtcagggaatgatggggaaaggggtaggaggtgggggagggatcaccccctcctctaaaacacgagcctgctgtctccaaaggcctctgcctgtagtgagggtggcagaagaagacaaggagccagaactctgactccaggatctaagtccgtgcaggaaggggatcctagaaccatccggttggacccagcttaccaagggagagcctttattcttctttcccttgcccctctgtgccagcccctcttgctgtccctgatcccccagacagcgagagtcttgcaacctgcctcttccaagacctcctaatctcaggggcaggcggtggagtgagatccggcgtgcacactttttggaagatagctttcccaaggatcctctcccccactggcagctctgcctgtcccatcaccatgtataataccaccactgctacagcatctcaccgaggaaagaaaactgcacaataaaaccaagcctctggagtgtgtcctggtgtctgtctcttctgtgtcctggcgtctgtctcttctgtgttcttccaaggtcagaaacaaaaaccacacacttcaacctggatggctcggctgagcacttctgtgtgcagaaggtccaaccagactctggggtaccccggccctccctattcccttgcctcctgtctcccgctttttatagctccctatgctgggcttctctggagagtgaaatctttgcccaaatcaatgcgcattctctctgctgagtcatctggcgacagcagttgagttcacccgccaacacatgggcccagctatgtagccgaaccctggctctggaagtgccagggactttgtgcataagtatgtaccatgcccttttttcacagtcctagctctgcagaagtgcagcctgaaggcctgtctgctgagaggacatgccctggagccctgaaacaggcacagtgggaggaggaacggaggatgacaggcatcaggccctcagtccaaaagcaaccacttgagaatgggctggagtacgaaacatggggtcccgtccctggatccctcctcaaagagtaataagtaaaatataaacaggtaccccaggccgttctgggtttgggttgtaatgggatccatttgcagagaactattgagacagcccagccgtactgtgacaggcaatgtgggggaggaggttgaatcacttggtatttagcatgaatagaataattccctgaacatttttcttaaacatccatatctaaattaccaccactcgctcccagtcttcctgcctttgcgccagcctcctgtctggccatgcctgaagaaggctggagaagccacccacctcaggccatgacactgccagccacttggcaggtgcagccaaacctgagctgtcccagaaagggacattctcaagacccaggcaccctgatcagcactgacttggagctacaagtgtcatgccagaaaagtctctaagaaaaccttttcagggaaaagggggtgactcaacaccgggcaagtttgggaagccccacccttcgagtgatggaagagcagataggaagcctcagaagagagacaccggcacccaggtaacgttcctcatgtggtctctgtcacactaggtgctcttccctggacatctccgtgaccacactctcagttcttagggagatgcgggtgctctctgaggctatctcagagttgcagattctgaggcctagagtgactacagtcagcctaggaagccacagaggactgtggaccaggagggcagaagaggagaagggaagaaaaaccatcagataggacttgcaatgaaactaacccaagacaatcataatgcagacaggaatgttaaaggcgttcagcagctggccatgacacccatctgtccctctggccaagtcagcaagcctggaagacctgggactcctgcccatatgtcctaagctccccacccacccactcgttcactgtccttattctctctctaccttcagccacttagtttcctaccttaagtcctagaattcttgctcaagagcatcaaatgcccctgccttggagctggtgggatggctcagtgggtaaatcacttgccaccaagtctgatggcctgagttcaatccctgacatgcacattggtagaaagagagaaccaaacactaaaagctatcctgagctcttcacttggaccatggcatacttgtatgcatgcctgtgaaggcacgcacacacgcacgtgcacacacgcacactcacacacaactgttcttcagaaagtttatttaaaatgtactttactacccctctgtgtactctgcatccaaatggtgttacccactgagacagagtacacaggccatcactgtagctgtctccctgacatccggagctctgttgggaaggccaagaggatttccatgaaatgggggaagaggagggtggatcactggccaacagggaatacacatcacagatgtgacatgcatcttcctgccttcttggcacaaaatacaactcctgcctgagcatatagcttttgtgttgtggagcatgtacatcttcccataaacactcctgatagaacacgaggctgggttccttctgagtactagcatggacatctctggccctctcctgctcagtacacaaggtactcagggtaacaatggtacagctgtcctcaccaaggcccactgttcttggtctccagagactatgtagccccaaatctttcctcattcttggacttaaagtacctcttgcctggcagatggaagatccagggccaatgggaaaccaaggggtagaaacttacccctgcaagcccagtaactggcagctaaagcaaaagaagtgccatggtttgggggccagaatgggcgatatagtgaggctagcctggactgaagagtgggctgctgtcttggggagaggggagttagatattgcagtagtccaaagtgaggatggtgcctcggacagaagctgcagaggagaaaataaagggaagtggggacttcttggaaagctgcttagagttataacagagggctggtgaaaccagaacaaaacctgtgaactttggcagaatctgtccccactttgctactggactggttatacaagctgctaccactgggggaaaccaagtcacaggtatataagacctctaacctcccatctcaagagtacaacggccaccggatcctctggacctgaagttacagatgtttgtgagctgccatctggtgccaggaattgaacccatgtcctcgggaagaacagcaggtactcttaagccctgagccatctctccagcctcccttgtactatctttatcatttgaccctctcagggtcggagtaatcttttcttttccattatattcatcctttaaattgctacaaagatttaatgaaaagacacatgcgggctggtgagatggctcagtgggtaagagcacccgactgctcttccaaaggtccggagttcaaatcccagcaaccacatggtggctcacaaccatccataatgagatctgacgccctcttctggtgtgtctgaagacagctacagtgtacttacatataataaataaataaattttaaaaaaaaagaaaagacacatgcacacacccgcacgcacatacccacaggcacatacacacacgcacacgcacacacacatgcacacaccctagcatagtggctggagtatgtgagaagtgaatttctgtcacttgtttgtaagccatcctgtctatggtatttgttacagcagatagaatggactaagataacattaaatactgtcttaaccaaaagtcacagcaaagctgtatggggaagaggggaatgcgaattagcagcaggtacaaggggcacagctttaaccccagcactcaggagacagaggcaagcagatctctcagagtttgaggccaccttagtctacatagtgagttctagggcagcaaggactatagagtaagaccctgtctcaagaaaaataataaggaataagagataggatgaaggagaaaggggaggaagaggagaaggaggaggaaaaggaggaagaggaagaggaggagaggtatctcagtgagttagagtgcttgctgtgaaggtcttgagttcaaattctcagcactcatgtgaaggccaagtgtagctcccagcattggggagcagagacaggcaaattccagtacctgtatctagccagcctagctgaaatggctagaaccaagtttgattaaaaacaaacaaacaaaacacacacacacacacacacacacacacacaaacttgtctaaaaaaaaaagtggagaggaatagagaaagacactgaacatcctcctctggcttccgtgtgtgtgtgtgtgtgtgtgtgtgtgtgtgtgtgtgtgtgtgtagatagatagatagatagatagatagatagatagataataaatatttttaggaatattttagggaataaataaatgagttaaatgtaatcttagtgatgaaatcagaagttcaatgtcatccttagctatgtagtgagttcaaggcctgactgggcctgtgagactctgaatcaaataaataaataaataataaataaataaataaataaatagggaactggatttggctaggcaggaattgagcctacaaacgagcttaaagctgagaccaaaagataactcagtaggaaaaggtgcctgtcacactgcatgccaacctgagtcccatctccagaggcacactcacacacatataacaaacacgtaaacaagcttaaaagcaatagggaacacaacatcaatttttaaagtgtttgtacatactagaaaattttaaatatacaagcatcctggttggttttcatgtcagtttgccacaaattagtcaccttagtggggagactcacctgaaggactgtcctgatccccttaattgatatggggagagtcactgggattgtgggcgacaccttctggtagcagcagagataaaagggagattgcagaaggaagcctccctcctgctgctgagttgactagtcctgaagctgtttcgatgatgatgatgatggtggtggtggtggtggtggtgatgatggtggtggtggtgatggtggtggtggtggtggtaaggaggaggaaaaggaagatgaagaagaaggaggaagaggaggaggaggtggaggaggtgatgtccagcactccccaacttttattgagcaatagctgtttccaggaaacttccatgtctgatatcaaacccaaaccaatgaggcaccctgccttgcagactgagtaactacctattgccactctctggcagggagaaacagttattctagcactactctggctatcaaggctccagccttaaggatcaagtaactaccaggtactcagctctcaggtgtgcccccactgttatccaaaagctgacttactaaattctgatataaagattacacataaaacaagcaaatagaagagctgagtaattaagatggctacttaggggaataagaccctaggatggagaaatggactgctttaaacctaataataacttttgtctctataagaaataatatgcttaaagaattctattacaggctggtaggatggctcagtgggtaagagcacccgactgctcttccaaaggtccagagttcaaatcccagcaaccacatggtggctcacaaccatccgtaacaagatctggtgccctcttctggagtgtctgaagacagctacagtgtacttacatataataaataaataaataaataaataaataaatcttaaaaaaaaaaaaaaagaattctattactgccctgccagttctatttgaggatttgcctactaatggccatttttatcttagggaggggttgagaggaagtgagaaggaatctcaatcttcaacaataacatcatatttgttaagatagaaaaagccaaggaagtgttgaggttgagagccagatatgagctgtttgttacgtcattggacagtcatgtgcaagtatggagaggagagctggatgtgggtgtggttctgcagacaagagtgcttgcctagcatgcataaagccctgggtttcatccccagcattatatacattgatcaggcatggtgacacatgcctataatcccaacactagggagatggaggcagatgaatcagtttaaagtcatcaactacacagcaagtaggaggcctatttgggatacatgaaaccctgccttttaaaaaaggatcaactaggttaactgctaataagattaaaataagaatcaagagtgttggctcgtgcctgtaacccaacacttcaggctaggacaggaagactgctgtgagatcaaggtcagcctgagctgagtgaatcggagagaagaaaagagagaggacaaagacaaacacagagtctataaatatgacttgtagctgggcttggtggcacttttaagagaccaaagcaggattccaagttcaaagaatgcctgggctacatagtaagatcttttcatggaaggaaggaaggaaggacaaaaggggaaggacaaaaaaggaaggaagaaggaaagacaaagaacggaaggaagaaaggacataaaaaggaaggagggaggaagggaggaaggagggagggcgggaggaaggacaaagaaaggaaggaaggaaggaaggaaggaaagacaaagaggaaggaaagaaggacaaagagaggaaggaaggaaggaaggaaggaaggaaggaaaaaaggagaaggaagaactaaggaaggaaggaagaactaaggaaggaaagaagaactaaggaagaaaggaaggaaggatggacaaaggaaggaaggaaggaggggcaagcatactggcgtgcacccctttactttaatcctaaggaagcagaggcaggtggatctctgcttccagccagcctgggctacatagtgagacattgactcaaaacaacactaataacaataatgaagcaatattatggctgatgaccaggctgtttgtggctggagtagcaattctccaaagacagctgctaaatcacctcggcctgtgtgtaagcttcagttgcacatagcatggcctggtgtctcacctgcagtctctaaggtcatgcgctgtgccttccgaagttggatcatgagatacctgcgactggttctcctggaatgctgactccagggacactccctcttggaacccagcttccatacggagaaacccagtcatgcagatgtgtaggcatccattcatcaggaccagctaacttaagctccagccggcagccaccattacctgccagccattccatgagccacgagatgcccaagggcccagcttccaacaaccttatgatttggcaagagccaccttgggagatgtagtagcagcagccaggctgaagtgggtgagcaaaaatgaatgggagattgttcacttgtttttcagggagtgttgctgtgactggagtcagctggagtctggaggtttggtgtgttttaaagacaggcaatcatggggctggagagacggctaaatgattaggaacacttgttgctattgcagaggaccccacatggtagctcatcatctgttccagctccagggttctgcaaccaagtacacacattgctacacatgcaaacctttaaaaaaataatcagtactgccaagtggtggaggcacatgcctttagtgccagcacttgggaggcagaggcagatggatctctgagtttgaggccagcctggtctacagagtgagttccaggacaaccagggctacacagaaaaactctatctcaaaaaaaaaagtaagaataaaaaataattacaaataagggtaataaatactacatcatggttgtctgttgatgttttggttctgaagatcctaaagacagagaagactaaagagaagaaagtgaagaaaatagagtctttggaaaagcaacaggggagagacctagagctacaaataaagacaaggactaggtgtgatctacatacaatcccaacacttggtatgcgtagtcaggaagaccaagagttcaagaatctcatgtagacatagctagcttagtgacagcctgggctacatgagacccagccacacccacaaaaaagatggtgcataagcataggtttgtgaatttgaaggttcaaggcaccggccataagataatgatttgaatgtgtcctctaaaagttcacatatcagaaatgtaatctccaaattcataaaataatggtatttggaggtcaggctggtgataattagttttaattatcagcttgatataatctagaatcacctagaaaggaagtctcagtgaatgactgtctaaactaggttggcctgtaagcatgttcatgcaggattatcttgattacattaattgagatggaaagactgcccactatgggcaacaccattccctaggcttagggttctggataaaaaacagaaaggcacctgaccactaagcatgcatacattcactcttcctttgctgatgactgtggatgtgaccagcttcttcaagtttttgctgccttgacttccccgagtgatgggctataatctggcattgtgggctcagagaaagcttttcttcccctcagctgcctttctcaaggtattttgttgctaaaactataaactaataagacaggacctctgtgagttaattaagaaggctgtgaggatggggttttctcatgatggtactaatgcctttatataagaagcctgggtgtggatcagtggtacagcaacagccctggattccatccctaatatcataaatatatatatatcagaggaaagagacttaggcagcacatttgctcagtccttccattagaacctcctgtcccattatggtacaacaagaaagtcctagataagcatggtggctcatgcttttaatcccagcagtcagaaggcagagtcaggcagatttctatgaatttgaagccagcctggtctacatagagagttccaggacagcctgagctacaatgagaacttgtctttaaattatatatatatatatatatatatatattcttatagcaatagtcttggaccactcaggtcttagaaccttgagctaagtaaattttccatataaattacccagtctctgagattcagtcatagcaacagtaaagggactacaacagggctttccatgcaagagtctctgcttctcagacacaaggtatggccaggctgaggagtgggagctgtgagaggtggaagggaaatggagaagtttggaaaaggaaaaaaaaaatcagcagtgcgacagagagcttacgtgatgggctggggacactgcttaattggtaacatgctcacttgacaagcacaaagcctgtgtccagtcctggaacacatgaactgggattgtggtgtgcacctataatccaagtagctgtgatgtggagacaagaggatcagaagttcaaggtggggctggagagatggctcagcagttaagagactacatttccagaggtcctgagttcaattcccagcaaccacatggtggttcacgaccatctgtaataggatccgatgctctcttctgatgtgtctgaagacagctacagtgtactcatatacataaaataaataaatgattctttaaaaaaaaaaaactcaaggtggttctcggctacatagtgagttcaaggtcaacctgggatacatgaatacttgcctcaaaaagggaggaaagaaaagcttaaatgctgtcctccccagtagttagagagacctagctttaacttttttttttaccatgttaccttaacctcttaaagctcaatgctgccacaactgtgatgggaagaaacaagagaaatgataaaatcacattcatcagaaagtgggagcaggagcaaaattcttcctgaagtcacacacaccctcaatcatgaactcttgaacgagtttcagtcaccttgtgcctgggctgaaaggacagagaaatgtctctggccagtggtctacagggaaaacacaaatggaaggaccaaagggttgccatggctacatctggtcccagcaagaggaagatgttcctctaggaacagagggaagactcagaaaggaactaaggacctcacaccagcatctaaggagctaaattctgccagccacctggccaaacatgggttctgtccctgaggatcctggcagaagccccaacacagtgctattcttgatttggtccctatgagaccctggccttgtaaacacccagcctatccatcaggacttctgaccttaagccagtgacaaagaatggtggtgttttatgtgtgggtgtgtgttatgcagcaatcaaaaattaattcagtagaaaaggcatcaaagcccaggaacatggtggaaatgctgtacagcacagagaagtgtggtcctcaacctggagtggccctaatcagttcatcttcttctatctctttattcttctggaaaagtattgagggggtgggcaagactgctcaatgagggccaggtgcctccagacatgagctgcagaggagagggcagaagaatcaaggaggttggcaagaaatgtatatagcaagacgtcagagactaaactgggcacggatatgaagaagtctgtgggtgggcagatgacgcacagtgagacagtgggggtcaaagaggcccacaggctgacagggagactaatatcagaaagaatgttggagcagagtgtagtgggagacagtggccaggagctgtggatacgacacccagtgttctggaggcaacagcaggaagcagagcatcagcagaggccagcggttcgaagcctgtctggagtgggtaaaattgtgtcccctgaaaagatgcactcaagcccttgtcctcactccctctcaaagtgaccatctctggatatatgctctaggcagctgcaaccaagtcaagacaaggtcatttagattaggatggccccctgctagcctcataaaaccagaggaaccaggacaaagagaaatgggaaaaccaaccacaggaaaatgggcagagagactgaagagtcatgtcaccttgggccaccggaagctgggaagggtaaggaaggcacctcctctcaagctttggggacagcacactgacaagcgcacactttgaataggacttttttttttttttggggggggggggtttgagacagggtttctctgtgtagccctggctgtcctggaactcactctgtagaccaggctggcctcaaactcagaaatctgcctgcctctgcctcctgagtgctgggattaaaggcgtgcaccaccacgccctggctgattaggacttttgtactccaaagaatacacatcttgacagatcttaagcccctcccctcagccatggctgcttttttttaaggtggccgtaagaaactaacacccacccctctatcaaggatgcatgcacagtgagggtaagagggagggcagtggctacaaacacttataccaccgtgagatctcaattaggatctcagcacccataggacaacccaggcatggtgttgagaaagcctgtaacctcagcactgtgggagcagggacagggagatagcttggggtttgatggctaccagtctagcagcaaagttgagtttagggttcagggagtgaccctgtttcaaaggaataaagcagggggtgagagagaaggagaaagacatttgatgcccacttctggtttccacacacgtgtatctgtacacatatgcacctgccctcctcctctctatacacatgcacacacacacacacacacacacacacacacacacacaccacacacctggtagaagaagagaacaggctctaggaagctgagcactgacttctaaatgcatgctgtgctaatgcttcccatacacatacatagtactcacacacaagaaaaagtgtttgcaaagctataggactccaaatctacagaacaaggaaggacatgcaacagggacacattggagtcatcaccttgatactgtctgccattcacaaggtcttttcatagcctgctccttgtcccctggcagcccaaattgtctcagacagttgcccccagaccagcaggtgctacagtgggtacaaaatcatagaaaatgcaaatgtccaaaactatcttaaacctcctgagtcagagaaagggttcttctatcagcctttctagagggttctgattcaagttgaaaaaccactgatgtaagtaactacgtttttccttttttctttttttaaattgggatttgaacccagggcctcaagcaagcgctctacccctgagctgtgtcgctagcattcatgacaggtacttcatattcttaaggcaatatctctctaggttacccagtctgattcttgacctcaccctgtatcctaggaaagtcttgaacttgcaagactttagcctcagctctgattagctgaaactacagatctgcaccacacacttctgacttattaactatttttttctaactgccctttgctcagcaaatcctgaacctttcctcagccagccttactagctggcctcgtttcagagtcactaaaaaagaggccatcctggatctccacctccatccacctcctgtgcctggtccaggttctatgcctctctccccttactttaggtgagccattcactcctaatcaaggccagcccctctgctatcaactcaacaccatccctttatctaatccagatactgctctggacattctatgtagtgagactttgtctcaaatttaattaattaattaattgtggcacaagcctgaaaacctgagttcccatctctagaaccaagtaaaaagtaaaggctgcagaactctcttgttatcccactgtttcaacagcaagatgggagacaaagagtgggccagttagcctggagtatacaaaaacagagattctgcctcaaaacaaggtggaaaggaagaattaattctcaaaagttgtcctctcatctctatatatgcaccatggcatggtaggccctcattcttgttcacctaacacacacacatacacacacacaccttgaaagggagggagtatctctcttcccattctttcttgaactcatacaaagtcagattttcacctgatctctaccaaactccatcaaatatattcctggaaagaaggaggggggggagaaggaggaggaggaagaagggagggagggaggaagggagggagggaaggagaagggaaggagaagggaaggagaagggaaggagaagggaaggagaagggaaggagaagggaaggagaagggaaggagaagggaagggaagggaaggggaggggaggggaagggagggaaggggaggggaggggagggaaggggggaaggaaggaggagaaggaagaagaggaaggagaagaaggaagaggaggaggatgaagaggaaggggaggaggaagaggaggaagaggaagaggaaggggaggaggaagaggaggaaggggaacaggaaggaggaagaggaggaagaagaggaagaggaagaggaagaagaagaagaagaagaagaagaagaagaagaagaagaagaagaagaagaagaagaagaagaagaagaagaagaagaagaagaaacaactaggtacctagatcaatgcttctccaacttgaaccagaacttcttcaaccttctaaatcaatctgaaactcattcacttaaccactgacatttgacccattgctctctccctcctcctccatgagcttcctgagcatccaacatgctccctgctgtctctcaggacactctttcaggccatgggccattcattcctcagtctctcagatggctccaaccatctttgaagctacttaatgtaagagaatcccagaatccagacctcacacccttctctgtctacatttgccaatctcttttcatatcatggttttaaataacctgagggctctcaaatttataacccccagcccagatcacatcagatttcaacatctg

SEQ ID NO: 44 - protein, 162 aa, Mus musculus
MNPAISVALLLSVLQVSRGQKVTSLTACLVNQNLRLDCRHENNTKDNSIQHEFSLTREKRKHVLSGTLGIPEHTYRSRVTLSNQPYIKVLTLANFTTKDEGDYFCELQVSGANPMSSNKSISVYRDKLVKCGGISLLVQNTSWMLLLLLSLSLLQALDFISL

SEQ ID NO: 45 - DNA, 2353 bp, Mus musculus
catgtcgctggtatggagtataggctgttgctatgacaggaactcaggggtcttaactggcttgagcgctgggagggggcaagcagccaggccttgtctgtaagctgaagacctggcagtgctgagctggtcaccccccaggacctccttttgtgcccaacgagtgactcaccttggcatagacataatggtcaggggtgggcacgcagcctgcttcccgctgtgctccaggcctccttcgatgctttccgagaagtctattgagctgggagcttgtactgcacccggggctgacatcctggcatcctgggataaaagcagcccacggggctgcccttgccatatgcctcactggcggcagaggacaaggctctattcagcaagtgccctggagtagacaccagaagcccaagcatgggcagaggaaggcaggggttggggggagcagagctgtctgtgttccagaagcccaaggacacagatggctaaggcgcctgggagggacctgagtggaagagatagatgggcctgaagtctcaagcagcaacagcctcctccccgccattggtgagggtggggtttggtttcccggacctacatatccctcagaggcctggtgtgtaggaatttaaaggaggtaaatctcctgagagaatgaggggtacccaggaagacggggtgttacagaaagactccagcatgcacagccaactcactcaaaactactctgtcaggggctgccgggggccaggctcggggtggggggtgggggggcaaagagaagctggaccagggagaaatggcccactaggctggatatgaggccacagaggggctcaggaatgaagcctgctgtcttaccctattaggatctgcgtgcataccttctgctgtgcactctaaacacacagccagaggctcaagttgaccctggagtcacagagagggctccaaccttagccctccactcctgaactccaggaatgagaagatagagttggagcgattcaggggagaggactctgttgagaatgggggccacaggaaactgtaatataggttgatcccggaggaagggaataggttcttcaagttcctagcatctcacaggcccccagagaaggacagagttggggtggtcctggcttacaggctctaagaactggaagctgattaccccaccaagctgtgcactctctgtctctgtctctgtctctgtgtgtgcgcgctcgtgcacacttatcacacaaatgttcatgtgtgtgcacatagatgagttgagaccagaggtcaacctcaggcactgttgccttggttttctgagagagcatttctctctggacctggaactcgccaattagtgagagccaggaagtctgctgattttcactgcccagcactggagtttacaagtatgcactgtcaacccaggccttttgtattcattctgcagctagaacttgggtgggtcttcatgcttgacaggcaagcaatttatggactaagctgtttcctcggccctctcttgacccatttaccagaaagggggttccttgatcaatggcgaagccaggctggtgttcccaagaaagccttgactctgggtacagtgacctcagtggggtgagaggagttctccccttagctgggctggggcccagcttcaccccctcaggctattcagtgggggtgcttccaggaagtcaggggcagatttagtccaacccgttcctccataaaggccctgacatcccaggagccagcagaggcagggcaggatggagcggagacgcatcacctctgcgcgccgctcctatgcctccgagacggtggtcaggggcctcggtcctagtcgacaattgggtaccatgccacgcttctccctgtctcgaatgactcctccactccctgccagggtggacttctccctggccggggcgctcaatgctggcttcaaggagacacgggcgagcgagcgtgcagagatgatggagctcaatgaccgctttgctagctacatcgagaaggtccgcttcctggaacagcaaaacaaggcgctggcagctgaactgaaccagcttcgagccaaggagcccaccaaactggctgatgtctaccaggcggagcttcgggagctgcggctgcggctggaccagcttacggccaacagtgcccggctggaggtggagagggacaaccttgcacaggacctcggcaccctgaggcagaagtgagaagggggataggggaaatggctagtgagcagagagactgaagtgcaagggctgcctgcctggaaaaaagggcactgcctcccccaggggagtctctcagctcctgcatttccccatgt

SEQ ID NO: 46 - DNA, 16922 bp, Homo sapiens
gttatgaaatggaagctctaatttgtcaagcacctgctgtgttcagctttcactgattttattcttacaactacctggggagcaggcatggttgaatccccattttacagacgtggaaatgaagactcaggcaaggttcttaaagtcatcaaagactgcacagctactgagtggccaaggctttgctccctctccttgaggacagtgctatagcagtcagagtcaagtctagagaacaaagcgggtgatgcccctgggtgaaacaaatttgggttttagaaaactccgcctacagctttctccctgcctctaaactgcggtggctgtggcggctcacacctgaccagctgagcaccaccatctagtggccaccaggctcagttcccttcctcgggacaggccatggatgaacagacttgctggtcaccaaggctcagccagagagactgtgttctcttcacccagcttgctgaccaccacgctgtgccaggcactgggccagggccaagggcccaagcagaaccagcgggcagcagtcatgtggcaaaactgccgtttgaattagaggctggcctatttggcaaacaccaggcaccctgtgtgtgggttgatgccacatgtggggctggtctgtcccaggaggcatctgggctttaccctgtaagcagaaggtcatctagcagattctgatgtgagggaggggctcaggaaggtcatcttgtcctcacctcagcttgaagcaggcacagcttggctgtaggctccttgaaagggcatccagacttctctctccatggctggtattatcctgctgtgcagtctagaaggcagtctagggtttggccagaggctttagaagttggggccttccatttggggaagcaggagaggaggcaccccagagaatggggcctgagccaggctaggaggatggggggtttgggtggaggctggacgggctgagggcctacccttctctgtttgctgtgtctgcctgcagcagatgcaaatcagagtgtggtttagaacagagagaggacagtcacagcggctcacatctgtaatcctagcactttgggaggctgaggccagaggatcacttgaggccaggagttcaagaccagcctgggcaacataacaagacttggtctctgaaaaaaaaaattatctgggcacactgtcatgtgcctgtagtcccagctccttggcaggctgtggcaggaggattgcttgagctcagaagtctgaggctgcagtgggctatgatggtgccactgcactctggccctgggtggcaaagtgagactttgtctctaaaaaaaaaaaaagagagagagagaacatactgtcatactgtccaccactagggcttggtacatgaatgcatatagtggaaaatcatgcagctgttaaatgatgatgcagacaaatatttattgatgtggaaagatgttcacaacatattgttgagtgagaaaagcaaattacaaaataatttgtacggcatgagcccattttgaaaaaaatgattatatatgtgtggaaaaaatctggtagtgtttatctccaggtggcaaaatgatggctaattacatttttttgtattgcttatctatagtttctgacctttctacaatgaatatttaattaaaaagataatgaaagtttggaacacaaccacgaccaaatctgtctgtatctttggttctctgtgcagacacctttagatagttaagggttggccaggcgtgatctatatgtaccggttgtataggtcatgcctgtaatcccagcactttgggaggaagaggtgggagggtcacttgagcccaacagttcgagaccagcctgggcaacatggcgaaaccttgtctctagtaaaatacaaaaatgagctgggcatggtggtgtatctctgtggtcccagctactagagaggctgaggtgggtggatcgcttgagccagggagattgaggctgtagtgagctgtgatagcaccacttcactctagcctgggcaacagagcaagaccctgtctcaaaaaacaaaaacaagatagttaagggttgagtgtgaggaaaggtctgagggttgagaaggggtggaggatgcacctgggcctatgacaggggtccacggaggtggctgatggcaaaagctgggggactccaactgctgatgctgaaacaagcttgtgtctcacatacacagggacagttcactgagcttcaatgacaggcacctcctgctcatcacatcttttctctctaggacagctttgcccttattttaactagacttcccttgaaccaaaagggaaggctacatgctgtgacttgctgggcagcctggaaaggcgggccactcctagccacagagatgagacagagttcagacaagagcttatccccagtcttccttttctattttgtttattttattttatttttttatttattgagacagagtctctgtcacccaggctggggtgcagtgatgcgacattggcttactgcagtctccacctcctgggctcaggtgatcctcccacctcagcctcccgaatagctgggatcacagtagtgcaccaccatacctggctaatttttttgtattttttgtacagacaaaatttcaccacattgcccaggctggtctcgaactcctggactcaagcgatccgcccacctcagcctcccaaagtgctcggattacaggcatgagccactatgcccagccttgctcttcctttaaagcctcctgtccttccccaggtccccagttcatagcaggatcaaaggtcactgggcgctcaccccgtcttcaagatgctctttcctatgtcactgcttacgcccaggtcagatgtgactagagcctaaggagctcccacctccctctctgtgctgggactcacagagggagacctcaggaggcagtctgtccatcacatgtccaaatgcagagcataccctgggctgggcgcagtggcgcacaactgtaattccagcactttgggaggctgatgtggaaggatcacttgagcccagaagttctagaccagcctgggcaacatggcaagaccctatctctacaaaaaaagttaaaaaatcagccacgtgtggtgacacacacctgtagtcccagctattcaggaggctgaggtgaggggatcacttaaggctgggaggttgaggctgcagtgagtcgtggttgcgccactgcactccagcctgggcaacagtgagaccctgtctcaaaagacaaaaaaaaaaaaaaaaaaaaaaagaacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggactgggcagggttctgaccctgtgggaccagagtggagggcgtagatggacctgaagtctccagggacaacagggcccaggtctcaggctcctagttgggcccagtggctccagcgtttccaaacccatccatccccagaggttcttcccatctctccaggctgatgtgtgggaactcgaggaaataaatctccagtgggagacggaggggtggccagggaaacggggcgctgcaggaataaagacgagccagcacagccagctcatgtgtaacggctttgtggagctgtcaaggcctggtctctgggagagaggcacagggaggccagacaaggaaggggtgacctggagggacagatccaggggctaaagtcctgataaggcaagagagtgccggccccctcttgccctatcaggacctccactgccacatagaggccatgattgacccttagacaaagggctggtgtccaatcccagcccccagccccagaactccagggaatgaatgggcagagagcaggaatgtgggacatctgtgttcaagggaaggactccaggagtctgctgggaatgaggcctagtaggaaatgaggtggcccttgagggtacagaacaggttcattcttcgccaaattcccagcaccttgcaggcacttacagctgagtgagataatgcctgggttatgaaatcaaaaagttggaaagcaggtcagaggtcatctggtacagcccttccttcccttttttttttttttttttgtgagacaaggtctctctctgttgcccaggctggagtggcgcaaacacagctcactgcagcctcaacctactgggctcaagcaatcctccagcctcagcctcccaaagtgctgggattacaagcatgagccaccccactcagccctttccttcctttttaattgatgcataataattgtaagtattcatcatggtccaaccaaccctttcttgacccaccttcctagagagagggtcctcttgcttcagcggtcagggccccagacccatggtctggctccaggtaccacctgcctcatgcaggagttggcgtgcccaggaagctctgcctctgggcacagtgacctcagtggggtgaggggagctctccccatagctgggctgcggcccaaccccaccccctcaggctatgccagggggtgttgccaggggcacccgggcatcgccagtctagcccactccttcataaagccctcgcatcccaggagcgagcagagccagagcaggatggagaggagacgcatcacctccgctgctcgccgctcctacgtctcctcaggggagatgatggtggggggcctggctcctggccgccgtctgggtcctggcacccgcctctccctggctcgaatgccccctccactcccgacccgggtggatttctccctggctggggcactcaatgctggcttcaaggagacccgggccagtgagcgggcagagatgatggagctcaatgaccgctttgccagctacatcgagaaggttcgcttcctggaacagcaaaacaaggcgctggctgctgagctgaaccagctgcgggccaaggagcccaccaagctggcagacgtctaccaggctgagctgcgagagctgcggctgcggctcgatcaactcaccgccaacagcgcccggctggaggttgagagggacaatctggcacaggacctggccactgtgaggcagaagtgaggaggggatggggaaggggggccttgtgagcagaaggggctgaatccccaagaaggagtgcccgagaagtctcagggaggggccgaacctccctgctccctgggcctccctacctcttgatggggcactatccttgccccccaacatgatgggagggaccagaaacaggcccagggccccggggatctgatgcccgcatgccttctgccaggagtccagggtcccctcagcacctccctactggggaaagcagtgcaggagcagcggggcccctgtgtttcattcatggctgggctttgtgactgtgggcagcgagctcacctattctgagcctgtgtccatataaaggaggagttggaagcggagaaggttgatgtccatgagggagattggattctggggtgaagaaagtgagggaaagagcaggcaggtctgggcgcaaagcacaggtgactgcctgccaccagcttgtgacccccatcaagttactttgacttgcacagctgtgaagcggtggtcataataaaattcatttcaaaaggtggttacctgggatcagaggaatccccaggggcatggcgcttcactgagctgacaggacatgcatgtgtgccttcaagtgcaggaggacatgtgcgtgtgtgtgtgtgtgtgtgcaacagtgagtgtatgcttgtggatgcgcctgtgtgagcagaagcaggtgcaccaaccctgataaggcaccttagtaatgagttaaggcaaaagcccacatctgctcatcctccagacaagtcctctgtctaaggccccccaacccttaatcctcctgctgctctactggtcctgggtgggggtggtctctgtgacagctgcctcaagggagactgaggcaggtattcaagtgtcctcagaagagcctggacccaggaatgtgtccccccactccaggctccaggatgaaaccaacctgaggctggaagccgagaacaacctggctgcctatagacaggtcagggaggtggaggggaggggcaggggagggaggaaaggattgatggccacccacccaaacacagcccccaagctcagcccgccagagcaatgtgctgctccccactgccccagaaggaggggtaaggcaaggctgaggaatggggagaaggagcctggggccccagtggccctctactcctcacccctcctgccactccaggaagcagatgaagccaccctggcccgtctggatctggagaggaagattgagtcgctggaggaggagatccggttcttgaggaagatccacgaggaggtgaggccagggcagagggaggaaagctcaacacagagattgcaactgagagagggagagacagagacagagaagctgagactgagagagacactcagagagaggagagaaacagagattggcaggtagagacaggaaatgaaagacaaacagaaagacactaagggagacggaaagagacaagagagcattcgaactcctggccgaggggcaggcagaagaggacacaatggcgtggggaaccccagctgtggcctgtaccctgagccctggtaccgcttctctcaccctggtcaggaggttcgggaactccaggagcagctggcccgacagcaggtccatgtggagcttgacgtggccaagccagacctcaccgcagccctgaaagagatccgcacgcagtatgaggcaatggcgtccagcaacatgcatgaagccgaagagtggtaccgctccaaggtagccctgcctgtgggccagcctgcctcctccaggcagcccttcttgctctgtacaagagaatgggggaaggagggtggaggaagctgggagaatatcctctccaggtgactgactttcaccagagaagtgagatggcttgcccagggtcacacaggcactattgctttcaaaagtacccattattattactgctactattgctattattagtagtagctgatactgttctgttctaagccctgtgcttttcatggattaccttactatgaagtaggtgctgttctttgacagatgagagagccgaagtccagagaggttaactaatttgctcaaagtcacacagctcctaagtggtagagttggatttccaactctagcacatctcacttgggagcctgcattccatgcctctgccagtctgttttgggttactgagcctctaccatatgccaggcaccccccaggacacagagaggtgtctgacctggaaggtgtgatattgacgtgcacccaaacatagtgcccgtagagaacaccacagctcagagcttatatgtagtgtgggagccaggaaagacactgtagagggggcatttgggctaggcaggacaggcacgctgtggacaggaagagaagaagggagggccattcctagtggaggacagagcctggaggcagaaaagcacagagaggcagggccccaagatgctagcctagccagggcacctagctgaggagtgtgagtggcagaagccactggtgtctgagcagggtgagggcccgatggggctgggaggacctccctgcaggcaggggagaaagggctattagaacccagggtgaggccaggcgaggtggctcacacctgtaatcccagcactttgggaggccgaggcatgcggatcacctgaggtcagcagttcaagactagcctggtcaacatggtgaaaccccgtctctactaaaaatacaaaatcagccgggtgtagtggcgcatgcctgtaatcccagctactcaggtggctggggcaggagaattgcttgaacctgggaggcagaggttgcagtgacccgggattgtgccactgacttccaacctgggcgacaagagcaagactctgtctcaaaataaaacaaaaaaaacaaaaaaacaaaaacacacaaaacacccaggcctccgctcgcctaagacgacagcgttcagtcccgcagatgcagctgtcagtgtggaaggttctggattctgtggcactgttcattcattcgcttgctgaccaggtgttgtgctaggtgctgaggtctggcagcccgctaccctgcccgcatccatgggtcacagttgttgggaaaagtcccagccagggccaacgtcctggggttaactggggtgctgctcacctccctgatttcccagagcccgtgcctgcccatgggcccacacagccccccatatgtgtcccccacctagtttgcagacctgacagacgctgctgcccgcaacgcggagctgctccgccaggccaagcacgaagccaacgactaccggcgccagttgcagtccttgacctgcgacctggagtctctgcgcggcacggtgagcgcggccagggcaggacggggacgggcggagcagggacggccaccccaggagatgccaggggagaagggccagggacgaggacctgggtcgagggcggggcctgaacgttacccccgggctagaggcggggcctggggccgggggccgggcctcgaaacctccccaagtcgggtcggggctccgcgggaccggccggctccatcccacagaacgagtccctggagaggcagatgcgcgagcaggaggagcggcacgtgcgggaggcggccagttatcaggaggcgctggcgcggctggaggaagaggggcagagcctcaaggacgagatggcccgccacttgcaggagtaccaggacctgctcaatgtcaagctggccctggacatcgagatcgccacctacaggaagctgctagagggcgaggagaaccggtgagccctcatcacagcccctggggaagtctccctgctgcctcacggtagagctcatcttgctcccttcagggactgagtctttttcagccttgtgtgcagatcccttcccacctcaatgcccagcacagtgcctgcctcacacatagcaggcagtcactagatgtttgatgactgaatggatggatggattcaaccaatattgattaggcctcaaggccaaggacttgggtcaccctccaaactaagaggcatactccccatcttcccagccaagattggactcctcacctcacccatctcttctgcctccttggaactttcttcaccactttcccctcccttttccatcttccttcagatagacatgcgcccctcccagataacactcaagccccttgcagacgtttggaactccagatagcacgttggctgctggggaaaaagctaggagatggagttagactttcctggcaaggaaaagggcgttcaagttgtcatgatggccccgcaccaagacccagcctaggggtgtcccaggcagcccgggcagccctctccctctgctttctttcaggatcaccattcccgtgcagaccttctccaacctgcagattcgaggtcagtacagcagggcctcgtgggaagggcactggagtcctgccccctcctccagggcctgtaggttgctccagactgggactgaggatcagggcaaagggatccagctctccctgggggccttcgtgacactgcagcgctcctagccagagcctatcataccagggtacttctaggtggggcttgcagctgcccctgtcctgctaggccctggtccctcttcccctccctgcaccccattcgacagcagaactgggtgagagcttgacatctgccctgtctgcagatccctgagcaagcactgcccttctgagtgttttctgtttttgtttttttaactgcttgtcactacagggggcaaaagcaccaaagacggggaaaatcacaaggtcacaagatatctcaaaagcctcacaatacgagttataccaatacaggctcaccagattgtaaatggaacgccgccggctcgcggttagctgcctgcctctcagacacggcgctttgcccagcttgacagggagtgagcctcacccaccccatcctcccaatccccctgagttccctcttcccaggcttcccctaaagggcctggactgcgtcattttcccaggaactgcagtgcccagcccaggacgtggtacagagtaactgtacattaaactggcagagcttgttagtggtaaaggtggtgagtccttgggtgcgcagtggagctgctctggggcctctgagcaagcagcagcctctgtctcacctcttcctgtcactgggagggccccttgggtctcgctgtgcctggacgccaggctctctgctttattctttcatccctgaggctccatcgctcagctcagtgctgactcagttcagaggattcttccctcaggaccgcagctcttgcagtgaataaagttttatgttccctgctcttaatgttaaatattagtgaatgtgacgtgttatatttttctctctcgggcggggcggggggaggggggagctaaattactttttacttgcattggccctgaaatgattcttagctaaggaacaaaattcctcttcttctggactagaatatttaggaccactagatatgcatgtctcaggacttaattcagcatcagaaaggctaaccaggctgactagtagaggtaatactgccaaatcctagatagtcacaggtcactaaagcacagataaatggcagagagaagtaggaaagaggaaaggggacaggaaagggacctatgtgtattgagcactaatatgtgccacatgtttaacagcctttattttagttaattgtctcctctctcccacctagagaagtaggccccatcatccttattttacagatgaggaaactgaggctgggagaggtcaattaacttgcccagagtcatgtggctggttggtggctaagttgggacttcttgagctctggtctcctgatccttccttccttccttccttcttttcttccttccttccttctttccttccttccttcggcagagcgtccctctgtcactcaggctggaatgcagtagtgcaatcttggctcactgcaacctccacctcccaggttcaagcaattctcgtgcctcagactcccgagtaaggggattacaggtgcgagccaccacacctggctaatctttctttttctttctttctttctttcttccttccttccttccttccttcctttctctttcctgcttgcttgcttgcttgcttgcctgctttcgacggagtcttactctgttgctcaggctggagtgcagtggcacgatcgcagcttactgcaacctccgcctcccaggttcaagtgattctcctctctcagcctcctgagtagctgggactacaggcacctgtcaccatgcctgactaatttttgtatttttagtagagacagggtttcaccatattagtcaggctggtctcaaactcctgacctcaggcgatccacctgcctcggcctcccaaagtgctgggattacaggtgtgagccaccatgcctggcctattttttgtatttttagtagaggtttcactatgttggccaggctggtctcgaactcctggcgtcaggtgatccacctgcctcagcctcccaaaatgatggcattataggtgtgagccaccgcacccctcctggtctcctgatttcaataccgtgctgggttttcagaaagccaggtgccaggccagggcttgcagaggggctgaaatgaagccaggccagaagacagagcagccaggactctggcagcagcactattgggatttgggggctctccagcccgtttggaagagcctaggctctctctgctcggttgcataggttctcatactcatgatggggaggggggtgcgatggggaagccgggcatgggagaggatggctcatgccctgaaagtccatgagaggcctcctctccccacatctgcagaaaccagcctggacaccaagtctgtgtcagaaggccacctcaagaggaacatcgtggtgaagaccgtggagatgcgggatggagaggtaaggagggatttgggcccagtcaggctctggctggccccagggattctcaaggccaggccatggaggaaagcctggggctggcacatagaaggttcccagcaactcccagtagctccccagggaattctggaggagagcaaggaaactgaatgtaattccgtttcctcagtccctccataggctgttctaaggggagccttggtaccaaagccactagatgggatcctaatacacactctctctttctcaccctcaaactcgggggccttgcttgccagggagagaaagagaattaaagttagtagctttcacttccaactctagcagacacagttggggatggggagggctttccatttccagcttggtaaaaggaaactaccaggggaatgggaagaggggatttggcgtatccgccacgccactccaaccacagtgggagctcatcttactccagcagcttaccactcgccaggcatgacgctaaatgctttcccagtgttatctcaccaccccctctgtccaccacgcaaggcagctgtggttactatcaagaaaagtaagacctgggaagtcggggacttcccaaggttacacagcctcgtggtggtggacctggggtctgtgtgaactcctaactgttgcactgtgcacgttccctgtcccctgcaggtcattaaggagtccaagcaggagcacaaggatgtgatgtgaggcaggacccacctggtggcctctgccccgtctcatgaggggcccgagcagaagcaggatagttgctccgcctctgctggcacatttccccagacctgagctccccaccaccccagctgctcccctccctcctctgtccctaggtcagcttgctgccctaggctccgtcagtatcaggcctgccagacggcacccacccagcacccagcaactccaactaacaagaaactcacccccaaggggcagtctggaggggcatggccagcagcttgcgttagaatgaggaggaaggagagaaggggaggagggcggggggcacctactacatcgccctccacatccctgattcctgttgttatggaaactgttgccagagatggaggttctctcggagtatctgggaactgtgcctttgagtttcctcaggctgctggaggaaaactgagactcagacaggaaagggaaggccccacagacaaggtagccctggccagaggcttgttttgtcttttggtttttatgaggtgggatatccctatgctgcctaggctgaccttgaactcctgggctcaagcagtctacccacctcagcctcctgtgtagctgggattatagattggagccaccatgcccagctcagagggttgttctcctagactgaccctgatcagtctaagatgggtggggacgtcctgccacctggggcagtcacctgcccagatcccagaaggacctcctgagcgatgactcaagtgtctcagtccacctgagctgccatccagggatgccatctgtgggcacgctgtgggcaggtgggagcttgattctcagcacttgggggatctgttgtgtacgtggagagggatgaggtgctgggagggatagaggggggctgcctggcccccagctgtgggtacagagaggtcaagcccaggaggactgccccgtgcagactggaggggacgctggtagagatggaggaggaggcaattgggatggcgctaggcatacaagtaggggttgtgggtgaccagttgcacttggcctctggattgtgggaattaaggaagtgactcatcctcttgaagatgctgaaacaggagagaaaggggatgtatccatgggggcagggcatgactttgtcccatttctaaaggcctcttccttgctgtgtcataccaggccgccccagcctctgagcccctgggactgctgcttcttaaccccagtaagccactgccacacgtctgaccctctccaccccatagtgaccggctgcttttccctaagccaagggcctcttgcggtcccttcttactcacacacaaaatgtacccagtattctaggtagtgccctattttacaattgtaaaactgaggcacgagcaaagtgaagacactggctcatattcctgcagcctggaggccgggtgctcagggctgacacgtccaccccagtgcacccactctgctttgactgagcagactggtgagcagactggtgggatctgtgcccagagatgggactgggagggcccacttcagggttctcctctcccctctaaggccgaagaagggtccttccctctccccaagacttggtgtcctttccctccactccttcctgccacctgctgctgctgctgctgctaatcttcagggcactgctgctgcctttagtcgctgaggaaaaataaagacaaatgctgcgcccttccccagagtggactctgatctgttcatgagagggcgggactggggccaagatgtagcctttgacaagaccaactcatttcttattactgatcatctctggggcccatgccctcaccaaattccacccgcagccaaagaggacatacaccagctccctccactcttttcttccttcctctccctgctacctgcaactcaaccagcacaatcttcataggcaagaaagcaaagcagctcaaacatgattcaacactgatcagtgtttaccactggataaatctgagttcacactttccttctctgacctaaatgtgaagtcaggaaacacatgtgccctacttccatcctgagctcagtccccaatctcccaccagcctcaggcccctccacttctcagatcaggtcccagacctgcccatgaaaatggggagcaggctgtaacagatttgtccacatgttcctaccacctgtcccaacccagggtacccacccagagacatctggtatcatttaacaaacacattgaaggacaactggtcttcagagctgaagagagctcctagggggagaagctgggacaacagtgaaataagtagcagcagcaacgacagaagtgaatggtgacaaagactgctgtgatgagcaggtagcctatcagggtgagctccacagccgagcgagtctcaggatctgagaacgaggctgggtagtgcccatgagatgtcacacccagccggaagccagcaactagcacaccctgcctccagcaatagtagatgccccggtcatccagctgggtgaagcggatgtggagctggttgccgtggtcaatgaacaccctcatggacctgttgacacccttcaggtactgtgtgcggtagaggtgctggcggtctttgtcccaggccactgcatgctctggccgggccccaggacaggagatgatgagtccatggcccagtctctgctggtggaactgaatgggcacctggggcacccagggccggctgcccactttggacacatagttaatgatggccagcacgccctcccggatggtctttgtcttctcacagggtactaagcagctccgaaccagcacctcaggcgtgtggtccctggccttggtccgcagcttccttggcacagcccttgagccacaagacaccacatcgggcacggccttgaggtagcgtggggagaggtctgggctctgcaggtagcagaggccgatgcgccactgctccccacgcactccgcagcggtcacagggggtccattcccagaaggtggtgaagacatggaggtgcccatagtattcatctgcaaagggctcctggcccttgtcctggaaagtggccaccattccctcactgttctggatgtccacatcgtaggcgtaaaagtagtcccccttgcgggtgccgcagaagtacaggcctgagtcctcagactgagccctgaaaaccaacaagctgaacatgcggatgctgaagcgggtcagcatgtcgctgcccacacgtacctgggctgcctccgtcagcacccgcccatcaaagtccgtcagcactttggtgtggctgctacctaggtgcttttggtagaaccagactacagctggcacctcttcgggtttgcagtgacagggaagctcaaagctcatgtcggccaggtaggctgcattttcaaacatcaggaaagcagggcagggggtcctctgaaaaatgttttccttctccacaatttcaaaggcctggagcccccatgcccacaggagcacagtggtgagggccaggtgcatacctgaaggaggcaggggtcagaggggcagggcaaaaccagggcattaaaggctcatagggctcctagaaagctctgctaagcggaagcctctagatgaggaaaggattatgcagccaggaaaagcagcaacaatctgcagaggaagccgccaagtgcaaggcaatttattcccagtggatgtacaagatgcccttctaacattc

SEQ ID NO: 47 - DNA, 38916 bp, Mus musculus
acagaagccagcaccgcctgcctcccctgtcactcatacccacaagaagagtcatccctcctgcgttcctccccagctctgctcctaatgcagggagggcttgtgctgggttcacgttccaatgtgtccttcacccccagggacccgacgtgactccagagagtgagaacctcaccctgtcctcctcaggggctgttgaccagtcgtcttgcacagggactccgctctcttctaccatctcctccccagaaggtacctaagggagtaactgcatgcgcctccctcacctggtccacagccgtgacttcctctggggatgggagcagtgagctggtgattaaaccggtccacactggagaaggcaggcctcttatccaccctcctacatgacgcgatcccctcggtgatgtaacagcaggagcaagccctcagtccttgagtttcagagcagagtcttagaagaccttcagtctgagcctttctcaggacagggcatgtggaggcatttgtatgcctcgttgtggcctagtgtaggaagtgtatactgctccttggtaattatggtcctgtccccgccctgacttctgtgtccacagacccagccagcagcagcctggcccagtcagtcatgtccatggcttcctcccagatcagcaccgacacggtgtcctccatgtctggctcctacatcgcacctggtacagaagaagaaggagaagccctgccttccccccgagctgctagcagggccccttcagaaggagaggtaagggaagagttctccttgacctgccttgctctcgcttcacacaggagtgccaggcagcttggtcaaggcccatgtgcgggcaggaatgctcagggaggacagcaggctctcccttagatcccgctcagtgggccaccagccttgtgctgaggacagctgtgggcttgggtttgtggccttaagagcttaactggagagcctgtgatgaccacaccctggggcttcgttgctcttttacaggagacaccagcagagtccccagacagcaatttcgctggccttccagctggagagcaggatgcagaggtaacctgaacgtcaggcagcagctccttgcccacagccagggtctcctactgccggggttagctgggaatgactgtggcaaaggagcactggaaaacctgccctggaggcatgcctagatcatttaggagtgtaggtagaggggagagtggcctccacctctggcctgctgcactcttctgtcagcaggggacactggttccatgtatgagccacagagagtgaccttcccttgccagaccctgacaggcctgggatgtgctggctgtcctgctgttagtctgtttaccagggctgctaggtctccagctgtgggtggagatgccctgggtatgctatgcaccatcactgtgcaggccagcctccatcactgtgcaggccagcctttactttccttctcagagggcctctcaggacattgggtagtaattggccaatgatgaatttctgaaagctttgtgacctcatgatttttatattctattatattttgtctcatttgctggtttgagacagggcttaactatgtataccccctaagtattcagtctggcctagaactgccgttgtagaccaagctggctttttttgtttgttttgttttgttttgttttgttttgttttgttttgttttgagacagggtttctctgtgtagctctggctgtcctggaactcactctgtagaccaggctggcctcgaactcagaaatccacctgcctctgcctcccaagtgctgggattaaaggcatgcgccaccactgcctgaaccaagctggctttttacttcaggtgacctccctgcctctgtctcccaggtcctgaaattataggcctatgcccatacctgatttttatttgcttaatttaactcttatttgtactttagtataagcgtatttcttacaacccatcctaagcatagacttgaagttgatcaagcatcccctggccatggtgggaagtatgtgctgtggaggtagaccgtggaaatcatgacggcgggcaggacgtgatttgtcctgtctgtgcttggtgcctctaacggtagactcatggggcttttcttgtgtcctagggaaatgatatcatagaggaagaggacagatcacctgtgcgagaagatggtaaaagatctgtccctgctctcactcatgctgcctttctgggtactgctaaggggggaaggaaggaaggatgactgtggctttagtggcggtgctccgctgtgggagatgctgtcaggcaaatgggatatcggtgaccttagtgacatgctaagctcctgctgagacacagactccacagggtgggggtggcgtggatggtgccgcagaggacaccacgtgtggcagctctctagctgagcccttgggctgccctaaccccaagtggagtagtggtctcttccacatgagaggcaggactctggtagcttgtctggttggctttagtggagaacacctaagaggtcggccagcagctcccttgtctcaaggatgtggctgacttgaagcacggccccctgcagggctggctggggtggggggcagcaccacaggctgtggtttgctcttcagttagatttgtctccagtgagcctggaaacgcagcatgccctgcaggccttgggaggaggaagaggggtggttagggaagtcactctgcccatcccagaagccagactccctgtcctctctgctcccaggcagatgtgtgccgcggcctgcgtacagtagccctggatacggttgctccagcagaggtcatcctagatgctcctgctttagcacgggccattgccattgcttgaggggtgtcaggggtgcatgccggcttgactgctcccagggagcacatctgcccagggctaggagcttccctgcctctcaagagatttcagaacccaacatggcttccccaaggaaccttttcttctaagatgcacatggtggccagcttgttggcactcacctcttcatagtctccatggagggtttgcctcctttcttttcactgagaaagaaaggaggagtttaaaaaacaataatgcctacaactctcccttgtcctttcatatgacctcagctctctgggcagtctctgagaacctccagtaacaaacagcctcagcctcctgagagtgaaaatcaccattctgtgaacagtcattccgatgtgctgtgcagaactgcgcaacccagagccatgagtagtgcactgtgtctgcttctgcaggcccttcggtcataagcacacctcactcatgcctgggagtcacaggcctgaacaggggaccatctcacctcagggcctcaggccacagtgtcatcacctcagggcctcaggcctcagtgtcatcaggctagcccgggtgcagctgcccttctgacctatgtctggcagccttccataggaaccagacttattaaggaacaactagaaaggggcacctaaagaaatgagccagggtgtccgcgagaacccgacatgcccacacaactcagttcacaaaccagggtgcccgcgagaacccgacatgcccacacaactcggttcttctgtccactctttacttgattttatcgtctttttttttttgacacatgattttattttatttttttactttttgtctccatcttaaaacttttatttttttttttaaggacagcgaagtagagaaatcctagccagcccctgtctaactgcattaactccgttacaggccagaggacatgcgcatttctaggcatggagtgtgacaataacaatgactttgacgtcgcaagcgtgaaagcactggacaataagctgtgctctgaggtctgcttacccggtgagtggcagtgtgctgaacatgaacttggtgggagacgcccctccgctcgcccccgctctccccgtggaggccttggaaaagaagcctcagcttttagaatagaaaccgtagctcttccagggacgtacgtttgaggatcacaaggaaatctaaaagcatctgtaggtcagtgaatggctttctgatccgacgagccttaggtctccagtgatcacacgtgcaagactcggatcccagcggagaaggcagagggcggggatgacttggttctctaaggccccaacttctcagcgtgccctgtttctttctctgacctcaaacacagtctgcaaagcagccatgctaattgtacattcaagcccacgcatgctagcccatgcctacaaccccacacttggcaaacagaggcagccccatcattgcaagctgcaggccaggagctacgtaacaagacctacctagtctctcaaaaaataaaatcaagcttggtgtggtggcgcacgcctttaatcccagcactggggaggcagaggcaggcggatttctgagttcgaggccagcctggtctacagaccaagttccgtgacagccgaagccacacagagaaattgtcttgaaaaccccaaaataaaataagtcacagttgagcactgacttcagtgtgcccatcctgcagcagccaagaccctgcctgggacagatgtcccttacacttccctggatcgtccccgcagtgtcccccactgaggacctgcttcagcttcagaacagccactccggggtccagtgctttactcgctcagcgaagtagctgaagtgttaagccgcagggccttctgtggctgccactctccttcagcctgtgccacccatggtcccagcaggccctggccccatgcaagcctgcggcaagggaagccagttgctcgggagaaactttctccagcctttctctctttagtttgtcctgaagatactgcccaggtcccgggcttccgtgagacctgagagacagcatcaggtcacctgcttctcccgcacaggccccaggcgctctcccttcctgtccaagcatggtggagggcgttgatccatatggccacctcctgcccagttaaggctgggctggagctgctggagaccaggcacgctcacccctgccttcccactcctggccttgcttccatccgtccatttatgttccatgtctttatgccaagtgtaggtctgtctgtctgtctgtctgtctggtttgctcatttttaaactcatttctgttttctgacttgtgtgaaagttcatgttcacgctgtgccagcactggcctcctgcctctgctcagcccgaccagcaccaagcttactcactggtgtgcgcatttgaggggctctcgctagggctctagacaagcttccagttcctaactgacaagggccgatgcgcaatacactgagttctaaagagaaaacccgtgggagctgttcaacaaaaacagacacgcttctgtggcaaagaccgggataggcagcctcggttggtcagagggtgaagatggtgcctgtgcctagccctgcctgggtctgtggtggctttctgccggccagcctttcctcaagggcttctaggctgagccccgcttttggcagagggagctgctgccactgttggagcccacggcgccatggagaccaggcttgttctttctgcttcctcctcgccaagcccccttcccatcaccctgcttgtcagcgcagtgcatcctcacttacagggaggaggcagcacggacacagcagcgtaagcgggcagcgagcagcacgccttccgtatgggaatgtctagggtcagcaggtttgagattaaacggagctcccagtttggccaaacatggaatcagtcacctgtggcagggtcaggagagagactcgggtataaacagctcttaagtctattcgaattaagtctctgtagcaaaagtccaagggctcttgtggagaggaagcgtgtcccctctgctgcatctgttagaatcacaccagtagcaaaggtgccaaggtactcggaggtgtctgtttatctcctgggtcaaagccaggagagtcgtgtgtagcctctccggactaagttcacactcctccacggcatgctcatctgacccggccgtgagagctgagggctggctcgagcctctctgttctttaatgctcttttctcctgtgaggagcacttcccacagaccctgcccctgcagtctgtgcctcttcaataggtgccacccccttcatgaagagcagacaggggaaggccgagaagggcacagggtgcttagattgtgtgtgtcaccctctaccaagaaagccatcatcaccatccttacgtgtgtgagggccttggagagggtctgtgccttccgactcctctcccccccccccccccccccagtccccaagaagcttccagggcttcaaaggcccaagactgaggccatctttctggttccttatgctagagcctaacttgacaggcgctgtgataagagctggaactctatgaccctgcagtctgtgtggcttcaaagagcagtccgtggctagcaggagagtgtgtggggcagtgagcatgctcacaaggtgctgggtaggctatgtgatgggtaaggaagagacaagggactttcagtgaaactacatacatgattgagtgacacaggagtagacctgtgcagtaaaatctaaacaaatagagacctgaagtgaaacccactcctgtcttcaagcctcatgatataggatgcagtcatgtggggctggggagatggctctgtggttaagagtactgactgctcttccagaggatctgagttcaaatcctagcgcccaagtggtgcctcacaactgtctataactccagttccagaggatccaacaccctcacacaggcatgccatacatgcaggcaaaacaccaatgcacataagataaaaaataaatcattaaaaaaaaaaaacagatgtggtcatgcatacctttcatcccagcactcagaaggtggagccaggcagattttctgagtttgaggggagcctggatctacatatcaagttccaggccaatgacggctacaaagtgaaaccccagactcttgtttttaaaatgagatgtctgcagtgcacactctgaggggaaatctaagttacagaataagcatattttaactactctagcccgtaagttaatccgcacatgagtagaggcagcagagaggtatgccaagtgtccctaaagcggcctctggaggtctcttcctctcatttctgattccccaaaagggaaccaaatacaaagcaccactgtccctcccctggaaggggttctagtctccaagctccattttcttgccagagagcctggatccaggcagatgggagggcacactcagacatctcctcgggatccaggctgccttgtgagtgggctgtggagaggcaagcatgtgagggccaccatagatcctgtggctcctccccagggcaaaggacatctcgttccctcgccatgtcccttcattgcacatggcccatctgcagcctagactgttctgcaggcaacctggagccctgaccggagttcctgggtgggagatgctgcttaaggacgtgtgtgagtttggtcgggacagcgttaggccctgagtcatggatgctggcagcagtgaccctggtggtagtgatgaggctgctgctgtttgacttgtgtgcgcttgtgtgtcttctctcttgtttaccatttgcttttggtctctcctactgaacagccgcagctccggagtcctgccccataaacattgaggaataggtatgacatctaccaaattatataactgaacagaccactcatttgacctggggtctcttccctgatcctctttcccacgccccagagtgccacctgcagaggggaagaaactccttggactcatgctcaggagtggagagcctgtgggcaagaccctgtacccttcctttcccagggctgtactgaggcctgttactatatagcctcacaccagaccactcatgcaaaataagcaatcagtacctgcttcctcctagcatatgtgctattaaggactttcttcgtgtcttaggctcgctgaatctgaggctgactttggccatttgtaagttggactcaggactgcaatatacagttagctggcctcacttaatagaaaagccccacgcccccatctccgtcctagggccacactacctttctgagctcccgggtgcaaacctgtaaatggttcctgcagaagcttgccgagcagagccaacaagatgaagtaccttccctgaacatcccgagcctggaaaggagcagggcctgccattgtataaaacatagtcgctctcctggagagaacaaaacagagctaactcgccagctagagggacctaggaaacacatgaggctcccccaaaccaagctggcaggaactgctcacaggtcatttctagtcaggtatgtggctgggcacccttacgtagggccccatgcgggcctcacagccaatctgtgagtgctacccacttgtctcactgggcatacgagaaggccaagccccctaaggaaggtgacctgccaaagatcagaccgttggaacctatgaactgatctgaggcccgagtggatgcaggtgtgggtgtgactaggaagagaaaacaaggtaggtaggttgcatcagagcatgaaacagaagcgtcagggttgagtctccacttggagccacagatgcagccagaggctgggagaagccagggtgataaacagcatcttggactgaaaggtgctcaggcgaggaagtggtacaggtgaaggtgtgggcatacaccgtctcccacccaaggcttcccttccctggcccctgctatgctgcagccccacccccgcccctacccccctgcagacacaggctataggtcagggtgtgctctctgtacacccaccttccatgcaggaagcagcccagttcagcctcctaactaacactcccaaggccagaaccaagtatgtaacatgcaagctggatgctaggtaagttcacgaatgaagaacacctacagtctccccatctgtccatgggttacaggcagcgactacctagctagggaggggcagccgtggcacctgccagcacctgctcctcactcttcctctcaggagtggctcatgcagccagatgtctcacgtcacacacatcctgctagattctgtgttgactcctgtgctgctgtactgggacaccatgtatgcttggcctaaggaatggctcccagaacctctgttgttccttctccaggtacctggcagcatgaggataacactgtcaactgtcgccatacccagcgccggcgcttatcctccagcagcctggaggaccctgaggagaacaggccttgtgtatgggatcctatggctgtctgagggcatctgcacctccacctgggcgtcccctcctgtccttgccttccatctgccctcactggaccacgagccttctggtcatcttcaacaagactcgtggacttcctactcacatgaagggaggacgatgcgcaaccctccacccacctgcctcatcgcctgtaaccgcgattcttaccctctcagaaagaaccagaagccttccctctgtgggctaatacgtgccagccaaacctgtgggccagctgagctgagggtcagggctggttgtttctgtaggctttctcttctgagtggagaccaagaagactgaagagtttggcagccttgttagacgcccatggacaggttttggtctctggcactcataaacaattagaaacctaacttgaactggttgggaacttgctccatgaggaaggccatccactccggcctcccagaacaccaggagctccttgcccacgcctaagggagagggtagccaccgaactattggttggaagccagatctaacagagttggtctcctgcacctccggtgtccacagttaggaagataccccttgcaaggcctgtcctcctccagtgcagtcaggaaggcctactgggccacttccctgcagtgcttgggcaggctcttcccatatcgtcactacagccactgagggggtcttgaacccagctaaccacttctctgccctctttgtaatagctgaagaactaggctaagagctgaggcccagtggttctcaaactggtgcacaggaacaccaaaggaatggtggaggctctggggccagaggattaggagacctccctaccctagtgcattcctcccctgcctttcagatgggatagatgagaggtgggctcaccagtgctgagagatgctgtcaggttagcactcaaagccatagcagcaagctggcaggaaattgttgggttcagtgggttttaacatggctctaaagtagatgcctatctagtggcttttggaacagaaagtcgttcagcggaacatgagatggtttggggatcaagattagtgacagtcacttatgagggtgcgatacgaggagctttagacttgcaagtgtgttctttgaaaggctttccccaaaatgccagtgcccaggatcctgaggggaggtcgggggtggagagtccggctgcaaacttgccatgtggctgggaatgaccttgaacttcgggttttcctgccggtactgcccaagagttaggatcacaggcctgcaccaccgcacagctctcctttccatggtaatcccactgttaggggctctgctgcgctgcgggacgtgggacaggctgagcttcagatcccatgttgccccttctgaacaccctgcttcccgtcttttacttcctcagcatcccgagggcccctccctcccacacttaaggtttacaccagtgtaggctgagcaatatccacacctcctcaaaaaccagaacagaaacaacagggaagttccctgagcctgggacggctgcacggctgcacgtagcccttctcccctggcaggcagtaggtaagcggtgttcacagtgtggtgtgacctgggaggaggggaagaagctgagatgaaggagattcgggtaaacagtagccacagtggccaggaaccctcgaaaaagccattataatcagcccttccgtctcccttcacaagtgctcagcctgaattccataatccaggcacccaggttagcataccctttgctctgccttgctcattccgcccctcactctgcccttttacttcttggcaagcagaaatagcccattcacagggtggcccactgtcagctgccacacaacccagtccatttgggaaccatccctttcttggggctcaaatgtacagagtcaggcatgttctcctggtgccgttggctcctcatcacacagtatctgcacctttccatgcttggtactgcccagatcttggcctcctcaaagagcctttttgctttacctaatttcatcccaccagagtgtctatcccctgtgacatctgccaggcacagactcaaaccctaagccatccatcccactggggaactagtcctgtccatctggtgttggcgtgctggctgcacactttgttttagggcctgaacacctgtgtgtatgtgtgtgtgtatgtgtgtgtgtgcgtgcgtgtgctctgtatgtgccatctgtgtacccctcacattcatggagtacacttataggcacattacagtggggacatcaagatgcaatatttatggattgctgcatgattcttaaaagtgaataaaactgtttacaggggcaagggagccattaattacaacgtgaattcgtttgtgaacgtattcgtttcagtcagtggctcaggctctttttagtatttctgcagacctgggtacaaggccagctccattcctgactgccctacctgcttctgcatctgccttaaacccgatcacttccaatcctgcagctcgtcaattattaaagaaccccaaagctgatggcaatagggaaagctagggcccagaatcccttatgcttagtagttcccatcacttaaccagttcctggctaaatcagtagaacaaatgacctctttgagaatcttgcaggcccagggcttttctgttttttgccggctggctctgcttcctagactgtctgaaagggcagcacctccgcagatgcccctggaatgccagcttccgggaacttgagcagggtgagtgggcgagctgcttaccgggagacgctgggttaccctacccgcctcggtgtgtctgcgagctgtgtggccgtttctgcggattataagcgacaggtggcatcagagagtcaaacttgcctgtgggcaccagagaagacactcggggaccagggaaaggccccagaaaccagacaaaggccactggcagcccggtgagggcaaagacttcaagggcacggttccccgagacttggtctgcagaatccggggctgggtcccctcagcggtcaaacccgacgcgttgggggaggggttcccggggcctcgagcgggagccgagcacccgccgtgagacccagcgcggggccctcgggcgggaatgcacagagtcggatctcggtggcgggtagggggctgggtggcgcgggagagggcggcagaaccagagagttccagagagttgccgccagaggagctgcaagggcccgcggtctccttggagacgtactggccaatgacagcagcggaagctgtttatgggggcggggcgtcgccatggcagcagaagaagccttccgagcgtctacttaatgccggtccccgggccgtcggcgctcagagaagcgccgtcgggcgaggggggggggcgcgaagaggggcgccagcacagccctcgagctggggacagagaaccgcgggaggtgggaggaacttcttccagaaccttcctctggctggctgcgctctgagccaggtaagggactgggagggctgtggaccacgggatgggagggcgtcgggacctgctggctatccagtccatcggtcgcccacagaagtagtggggccacgaaaatgaaatctgaggagttcgggagatcgcttcctgctcctccataactcggtcctgtgaccttcctttcccgagacccttcatcctaacacctcttttcaaggatcagatgacctactttgccccagccgcgtcgtactgacccccctggactattgggatgtgatcttagtttcccactctttggggaaaagagttagtttgggccttcctaccctcttccctcattgtggccaacaaacccttcaagggcagatggagagaggtggagacgcaggatcagaccagggtcctgtgtttccctgacctcgccacaggctgcggggatgaatgtgaggggctccccccctaaggcgcctctttcctctcggggtaggggaagagtgttcagttccgtggtgctcccgcaggcctcacattcgtgttcggaggaggcgaccccctgggtcgcagaatggaagtgagtgttctggagactgtgtaactggagtctgggttaccagcgggccagagagcgcggcgggccactccggctcagcctgtggatgttgaccgccccttcggagagtccccgcagacatggcggagagctggctgcgcctctgcggagcgggtcccggagaggaagccgggccggagggcggcatggaggagccggacgccctggatgacagcctgaccagcctgcaatggctgcaggaattctccattctcaacgccaaggctcccactcttcccccaggaggcacagacccccacggctaccaccaagtgccgggcttggtggcgcccgggtcaccgctggcggcagaccctgcctgccttgggcagccgcacacacccggcaagcccacatcgtcgtgcacatctcgaagcgcgcccccggggctgcaggccccgccccctgacgacgtggactatgccaccaacccacacgtgaagccaccctactcctatgccactctcatctgcatggccatgcaagccagcaaggccaccaagatcactctgtcggccatctacaagtggatcacggacaacttctgctacttccgccatgcagaccccacctggcaggtagggggaggagaggtgcgggtggtggccactccctcctcacgacctccttctggcacttagccaggctcgcataccagatctgcaaggccaaggagacacctggctgtgaggtcaggcctcatgagaggcctgaccactctgccaggttctgaaactgctcccttgtctggagccaaggccctggaggtacagacgaagttgtagaagagggaccaatagggcccggtgtagcctcatgtgggatgtaacactaaggcacattcttgtcccgtgtccgaaaattcctagacaatgatctgcccgaggaattagtcagaactggactctgaaatggaacctaggtgacctccctgccaacttctgcaccctgcgtctagagagtagggcagagatgagcccctctgggtacaaagatcagttgtccaggtggaggggtcaggaagtcatcctgaagccactgaccagcctcccacctcagctcctccccagtttcctgcccccacccccaccctctcagccagcctccctaggggggcaattacctgagccaggtgctttccacaggggcacttctgctgcagtggcttcgggttctaaatctccagaacctggcacagagcagccaatgggccaggctgccgtgtaaacatctcagccctgctggccaaagggtgcccactcactgcccacctgccttgctagcatagcctgggcccctggacaccagtgtggtgtgagcagaactaagctgctgcgtgtggagagggcaagataaggctcagtagacagagacagcactgtgaacatcctccagtatcaagatccatgttctagagacgaggcctgaggttgcccactcttccctaaggcagccaaggagcaagagggcacgatcatagtcctaacccattccaaatggctctaaccctgggtcaaaagattccccaatcttctccttattcaatgcctttgccaactcagaaggcgcataacgataccacgatatcaacaagtttgtacaaaaaagcaggctggcgccggaaccgaagttcctattccgaagttcctattctctagaaagtataggaacttcgaaccctttcccacaccaccctccacacttgccccaaacactgccaactatgtaggaggaaggggttgggactaacagaagaacccgttgtggggaagctgttgggagggtcactttatgttcttgcccaaggtcagttgggtggcctgcttctgatgaggtggtcccaaggtctggggtagaaggtgagagggacaggccaccaaggtcagccccccccccctatcccataggagccaggtccctctcctggacaggaagactgaaggggagatgccagagactcagtgaagcctggggtaccctattggagtccttcaaggaaacaaacttggcctcaccaggcctcagccttggctcctcctgggaactctactgcccttgggatccccttgtagttgtgggttacataggaagggggacgggattccccttgactggctagcctactcttttcttcagtcttctccatctcctctcacctgtctctcgaccctttccctaggatagacttggaaaaagataaggggagaaaacaaatgcaaacgaggccagaaagattttggctgggcattccttccgctagcttttattgggatcccctagtttgtgataggccttttagctacatctgccaatccatctcattttcacacacacacacaccactttccttctggtcagtgggcacatgtccagcctcaagtttatatcaccacccccaatgcccaacacttgtatggccttgggcgggtcatccccccccccacccccagtatctgcaacctcaagctagcttgggtgcgttggttgtggataagtagctagactccagcaaccagtaacctctgccctttctcctccatgacaaccaggtcccaggtcccgaaaaccaaagaagaagaaccctaacaaagaggacaagcggcctcgcacagccttcactgctgagcagctccagaggctcaaggctgagtttcagaccaacaggtacctgacagagcagcggcgccagagtctggcacaggagctcggtacccggaagatctggactctagagaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataagcttgccacaaccatggaagatcccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgctggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataataataaccgggcaggggggatctaagctctagataagtaatgatcataatcagccatatcacatctgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatccggaataacttcgtatagcatacattatacgaagttatgtttaaacggcgcgccccggaattcgccttctgcaggagcgtacagaacccagggccctggcacccgtgcagaccctggcccaccccacctgggcgctcagtgcccaagagatgtccacacctaggatgtcccgcggtgggtggggggcccgagagacgggcaggccgggggcaggcctggccatgcggggccgaaccgggcactgcccagcgtgggcgcgggggccacggcgcgcgcccccagcccccgggcccagcaccccaaggcggccaacgccaaaactctccctcctcctcttcctcaatctcgctctcgctctttttttttttcgcaaaaggaggggagagggggtaaaaaaatgctgcactgtgcggcgaagccggtgagtgagcggcgcggggccaatcagcgtgcgccgttccgaaagttgccttttatggctcgagcggccgcggcggcgccctataaaacccagcggcgcgacgcgccaccaccgccgagaccgcgtccgccccgcgagcacagagcctcgcctttgccgatcctctagagtcgagatccgccgccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccccggatctaagctctagataagtaatgatcataatcagccatatcacatctgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatccgggggtaccgcgtcgagaagttcctattccgaagttcctattctctagaaagtataggaacttcgtcgagataacttcgtatagcatacattatacgaagttatgtcgagatatctagacccagctttcttgtacaaagtggttgatatctctatagtcgcagtaggcgggccttatgcgcttggctgggtaataatcaactgaggtggtttgggtgggaagaggagattgtagggggtgaaggtctaggccagcagctggctcaacaaacaaggagtcactaaatctcccaacacacacacacacacacacacaaacacacagtccttggtcacccagccccctcccttaacccacccacttcacaaacagagggagatggcagtccaggccgtgggggagctgagggctttggccaggacctgcaagcaacagggtaagggagggagggaggatgacagagggtggcactgagagccaccctgtggctcagagctcggatgactgagcaggcagatatcccagcaacagagaacaaaagcccccccccccccccatgcactccgggctcctcataccctgtggctggtgtcccagagtccccgagctacagctgcctggtcagggcatcggatccgcccctcccccatctgggaggatgcaggagcaccgggtgccacttcactgccggtcccctgacccctatgccttttctcttgttctctctctgtatccagaattccatccgccacaacctgtccttgaacaagtgcttcatcaaagtgcctcgggagaaggatgagcccggcaaggggggtttctggcgcatcgacccccagtacgcagagcgcctgctcagtggggccttcaagaagcggaggctgcccccagtccacatccaccctgcctttgcccgccaggcctcccaggaacctagcgctgccccctggggtgggcctctgaccgtgaacagggaggcccagcagctgcttcaggagtttgaggaggccacaggggaggggggctggggcacaggagagggcaggctggggcataagcgaaagcagccgctgcccaagagggtggccaaggtcctgcggcctcccagcaccttactgctgacccaggaggagcagggtgagctggaaccactcaaaggtaactttgactgggaggccatctttgaggctggcgcactgggggaggagctgagctcactggagggtctggagctaagccccccgctaagccccagctcacatggagatgtggatcttacagtccatggccggcacatcaactgccctgctacctggggacctccagcggagcaggctgctgacagcctggactttgatgagaccttcttggccacatccttcctacagcatccctgggatgagagtggtagtggctgcctgcccccagaacccatctttgaagcaggggatgccaccctggccgctgacctgcaggactgggccagtgtgggtgccttcttgtaaaaggtcaggccccaccccatctccggacagtgctcaagtcagggcccagaactgcctcccaagacaggcccgtggacatctcagcacccaggcagggactgggccaggtctctgaaggccacactgctgccagctggggatgtcctcatatccaagcccagaagactgggaactggggaccctgcccctcccccatcagcaacccccatacacacagtgtttcatccacccatgttccctcaagcccaaggattggcaaaagcacccctgcccctgcctgccccccatccccacccttgcactctgagagctgaagcccagaagggtcctaccaggccagggaggaactgaacagggccctcaccaaagcttcgcctcacccacttctgctgcttctctgggtctctacccagagaaagttcccaggtacaacaaacgctcattagatgataacaaattaattcccaggaggcccctcccggcagagctttcccaggggccctggcagccttggccagggagagagagtgagggcaagagactggggaagggaaaagaaaggaggcagaaatccggtgggggcagcgaagtggggcgaaatggtctctaagcccgctagggcctttccctggtccatcacctgcacaagggaggctacaaaccttccagcctgacttgaggctaagagcatcaggaagatggggtgtggggtggttgggtcaggggcagccaagctgctagggggtggggggagagactaatatagtgaatgtagttatagctaagacttaactggggaaaggatgcccggctcactgggaacactggggccaggaaggggacaagccctaggcccctgtctatactagggggaccaggccttgctccaccttgacccctggagctgggacctgaatctaccctccacgtggagcaatttaacctttttcatgtaaagttctttacaataaaatgtttttcaaaataaaatttaaaaaaatctgaacatggccgtatttccagctttctttgggaaaggagagagccaaatgtggtggctttgggaggaaaggattggtgtttcttctccatgtcaagcgggaatggccagggaaacaagacagaaagggtttgtagaaggtctaacctcaagctaggtccctaggaaggagagggcctttctcagggcccttccattctgtacattaggacctccatctgcacaatgaggcagttgggcctacaggcccagtcgaacaagagtcagggaggagattggccccgcctcaccccaaggattcacaaaggccagtgtgccctggcccaagccgtcccccgccatgagagcctgcccaggcttgcctgcccaagagtcccgggactctttccagcaataccctccccgggtccttccccagagggacagggtgtgaggtcagaggagccccaaggccccatcctgctctgatgtgtgtttaccagacccagtgcaggctcggggaggcagaaggccgccttaaaatgagacttgccctcccaactgtgaaagtccaggctaccagagctctgacagcaaaacaggatgctcctgtctggatgggcccctcatcttaggtttgtgtgcctgagacccatttctctaccactaccactcgattccaccctacatagcatccttgtgctcacccagcccttctctgtggacagcttgtctcgtgcacaggtgcacacacatgaacaacaacacgcagaggagcagcattgcccgggcgctgtccagagcaagcctgcccagcctccatggcctcccctttctttcttcctccatccttgagcctggccccaggccaacctggcattcccctgctttacacgccgctgctttcatttgctaattattccaaatattccagaagggctcacactgagcagggcaggctggccgggcctatcccttcacccagcacacagaaggggccattccccaaccaaagcttctgttctactcccccccaccccacccccagtcgcctcccccacccccttcctctctacagaccacagcactaaaagaacaaaggcagccccagccttctcccgtttgcctcaaggatcagccttcggccctggccaaagtgggcacaccctggattgacagtggggcgagttactgcctgccccaggcttatgctgggaagaagagaggcaatgtctggggcaagtagaaccgcccctctcatgaatacaaggccaaagactaacggaagacacagagcagagcaaggcgcgcccttggctacacttcccagtcactccgagttcctgaaaattctgatgcccaggctgtaccttggaccaatcagaccagagtctctaatgggcccagcacagattttcttaagtccccacatacttctactgcgctgtgggtggggggtaaggtgcagcacttgtcttctccagagcaggacagtgggtgtcttcctccgtggctgtctgcctcattaccttgagacagagtctctcgtgtggaaccacagcttacagtgccaggtcatcaagttcttttgcaagggaggtgtctccgtccccagtgatggggttattagtcgtgtggtggcaatgcccagcttttaacacgggtgctcgctcgggacatcttctcaggacctcatgcttacacgatcaagccctcttgcccctgagccgtctaaacctcatctcatataggctcagtataatctcagcactaactgccaagcacagtgcccaggaccactaagcatttcacacacatttccctaaaactaagagatatacataaggctactcgggccacagtatcaccagccttgctgggccagcatcaccagccttcaactcagctgcctttcctgacagcctctgggctgtgtagctttgtgttcaggactccagctctcaaaatgggtccctcttccgcatgtattcgggagctggtggcagttttgtgtctggtttttatttattggttagtttattagaggcgctgctctccatggcagctgattgggcacgtttccctcagccgtacatgaagggtcccgtttctttcacgataatgggtttatttatatttttcatattttctttttaaagatttatttatttaatgtatgtgagtgccctgtctctctcttcaggcatcagaagaggacattggatcccattacagatggttgtgaggcaccaagcagttggtgggaattgaactcaggacctctaggagagcagtcagtgctcttaaccattgagccatctttccagcccttgtttttttttatttatttatttatttatttatttatttatttatttatttattttgatttacccgttgtcgtgttggggtttggccctaggacgtcacaagcactcggtgaacgctccacgctgcgctggagtccccagctcctgctggagttccttttcctttgtaagatgcagcctcacagcgggcagtggaggcgcacacctttaatcccagcacttgagaggcagaggcaggcagatttctgagtttgaggccagcctggtctacagagtgagttccaggacagccaggactatacaaagaaaccctgtctcaaacaaacaaacaaacaaacaaacaaaaaagatgcagcttcactacgttgctagctccagccacgcactcactctgtcctccacaggtcttgacctttcacctcccgctgcctgcaacctccctagcatcagtcattccaggcctgcaccactgcccggccacctgcctggtctttaattctaggcttcaaaccccagttttgcagcaccagtccccgaacccatgtctttttaaatattgggggaaagccaggacccacccttcctgcccccagacttccggtctttcccagcttctccttcatccatgtatcctctccactaccacctgggaccatgacaggactgaccatagaaaggccacaagcttggagcttgggggcctcccactcctggctctgagttctataggacaggacactaagggggttcagcggctctcatctgaaagtccccaggggcatcccttgcccttggtagtgttcgaagctaaaccctcttcagagctgaactggatctgagccgctccagagttgggctggagtccacccccaaccctgcgcagcaaggagggcaagggacaggaaggaagtggacactggccaggagtcaggtgtagctcctgctctgtccttggcggagccccaaaggtaaacaggtgtgtaaggattccagcagggggtaatgggctgcccctggggcaggtctgggcctgtcctgtggggaggtggaagggcagcattgttaacaatgggatgggacagcagcccaggtcctgcagacacattcttgtgtgaggagaaagaaaacacaggtgacagggggaggtgggggggtgttggggaggggaggggactttgggaggacaccagagccaggcctgtgctaaggagtgtgaactgcagctcagcaggagatgaagtacacagaagcccttggtgggggagggggaggaccgctgccttcctcccatgggacatgtgcccatctcattccgtttctcccaggtgagataggtcagagccacctggacaggcgggcaagctggggcctgagctggcaaagacacctcaaaggacactgtccccagaagctgagaagcctgaactggcctggcaaggtcaggtccagattcagcaggaatcaaagagggacgctggaaggcctgccctgctgacctcgctggccatatggatggtccaagtgcccaccttctcttctgtgcgaggcagcctcagttctgaggcccaaagttctttccctggaggcagctccgctaagctcctaagaaagcaaagcccccaggcagggcctgccactgccacccccatcagaaccaccctgctgggatctgcctctgctgaggcttccatcctgcatgctcccaatctccatctcggttgatcttggaagctaagcagggttgggcctggtgagtacttggttaggggactgtctgggaatagtgagcagaataggcttagccgtggtggaacactggtgaggcaggaggatcgctgtgagtgtgaggccagcctgatctacatagcaagttccaggacagtcagagctatgtggagagaccctgtctcaaaaccaaccaaacaagaaacgacaacagaaaaaccaaaaccaccagccccctcccagcgctgctgtgtgtccaaggagttaagctggggccaggaagctgcccaacgtcctccttgcccagctctacccacaggctctaactgtggcctcgcccccttctgccctctctccgtacagtatcaaggttccctctaacttgccttaatcgaagagtgccggagatgaactgggtacgttgggctagggagggtaaagtgggtttcagactgagttattaattccacatgcctgttcctgtgtcagcaaagtcctgtgggcagacaagcagtccacccagcccagtctacactgaaagcattaaacacagtggccagtgtgctgaatcgtgaccatatgaacgaatggatcattggagtgaaaaacagaaacacagcctttgcaaaaatgcttttaatgtaacgggttcctggcacacacctgcactgaggacatccccaaaactcgtgaagctgctgtggcttgacccaagggtatacagagtctggggcagagtacccaggcctggggagaacttggaggctagcctgcactgccttgttgtagaacaagagaccggtttgctgccctacatatttgtgttccttggctgtggaggaggcagcggaggccttgagggttgaccgaggctaccttagctcaggatctggggaagatgtgagccagatgaagaggaaatgtcgtgaaagggaaagacctggtcctctggtctccaatcctctagggcctggttgcagccatcttaggaggcagtgtgggagccacagaagttagggatggaatgggaaggcagcacacgggatgctccatccaaacacatcggggcagcatgtaagccattcatccattcataccctggccacaaggtcaccaggacagagctgtgcctgactcagctgtccttgcacacctgtaccccactgtagaccaggagcaaacagagcttctgggcctgacactcctattatcctggatgtgctggctcgttcctatagcaacctgacacagtccagggccgttttggaaaagggaacctcagatgagaaaatgttcccaccagagtggcctgtagacaagcctgtggttcactttcttgattgatatgggagggccccgcccacagtgcagaggtctcatcctgtggctctcaaccactttgggggttcaatgaccttcaggggttgcctaagaccatcagaaaacacaaatatttgcattacaatttatttatttatttatttatggatttatttatcttatttatatgaatacactgtagctgtcttcagacacaccagaagagggcattggatcccattacagatggttgtgagccaacatgtagccactgggaattgaacttcaggacctctggaagatcagcagtcagtgctcttaactgctgaaccatctctccagccctacgctacaaaatcataacaatagcaaaattacagttatgaagcagcaacaaaaggaattctgtggttgggggtcactacaaccagaggtactgtgttcaagggtgtcaaggaaggttaagaaccactgctgtggcgggtggggccttccctagccaggtgctccgggagtgtgtaagaaagctgcaggctgactgcatgggtgtgcgtgctatgcttgtgtagcttgttgcttgtgaaactcctaacagtgagagcacctttgcctgctttggggacccttttctccaactgggtcgcctcatccagccttgacactgggtaggcaccctgagaaccaagactgtggccccagcagaagaggaacatctcagcgtgtcaaaaaaaaaaaatggtaaggaaggatgggggagtgaggtcactcagatggacagcaggaactaagggctggcggggagggcagcagggtgtgaggagtgcgggcggctaagtccagagtgaggtgggaaggcagacggggaggggagcagaacaccaggaggcctggccagccccccaatatctcctatttccagctgcccagcttcagacaggtggacaggtggacaggtggacaggattcctgccctcagagaggatcctttggtattggacacaggggtggggtcagccattcgccagagggagagtctacacaggcttctcccatcccgcagtaccttgagcctcagtcactacaagataactaattgcctttgccaggagaaggcagattcatggggttctgtgctcaaagtatgatccctcccaggagatgaatggcctaggacccccttcctacccagaagcagggcacatacccctcaaatgtgccacagcacttcaggtccttcatctgcttggctgacacctggccactggtcaggccacccagtctccttggccactgtcctcgatacagtgtgttctccatctgcagttagtgagacctcttctcactgagttacttacttgtttttagagtacaggggatttaatgcagagccacacacatgttaggcaagtaccctgcttctggtcagcgctcccagctcttcgactcattttaactctttcctgccttttgaattttaattttgttgtttgttatgtgtgtttgtgtgtatttgcgtatgtgagtgtgtgtatttccgagtatgggtgtgtgtttgaatgtgattgtgtgcatatgtgtgggggtgagtgtgtgtttgtgtgtatgagtatgtgtatttatttgtgtgtgtgaatgtgtgagtgtgtgtgtgtatatatgggtgtgactgtgtgtgtgtgtgactctgtgtgtgtgtatatgtgtgtgtgttgggggtcacccacatgacagtacatacatgaaggtttgtagagttagttctctccaggcactgtgggtgggaggagcactgtgggtgtgaggagccctgtgggtggaaggagcactgtgggtgtgaggagcactgtgggtagggggagtgcctggcgtcccttggtccagagcctggttctgctgccgtggagaatgaatggtcttggtggaatggactctgtgggtaaagcatgggaagacatggggtacagaccaagtacccacactccagctctgtgtggtgaccttttgcagagtctggggagaaaaccatgtgacctctctctgtcacccctcctgaaggcttcaggagtcctgatccattgtcagttcagatttgttgaaatctcttgtctgatgctatctcctctccccactgtccacctcctcctgcccctcattctgcactgaagtaaaggtcacaaaaatcaaatgctcagatagggagaccaccaggtttttgtttgtttatttgagggagctgtgtgtgtgtgtgtgtgtatgtgtgtgtctttgtgcctgtgtgtttgtctgtctgtgtgtgtctgtctgtctatgtgtctgtctcatgtgtgtgtgtctgtatgtttgtgtgtgtatctgtttcatgtgtgggtgtgtcggcctgtttgtgtgtctgtctgagtgtgtctgtctttgtatatcagtctgttagtgtctgtctgtctcatgtgtgtgtctgtgtgtctgtctcatgtgtgtgtgtgtgtgtgtgtctgtctcatgtgtgtgtctgtctcatgtgtgtgcctgcacatgtgtgtgttaggaagccagagttccatgtctgtctctccccccacctttagatgggttccaaagattgacccaggcccccaggcttttgctgtaggcaccgtcacccatggggccatcttgccagctccagtttgtccctttttatggccaaatagtattcagtttgtttagccattctcctctgatggatatctgagtaacttccagtgtagggctattgtaaacaagcttttatgaacattcctgaacaaggcttttgtagctctctctctctctgtctctctctctgtctcacacacacacacacacacacacacacactcacacacacaccacacatgcaacacacacattcctttgcatttcatttcatttggcaggtttgggggaaagacctacacgtgtgggctatctgccatgttcaagtgggccacgtcaaggcagccttaaaagagcaggcatgggtattcagcagaaatgcaaagagaaaggcagagctaagggaggtgtgggggtgaatcccagcaggggtgtccagggcgctgatggctacttcatatggggctaaggactctgtcaaatgcggctcaggcaagaacacctcaaagacttcctacctccccaacacacacacactgacagtcgcttgacgctgtcacctgtcacccagcctcagcctcctcagtgctgccatcatgtctatgcactactgtgagggtcacactgggctccccccatcttccccctttcccttttcctttccttctttagtcccaggagcccctagtccaaccaggtgctcagcctttatctggtggttccgttgggagctgactgtcaccttcctacctcatccttttagtagccccaactccttaagtcgtaggacaagtgtcgggcactgcctgccttctgcctaaccaggacctcgccttcctctacagaaacacaaagcctcctccttggggctcctggcaacacttctcaggggagagtctgagtactgagtcctaagcttggggaggtcgccacattagctgcccaggcctctggcaaacctccggctcaaaatgtctggttttggtgagccctgaaccctgatccctagacgggaggccacagcgctggtctgtaacagcaagatagcatcatagaaagaggcaagggcgctcttcagtctttgggacatggcatccttctggggttgcctacagatatggacactgagatgcagaggacctgcggatgccagcacaggtgaggccagcttccaggcatgctggcactgtgtggacaccgccatcttcttgctttcctattactacaggggcagaatcccctgccccatgggaagctcagggtgagtggggcagggactggttcctgtgatatctgccaagagtagtgcagagaatgtggcacagtggagtgggggccgcaacacaagactcagtgacctgtcggcaactggagctttttgccgagagggcagtggggacatggggagatataagtcaggcttgtccctgcctgggcctacacttccccaggcctcaacctagctctcctttctggccttattttaaagcccctattagttgggcctggtagtatatggaagtggagaaagattatgagttaggtcagcctgggctacacgcaaagatagtcccaagaaccgtaaaatgttcgaggccagccaggtctacagagtgagttccaggacagccagggctatacagagaaaccctgtctcgaaaaaacaaaaaacaaaaaaaccccaaaaaacaaaaacccaaaaacgatagtctttattaaagacaactggtggtatgtgttggcatcaggacctcccaaacctatgacatcacataggtgacagcaaaacctgcagactggttgccaaggcaacaggcaccctattcccaacatccacttggctcactttccacctttacctgagttacgtcattctcgatataaataaggagaactcccctccctcacactctctccccctcttctctttctgccgccaccttctctctcttctcccaataaaacctcgtccacgtggaactgtcttggcctagcgtgctgttgagatgcaacccgccattctaacacatcagtgtgtgtgatgtctttccaggtcgggtgtacactgatggcggctgcacacatctttctttggcctttctgttcattcattcattccatcagtcactcgacgagcctcaggctggaagactctcctgtgggtgcgcagggcgctcttaaacacactcagtaaaaccgagccttggtcctagaacccttgcttcagggtagttctggatcaagggactaagacctcccatttcccaccacagaggaaggatctgcaagtgttgtgctggggaggaaaaaggccttctctgcacccccacccctatccagcagctccggagaccccagcctgaatgtggtctgcctctgcccagccacctccagtacctggggcactggtgtggactcttcacttgtatcccagtcacaacgaagccacattcttctttatctgtcttgagacagggtttctctgtgtagccctggctgtcctggaactcactctgtagaccaggctggcctcgaactcagagatccacctgcctctgcctcctgagtgcagggactaaagacatgcggatcaggtgaaaccacattatcttacctcagtcagctgctctaaagtgccttagctttcttagtcgatacctgttcttactcctctggtgacagagccatctctgccttacttctcacagaatagatatcttcctgggacccaccttcccacccttcagaatccaggacacatctgtgcctctctggtgggccattcccctgtggtggtttttcctttaaccagtccccacagtccttgtccctcctgtgactggcaggacatagatttgctgtgaatcccaaattctcaaaaggactttcttttctgcagtcttagcagggaagggggatctcttcctgtctcttccctcccaggctttccctagttttctctctacctacacagacaggttattgaaaaccccagcgatgtttgtgatcttgcacaggaacaagggcaaacgtccctatgcctggagtcagggagcctctgtcctcatgtgtggattgaaccccacgctgccaaacaatgcaaatcaggatgcccagcatgtgaaagcgctgagtcacattctgggaacaggagccctggggaccatgcccttcagcagtgcgtgctgccgcgtttgcgttgcaggagtgccccgggggctgcgctgggctgcgggaacatgcttgtaggtgcaactgggctatggaatcgcgctgggagctgcaattgggctatgggagcactccggggactgcaactggactgcacctgggctgagggagcacgcccagggctgtgagggcgcccctgtccaggccagagctgatctccccgcagcaccagctggactaggagactagcgatcacgggattcggggcgcgctgggcacccactaaacttccggggccagaccgcaggcccctggtccaaaaagaactgccccggttccagacgacgcctgcacttcctctctccctagcggcgcgcgccccctggtggacgccgtcctgctcgctgcttccccaccgcgctgggctctaagctccccgcaggagagtggtgacccccttcccagagccagcccaggataggagaatagtggaaaatgccgtggtctgaaaagataacggtgtgagctagtgtgcatacgtgtgtgagcatatgaaatgatacatgtgtaaataaataagcgttgtgtatgtgtgagtggatgtggtatggatgtgcacatagtgtaaatggggcactgggtgggtggctgatgtgtgattaagtgatccatcgtggagggcaggggcagggtgtatgtctagtggcctccagtgtccaggcacggttgggtaactcctgaaagcctttttgtgatactctagggattaaacctagggctttggaagttagccaggccacactcttcttaactttgttttgtgttctgaaacagggtcttaccacgaagtcttagctggcctggaacttcctatgtagaccaagccagcacagcgggtagccagcttctgcctcctgagtgctggggctaatggtgtttgccaccacacctggctctttttaacttctatatttacatttgtaaattctctgagaatttcattcatgagtactgtatttacatcattcccaccatgcccttccccctctagctccccccaggggtgagaccttaagaactttctcccactgatgaaccaactggtgtcactgtgttggtcttgtctaggtagccatattgttgagatttcttgggtgtaatttcccctgctgtgtctacaagacactgtcttgttggtcctcgggttcttacaaccgttccgtgaatcagtctctctctctctctttctctctctctgtgtgtgtgtgtgtgtgtgtgtgtgtgtaggttgcattacagctttaccaggttgagttgtgcaccccacagactctaattctctgcattcagataaagtcagcttgacaccagctagagtcatcagaggaagcagtttccagttgaggaaatgcctccatgagatgcagctatgcgcgtctgtaacagtctccatctgaaaaagaaaacaaaatcattctttgatgaaaagcaggagctacagtcatctgatggtctaactatagaaaagttatttattttatgtgtgggttatatgtgcatgtctgtatatcacataaatgcagtgaccatggaggtcagaagagaacgttggatcccctgaaacaggtactacatacaccactgctatgtgggtgctgggattagaacctgggtcctctggaagagcagccagcattcttaactactgaaccacctctccagaccctggcctacatatttaaactataattagaaagtatactggtttaggaaaatgactgtagttggttctcatatagggcctataaccatgaacccacatagcctggtcttaaagtctgtcctggctggctttgtgtgttagcttgacacaagctagagtcatcagctcagttgaggaaatgcctccatgagacccagttgtaaggcattttctctattagtgatcaatgaaggagggctcaacgtattatgggtggtgccatccctgagtttgtggtcctgggttccatagaaagcaggctgagcaagatataaggagcaagccagtaagcagcacccctccatgacctctgcatcagctcctgccttggttccagccctgcttgaggtcctgtcctgacttcctccagcgatgaatcatcatctggaagtctagctgaattcacactttcctccccgacttgctttttagttatggtgtttttttcccctctgtagaaagcctgactaggacaaagcccttctgtagtgcaggcaggtcctgaatataagagaatcctgcttcagcatcctggaagctcagcccaggcgagcacctcttggatctgtgttacacagctctgacccggcctagcttagattcccactttaagattcagaggtgtgctggagcgatgactcaggtgttaaactcactggctgctcttccagaggacccaggttcaatccccagcacccgttatggcagctcacatttgtctgtaactctagttctaaaggatcagacaccttcacacagacataaatgcatgaaatgaggggtacaaaagatccaaaggaagccgggtagtggtggtgcacacttgtaatcctagaactcgggaggcagaggcagatgaatctatgagttcggggccagcctggtctacagagtgagttccatgacagcccaggctctcctaccccaaaaaagatccaaaggaaactgagctcagtggctcagacctttaatcccagaactagggggatagagtcaggaggtttgctccaagctagactacacactgagttccaagtcaccctgagctacagttcaaaatcctatctaataaaatatagcaataagccgggcgtggtggcacatgcctttaatcccagcactcgggaggcagagacaggcggatttctgagttcgaggccagcctggtctacaaagtgagttccaggacagccaaggctatacagagaaaccctgtctcgaaaaaccaaaaacaaacaaacaaacaaacaacccccaaaacaaacaaaaaaaaaccatagcaatagaatttggggagatgcctccatgggtcaaaaaatccacatagaagccaggcatggaatgtttgtaagtcagagacagtggaatcctggaggtttgctacctagccagaattagtcgttaagggagggatatttaagaggatgctcttaaaaaccctttgaataaccctctgttatttaatttgggtactctgggtgccgaggccag

SEQ ID NO: 48 - DNA, 11967 bp, Homo sapiens
ccgtgaggtcagtacgcatgtcccatctgatgggtgagaaaaccaaggcccacagtaaactgatgtgcccaaggtcacactgctgtaaactgggagtggagcccaggttagcgtgactacagatggagtcacaacacggccaggaagtgagcccacaggaggctgtggaggattggatccacagcacaagggaaagagaagcaccaagaatgggtttccagacaggcgctggcactcctcatgtgggggcagcagcagatttggggagcggatgcgtttagtgatggtatctgtgggaccccgaggctcttgggctggtgtctgctgggctgatggcaccagctagtagcaaacctcaacagctgcgtaggtgcaggaggagaggaaaacggcaggtgacggggactggagcctcacactgtcctccctccaggcagccttccatcccaggaaccctgctgggtggccagtcctttcctgccccgcctgggcccccacagaaaacattaacactcggccagaacgaacagtcttctcacttccccccacctcttcctgccccaatgaaaatagctcagcaaggcccaatttccttttcctgtctattgtaacatataggcaaactttaaaaccaagtaattaaaatacaaaagtggatcccagggtaagttcgcaaaacaaggaacatctttcatgtccctccctgtccgggagccacagtggcagtgactgtacctagcccagggggcaggagcaggctctgcacctgtcagcacctgcttctctccgttctcacacacgagtgctgtggctcgcaccgtcatcggagtctcaggtgacacgcatcctgctccttccgtgccggctcctgggctgccctgctggagacaccatctggatttggccaaaggtctggctcctgtaatgttcattttctcttccccaggtgcctggcaggctgatgacaatgccgtcagtcggaatgcccagcgccggcgcttgtcatccagcagcctggaggactctgagacgaggccctgtgtgtggggccctttggctgtctgagccccagcctctgcacttgggctcccctcctgccctgcattccatccatcctcactcagctgctgcctactgggcatcctcagcaagatgctggagactttttaccctcctgtgacagctgttacaacctgtaaccacaatccctcacctctcagagggaactggaaccctcctttctccgtgtgacctaaagccactcaatgaactgcagctcacgagaccttttgcagggactctggaatgtcctcacggtatattgatgttcaggtggagctggggtccatgggctaatttcttacacatcttttttctccttagggggtagaacaagaaggcttagagtttggcagctttgttacaaacatccctctggcatttcctggtctctggcacatcataaccaattggaaacctgcagtttggggtagggctggaggatcgctcctgtggaaagggagagccatactgcacggacgtagcgtttccctctacaagcactaggagcacctttctcaaggacaagaaatggtagcaaccaaaaggttggttgaaggaaacaatccttccttctggtggcagagccagtcttctgggcctcacatgcctgtgcggttaagaaggagctcttggccgggcgcggtggctcacgcctgtaatcccagcactttgggaggccgaggcgggtggatcatgagatcaggagatcgagaccatcctggctaacacggcgaaaccccgtctctactaaaaatacaaaaaattagctgggcatggttgcaggcgcctgtagtcccagctacttgggaggctgaggcaggagaatggcctgaacccgggaggcggagcttgcagtgagccgagatcgcgccaatgcactccagcctgggcgacagagaaaaaaaaaaaaaaagaaggagcccttgaatctgagaatcccccagttaagatgatttcccttaaggagcatttctcctgatcaggtgcattcagggaggcctgcaactccatcctaaggtgacatttcaagaaacacatgtgttgaccacctctgtgtgccaagcactgggtgaggagtggtcgtatattaccataattcatcctcactcaattgggaggtggcattcttgtccctgttgcccccatctctacccccttttgttaatggttgagagaaatgtggaccagtgattctcagtttggtgcccaggtccccaaaggaagtgatggagagccttgagctaagttcaggcagcagcatgtggccagcagcagtgtgctgggagcagaggttggaatcagggctcagggccttcacttcaagggccctccccagctactcgggaggctgaggcaggagaatcgcttgaactcaggaggtggaggttgcggtgagccaagatcgtgccactgcactccagcctgggctacaagattgaaaagatcgaaactctgtctcaaacaaaaaaaaaaaaattcaaggctgtttccaaggcagcctcaaaaagagcctggtgcccagggggtccttcgtttctgtgtggctgcagaggccaggagtgcagtgactgtctcactgcctagtggttgtggctcacatcctgccctgatggagaggggggcctgtcgtccaaccctctctgaaaaactgcagtagactttaccatttcaggccccacctaatgttctttccttccttccccctacattcttcccgcccctttcatagttaagatgcataagctgagcaataaccccacttcctgatttccctgaaatatgagcccagaagcagcaggtgacagggagcttcccttaccctggggtggtggcacacagaccacctcctgtcctgtcccctgcaggtagtattcacagtgtggtgttacctgggaggagggaggaaggagctgagatgaaggagattcaggtaaacagtagccacagtggccaggacccccttcctgaggatccctgtccttcctcgcagcaggtgcttagtcctctcctagggagaagcacggaaagattcctcaaggcctcagtcccatgtccaaggcatacctaggttagaaatgtcctttacttgtttctcagttactatttctcctcttcctccctccccctgccttctcttggctgtcatttagctctctggcccacagaaatactccatccaaggagctggtcctcagtcagcctgatgaggacaggtcagtggccaacccttttgccccagtccttcatctggagacagtcacatagtcacgtcaacagttcacccaaagagaagaggtggggcctggcttctcccacagggcacagcctaactatggagacccagggctccttcctgctgcactaggcctgtgctctccctattgccttcaggtggtgtgatcccggcttcctcacagagcccttttttccttctgcccagctccatcccaccagagtttctacccccgctgaggcatctcctgggcacagaccaccacactctaagccggctggcctgggagttagggtcctgcccacctcacctgggagttggggtcctgcccacctggagtttagcacgctggtcgctcactttgctttggtctgcacacctggtgtgtgcatgtgtatactgcttgtgccatctgtgtacccctctccctggagtacacttgtggtcacactacagtggggacatcaagaatgcaatatttatggattgctgcatgattcttaaaaatgaataaaactgtttacaagggaaggagagctaactgcaaagtgagcttatctgtgaaagtttctgtctgactttcgccagcactctttaatacttttactcaagaggatctggagttggaggagtgggagaggcaagggctagggaaaggcatgttggcgggggagtgatgcgccatagccgggccttcttcatggtctgggcaccagcctccccgaccccggctctccagctcctgggcagaggcggggtaatcaacccagcccccaccactgcctctgcagccgccttaaaccggagaacgtccaaccctgcagctcctcaataattaaagagcaggaaagctggtgacaggagggaggccaggggaagggaggaggaagggacacagtccccagagtccctctactcttaatccctgtgacttaaaggttggtgaaattaattgggcaaatgaccccgctaggagaatttgacaagtccatgacttcagagttttacccacgggctctgcttcatcagctgtctccagaggagtgagtgcgcttctgcagaagcctctggaatgtcagcttcctggaacctcgaggatacgggtggggaaggagctgcttactgggacacactggattaccctacccccttggtgtgtctgccgcctctgtggatgtaagcggcaggtggcatcggaaagtcagacctgcctgggacaccagagaaagaccctaagagtcgggggagagggaaaggctgcagaaacatgagaaagtgccactcgtagcggagaggaaagaaaacctcaaggatacggttcccgagcctgagcccgctcccagccttagccccccaaccccaggctccgtcccaggagcagtcgacgcctcaaagcgtacgggacaggaagggggtgggggcgagagtcttggttccccagaacgaacggatccatagcggttcccggtgccgcaggccgcgcgcttctccgaccccagcgcggggccctcgggcgggaatgagccgagccgggacttagcggcgggtagggggttgggtggcgcgggagagggcggcagaaggagggagttcgaagagagttgccgccagggagagtggcaagtgcccgcggtctccttggagacgtgctggccaatggcagaggcggaagctgtttatgggggcggagcgtcgccatggcagcaggagaagcctttggagcggctacttaatgccggtcccggggccgcggtagctcagagaagcgccgtcgggcgggggggcggcaagaggcgctggcagggcccccgagctgggggcagagagccgcagggggtgggagcaacttcttccagaaccttcctctggcccgggctgcgctctgagccaggtaagggatggggagggctgcaggccagggggaagggtgggcaacggtaccaacccgctctctcattcgtccttaacggaagtggtgggcccagggactgacatcggagaagtctgggggaaactcgcctcctgccccccgttactgttccgtccccttccttcccaatgatcccctatcataatattccttttcaaggatcagaggacctccaccgcccccgccccgtcgtaccgccctcccaaacttctcgccccacttccatttgttgcgaggaggaagggggtcctgatttcctgctcttcgggagaaaaggatagcctcggatgcccctaccctcttcatttggctaataaaacctcgaagaggaaaggaagagggaggcagatctgatgggggtgggaccccagggccttgcttctgtttcccatacctcggcactaaaagatcctccccagctgcggggatgatgttagagagggcgtccccgtacacacactgtcccctcgggggtggggtagtgctcagctcccaccgtggtcctcgcaggcaccacatacttattcggaggaggcgcccccggggtcgcgagatggaagcgcgcgtccgggaggccgtgtaacgggagctgcgggactcagcgggccagagagcgcggcgggccacccccggctcagcccgtggatgctgaccgccccctcggagagtccccgcagacatggcggagagctggctgcgcctctcgggagccgggccggcggaggaggccgggccggagggcggcctggaggagcccgacgccctggatgacagcctgaccagcctgcagtggctgcaggaattctccattctcaacgccaaggcccccgccctgcccccggggggcaccgacccccacggctaccaccaggtgccaggttcagcggcgcccgggtcccccctggcggccgaccccgcctgcctggggcagccacacacgccgggcaagcccacgtcgtcgtgcacgtcgcggagcgcgcccccggggctgcaggccccaccccccgacgacgtggactacgccaccaatccgcacgtgaagcctccctactcgtatgccacgctcatctgcatggccatgcaggccagcaaggccaccaagatcaccctgtcggccatctacaagtggatcacggacaacttctgctacttccgccacgcagatcccacctggcaggtagaggaggcacgggcggcggccgctccctccccattccctcctcgctccccacgctgcactggattcatatcccaaatctgcaggccaagtaggctctgcggtgcggccagcgccccagaagggagccctgcaaaggagcccagttcttgcaactgcccctcctttgacttccagagagagagagagagagaagggagaatgtccagaggtggagggctttaggggtgcagaggctgagattcaaggggaaataacctgccattcatccagcaggcccaggccaaagaccagggctagactctgcgtggacagaatctccttggggagagcgatgaactgaaggcgcattcttgtcccaagtcctagaattcctagacactgccctgccccgggatggtttgtcggggccgggagtcagaaacggaaactggaacgtggcctccctgccaggctctacgtcctgcccttttgcagttggcttggggagggtggaatggaaggggtgccttagcatgtgagggccaggtgtgtcccaatggagggtggagggggtaaggggccttcctaaagctgctgtcccagccccccacctcaacctctgctcagtttcctgcctccaacccccgctgtcagccaacctccctggggggacaattacctgagccaggtgccttccactggggctctgcctaccacactcacttcgtgttccacgtctctggaaccaggcactgagcagccaaggggcccaggccaccatgtaaacatcccagccttgctgccgggcattatccacccacctgccctgccgtcccagcccaggacccctgtcccccagcaggctttggacaagattaagcacctaagaatggacaaggcaggaggatggtggatttgaggctgggtaggcagtgccctaagagtcttcagccatcaccatcaagggctagttggagtcaggggccccccaaggacgcccagactcttccctgaagcacccaaagcgggcaggccacaggaccgcagtcccaagccatttccaatgcccctgaccctagagcagggtcatttgcagctgcaccgaaaaagcagagaccctcagccccaccctaccctaagcctgtgcagctgcgcttcgagaaacgggccccaggaatgagtgctatgctcttagctcttggctgggtaataatcaactgagcctggagtggggggagggggccgtggggggtgaaggcccaggccagcagccggctcaacaaacaaggaaagtcatcagatcccccacctctcatccctgcgcgctctctctctctctcccccttcctctccactctcggctgcccagccccctccctggacccactcactcacagagagggccagccaggcccaggcaaagggagacggtggtccaggccctgggggagccgagggctttggccagaggctgtcagcggcagggtgagggagggagggcaggcgacgggggtggcaccgagagccaccctgcggctcagagctcggatgactgagcaggcagatatcccggcaacagggaacaaaagcctccgacagcactccgggctcccagtgccctgtggctggtgtcccagagctgcagccgcctgggcagaccagtgtgtctgtccccccctcctctcccctgccagagaaaagatggggggtgcgcagcagcgccaggtgccatttcatctccccgtccccactgacctagcggttcctctctctcttgctctctccctgcacccagaattcaatccgccacaacctgtctctgaacaagtgcttcatcaaagtgcctcgggagaaggacgaaccaggcaaggggggcttctggcgcattgacccccagtacgcggagcggctactgagcggcgctttcaagaagcggcgactgccccctgtccacatccacccagcctttgcccgccaggccgcgcaggagcccagcgctgtcccccgggccgggccgctgacggtgaataccgaggcccagcagctgctgcgggagttcgaggaggccaccggggaggcgggctggggtgcaggcgagggcaggctggggcataagcgcaaacagccgctgcccaagcgggtggccaaggtcccgcggccccccagcaccctgctgcccaccccggaggagcagggtgagctggaacccctcaaaggcaactttgactgggaggccatcttcgacgccggcactctgggcggggagctgggtgcactggaggccctggagctgagcccgcctctgagccccgcctcacacgtggacgtggacctcaccatccacggccgccacatcgactgccctgccacctgggggccttcggtggagcaggctgccgacagcctggacttcgatgagaccttcctggccacatccttcctgcagcacccctgggacgagagcggcagtggctgcctgcccccggagcccctctttgaggctggggatgccaccctggcctccgacctgcaggactgggccagcgtgggggccttcttgtaagaggccaggccctgccccacctctggacagtgcccaagtcagggtccagaactgccccccaacacaggtccacagacaccccaccacctaggcaggggctgggccagggctccaaggcttgccccagaggccacatggccaccagccccagctgccatcagattcaagcccaggaggctgaaaacgagggcccaggaccagaatcgctgcctcctctccccagccccaccttgtacacacagtgtttcattgctccgcgtcttcccagccccagaaaccggctaaaggaccctgcaccatgagagccgaggcctggaggagcccgggtcaggctggggaggaacagaactgggccctcccagagcacctccgcttcccccctgcttccccaggtctctatccagagagagtccccaggtacaacaaatgctaattagatgacagcaaattaaccccctggaggcttctcctggcagagcctccctggggccggggcaggctgtggatggggcggagcagggcagaagatggactgggggagggggcagagagaggagaccaaaatgaggtggtggcacagggtggggcaaggagatcctctctaaggcctctggggtctttgcctggccccatccctagggggcggggaggggacgtaaatccctaatctttaagcccgacttgaggctgagagcagctggaagtttgggtttggtggtttgggggccggggcagccaagctgtatggggcaggacagacagactaatgtagtgagtgtagctgtagctgaggcttaactgggagggatgccgagcttgctggaactactgggaccaagaagcggggtaccccacgcccctgcctgcactcctcgggggcgtggggcgtgccttgctccacccggactccctgggctgcgtcccacatccaccctcctgccccgtggggcaatttaacctttttcatgaaagttatttacaatgaaaagtttttaaaaataaaatttttaaaaatctaaatatggacctgctcaaaactttctttgggaacaggtgagggctcagtgttctggagaggcagcgcttctctttgctggagggagggatggctgagaggaagatgcgggggcacttacggcctgcaaagagcactaaccaactccccatgacctcagcatccccggggaggaaggggctgaggggtggctctcggttgtcccctgcagcctaggcctcagtgcctccatctgtgcaatggggagactggggtccacagccctgggggatcaggagacaaagggaggaggagtggcctcagtccccagacccagggaatctcaaaggctaagtgtgccctgggctcggcccctgctgtcccctactgcgagggcctgcccgggcccgcctgccccagtcagccatccccggcctgggctggtctccagcacctccctcgcttggtcctctcctggagggacccagctccagggcccccacttgccacacaacctggcccaatgcaggtttggagaggaggaaggccaccagggaagggagtgagctcagacccacccctcccacctacactggcaggaaccagactaccggagttctgatggggaaactgaggcatgggaaatttgtgtctgggtgacactcagccagcacctttcaatcccccgccccccttgctttgcccgggagccaccttccaactcagtccccgccacatatacaccctcatgggtgcacgacgcctccagaaagccacttcctcgcacacgggcgcacacacacaggggcacaaacaggcaacgtcccaagcattcctgcccagcctccacggacccctttctcccaaagcccctttctttcttcgtcctcggggttggctctaggccaacctggcattcccctgctaaccggctgctttcacttgctaattattctaaatactgcaggagggcacgcagcctgcagggtccaggaaggcaggctgagggctgccccaattcccttcaccccgcaagcaactcccctctccttttccccactgtccgcccccttcctctccatggaccccagcaatgggggaacaagctgggctccagccttctccccacaggatcagcctccagctttggactgacaggctgggcgtgtagctgtgtgccccaggcctgtgctggggggaaggggggcaatgtctggggcaggcaggacaccccactgggggagcaccaggcctgagaagcagatcgaggtttgccccagcttgacagtcacctggaggctcctgaaaaatcccactgctgggctgcaccctggaccaattagaccagaatttccggggtgggacccaggcatcagcatttgtaaggtccccagatgtttccaatgtgcggcaaaaccgagagggggccctggagagggcatgactttcagcaggccaagctggggagtggcgagagtgtgggctaggagctcctcactcctattcacccctgccttgcccctcagtggggtggctttttcgtaaaaagatgttcatttacctctttttttttcttctttttttgagtcgtctggctctgtcgcccatactggagtgcagcggcgcgatctcagctcactgcaacctgtgcgccccgccccaccgggttcaagcaattctcttgcctcagcctcccgagtagctgggactacaggcgcccgccgccacgcccggctaatttttgtagttttagtagagatggggttttaccacgttggccaggctggtctcaaacttctgacctcaagtgatccacccacctcagcctcccaaagtgctgggattacaggcgtgagccacctcacccagcccatttacctcttatttatttccaggttcaatgtaatcccaaccaaaatatgttttgcaccatttgacagtacgttgaagacctgactccccataccactaaatacttcagtaagtatttccttaaaacaagggcacttctggattttgtttgttt

SEQ ID NO: 49 - DNA, 2599 bp, Mus musculus
agagaagcgccgtcgggcgaggggggggggcgcgaagaggggcgccagcacagccctcgagctggggacagagaaccgcgggaggtgggaggaacttcttccagaaccttcctctggctggctgcgctctgagccaggcctcacattcgtgttcggaggaggcgaccccctgggtcgcagaatggaagtgagtgttctggagactgtgtaactggagtctgggttaccagcgggccagagagcgcggcgggccactccggctcagcctgtggatgttgaccgccccttcggagagtccccgcagacatggcggagagctggctgcgcctctgcggagcgggtcccggagaggaagccgggccggagggcggcatggaggagccggacgccctggatgacagcctgaccagcctgcaatggctgcaggaattctccattctcaacgccaaggctcccactcttcccccaggaggcacagacccccacggctaccaccaagtgccgggcttggtggcgcccgggtcaccgctggcggcagaccctgcctgccttgggcagccgcacacacccggcaagcccacatcgtcgtgcacatctcgaagcgcgcccccggggctgcaggccccgccccctgacgacgtggactatgccaccaacccacacgtgaagccaccctactcctatgccactctcatctgcatggccatgcaagccagcaaggccaccaagatcactctgtcggccatctacaagtggatcacggacaacttctgctacttccgccatgcagaccccacctggcagaattccatccgccacaacctgtccttgaacaagtgcttcatcaaagtgcctcgggagaaggatgagcccggcaaggggggtttctggcgcatcgacccccagtacgcagagcgcctgctcagtggggccttcaagaagcggaggctgcccccagtccacatccaccctgcctttgcccgccaggcctcccaggaacctagcgctgccccctggggtgggcctctgaccgtgaacagggaggcccagcagctgcttcaggagtttgaggaggccacaggggaggggggctggggcacaggagagggcaggctggggcataagcgaaagcagccgctgcccaagagggtggccaaggtcctgcggcctcccagcaccttactgctgacccaggaggagcagggtgagctggaaccactcaaaggtaactttgactgggaggccatctttgaggctggcgcactgggggaggagctgagctcactggagggtctggagctaagccccccgctaagccccagctcacatggagatgtggatcttacagtccatggccggcacatcaactgccctgctacctggggacctccagcggagcaggctgctgacagcctggactttgatgagaccttcttggccacatccttcctacagcatccctgggatgagagtggtagtggctgcctgcccccagaacccatctttgaagcaggggatgccaccctggccgctgacctgcaggactgggccagtgtgggtgccttcttgtaaaaggtcaggccccaccccatctccggacagtgctcaagtcagggcccagaactgcctcccaagacaggcccgtggacatctcagcacccaggcagggactgggccaggtctctgaaggccacactgctgccagctggggatgtcctcatatccaagcccagaagactgggaactggggaccctgcccctcccccatcagcaacccccatacacacagtgtttcatccacccatgttccctcaagcccaaggattggcaaaagcacccctgcccctgcctgccccccatccccacccttgcactctgagagctgaagcccagaagggtcctaccaggccagggaggaactgaacagggccctcaccaaagcttcgcctcacccacttctgctgcttctctgggtctctacccagagaaagttcccaggtacaacaaacgctcattagatgataacaaattaattcccaggaggcccctcccggcagagctttcccaggggccctggcagccttggccagggagagagagtgagggcaagagactggggaagggaaaagaaaggaggcagaaatccggtgggggcagcgaagtggggcgaaatggtctctaagcccgctagggcctttccctggtccatcacctgcacaagggaggctacaaaccttccagcctgacttgaggctaagagcatcaggaagatggggtgtggggtggttgggtcaggggcagccaagctgctagggggtggggggagagactaatatagtgaatgtagttatagctaagacttaactggggaaaggatgcccggctcactgggaacactggggccaggaaggggacaagccctaggcccctgtctatactagggggaccaggccttgctccaccttgacccctggagctgggacctgaatctaccctccacgtggagcaatttaacctttttcatgtaaagttctttacaataaaatgtttttcaaaataaaatttaaaaaaatctgaaaaaaaaaaaaaaaaaaaaaaa

SEQ ID NO: 50 - DNA, 2353 bp, Mus musculus; identical to SEQ ID NO: 45

SEQ ID NO: 51 - protein, 150 aa, Mus musculus
MERRRITSARRSYASETVVRGLGPSRQLGTMPRFSLSRMTPPLPARVDFSLAGALNAGFKETRASERAEMMELNDRFASYIEKVRFLEQQNKALAAELNQLRAKEPTKLADVYQAELRELRLRLDQLTANSARLEVERDNLAQDLGTLRQ

SEQ ID NO: 52 - protein, 306 aa, Mus musculus
MASSGSVQQLPLVLLMLLLASAARARLYFRSGQTCYHPIRGDQLALLGRRTYPRPHEYLSPADLPKNWDWRNVNGVNYASVTRNQHIPQYCGSCWAHGSTSAMADRINIKRKGAWPSILLSVQNVIDCGNAGSCEGGNDLPVWEYAHKHGIPDETCNNYQAKDQDCDKFNQCGTCTEFKECHTIQNYTLWRVGDYGSLSGREKMMAEIYANGPISCGIMATEMMSNYTGGIYAEHQDQAVINHIISVAGWGVSNDGIEYWIVRNSWGEPWGEKGWMRIVTSTYKGGTGDSYNLAIESACTFGDPIV

SEQ ID NO: 53 - DNA, 17566 bp, Mus musculus
agatctgcacccaggagcttcatgttaagttctggcattttctggaagcgccatcttcccacatgtaaaatggggtaataatatctacccgtcccagggcaggacaggaggaagaatcaaagcaggactaaggtgcctgctccagcaaggtgccaagctgggatgactcacattatctctgggaacagcccagggctgccagttactcaggcagagaaccgtgtagtggtttccggaaacatgcccagctcacgagcacctgcttcactgccccaagcctgggtggcagagacacacgactgctcataaaacacctgctggaagcgacgacatgtgtgtgagcagagcctgcaaggcaggaaggtgcgggccctgccctgactctttctttccaggcccaaagtaggagatgaagtcactgtgcccagaatgacctccccgacacctgatgtggtgtcaaaaggggacacacctttcaggctgtggctccatgctgccaggccattgagtcggctgcggggggggggggggggggcgccagccaccgggatgaactcagccttctgcacgcatgggaccagggcagagatagaccacataatttacacttcccgggtcctagcaacctttacccatgtgcccttcagagaagcccttaggactgaggtccttcaagaacagcagcagtaaatattggaatttgcaacaggacacacacacaaatacacacacacatcaaaaaaaaaaaaaaaaaaaaaagacccgtgaaaagggtgcccctttagcatggacttacaaaattataaagcagggttgccagtggggtctttagagcagggtggttaaatattactccagcagcactaaacttcaatgtgaagagcccttggcagctcggctatgctgccatcacaaggggctggaataacatacagagagtgcgtcataggcctgtttctttcttggcttaaaaatcttgggcccccttcccctctctctaaccaacctctcttctcgccacctcccatgctctgatagggaagcccaatgggcttctgctcacccatcttggacaatgtgtacctcactagcccgtaatatcgtaaccccttcccccactcctttaagcagtgtttagtgagattcgtcagataaatgcttccttgtgacagcgcttacagagcaagacgacagacgggtgcctattagccttcacgacttcctgcgggccacgcagatgacactaaggtcatcgcacctgctatgcaattttctgagcctgggaaaaagtgaagcctcctaagctcgtggacctacgagacagggccggcgaggggaggtcacatgccctcgtggcatgcccgtcatatctaaatagcgtctgagtgtttgctgtgatgatgcccaaacatgctcacagcaccccagggttgtcaacaccacatggccagatgaagatggtaagctcccttgtaaaccatcgttgagacatgagcgctcagtctggggccctcctgtctgctgactccaggaagactgtaagccctggaatgtgtgagtggactttgcaccatcaagaagaccctggcctatcttccttccccggccatcttatcggctgattttggttgggggttgtcctgtacctaaggcatgaagatggtggctgcaaagaagatgaaaaagtctctggagtcgatcaactctaggttccagcttgttatgaaaagtggaatgtatgtgctgggatataaacagactctgaagatgatcagacaaggcaacgcaaagttggctgtcctcactaacagctgcccagctttgaggaaatctgaactagaatactatgtctaaaactgctagaatattggctaaaactggtgtccatcagtacagtggcactaatattgaactgggtacagcgtgtggaaaatactacagcgtatgcacactggctatcattgacccaggtgattctgatattattagaagcacgccagaacagactggtgagaagtaaacaagaaagtttcctttaataaaactttgccagagagccttaaaaagaagaagaagaagaagaagaagaagaagaagaagaagaagaagaagaagaagaagaagaagaagaagaagaagaagaagaagaagaagaagaagaagaagaagaagaagaagaagaagaagccgcccctggctggcttctggggggggggggggacgacaacctctgttactcacaaaatattttcagctcttcctttcttccttctttccttcctttcattcattttaattaattaattagctaattaactaattcagaagttgagcacacagactgcccttccagagggccagagctctgttctcagcacccacatctattggctcacagcagcttctaactccagttccatggtgctgggtatgctcttccggcctccaaggacacccagactcatgcacacatataacatataggcacacatagacgacacattatttaaagtgtatattttgttaattttaaaactccactcattcatttgctcgttatctgtgccagggattgaacttgggaccgtgagcacactgagtacacactctcccgataaacgctgctgccctattgtgctcttcactagaccttgcctgtgggatgccatagaatcgccaagaccaattgtccccttcctgcgagagagagcccatgtggaacccacgtgggaggcgacactcccccagagtggaccgaagggctctaaggtcaatgcatggtcactactggggagaactggcttagggaactcaggtctctgcctgggccttgggaggccctcattcctaccaggctacttaccacccagcagaagggagaggttgccatgaccccccaggatgaaagatttcaacatacagatgccagtgttcatttgcctccgtgactactttatagcaacagggaacaaaactcttgcgttgttaagggtagccccaagtttgcttctgaggtcttgttctgctctctgacaggaggtatccagcaggcttttgagtcttgttggaatcctgggtgatctggaaaggcaggcagcaagacagccattatgatttctgactgcccctatccacagacagaggaatggctgtagccccactggccatcgcgcatcaggaatctcacacccaccacctggagagagcctgagataaaacaccagggaatgtttcttacagaagaagagatagctttgtggcctgggactgctgacaatagctcacggggcacagggggaaggggaaattctcccattacgaaacctaggctggccaggtcccacttctttctcagttcctcatcttggaagagtttggttagcagggaccctttccagaaccccagggactcacgatatatgtgccaagtgaaaaaaaaaaagaaaaagaaaaaagaaaagagtaatcctggaaggatccttctctgtgaaggagagctgtctcttcaggaaaagcctgggtcttggggggtggagaggaggggctcagaaagccaggctagagtccaatgctgggtgtgacccacaccttcttccagcctcaggatatctctgtgcgctcctagggagacctaacctaatgggacactgcaccacactccagagcagagaaacaaaagaatctgggctgagtgggccaatggaagcctgctgagccctaccctgctccaggatgcctgggtctctgaatagtaaacttaagttttgctgtcagtccgtgttatcacaatggctggtggcactgtcaggtctggggtctggttgtgtgaccccaggttctgctgatccggttgtttgattcctagtgaggcagattatctcctgggggctgccgagcggggcggggcgggggcaggagcagcggggaaggccccgccctcggggtgtgtccaacttgggcgcctcctaaaggaccgggcggggcgtcccgagcgcgtgggcctgcgggtcgggtcaagaggtcgaaggtgctgcgcgtgatccaggatccgaattggcccggagcaggagcatggcgtcgtcggggtcggtgcagcagctgcccctggtgctgctgatgttgctgttggcgagtgcggcacgggccagactctacttccgctcgggccagacttgctaccatcccattcgcggggaccagctggctctgctggggcgcaggtgggcgcggggctgtgcctagggaggaaaagggtcaacttctaggccaggcgcgcaccccctgacacttgggaccttgccccatcctccctcattaggcctgggaagcacaaatcccatcccttccttttgggatgattttcacatccggggagctctggggtttgctggtttgggcagttctcgcctctactgcctttgttagtgagcattagcgtatttcggagatgctctgtcacattcagcttccaatagtcctgggttctgctggtggctcttctgggtcaagttctgaaccgaccacagtttgggaagaaggccaacaggagaaatgactggatttgtggccctggtgggggtagggagaggggaggccctatgcgttccagagttggtttccattgccatgatgtcctcccaggacttatcctcggccgcatgagtacctgtccccagcggatctccccaagaattgggactggagaaatgtgaacggtgtcaactatgccagcgtcaccaggaaccagcacatcccacagtactgtggttcctgctgggcccacggcagcaccagtgccatggcaggtaggtggcctggcgctccatgctaagcctagggcagggccagtatcttggccaggtgggtggctgcacttcaccatccaatctacctttaggtgagccatgatagccatagccgggggctacaggggcccatggccctgtgccatgattcaggcttggatttcccctcagccagagtgaggtggctgtgtcattctggagttctctcctgtgggcctactagggcctctgtgggacaggcttgggctggaatctggtgacaagtggctcccaggaaccttcccttcctcagaacttgtgacttcagccattgagaaccatgggaccggtgttgcaaaatcactgagttcacagttggtttgtggggcatggaagtgaaactgcttctcagtagcagctggcatttgaggtgaacatgttccccagtacgaaatggccccccagctctcttcttcgaaccttagcctggccgcccaagagtcaagtagaagttcgttcagtaggaagagagaggccccagagggctgctgtgcccttccttggtagaggaagcaggagccagggagtgccaatctacctgtagcatggagattcagctttgccatccccagaggtggttcctggaaaccaatggggaagggagtctcctgcctgggagtgggactcagccagtgtcctctcttgattgggtatctttttggccaagtctgttgtaggtggagcctaggttgaggattaagaaaatgaaatttcattactgaaatggcagtgatcttgcccgtagggcttatgtaggtcttgcctgccagttggctctcagtgcccagcctaggaagcctctttcaggtcgggagactgcgttgtgagatggatggcagcttcagttcagcccatcagcctggactctcttttgaggatgaggggttcctgtctgagggcagtctgtaccccagcagtagccaggggcagatgtagctgtggcccctcatagagccacctctgagggctggggcatactgctgacttttcagaaagaggaagaaagaaagaaggtgggctgacaagaggtacttaccaagcaggcagggggtaaggagtgggcagccatagcagaggagtagagcaaagtccggggcgcttaggaatggtgtggccagactgctgttggggaagcgaaaggcccctctcagcagagcatagtgtggggtgagtgtgacatggagtgggaaagatccttgggtctctggatcgagttaaacgaggagggtgtagaacattgcaggatcaggagagatggccagaggctgttgggcctacccctcttgggtgggcactaggccctaccgaggttgtatcttcccagttgctacaaggcagtgtccttggggaaaatggctgcttcgttcttttcttcttcttttttttttttttttttttttggggttttcgagacaaggtttctctgtgtagccctggctgtcctggaactcactttgtagaccaggctggccttgaactcagaaatcctcctgcctctgcctcccgagtgctgggattaaaggcgtgcaccaccacgcccggctccgttcttttcttcttaagtctccgacatcttcgtcagtagcagtcagagccttgcttcctctttcgatgagtagacatcagagaggacttccgtggggccctggccgtttgccagggacagagcaggagactagtatacctttccccgtctctgggttaaggggcgtgtgggttctttaatgaagcgtttgccaatggttgatgctgttaagcatccaagcggcctctccttgaccagtcacttctgataggtgaccaagagacccttggtcccacccgtctctcactgccctgttgctcctgttgggttagctggagccagccccatcccccgcccacgtgctcctctgtgccctcaccagcctcacttccgcattgcctcacgtcgagttttttagctcctcaggccaggctaccaggcttcctaacatcctaacgatggggtggagcgagtccctcgctgtaccacacccggctctgcctccttcctaggacctttgcttcgccagtccccacacttctgagttctgggttcccagcagtgaccttttcctaactttcctgtccgtgtgctgctgccctctgacctccattcctgcggccttcagccccagacattcaggtcatcttactcaccaagcccatcctgcccctgaggacactcaagcaaaagcttagaggttatcaaactcaccttacgttcctcctgcaaactggtcccaggttctttagctgttccattcgtccctagtgagggcactacatggtgtctttcctaacaccttggctcctgtctggtcacagaccgtatgcttgctaaacctgagttccatcctatttctttatttgataccatctcaggattccaaagacaggtccttctctgacctccagagcctgcacagttatctggctcctcctccttcttgtcctcatgtcagcaggtgaggaatagtcaccttcatacccaagttgctcctctccttttctaatgtcctctcgctcctctccttggatgagagttttggttattgctctattgctgcaatgagacaccatgaccaaggccactttcaaaaggcagcatgtaactggggctttgcttacgaccgtcatggtgggaagcatggcagcaggcatggtgctggaacagtcgctaaggacttacaatctgatccagggcccagaggtagagaggtgcgggcttttttaaacctcagagcccaaccctagaggcacagctcctccaaggccacacctcctaattcttccccaaatagtccaccaactggagaccaagcattcaaacacatgagactatgggggccgttctcattcaaaccgccacggataaacatctcccaggctgtgctatgtacatgataacttagcccccggatggtccccaccacatctccttgcagtgttgtcctgcttgccctctgccttgcctccagtgccatggcacaagccccacaaagcagaactcgctcctgctggccagtcctactgtgtgtactgcacggtgcgcagaggtgtgaatggagatatcatgatgggactgaggctaaggacgcagagcgtggttgtgtagacaggccatctcaagaagcctgtgaagtctggagctggttggttggtcaggttggagctagacattaagtcaggtctagaagatggcaggcctgaggttatccagacagcccctgacagcgggtcttagcttgaggtcaccagagggacctgagaagaagaaagggacctcagcaagtcagagtgagaagcactcttctgaggtggagtgcagaagagccatgccaggcagacatgccaggcgcttcagaagaccggggtggcccctctgctgtcagatgaggatggagaagggatttggatggatgggtggtgtggagattgggaccctgacaagagtggcttgaaaggccaaagagagacgtgcacgcaatggtgaatccctctctcccagtgctgagagggaaagcgtggagccattgaactctgggaaagaggtctgtgcttgaaggtgccctctcatggaaaggtgacctgctgctggggatgcagagggagaaggctagagggagccgagcttgggggaaggagacaggagggagggtccatctgccttggacagctgggagcctgcttgcctgtggcacagaaagatatcccaggcacagaaacaggtctaaaatggtggcgagacaaggtctgcttgtgcgttgttccgtggcgagccctctgaggtggtggagtgtgactagggcgtgtgagggatggctcagagctgaagaccgtgtgaaggagggagagaaaacaaaggtaagctttgctttaggggaggagtgggtgggctatagggaaatggctggggtgcatggttgctttatacggcggcgggcgtgtaagcttaagagtgaagaccaagatggcttttcagaagcaggtcagccatcccctggtggatgttgtgatgatcctatctatctcttagtgggggtgggggtgggggggtggtagtacgtgagcactgagtgctggtgtccggccccccactgagactcatctggctcccctaggctgcccttgtccctggagatgcccaccccagccttccccgtatggtgtgtctggtctctagcctcctcacaggagtagtttcgttgccatcctatagtgatgctttacaagttcttgggacctctaaacccggccctgcctcatgtcacttgcagacagacaggcagatgtcatggcgctgaggaagaccagagaaagtctctgaaggaccgtggggcagagccctgggacgtttgctggggcagtcttggtgttggggggaacgtgggtgctgaccagagtgctgcctcttacagaccgaatcaacatcaagaggaaaggtgcatggccctccatcctgctgtccgtacagaatgtcattgactgtggcaatgctggctcttgtgaagggggcaatgaccttccggtgtgggagtatgcccacaagcatggcatccccgatgagacctgcaacaactaccaggccaaggaccaaggtagaccctgcctatgttcccacattgctacacacctgtctcgttccaagggagagtggctgagagcccacatttactccatatctgtcacactatgtggttgggatgctgcagaggactccagactggggacttagctcagtggtagagtgctggcctacttaccttacatgaggccctaggctcaatctcaattttttttttttttttttgtctttctacacagggtttctctgtatagccctggctgtcctggaactcactctgtagaccaggctggcctcgaactcagaaatctgcttgcctctgcctcccaagtgctgggattaaaggtgtgcaccgccactgccccacccaatctcaaatcttaaaagtaaacaaacaaacaaataaaataaagcagagcaccgcatgactggatgtggcatgtgcgtgtgcacacctggcatgtggatggtctcatgggccttacttacagagaaagaattctctgctgtaattgggtgtcctttgttctggatgagcaaacccagcagggaccagagatcccaccagggggacagacacagggtggagggaaggcaatggactgctttatggaataacccaggggccttatcccttttccttcattcccaaactggcgagtgacatccagctgaggttaggtccctttgaccttcactggtggttagggtcatcaggaccactgaccagtttcctcttcccagagccactatgttcctggtgcaactcacgctgggactggtgcctggggggtggggatgactaattaagttgatgatgtaatccttaaggagtttactgacccagggaaggatctaaatagatgattgttaagtacaggtcagcctcggtgtcttccttagtctctttccaccttgctctctctctctctgagcctaaagtgcatccagtttgctagcctggctggcaagcagacttcagagacccactagtcttcaccccccggggctgggaccacagacaggtggcatcatgctgactttctgtgggtgctaaggatctgagcgctggtcctgcaacagctctttactacctgactagtttccccagcctggaaacctgattcttttcttccttcattttttattttttattttattattattattttatttatttatttattggttttgttttgttttttgagacacgttttctctgtgtagccctggctgtcctggaattcactttgtagaccaggctggcctcgaactcagaaatcctcagaaatccgcctgcctctgtctccagagtgctgggattaaaggcgtgtgtgctaccacgcccggccccattttttacttttaaaaacaaactacaaagtattcagcaacagtgatatattgtagaatcgcaacatacaatgtaaaaagcaggttcctttctttttcttcccccctccccccctccaactcagaaatccacctgcctctgcctcctaaatgctgggattaaaggcgtgtgccaccactgcccggtcaaaagcaggtttttgagcatctccaagtccccacccagcatgttatgctttgagatttgggtacaatacatcactattttatcttaggattcccaagaatgctggaaaattccaaaattaggattactcctaacatacttttagcaaacttcacattctgttagacagctataatttttgtttcacaaatacaagtttagtaatttttcaaagagctatgtcctgcagtagtgttcaatatgaaacataatttacaattccgatctccccagtgaggcaatggccccacgtgaaatcactgcccattaggggtatgttctgtattaagaatgtgtgattcctccttgaaagaacaaaggacaaaatatcctagctatgtgatttcactagtaagcagtttgcccttttttattagccttccttgttctctccaaatgttacactgtacacacgtgcatttgttttcttacatgtatgtattatgggacagataggctctccctataaaggtgaacagggtttcctgggtttgggtcactttgcttaatattatacaagctaaagtccattcattatctctgcaaactttgtgattccatttttcttcagcatagtaatcattttatatgtgccaggtctttattatccattcacctgttggtggccagctagttccattccactgtctgccgtactgatccattccactgtcgtctgccatagtgaataaagcagcagtaaacttgggcaagcaaagaactcacggggaggctatggggtcagggtatgtgtgcagaggtggaacagctggatcgtacggggttctcagggtatgtgtgcagaggtggaacatctggatcgtacagggttctcagggtaggtgcccaggggtggaacagctggatcatacggggttctcagggtatgtgcccaggggtggaacagctggatcatacggggttctcagggtatgtgcccaggggtggaacagctggatcatacggggttctcagggtatgtgcccaggggtggaacagctggatcatatggggcctcaggatatgtgcccaggggtggaacagctgggtcatatggtagttttaattttttgaggaatctccaaactgatttccataatggctgtgttaatttgcatctccatcagcaatgaataagggctccttttctccaaaacatcctctccaacatttgcggtcaggtcgcttgatgacagtcattgtgacaatgatgaggtagtttctcaaagtagtttcaatttgcatttccatgctagctccatgactagggatattgaatttttcttcttcttgagacaaggtctcactatgtacccttggccctcctggaactcaatatgtagaccaggtttcaaactcagagatcctcctacccctctcccccaagtgctgggattaaagatgtctgccaccacccctagacctacttttgaaaaactcgatgcatgtggccatttgttgactggcaggcagtcttccttccttgctgttcaatgtttgaagtgcttagtaaattctggatgtttcaagtgtagctggtaaagattttcttctattctgtggatcctgtgttctgacgactgtttcctttgctgtacaaaaacatttattttcaggtggtctcatttgttatttggggctaatccctgttctactgatagtctattcagaaagttccagcctatccatgtatcttaagggggatttaacatatccttgtctagaagcttgtgtttcgggttttaagttgaggtctttgatcccacttgaattgatctttatacgggataaaaggtagaaatctaactttatttttcttatgtacatatgtagttttctcagcaccgttggttgactgggctttttaattgtttctttttttcttttttccgagacaaggtttctctgtgtagccctggctgtcttggaactcactctgtagaccaggctggcctcaaactcagaaatccacctgtctctgcctcccaagtgttggtctttttcctgatgtatgtttttgcctcctttgtcaagaattaaggggcatgttgtcaggtctgtccagatgttctgtccatctgtctgttactatgtcagcgctagttttgatcactatagctctgtagtgtaagtcgaggcgggggttgctttagctgttcctgtaaaccaaattctgatgcttctcaaggcaagaagagggtgcacgtcttcaaatcaagtacactggcctgctcagtcactagcctgcagtgcaattgcctggttcattttaaaaatctgattgcattcagtttttaattccagcatgctgtgagggcctcctggcctttaaagcagaagagaagggctactgtagagggctctgctatgtactaatcctgctatgtatcctgccatgtactaatccctgccatgtagtaatccaacccgcttgttctttctgttctgcccaggccggggtgggggaggggcttctctttgcagctgagagaagcatgtaattagggagcacctgagctggatggcttccgctgtcagctaatgaatggcacactaaatgagaaaagtggagaagatggggcgggcttcgttagcgggttaatgaggagctagtgtcatgctgacttcttgtgttgtagactgtgacaagtttaaccagtgtgggacctgcactgaattcaaagagtgtcacaccatccagaattacaccctctggagagtgggtgattacggctccctgtccgggagggagaagatgatggccgagatctatgccaatggtcccatcaggtaaggagaggtctcttcgggtagtggtgaagccacagctaggtctgtgcacattcctgtaccttgttcgtctttcctaccttgccattgtgaccccctagcttgagtaccacataacgttttttgcctgttggctttctcagaagtaaacaggagccattagccagtctgccccagggaactcctgtgtaagctagaagcccctgactggtgaatctagatgttggtgggaccactgaagcagggtttggactcctgtagcccgagtcacacaaacacccatgtgccacttacactgataacttttcacagctgcgggataatggcaacagagatgatgtctaactacactgggggcatctatgctgagcaccaggaccaggccgttatcaaccacatcatctctgtagctggctggggtgtcagcaacgatggcatcgagtactggattgtccgaaattcatggggcgaaccctgggtaagatagctcccctttcttaaggttagactggaatccacaaccctcctcagcttcacggagagctggctgcagctatgtcaccaggacacaagcaacgtcagggctgtgcaggactgctgctgtcccgtgcgtgtagcactgggtgttagaagcccctgaggtgccagtggctcctccccaagccccggtgctcgggaaacagttaaggctggggtcaaacagtcctgttggtggcgactgactggctacgccttgaggaccagctcatgcttgctcctgccatgtgtttcattcaccgtgtgtttcattcgagctgggtcaaaggtccaggggaggataagtggccacaagtggggtattaacagggagacaatcacattttcttgcattatttagctgcctgcagagttaaaacatttaactgagaggggccgtatcagagctttgctgcttatgtgggcctgagggagcctgtctggaccctcagattctatagggctagtctacggatggtggctgagggcagggcctgggtctacagaacagagggaccatgtcctgaggcactaatgcagagttcttgctccttacagggtgagaaaggctggatgaggatcgtgaccagcacctacaagggaggcacaggtgacagctacaaccttgccatcgagagtgcctgcacatttggggaccccattgtttaggtagatgtctctggaagcagcgctgtgaaccatgacagggaggggtgattaattactgacactggacatgtccagacagctataaacagtgcttgtggacatgaggaccagagtgtggactgcatcccgagaggagacggtaaaggatgaaacacaactgcactgggaccctccgccgtaccctccaggcctgcctcctccaccactgagccctccaggcctgcctcctcttctacagtgcttgccttcagccacccggagaagagagctatggtttaggacagctcaacttatcaccagatctggagccctggaatccatgggaggggggaacaagtccagactgcttaagaaatgagtaaaatatctggcttcccacgcttgtgtactttatttacacgagactagcacacacaacacacgcacactcccacacacacacatacaactcaacattactcttggaaacttaaagtaactgaccatctgggtcataggaaggaaacttacataaacccttcatgaagtgtcccagctgctagattcccactacccagaggcccactgggttcctgttcagaggtggcacacgcctgggatgtgacatgtagcacagagccacactgcgacacaggggggctgtctgccacagaagctacctttgagaagagttgcctgcttcttcccagcagaccctgcttccaggaggctggttgagcctcttcacagagcagctttctgaacgagtcctctggtccccccaccccaggctggcatggaagtttcggcttcagttacagatgcgctcagttcaccacgatgaagttcgacttgcagtgagctgtacagaaacacatggccactgcattagagcaggtgaggagtaaggtgggccatcatggcacagggtacctcgaggccttaccaatgaactccgtcacggggtcatgctcgccctctgccttgatggtgcccgcgatgctgtcattttccaagatggggaggaaaagctgcacaaagtctgaggtgtacggaggggcgatgacatccagaacctagtggggagacctcagggtgactgacctgctgatgcgggacaccgcactccataatagtcagggaggtgtgagaggccctgccgggcttttccagagacaccgcagcccccacctacaccacccagccagaagggctgggctagtgaagagtgacccgtgaccccacggtgcgctcactgctgacctcagtgacaaagtagcggatgagggagatgtccgtgtccagcttctccagacatttgcggatataactgaccaccggcaggacgtagccgcggctcagcaggtgcaccatcctgtccagcagggtctttttcaactcgagctgtggaagaacagagaccgggagcaggtcagagctgcaagtctccagcatcgagacactctggggtgcagctcacctgctccatcacgtctagctgggagtgctctgtctcaaaaagcttaacaagcagctgcagaacctgggggtgcaggagctggtggcaggtgctgatctgtgaacacccagaagacatcactgaggcgcctgccactctcagggcttattagcagaacatggggagtatacagaattgtcacacccacccaaagtggcttactgccgatggggacaccacctgtcagcttctaggtttcctccccctaacacttgataaaaacacatctacagtcaagtgaaactagatacttaaggaaaagctggtaggacacaaactaccccaacgcaaaggtctaagacacagagcactatgcatcttacaagtggtccttccgacctctcacctcatccaacaaggccaggtgaactggagtgtgatcagtctgcagctggaagtaccttggctccgatacggtccaatctacccacttgagcacgcccatcgccaccactggaaacctacaggcagagtggggaaactgaggctgaatatgccagagcagcaaacagctgggaatcagaaaaggcagtgtctgcggacacagtggctccccttcactctgccctgaggtagtggcttgtgacagaagtggtttgatgaggtcctcccagaagggatctgttctcagctacttccaaacccatcatccctccccatgaggaaagggccatgcggagggcgctgcctggcagcctgggctcccgtttacctttacttcagacccttccttagtccccagaaaagtgaaagtgcctaagacggtacagaccccaggacatacgtacgcagtcacgagtcactcctatccaggcaatgcttgttggtttaacagtgtgattacagatgaacccagtttctctaattctggatgtcctttctttagtctactctgtgaaggtctgctgggcagatttctggccaataagccctcctgctgctgcagctgctaagttccccatgtgtgcttaagcacgagaaacccaagtttcttactcagcgcaagagtacatgctctaaccatggaagcatgagggcttgaactccaaggaatggatggg

SEQ ID NO: 54 - DNA, 4879 bp, Artificial Sequence
  Synthetic
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcagatgctgcacacttgtggactaagtttgttcgcatccccttctccaaccccctcagtacatcaccctgggggaacagggtccacttgctcctgggcccacacagtcctgcagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgcgaggctgtgagcagccacagtgccctgctcagaagccccaagctcgtcagtcaagccggttctccgtttgcactcaggagcacgggcaggcgagtggcccctagttctgggggcagcgtgaaaagacggtatcgataagcttgattcgagctagccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaatcgaattcctgcagccccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcggggggatcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 55 - protein, 208 aa, Homo sapiens; identical to SEQ ID NO: 37

(19) United States
(12) Patent Application Publication
(10) Pub. No.: US 2024/0050593 A1
(43) Pub. Date: Feb. 15, 2024

(54) Compositions For and Methods of Enhancing Spinal Cord Tissue Regeneration

(71) Applicant: Duke University, Durham, NC (US)

(72) Inventors: Kenneth Poss, Durham, NC (US); Valentina Cigliola, Durham, NC (US); Adam Shoffner, Durham, NC (US)

(73) Assignee: Duke University, Durham, NC (US)

(21) Appl. No.: 18/267,898

(22) PCT Filed: Dec. 15, 2021
(86) PCT No.: PCT/US21/63643

§ 371 Date: Jun. 16, 2023

Related U.S. Application Data
(60) Provisional application No. 63/127,217, filed on Dec. 18, 2020.

(51) Int. Cl. A61K 48/00; A61P 25/00; C12N 15/86

(52) U.S. Cl. CPC A61K 48/0058; A61P 25/00; C12N 15/86; C12N 2750/14143; C12N 2750/14171

(57) ABSTRACT

Disclosed herein are compositions and methods of treating a spinal cord injury and improving spinal cord function. Also disclosed are compositions and methods of stimulating regeneration, promoting glial cell proliferation, promoting axonal tract regeneration, triggering neurite outgrowth, and triggering neuron formation in injured and/or damaged spinal cord tissue.

### CROSS-REFERENCE TO RELATED APPLICATIONS

This application claims the benefit of U.S. Provisional Application No. 63/127,217 filed 18 Dec. 2020, which is incorporated by reference herein in its entirety.

### STATEMENT REGARDING FEDERALLY SPONSORED RESEARCH

Parts of this invention were made with government support under Grant No. R35-HL150713 awarded by the National Institutes of Health.

### REFERENCE TO THE SEQUENCE LISTING

The Sequence Listing submitted 15 Dec. 2021 as a text file named “21_2034_WO_Sequence_Listing_ST25”, created on 15 Dec. 2021 and having a size of 228 kilobytes is hereby incorporated by reference pursuant to 37 C.F.R. § 1.52(c)(5).

### BACKGROUND OF THE INVENTION

Spinal cord injury (SCI) often causes permanent paralysis. In mammals. SCI results in formation of scar tissue at the lesion site, which acts as a barrier to axon regeneration and functional recovery (Bradbury E J, et al. (2019) Nature. 10(1):3879; He Z, et al. (2016) Neuron. 90(3):437-451; Sofroniew M V (2018) Nature. 447(7704):343-350; Tran A P, et al. (2018) Physiol. Rev. 98(2):881-917). By contrast, certain non-mammalian vertebrates like zebrafish can regenerate spinal cord tissue and reverse paralysis, including after complete transection injuries in adults (Becker T, et al. (1997) J Comp Neurol. 377:577-595; Bernstein J J (1964) Exp Neurol. 9:161-174; Butler E G, et al. (1967) Dev Biol. 15:464-486; Cohen A H, et al. (1986) Proc Natl Acad Sci. USA 83:2763-2766; McClellan A D (1990) Neuroscience. 35:675-685; Piatt J, et al. (1958) Anat Rec. 131:81-95; Rovainen C M (1976) J Comp Neurol. 168:545-554; Selzer M E (1978) J Physiol. 277:395-408).

For instance, SCI in adult zebrafish triggers proliferation of ependymal radial glial cells (ERGs), after which they are proposed to differentiate into new glial cells and neurons (Becker C G, et al. (2015) Dev Cell. 32:516-527; Cigliola V. et al. (2020) Dis Model Mech. 13). A subpopulation of glial derivatives form bridges between the rostral and caudal stumps, events that depend on signaling by fibroblast growth factors and connective tissue growth factor, the latter of which is expressed in an early population of bridging glia (Goldshmit Y. et al. (2012) J Neurosci. 32:7477-7492; Mokalled, et al. (2016) Science. 354:630-634). A variety of intrinsic and extracellular factors have been implicated in guidance of axon across the lesion site in adult zebrafish, among which are secreted proteins (Sema4D), ECM components (Tenascin-C), scaffolding proteins (Syntenin-A), transcription factors (ATF3 and 6), microRNA (mi133) and immune-cell derived factors (Neurotrophin 3) (Hui S P, et al. (2017) Dev Cell. 43:659-672 e655; Ji Z, et al. (2021) Neurosci. 71:734-745; Peng S X, et al. (2017) Neuroscience. 351:36-46; Wang L F, et al. (2017) Biochem Biophys Res Commun. 488:522-527; Yu Y. et al. (2013) Eur J Neurosci. 38:2280-2289; Yu Y M, et al. (2011) Neuroscience. 183:238-250). Neonatal mice also mount a microglia-dependent response to spinal cord injury that permits the growth of long projecting axons through lesions (Li Y, et al. (2020) Nature. 587:613-618).

The key for understanding the capacity for spinal cord regeneration is the identification of mechanisms that promote the rostrocaudal direction of axon regeneration to reconnect long axons with their targets. Accordingly, there is an urgent and previously unmet need to enhance spinal cord tissue regeneration, especially in adult mammals.

### BRIEF SUMMARY OF THE INVENTION

Disclosed herein are compositions comprising HB-EGF.

Disclosed herein is an isolated nucleic acid molecule, comprising a nucleic acid sequence encoding a tissue regeneration enhancer element (TREE), an encoded polypeptide; and a promoter directing expression of the encoded polypeptide in damaged and/or injured spinal cord tissues.

Disclosed herein is an isolated nucleic acid molecule, comprising a nucleic acid sequence encoding a tissue regeneration enhancer element (TREE); an encoded polypeptide; a promoter directing expression of the encoded polypeptide in damaged and/or injured spinal cord tissues; and a 3′ UTR noncoding region.

Disclosed herein are plasmids that comprise a disclosed isolated nucleic acid molecule, a disclosed transgene, and/or a disclosed transgene cassette.

Disclosed herein is a transgene cassette comprising a disclosed isolated nucleic acid molecule comprising a nucleic acid sequence encoding a tissue regeneration enhancer element (TREE); an encoded polypeptide; and a promoter directing expression of the encoded polypeptide in damaged and/or injured spinal cord tissues.

Disclosed herein is a transgene cassette comprising isolated nucleic acid molecule, comprising a nucleic acid sequence encoding a tissue regeneration enhancer element (TREE); an encoded polypeptide; a promoter directing expression of the encoded polypeptide in damaged and/or injured spinal cord tissues; and a 3′ UTR noncoding region.

Disclosed herein are transgenic animals comprising a disclosed transgene cassette. In an aspect, a disclosed transgenic animal can be used for identification and/or validation on a putative TREE.

Disclosed herein is a vector comprising a disclosed isolated nucleic molecule.

Disclosed herein is a vector comprising an isolated nucleic acid molecule, comprising a nucleic acid sequence encoding a tissue regeneration enhancer element (TREE); an encoded polypeptide; and a promoter directing expression of the encoded polypeptide in damaged and/or injured spinal cord tissues.

Disclosed herein is a vector comprising an isolated nucleic acid molecule, comprising a nucleic acid sequence encoding a tissue regeneration enhancer element (TREE); an encoded polypeptide; a promoter directing expression of the encoded polypeptide in damaged and/or injured spinal cord tissues; and a 3′ UTR noncoding region.

Disclosed herein is a vector comprising a disclosed gene or transgene cassette. In an aspect, a disclosed gene or transgene cassette can comprise a disclosed isolated nucleic acid molecule.

Disclosed herein is an AAV or an rAAV vector comprising a nucleic acid sequence encoding a tissue regeneration enhancer element (TREE); an encoded polypeptide; and a promoter directing expression of the encoded polypeptide in damaged and/or injured spinal cord tissues.

Disclosed herein is an AAV or an rAAV vector comprising a nucleic acid sequence encoding a tissue regeneration enhancer element (TREE); an encoded polypeptide; a promoter directing expression of the encoded polypeptide in damaged and/or injured spinal cord tissues; and a 3′ UTR noncoding region.

Disclosed herein is an AAV or an rAAV vector comprising a disclosed plasmid such as, for example, a hb-egfaEN-cfos:EGFP plasmid construct and a cfos:EGFP zebrafish plasmid construct.

Disclosed herein is an AAV or an rAAV vector comprising a disclosed isolated nucleic acid molecule, a disclosed transgene, and/or a disclosed gene or transgene cassette.

Disclosed herein is an AAV or an rAAV vector comprising a disclosed gene or transgene cassette.

Disclosed herein is AAV or an rAAV vector comprising a gene or transgene cassette comprising a disclosed isolated nucleic acid molecule comprising a nucleic acid sequence encoding a tissue regeneration enhancer element (TREE); an encoded polypeptide; and a promoter directing expression of the encoded polypeptide in damaged and/or injured spinal cord tissues. Disclosed herein is AAV or an rAAV vector comprising a transgene cassette comprising isolated nucleic acid molecule, comprising a nucleic acid sequence encoding a tissue regeneration enhancer element (TREE); an encoded polypeptide; a promoter directing expression of the encoded polypeptide in damaged and/or injured spinal cord tissues; and a 3′ UTR noncoding region.

Disclosed herein is vector comprising a nucleic acid sequence encoding a tissue regeneration enhancer element (TREE); and a promoter directing expression of a endogenous polypeptide in damaged and/or injured spinal cord tissues.

Disclosed herein is a vector comprising a nucleic acid sequence encoding a tissue regeneration enhancer element (TREE); a promoter directing expression of a endogenous polypeptide in damaged and/or injured spinal cord tissues; and a 3′ UTR noncoding region.

Disclosed herein is a vector comprising a disclosed plasmid such as, for example, a hb-egfaEN-cfos:EGFP plasmid construct and a cfos:EGFP zebrafish plasmid construct.

Disclosed herein is a vector comprising a disclosed isolated nucleic acid molecule, a disclosed transgene, and/or a disclosed transgene cassette.

Disclosed herein is a vector comprising a disclosed gene or transgene cassette.

Disclosed herein is a vector comprising a gene or transgene cassette comprising a disclosed isolated nucleic acid molecule comprising a nucleic acid sequence encoding a tissue regeneration enhancer element (TREE); an encoded polypeptide; and a promoter directing expression of the encoded polypeptide in damaged and/or injured spinal cord tissues.

Disclosed herein is a vector comprising a gene or transgene cassette comprising isolated nucleic acid molecule, comprising a nucleic acid sequence encoding a tissue regeneration enhancer element (TREE); an encoded polypeptide; a promoter directing expression of the encoded polypeptide in damaged and/or injured spinal cord tissues; and a 3′ UTR noncoding region.

Disclosed herein is a pharmaceutical formulation comprising a disclosed isolated nucleic acid molecule and a pharmaceutically acceptable carrier.

Disclosed herein is a pharmaceutical formulation comprising an isolated nucleic acid molecule, comprising a nucleic acid sequence encoding a tissue regeneration enhancer element (TREE); an encoded polypeptide; and a promoter directing expression of the encoded polypeptide in damaged and/or injured spinal cord tissues, and a pharmaceutically acceptable carrier.

Disclosed herein is a pharmaceutical formulation comprising a nucleic acid sequence encoding a tissue regeneration enhancer element (TREE); an encoded polypeptide; a promoter directing expression of the encoded polypeptide in damaged and/or injured spinal cord tissues; and a 3′ UTR noncoding region, and a pharmaceutically acceptable carrier.

Disclosed herein is a pharmaceutical formulation comprising a disclosed vector and a pharmaceutically acceptable carrier.

Disclosed herein is a pharmaceutical formulation comprising a disclosed HB-EGF and a pharmaceutically acceptable carrier.

Disclosed herein is a kit comprising one or more components and/or reagents for use in one or more disclosed methods.

Disclosed herein is a method of treating a spinal cord injury, the method comprising administering to a subject in need thereof a disclosed HB-EGF or a disclosed isolated nucleic acid molecule, a disclosed vector, a disclosed pharmaceutical formulation, or any combination thereof.

Disclosed herein is a method of treating a spinal cord injury, the method comprising stimulating regeneration of injured and/or damaged spinal cord tissue in a subject in need thereof by administering to a subject a disclosed HB-EGF or a disclosed isolated nucleic acid molecule, a disclosed vector, a disclosed pharmaceutical formulation, or any combination thereof.

Disclosed herein is a method of stimulating regeneration of injured and/or damaged spinal cord tissue, the method comprising administering to a subject in need thereof a disclosed isolated nucleic acid molecule, a disclosed vector, a disclosed pharmaceutical formulation, or any combination thereof, and administering a therapeutically effective amount of HB-EGF.

Disclosed herein is a method of treating a spinal cord injury, the method comprising: promoting glial cell proliferation in injured and/or damaged spinal cord tissue in a subject by administering to a subject a disclosed HB-EGF or a disclosed isolated nucleic acid molecule, a disclosed vector, a disclosed pharmaceutical formulation, or any combination thereof.

Disclosed herein is a method of treating a spinal cord injury, the method comprising promoting axonal tract regeneration in injured and/or damaged spinal cord tissue in a subject in need thereof by administering to a subject a disclosed HB-EGF or a disclosed isolated nucleic acid molecule, a disclosed vector, a disclosed pharmaceutical formulation, or any combination thereof.

Disclosed herein is a method of treating a spinal cord injury, the method comprising triggering neurite outgrowth in injured and/or damaged spinal cord tissue in a subject in need thereof by administering to the subject a disclosed HB-EGF or a disclosed isolated nucleic acid molecule, a disclosed vector, a disclosed pharmaceutical formulation, or any combination thereof.

Disclosed herein method of treating a spinal cord injury, the method comprising triggering neuron formation in injured and/or damaged spinal cord tissue in a subject in need thereof by administering to a subject a disclosed HB-EGF or a disclosed isolated nucleic acid molecule, a disclosed vector, a disclosed pharmaceutical formulation, or any combination thereof.

Disclosed herein is a method of treating a spinal cord injury, the method comprising improving spinal cord function in a subject in need thereof by administering to a subject a disclosed HB-EGF or a disclosed isolated nucleic acid molecule, a disclosed vector, a disclosed pharmaceutical formulation, or any combination thereof.

Disclosed herein is a method of treating a spinal cord injury, the method comprising administering to a subject in need thereof a disclosed isolated nucleic acid molecule, a disclosed vector, a disclosed pharmaceutical formulation, or any combination thereof, and administering a therapeutically effective amount of HB-EGF.

Disclosed herein is a method of stimulating regeneration of injured and/or damaged spinal cord tissue, the method comprising administering to a subject in need thereof a disclosed HB-EGF or a disclosed isolated nucleic acid molecule, a disclosed vector, a disclosed pharmaceutical formulation, or any combination thereof.

Disclosed herein is a method of promoting glial cell proliferation in injured and/or damaged spinal cord tissue, the method comprising administering to a subject in need thereof a disclosed HB-EGF or a disclosed isolated nucleic acid molecule, a disclosed vector, a disclosed pharmaceutical formulation, or any combination thereof.

Disclosed herein is a method of promoting glial cell proliferation in injured and/or damaged spinal cord tissue, the method comprising administering to a subject in need thereof a disclosed isolated nucleic acid molecule, a disclosed vector, a disclosed pharmaceutical formulation, or any combination thereof and administering a therapeutically effective amount of HB-EGF.

Disclosed herein is a method of promoting axonal tract regeneration in injured and/or damaged spinal cord tissue, the method comprising administering to a subject in need thereof a disclosed HB-EGF or a disclosed isolated nucleic acid molecule, a disclosed vector, a disclosed pharmaceutical formulation, or any combination thereof.

Disclosed herein is a method of triggering neurite outgrowth and/or triggering neuron formation in injured and/or damaged spinal cord tissue, the method comprising administering to a subject in need thereof a disclosed HB-EGF or a disclosed isolated nucleic acid molecule, a disclosed vector, a disclosed pharmaceutical formulation, or any combination thereof.

Disclosed herein is a method of improving spinal cord function, the method comprising administering to a subject in need thereof a disclosed HB-EGF or a disclosed isolated nucleic acid molecule, a disclosed vector, a disclosed pharmaceutical formulation, or any combination thereof.

Disclosed herein is a method of generating a disclosed non-viral vector or a disclosed viral vector Disclosed herein is a method of generating an AAV vector, the method comprising: employing triple-plasmid transfection protocol.

Disclosed herein is a method of generating a disclosed hydrogel.

Disclosed herein is a method of identifying one or more putative TREEs, the method comprising isolating the nuclei from a first population of spinal cord cells and a second population of spinal cord cells; analyzing chromatin structure and function of the isolated nuclei to obtain a chromatin profile for the first population of spinal cord cells and for the second population of spinal cord cells; and comparing the chromatin profiles of the two populations of spinal cord cell to identity one or more putative TREEs.

Disclosed herein is a method of identifying one or more TREEs, the method comprising obtaining a first population of spinal cord cells; isolating the nuclei from the first population of spinal cord cells; analyzing chromatin structure and function of the isolated nuclei to obtain a chromatin profile for the first population of spinal cord cells; obtaining a second population of spinal cord cells; isolating the nuclei from the second population of spinal cord cells; analyzing chromatin structure and function of the isolated nuclei to obtain a chromatin profile for the second population of spinal cord cells; and comparing the chromatin profiles between the two populations of spinal cord cells to identify one or more putative TREEs.

### BRIEF DESCRIPTION OF THE FIGURES

FIG. 1A-FIG. 1L shows hb-egf genes are induced in zebrafish spinal cord after injury and required for regeneration. FIG. 1A shows strategy used to identify regulators of spinal cord regeneration. FIG. 1B and FIG. 1C shows in situ hybridization for hb-egfa and hb-egfb, respectively, on longitudinal sections of zebrafish spinal cord at 1 and 2 wpi, and in sham-injured controls. Dashed lines delineate spinal cord. FIG. 1D shows a cartoon indicating the exonic regions deleted to generate hb-egfa and hh-egfb mutations. FIG. 1E shows transverse sections of wild type and hb-egf double mutant (dKO) spinal cords at 1 wpi, stained for ependymal cells (Sox2⁺, white) and EdU (red) incorporation. FIG. 1F shows the quantification of proliferating ependymal cells at 1 wpi (n=3). FIG. 1G shows tissue sections indicating expression of the glial marker GFAP (magenta) and the axonal marker acetylated α-tubulin (yellow) in wild-type and dKO spinal cords at 4 wpi. Dashed lines delineate sites of tissue bridging. FIG. 1H shows the quantification of tissue bridging from experiments in FIG. 1G (n=4). FIG. 1I shows sections of wil-type and dKO spinal cords located rostral or caudal to the transection site, after anterograde axon tracing at 4 wpi. FIG. 1J shows quantification (n=3). FIG. 1K and FIG. 1L shows the capacity of wild-type (grey) and dKO (red) animals to swim against increasing water currents inside an enclosed swim tunnel at 2, 4, and 6 wpi. The dashed lines in FIG. 1L indicate uninjured animals whereas full lines indicate injured animals. Unpaired t-test with Welch's correction was used for comparisons in FIG. 1F, FIG. 1H, and FIG. 1J and Mann-Whitney for comparisons in FIG. 1K-FIG. 1L. Scale bars 200 μm in FIG. 1A, FIG. 1B, and FIG. 1G, 100 μm in FIG. 1I, 50 μm in FIG. 1E. In FIG. 1, r=rostral; c=caudal; d=dorsal; v=ventral.

FIG. 2A-2K shows that hb-egfa paralog is required for zebrafish spinal cord regeneration. FIG. 2A shows sections of spinal cord tissue indicating hb-egfa:EGFP BAC reporter expression (green) in sham, 1 and 2 wpi animals, acetylated α-tubulin (red) stains axons. FIG. 2B shows EdU (red) incorporation assay for cell cycling at 1 wpi. FIG. 2C shows the assays for Sox2 (white) expression at 1 wpi. FIG. 2D shows the cross-section of hh-egfa:EGFP spinal cord at 1 wpi showing hb-egfa expression in ependymo radial-glial cells. GFAP (magenta) stains glial cells. FIG. 2E shows the expression of hb-egfb in sham injured and injured adult spinal cords at 1 and 2 weeks post injury (wpi). FIG. 2F shows the transverse section of adult spinal cord showing expression of hb-egfb and the glial marker GFAP in sham-injured and at 1 wpi. FIG. 2G shows the experimental design for experiments with single mutants. FIG. 2H shows the quantification of tissue bridging in wild-type, hb-egfaKO, or hb-egfbKO fish (n=2).

FIG. 2I shows the quantification of anterograde axon labeling experiments in wild-type, hb-egfdKO, or hb-egfbKO spinal cords at 4 wpi (n=2). FIG. 2J and FIG. 2K) shows the swim capacity assayed in wild-type (grey), hb-egfa (orange), or hb-egfb (dark red) mutant animals at 2, 4 and 6 wpi. One-way ANOVA was used for comparisons in FIG. 2K and FIG. 2I and Mann-Whitney for comparisons in FIG. 2J and FIG. 2K. Dashed boxes in FIG. 2B-FIG. 2D areas magnified. Scale bars 200 μm in FIG. 2A and FIG. 2E. 100 μm in FIG. 2D and FIG. 2F, 50 μm in FIG. 2B and FIG. 2C. In FIG. 2, r=rostral; c=caudal; d=dorsal; v=ventral.

FIG. 3A-FIG. 3L shows the disruption of Hb-egfa polarity impairs axon growth after spinal cord injury. FIG. 3A shows the longitudinal sections of spinal cords from EGFP BAC reporter fish at 1 and 2 wpi, showing differential rostrocaudal distribution of EGFP fluorescence. FIG. 3B shows the quantification of EGFP and DAPI fluorescence in the rostral and caudal sides of spinal cord lesions at 1 and 2 wpi.

FIG. 3C shows the transgene used for generation of hsp70:hb-egfa-p2a-tbfp fish and FIG. 3D shows the longitudinal section of hb-egfaOE spinal cord at 2 weeks after heat shock (IS) showing TBFP expression, a proxy for Hb-Egfa, (cyan) along the rostrocaudal spinal cord axis. GFAP (magenta) stains glial cells. FIG. 3E shows cross-sections of wild-type and hb-egfaOE spinal cords located rostral (r) or caudal (c) to the transection site, after anterograde axon tracing at 4 wpi. Quantification shown in FIG. 3F (n=4). FIG. 3G and FIG. 3H show swim capacity assayed in wild-type (grey, full line) or hb-egfbOE (cyan, full line) animals at 2, 4, and 6 wpi, and in uninjured animals (dashed lines). Whole animal hb-egfa overexpression impairs recovery.

FIG. 3I shows the experimental design for application of hydrogel containing human recombinant (HR-HB-EGF) or Vehicle (BSA) at the site of spinal cord injury.

FIG. 3J shows cross-sections of vehicle- and HR-HB-EGF-treated spinal cords located rostral and caudal to the transection site, after anterograde axon tracing at 4 wpi. Quantification shown in FIG. 3 K (n=4). FIG. 3L shows swim tests in fish treated with recombinant HR-HB-EGF (purple), vehicle alone (grey), and untreated (dashed grey) at the site of a spinal cord crush injury. Scale bars 200 μm in FIG. 3A and FIG. 3D, 100 μm in FIG. 3E and FIG. 3J. Dashed lines in FIG. 3A and FIG. 3D delineate the spinal cord. In FIG. 3, r=rostral; c=caudal; d=dorsal; v=ventral.

FIG. 4A-4L shows a nearby tissue regeneration enhancer element controls hb-egfa expression. FIG. 4A shows a heatmap of ATAC-seq peaks with changes during spinal cord regeneration. Cutoff is p-val<0.05 and fold change >1.2. FIG. 4B shows a dot plot of differential ATAC-seq chromatin regions linked to nearby differential transcripts in 7 days post injury (dpi) versus sham-injured spinal cords. Each point indicates a separate ATAC-seq peak. FIG. 4C shows the browser track indicating chromatin accessibility (dark and light orange) and transcript levels (light and dark blue) at the hb-egfa locus, indicating the candidate hb-egO-linked enhancer hb-egfaEN with dashed lines. hb-egfaEN is located 15.6 kb downstream of hb-egfa and increases accessibility after SCI. FIG. 4D shows hb-egfaEN-cfos:EGFP enhancer reporter construct. An 800 bp sequence encompassing the 310 bp hb-egfaEN was placed upstream of a 96 bp c-fos minimal promoter to direct an EGFP cassette. FIG. 4E shows longitudinal sections of hb-egfaEN-cfos:EGFP spinal cords showing induced EGFP at 1 wpi and 2 wpi. FIG. 4F shows the quantification of EGFP and DAPI fluorescence rostral and caudal to the lesion site at 1 and 2 wpi in hb-egfaEN-cfos:EGFP spinal cords. FIG. 4G shows the assays for Sox2 and FIG. 4H shows the EdU incorporation assay for cell cycling in cells showing hb-egfEN driven EGFP expression at 1 wpi. FIG. 4I shows diagram showing Sox binding sites in the hb-egfa enhancer and promoter region. Binding sites are assessed using the motifmatchr package (p-value<0.0005). FIG. 4J shows transgenes used for modulation of sox2 levels and assessment of hh-egfa expression. FIG. 4K shows the sox2 and hb-egfa mRNA expression levels (pink and green, respectively) in hsp70:sox2 larvae after a heat shock regimen. Expression was normalized to beta-actin. FIG. 4L shows the Hb-egfa protein expression (white) in hsp70:sox2; hb-egfa:EGFP larvae after heat shock. Whole-animal fluorescence is increased compared to larvae maintained at room temperature. Unpaired t-test was used for comparisons in FIG. 4K, one sample t-test for comparisons in FIG. 4F. Dashed boxes in FIG. 4G and FIG. 411 indicate areas magnified on the right. Scale bars 200 μm were in FIG. 4E, 50 μm in FIG. 4G and FIG. 4H (r=rostral; c=caudal; d, dorsal; v, ventral).

FIG. 5A-FIG. 5N shows that hb-egfaEN directs injury-associated gene expression in neonatal mice and can improve mammalian axon regeneration by HB-EGF delivery. FIG. 5A shows circle plot showing conservation of zebrafish hb-egfaEN in different species. Percentage values next to each organism indicate conservation scores. FIG. 5B shows the transgene construct to evaluate the ability of zebrafish hb-egfaEN to direct expression in mouse spinal cord upon injury. FIG. 5C shows the experimental design for experiments to test hb-egfaEN activity in adult mouse spinal cord after systemic delivery of an AAV virus. FIG. 5D shows the longitudinal sections of sham-injured and 1 week post injury spinal cord of adult mice. hb-egfaEN does not activate EGFP expression. FIG. 5E shows the experimental design for experiments to test hb-egfaEN activity in neonatal mouse spinal cord. FIG. 5F shows the longitudinal sections of sham-injured, and 1 day and 4 days post-injury spinal cords of neonatal mice. hb-egfaEN directs EGFP in cells at the site of injury. Expression of the proliferation marker Ki67 (FIG. 5G), the transcription factor Sox2 (FIG. 5H) and the glial marker GFAP (FIG. 5I) in cells activating hb-egfaEN-directed EGFP expression at 4 dpi in neonatal spinal cord. (FIG. 5I). Transgene construct and (FIG. 5K) experimental design to concentrate expression of human HB-EGF at the lesion site of neonatal spinal cords after systemic delivery of an AAV virus, using zebrafish hb-egfaEN as driver. FIG. 5L shows the in situ hybridization showing expression of human HB-EGF mRNA in mice injected with AAV carrying hbegfaEN-hsp68:EGFP (top panel) or hbegfaEN-hsp68:HB-EGF (bottom panel) constructs. Spinal cord was assessed at 7 dpi. Magnified panel on the right shows site of injury in hbegfaEN-hsp68:1B-EGF mice after injury. Red arrows indicate HB-EGF mRNA signal. FIG. 5M shows the longitudinal sections of crush-injured neonatal spinal cords of mice injected with AAV carrying hbegfaEN-hsp68:EGFP (top panel) or hbegfaEN-hsp68:HB-EGF (bottom panel) constructs and stained for the serotonergic axon marker 5-HT at 7 dpi. FIG. 5N shows Quantification of the density of caudal serotonergic axons normalized to the density rostral to the lesion site in spinal cord at 7 dpi (n=1). Scale bars 500 μm in FIG. 5D and FIG. 5M, 200 μm in FIG. 5L, 100 μm in FIG. 5F, 50 μm in FIG. 5G, 40 μm in FIG. 5H and FIG. 5I.

FIG. 6A-FIG. 6C shows the expression of egfra and ERBB4 receptors after spinal cord injury in adult zebrafish. FIG. 6A shows the transverse sections of adult spinal cord showing expression of egfra mRNA by in situ hybridization after sham injury or 1 and 2 weeks after spinal cord transection. FIG. 6B shows the longitudinal and FIG. 6C shows the transverse sections of spinal cord indicating expression of ERBB4 receptor (yellow) after sham injury or at 1 and 2 wpi. Scale bars are 100 μm in FIG. 6A and FIG. 6C while scale bares are 200 μm in FIG. 6B (d=dorsal; v=ventral; r=rostral, c=caudal).

FIG. 7A-FIG. 7C shows that hh-egfa and hb-egfb expression was abolished in dKO mutants, and mutant fish are viable with normal baseline swim capacity. FIG. 7A shows the swim capacity of adult, uninjured wild-type, and hb-egfdKO animals. Uninjured wild-type and mutant animals perform similarly when swimming at a fix high current. FIG. 7B shows the in situ hybridization of transverse sections of spinal cord, showing loss of hb-egfa and hb-egfb induction at 1 wpi in dKO mutants compared to wild-type clutchmates. FIG. 7C shows quantitative RT-PCR indicating decreased expression of hb-egfa and hb-egfb in dKO mutant embryos compared to wild-type controls. Scale bars in FIG. 7A are 100 μm (d=dorsal; v=ventral).

FIG. 8A-FIG. 8D shows that hb-egfa- and hb-egfb-directed EGFP expression in spinal cord. FIG. 8A shows images of hb-egfa:EGFP while FIG. 8B shows hb-egfb:EGFP in larval zebrafish spinal cord injured at 3 days post fertilization (3 dpf) and imaged at 2- and 3-days post injury (dpi). GFAP-H2A-mCherry labels glial cell nuclei. FIG. 8C shows the expression of hb-egfa:EGFP at sites of tissue bridging in adults at 2 weeks post injury. GFAP (magenta) stains glial cells. FIG. 8D shows the expression of hb-egfa:EGFP at 4 and 6 wpi. Acetylated α-tubulin stains axons. Scale bars are 100 μm in FIG. 8A and FIG. 8B, and scale bars are 200 μm in FIG. 8C and FIG. 8D (d=dorsal; v=ventral; r=rostral, c=caudal).

FIG. 9A-FIG. 9C shows the characterization of hb-egfaOE zebrafish. FIG. 9A shows the timeline for heat shock experiments and assessment of ependymal cell proliferation, bridging, axon growth and swim capacity in hb-egfaOE zebrafish. All animals, transgenics, and controls, underwent daily heat shocks (HS). FIG. 9B shows ependymal (Sox2′) cell proliferation assessed by EdU incorporation in wild-type and hb-egfaOE adult spinal cords at 1 wpi. FIG. 9C shows the percentage of tissue bridging in wild-type and hsp70:hbegfOE zebrafish at 2 wpi.

FIG. 10 shows the hydrogel-mediated FITC release at the site of spinal cord injury. Live zebrafish underwent spinal cord injury and a single injection of FITC-loaded hydrogel was placed at a site anterior or posterior to the transection site, imaged 2 days and 21 days post injection. Red arrows indicate site of spinal cord injury. One representative fish per group is shown.

FIG. 11A-FIG. 11D show hydrogel-mediated administration of HR-HB-EGF. FIG. 11A shows ependymal cell proliferation assessed by EdU (red) incorporation in spinal cords of fish treated with vehicle (BSA)- or HR-HB-EGF-loaded hydrogel, shown at 1 wpi. FIG. 11B shows quantification FIG. 11C shows tissue sections indicating GFAP (magenta) and acetylated α-tubulin (red) immunofluorescence at 2 wpi in fish treated with vehicle- or HR-HB-EGF-loaded hydrogel. Arrows indicate sites of tissue bridging (n=4). FIG. 11D shows quantification of tissue bridging. Scale bars are 50 μm in FIG. 11A and 200 μm in FIG. 11C (d=dorsal; v=ventral; r=rostral, c=caudal).

FIG. 12A-FIG. 12C shows the bioinformatic analyses of ATAC-seq and RNA-seq. FIG. 12A shows a bar plot showing the proportions of dynamic peaks during regeneration located within promoters, exons, introns, and intergenic regions. FIG. 12B shows a heat map of increased transcripts in 7 dpi regenerating spinal cords versus sham-injured, linked to nearby differentially accessible chromatin regions. FIG. 12C shows gene ontology analyses of genes with associated chromatin regions after spinal cord injury. Compared to uninjured spinal cord, injury triggers enrichment of signaling pathways involved predominantly in central nervous system (CNS) development and morphogenesis. Pathways involved in developmental growth, with major functions in axonogenesis and guidance, neurogenesis and angiogenesis are also enriched, supporting the establishment of regeneration programs at early stages after injury. Hb-egf is a ligand for EGFR, which often links extracellular signals with changes in gene expression via a mitogen-activated protein kinase (MAPK)-signaling pathway. In agreement with a role for Hb-egf in SC regeneration, the MAPK-signaling pathway was also enriched in the GO analyses. GO analyses at one-week post injury shown in Table 3.

FIG. 13A-FIG. 13C shows that hb-egfEN directs EGFP fluorescence after spinal cord injury. FIG. 13A shows in vivo imaging of hb-egfEN-cfos:EGFP larvae uninjured and at 2 and 3 days post injury. Injuries were performed at 3 days post fertilization. GFAP-H2A:mCherry marks glial cell nuclei. FIG. 13B shows longitudinal sections of cfos:EGFP zebrafish, used as controls, at 1 and 2 weeks post injury. No EGFP expression is detected at the site of injury in absence of hb-egfaEN. FIG. 13C shows longitudinal sections of hb-egfaEN-cfos:EGFP zebrafish at 4 and 6 weeks post spinal cord injury. Scale bars are 100 μm in FIG. 13A, 200 μm in FIG. 13B) and FIG. 13C (d=dorsal; v=ventral).

FIG. 14A-FIG. 14E shows the characterization of hsp70:sox2 zebrafish. FIG. 14A shows sections of 6 dpf (hsp70:sox2) and wild-type zebrafish larvae stained for the transcription factor Sox2 after daily heat shocks from 3 to 6 dpf. Magnified sections on the right show Sox2 expression in the head region. FIG. 14B shows western blot showing Sox2 protein levels in brain, fin, heart and spinal cord of adult zebrafish after daily heat shocks. FIG. 14C shows quantification of protein levels. FIG. 14D shows qPCR showing mRNA relative expression level of sox2 and hh-egfa in spinal cords of adult zebrafish at 1 week after heat shock. FIG. 14E shows longitudinal sections of wild-type and hsp70:sox2; hb-egfa:EGFP zebrafish at 7 days post injury, after daily one hour-long heat shocks. Sox2 and hb-egfa:EGFP expression patterns between the two groups are unchanged. Scale bars are 200 μm in FIG. 14A and 100 μm in FIG. 14A, right panel) and 200 μm in FIG. 14D (r=rostral; c=caudal).

FIG. 15A-FIG. 15F shows CC47 infects neurons and cells with glial morphology in spinal cords of neonatal and adult mice. FIG. 15A shows the transgene construct and FIG. 15B shows the experimental design for testing CC47 transduction in adult spinal cord. FIG. 15C shows the immunofluorescence staining of adult spinal cord showing expression of EGFP, the glial marker GFAP, and the neuronal marker HuC/D at 14 days post injection. FIG. 15D shows the transgene construct and FIG. 15E shows the experimental design for testing CC47 transduction in neonatal spinal cord. FIG. 15F shows immunofluorescence staining of neonatal spinal cord showing expression of EGFP, the glial marker GFAP and the neuronal marker HuC/D at 14 days post injection. Scale bars are 100 μm.

FIG. 16A-FIG. 16F shows the characterization of hsp68:EGFP after CC47 mediated delivery in neonatal and adult mice. FIG. 16A shows the transgene construct and FIG. 168 shows the experimental design for tests of hsp68:EGFP expression in adult spinal cord. FIG. 16C shows the longitudinal sections of spinal cords from adult mice after sham-injury or 7 days post crush injury. FIG. 16D shows the transgene construct and FIG. 16E shows experimental design for tests of hsp68:EGFP expression in neonatal spinal cord. FIG. 16I shows longitudinal sections of spinal cords from neonatal mice after sham injury or 4 days post crush injury. Both adult and neonatal mice injected with CC47 hsp68:EGFP show little or no EGFP at the site of injury. Scale bars are 500 μm in FIG. 16C and 100 μm in FIG. 16F.

FIG. 17A-FIG. 17C shows that neonatal mice regenerate axons after spinal cord crush injury. FIG. 17A shows the experimental design. FIG. 17B shows longitudinal sections of spinal cords of uninjured mice collected at postnatal day 4 (P4) or postnatal day 17 (P17) stained with the glial marker GFAP and 5-HT, marking serotonergic axons. FIG. 17C shows longitudinal sections of spinal cords after crush injury at P3, collected at P4 or P17, and stained with GFAP and 5-HT. 5-HT-positive axons are visible caudal to the injury site. Scale bars are 200 μm.

FIG. 18A-FIG. 18F shows characterization of cells activating hb-egfEN-hsp68:EGFP construct after spinal cord injury in neonatal mice. FIG. 18A shows the transgene construct and FIG. 18B shows the experimental design for hb-egfaEN-hsp68:EGFP fluorescence characterization in neonatal mouse spinal cord. FIG. 18C shows the expression of hb-egfaEN-directed EGFP and the neuronal marker HuC/D in spinal cord at 4 days post injury. FIG. 18D shows the expression of hb-egfaEN-driven EGFP and the macrophage marker F40/80 in neonatal spinal cord at 4 days post injury. FIG. 15E shows the expression of hb-egfaEN-driven EGFP and the microglial marker CD68 in spinal cord at 4 days post injury. FIG. 18F shows the expression of hb-egfaEN-driven EGFP and bridge-forming fibronectin in spinal cord at 4 days post injury. In FIG. 18C-FIG. 18F, the scale bars are 100 μm.

FIG. 19A FIG. 19E shows the synthesis of the hydrogel used of HB-EGF delivery to injured and/or damaged spinal cord.

### DETAILED DESCRIPTION OF THE INVENTION

The present disclosure describes compositions, isolated nucleic acids, fusion products, pharmaceutical formulations, and methods of using the disclosed compositions, isolated nucleic acids, fusion products, pharmaceutical formulations thereof. It is to be understood that the inventive aspects of which are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention.

### A. Definitions

Before the present compounds, compositions, articles, systems, devices, vectors, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

This disclosure describes inventive concepts with reference to specific examples. However, the intent is to cover all modifications, equivalents, and alternatives of the inventive concepts that are consistent with this disclosure.

As used in the specification and the appended claims, the singular forms “a”, “an”, and “the” include plural referents unless the context clearly dictates otherwise.

The phrase “consisting essentially of” limits the scope of a claim to the recited components in a composition or the recited steps in a method as well as those that do not materially affect the basic and novel characteristic or characteristics of the claimed composition or claimed method. The phrase “consisting of” excludes any component, step, or element that is not recited in the claim. The phrase “comprising” is synonymous with “including”, “containing”, or “characterized by”, and is inclusive or open-ended. “Comprising” does not exclude additional, unrecited components or steps.

As used herein, when referring to any numerical value, the term “about” means a value falling within a range that is A 10% of the stated value.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about.” it will be understood that the particular value forms a further aspect. It will be further understood that the endpoints of each of the ranges are significant both in relation to the other endpoint and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the term “approximately” or “about,” as applied to one or more values of interest, refers to a value that is similar to a stated reference value. In an aspect, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction of the stated reference value unless otherwise stated or otherwise evident from the context.

As used herein, the term “in vitro” refers to events or experiments that occur in an artificial environment, e.g., in a petri dish, test tube, cell culture, etc., rather than within a multicellular organism. As used herein, the term “in vivo” refers to events or experiments that occur within a multicellular organism.

As used herein, a “biomarker” refers to a defined characteristic that is measured as an indicator of normal biological processes, pathogenic processes, or response to an exposure of intervention. In an aspect, a biomarker can be diagnostic (i.e., detects or classifies a pathological condition), prognostic (i.e., predicts the probability of disease occurrence or progression), pharmacodynamic responsive (i.e., identifies a change in response to a therapeutic intervention), predictive (i.e., predicts how an individual or subject might respond to a particular intervention or event). In an aspect, a biomarker can be diagnostic, prognostic, pharmacodynamic/responsive, and/or predictive at the same time. In an aspect, a biomarker can be diagnostic, prognostic, pharmacodynamic/responsive, and/or predictive at different times (e.g., first a biomarker can be diagnostic and then later, the same biomarker can be prognostic, pharmacodynamic/responsive, and/or predictive). A biomarker can be an objective measure that can be linked to a clinical outcome assessment. A biomarker can be used by the skilled person to make a clinical decision based on its context of use.

References in the specification and concluding claims to parts by weight of a particular element or component in a composition denotes the weight relationship between the element or component and any other elements or components in the composition or article for which a part by weight is expressed. Thus, in a compound containing 2 parts by weight component X and S parts by weight component Y, X and Y are present at a weight ratio of 2:5, and are present in such ratio regardless of whether additional components are contained in the compound.

As used herein, the terms “optional” or “optionally” means that the subsequently described event or circumstance can or cannot occur, and that the description includes instances where said event or circumstance occurs and instances where it does not. In an aspect, a disclosed method can optionally comprise one or more additional steps, such as, for example, repeating an administering step or altering an administering step.

As used herein, the term “subject” refers to the target of administration. In an aspect, a subject can be a human being. The term “subject” includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). Thus, the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig, or rodent. The term does not denote a particular age or sex, and thus, adult and child subjects, as well as fetuses, whether male or female, are intended to be covered. In an aspect, a subject can be a human patient. In an aspect, a subject can have damaged and/or injured spinal cord tissues, or be suspected of having damaged and/or injured spinal cord tissues, or be at risk of developing damaged and/or injured spinal cord tissues.

As used herein, “effective amount” and “amount effective” can refer to an amount that is sufficient to achieve the desired result such as, for example, the treatment and/or regeneration of damaged and/or injured spinal cord tissues. As used herein, the terms “effective amount” and “amount effective” can refer to an amount that is sufficient to achieve the desired effect on damaged and/or injured spinal cord tissues. For example, a “therapeutically effective amount” refers to an amount that is sufficient to achieve the desired therapeutic result or to have an effect on undesired symptoms, but is generally insufficient to cause adverse side effects. In an aspect, “therapeutically effective amount” means an amount of a disclosed composition that (i) treats the damaged and/or injured spinal cord tissues, (ii) attenuates, ameliorates, or eliminates one or more symptoms of the particular damaged and/or injured spinal cord tissues, or (iii) delays the onset of one or more symptoms of the particular damaged and/or injured spinal cord tissues described herein. The specific therapeutically effective dose level for any particular patient will depend upon a variety of factors including the disorder being treated and the severity of the disorder; the specific disclosed compositions and/or a pharmaceutical preparation comprising one or more disclosed compositions, or methods employed; the age, body weight, general health, sex and diet of the patient; the time of administration; the route of administration; the rate of excretion of the disclosed compositions and/or a pharmaceutical preparation comprising one or more disclosed compositions employed; the duration of the treatment; drugs used in combination or coincidental with a disclosed compositions and/or a pharmaceutical preparation comprising one or more disclosed compositions employed, and other like factors well known in the medical arts. For example, it is well within the skill of the art to start doses of a disclosed composition and/or a pharmaceutical preparation comprising one or more disclosed composition at levels lower than those required to achieve the desired therapeutic effect and to gradually increase the dosage until the desired effect is achieved. If desired, then the effective daily dose can be divided into multiple doses for purposes of administration. Consequently, a single dose of a disclosed compositions and/or a pharmaceutical preparation comprising one or more disclosed compositions, or methods can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In an aspect, a preparation can be administered in a “prophylactically effective amount”; that is, an amount effective for prevention of a disease, a disorder, an infection, a symptom, and/or a complication.

“Control” as used herein refers a standard or reference condition, against which results are compared. In an aspect, a control is used at the same time as a test variable or subject to provide a comparison. In an aspect, a control is a historical control that has been performed previously, a result or amount that has been previously known, or an otherwise existing record. A control may be a positive or negative control.

As used herein, the term “diagnosed” means having been subjected to a physical examination by a person of skill, for example, a physician, and found to have damaged and/or injured spinal cord tissues that can be diagnosed or treated by one or more of the disclosed nucleic acids, the disclosed vectors, the disclosed compositions, the disclosed pharmaceutical preparations, and/or the disclosed methods. For example, “suspected of having” can mean having been subjected to a physical examination by a person of skill, for example, a physician, and found to have damaged and/or injured spinal cord tissues that can likely be treated by one or more of the disclosed nucleic acids, the disclosed vectors, the disclosed compositions, the disclosed pharmaceutical preparations, and/or the disclosed methods.

The words “treat” or “treating” or “treatment” refer to therapeutic or medical treatment wherein the object is to slow down (lessen), ameliorate, and/or diminish an undesired physiological change, disease, disorder, injury, pathological condition, or disorder in a subject (such as, for example, a SCI). As used herein, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Treatment may not necessarily result in the complete clearance of an infection but may reduce or minimize complications, the side effects, and/or the progression of a disease, a disorder, an injury, an infection, a symptom, and/or a complication (such as, for example, a SCI). The success or otherwise of treatment may be monitored by physical examination of the subject as well as cytopathological, DNA, and/or mRNA detection techniques. The words “treat” or “treating” or “treatment” include palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder (such as, for example, a SCI). In various aspects, the term covers any treatment of a subject, including a mammal (e.g., a human), and includes: (i) preventing the undesired physiological change, disease, injury, insult, pathological condition, or disorder from occurring in a subject that can be predisposed to the disease but has not yet been diagnosed as having it; (ii) inhibiting the physiological change, disease, pathological condition, or disorder, i.e., arresting its development; or (iii) relieving the physiological change, disease, pathological condition, or disorder, i.e., causing regression of the disease. For example, in an aspect, treating damaged and/or injured spinal cord tissues can reduce the severity of damaged and/or injured spinal cord tissues in a subject by 1%-100% as compared to a control (such as, for example, a subject not having the disease, the disorder, the injury, the infection, the symptom, and/or the complication (such as, for example, a SCI). In an aspect, treating can refer to a 1%, 2%, 3%, 4%, 5%, 6%, 7%, 8%, 9%, 10%, 20%, 30%, 40%, 50%, 60%, 70%, 80%, 90%, or 100% reduction in the severity of damaged and/or injured spinal cord tissues. In an aspect, treating can refer to 1%, 2%, 3%, 4%, 5%, 6%, 7%, 8%, 9%, 10%, 20%, 30%, 40%, 50%, 60%, 70%, 80%, 90%, 100% reduction of one or more symptoms. It is understood that treatment does not necessarily refer to a cure or complete ablation or eradication of the damaged and/or injured spinal cord tissues. However, in an aspect, treatment can refer to a cure or complete ablation or eradication of the damaged and/or injured spinal cord tissues.

A “patient” refers to a subject afflicted with damaged and/or injured spinal cord tissues. In an aspect, a patient can refer to a subject that has been diagnosed with or is suspected of having a disease, disorder, infection, symptom, and/or complication that results in damaged and/or injured spinal cord tissues. In an aspect, a patient can refer to a subject that has been diagnosed with or is suspected of having an established disease, disorder, infection, symptom, and/or complication that results in damaged and/or injured spinal cord tissues (such as, for example, a SCI) and is seeking treatment or receiving treatment.

As used herein, the term “prevent” or “preventing” or “prevention” refers to precluding, averting, obviating, forestalling, stopping, or hindering something from happening, especially by advance action. It is understood that where reduce, inhibit, or prevent are used herein, unless specifically indicated otherwise, the use of the other two words is also expressly disclosed. In an aspect, preventing a disease, disorder, infection, symptom, and/or complication is intended. In an aspect, preventing damaged and/or injured spinal cord tissues is intended. The words “prevent” and “preventing” and “prevention” also refer to prophylactic or preventative measures for protecting or precluding a subject (e.g., an individual) not having a given complication associated with damaged and/or injured spinal cord tissues from progressing to that complication.

As used herein, the terms “administering” and “administration” refer to any method of providing a disclosed HB-EGF or one or more of the disclosed isolated nucleic acid molecules, disclosed pharmaceutical formulations, disclosed vectors, or any combination thereof to a subject. Such methods are well known to those skilled in the art and include, but are not limited to, the following routes: local administration, direct administration, oral administration, transdermal administration, administration by inhalation, nasal administration, topical administration, in utero administration, intrahepatic administration, intravaginal administration, ophthalmic administration, intraaural administration, otic administration, intracerebral administration, rectal administration, sublingual administration, buccal administration, and parenteral administration, including injectable such as intravenous administration, intra-CSF administration, intra-arterial administration, intramuscular administration, and subcutaneous administration. Administration can also include hepatic intra-arterial administration or administration through the hepatic portal vein (HPV). Administration of a disclosed nucleic acid molecule, a disclosed vector, a disclosed therapeutic agent, a disclosed pharmaceutical formulation, or a combination thereof can comprise administration directly into the CNS (e.g., intraparenchymal, intracerebroventriular, inthrathecal cisternal, intrathecal (lumbar), deep gray matter delivery, convection-enhanced delivery to deep gray matter) or the PNS. Administration can be continuous or intermittent.

As used herein, “concurrently” means (1) simultaneously in time, or (2) at different times during the course of a common treatment schedule.

The term “contacting” as used herein refers to bringing one or more of the disclosed nucleic acids, the disclosed vectors, the disclosed compositions, and/or the disclosed pharmaceutical formulations together with a target area or intended target area (i.e., damaged and/or injured spinal cord tissues) in such a manner that the one or more disclosed nucleic acids, vectors, compositions, and/or pharmaceutical formulation can exert an effect on the intended target or targeted area (i.e., damaged and/or injured spinal cord tissues) either directly or indirectly.

As used herein, “determining” can refer to measuring or ascertaining the presence and severity of a disease, disorder, injury, infection, symptom, and/or complication or the presence and severity of damaged and/or injured spinal cord tissues. Methods and techniques used to determining the presence and/or severity of a disease, disorder, injury, infection, symptom, and/or complication or the presence and/or severity of damaged and/or injured spinal cord tissues are typically known to the medical arts. For example, the art is familiar with the ways to identify and/or diagnose the presence, severity, or both of a disease, disorder, infection, symptom, and/or complication, or the presence, severity, or both of damaged and/or injured spinal cord tissues.

As used herein, “CRISPR or clustered regularly interspaced short palindromic repeat” is an ideal tool for correction of genetic abnormalities as the system can be designed to target genomic DNA directly. A CRISPR system involves two main components: a Cas9 enzyme and a guide (gRNA). The gRNA contains a targeting sequence for DNA binding and a scaffold sequence for Cas9 binding. Cas9 nuclease is often used to “knockout” target genes hence it can be applied for deletion or suppression of oncogenes that are essential for cancer initiation or progression. Similar to ASOs and siRNAs, CRISPR offers a great flexibility in targeting any gene of interest hence, potential CRISPR based therapies can be designed based on the genetic mutation in individual patients. An advantage of CRISPR is its ability to completely ablate the expression of disease genes which can only be suppressed partially by RNA interference methods with ASOs or siRNAs. Furthermore, multiple gRNAs can be employed to suppress or activate multiple genes simultaneously, hence increasing the treatment efficacy and reducing resistance potentially caused by new mutations in the target genes.

As used herein, “CRISPRa” refers to CRISPR Activation, which is using a dCas9 or dCas9-activator with a gRNA to increase transcription of a target gene.

As used herein, “CRISPRi” refers to CRISPR Interference, which is using a dCas9 or dCas9-repressor with a gRNA to repress/decrease transcription of a target gene.

As used herein, “dCas9” refers to enzymatically inactive form of Cas9, which can bind, but cannot cleave. DNA. In an aspect, a disclosed dCas can comprise dVQR, dEQR, or dVRER.

As used herein, “Protospacer Adjacent Motif” or “PAM” refers to a sequence adjacent to the target sequence that is necessary for Cas enzymes to bind target DNA.

As used herein, “CRISPR-based endonucleases” include RNA-guided endonucleases that comprise at least one nuclease domain and at least one domain that interacts with a guide RNA. As known to the art, a guide RNA directs the CRISPR-based endonucleases to a targeted site in a nucleic acid at which site the CRISPR-based endonucleases cleaves at least one strand of the targeted nucleic acid sequence. As the guide RNA provides the specificity for the targeted cleavage, the CRISPR-based endonuclease is universal and can be used with different guide RN As to cleave different target nucleic acid sequences. CRISPR-based endonucleases are RNA-guided endonucleases derived from CRISPR/Cas systems. Bacteria and archaea have evolved an RNA-based adaptive immune system that uses CRISPR (clustered regularly interspersed short palindromic repeat) and Cas (CRISPR-associated) proteins to detect and destroy invading viruses or plasmids. CRISPR/Cas endonucleases can be programmed to introduce targeted site-specific double-strand breaks by providing target-specific synthetic guide RNAs (Jinek et al. (2012) Science. 337:816-821).

In an aspect, a disclosed CRISPR-based endonuclease can be derived from a CRISPR/Cas type I, type II, or type III system. Non-limiting examples of suitable CRISPR/Cas proteins include Cas3, Cas4, Cas5, Cas5e (or CasD), Cas6, Cas6e, Cas6f, Cas7, Cas8a1, Cas8a2, Cas8b, Cas8c, Cas9, Cas10, Cas10d, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Cs2, Cs3, Csf4, and Cu1966.

In an aspect, a disclosed CRISPR-based endonuclease can be derived from a type II CRISPR/Cas system. For example, in an aspect, a CRISPR-based endonuclease can be derived from a Cas9 protein. The Cas9 protein can be from Streptococcus pyogenes, Streptococcus thermophilus, Streptococcus sp, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, or Acaryochloris marina. In an aspect, the CRISPR-based nuclease can be derived from a Cas9 protein from Streptococcus pyogenes.

In general, CRISPR/Cas proteins can comprise at least one RNA recognition and/or RNA binding domain. RNA recognition and/or RNA binding domains can interact with the guide RNA such that the CRISPR/Cas protein is directed to a specific genomic or genomic sequence. CRISPR/Cas proteins can also comprise nuclease domains (i.e., DNase or RNase domains), DNA binding domains, helicase domains, protein-protein interaction domains, dimerization domains, as well as other domains.

The CRISPR-based endonuclease can be a wild type CRISPR/Cas protein, a modified CRISPR/Cas protein, or a fragment of a wild type or modified CRISPR/Cas protein. The CRISPR/Cas protein can be modified to increase nucleic acid binding affinity and/or specificity, alter an enzymatic activity, and/or change another property of the protein. For example, in an aspect, nuclease (i.e., DNase, RNase) domains of the CRISPR/Cas protein can be modified, deleted, or inactivated. A CRISPR/Cas protein can be truncated to remove domains that are not essential for the function of the protein. A CRISPR/Cas protein also can be truncated or modified to optimize the activity of the protein or an effector domain fused with a CRISPR/Cas protein.

In an aspect, a disclosed CRISPR-based endonuclease can be derived from a wild type Cas9 protein or fragment thereof. In an aspect, a disclosed CRISPR-based endonuclease can be derived from a modified Cas9 protein. For example, the amino acid sequence of a disclosed Cas9 protein can be modified to alter one or more properties (e.g., nuclease activity, affinity, stability, etc.) of the protein. Alternatively, domains of the Cas9 protein not involved in RNA-guided cleavage can be eliminated from the protein such that the modified Cas9 protein is smaller than the wild type Cas9 protein.

As used herein. “promoter” or “promoters” are known to the art. Depending on the level and tissue-specific expression desired, a variety of promoter elements can be used. A promoter can be tissue-specific or ubiquitous and can be constitutive or inducible, depending on the pattern of the gene expression desired. A promoter can be native or foreign and can be a natural or a synthetic sequence. By foreign, it is intended that the transcriptional initiation region is not found in the wild-type host into which the transcriptional initiation region is introduced.

“Tissue-specific promoters” are known to the art and include, but are not limited to, neuron-specific promoters, muscle-specific promoters, liver-specific promoters, skeletal muscle-specific promoters, and heart-specific promoters.

“Neuron-specific promoters” are known to the art and include, but are not limited to, the synapsin I (SYN) promoter, the calcium/calmodulin-dependent protein kinase II promoter, the tubulin alpha I promoter, the neuron-specific enolase promoter, and the platelet-derived growth factor beta chain promoter.

As used herein, a “ubiquitous/constitutive promoter” refer to a promoter that allows for continual transcription of its associated gene. A ubiquitous/constitutive promoter is always active and can be used to express genes in a wide range of cells and tissues, including, but not limited to, the liver, kidney, skeletal muscle, cardiac muscle, smooth muscle, diaphragm muscle, brain, spinal cord, endothelial cells, intestinal cells, pulmonary cells (e.g., smooth muscle or epithelium), peritoneal epithelial cells, and fibroblasts. Ubiquitous/constitutive promoters include, but are not limited to, a CMV major immediate-early enhancer/chicken beta-actin promoter, a cytomegalovirus (CMV) major immediate-early promoter, an Elongation Factor 1-α (EF1-α) promoter, a simian vacuolating virus 40 (SV40) promoter, an AmpR promoter, a PγK promoter, a human ubiquitin C gene (Ubc) promoter, a MFG promoter, a human beta actin promoter, a CAG promoter, a EGR1 promoter, a FerH promoter, a FerL promoter, a GRP78 promoter, a GRP94 promoter, a HSP70 promoter, a β-kin promoter, a murine phosphoglycerate kinase (mPGK) or human PGK (hPGK) promoter, a ROSA promoter, human Ubiquitin B promoter, a Rous sarcoma virus promoter, or any other natural or synthetic ubiquitous/constitutive promoters.

As used herein, an “inducible promoter” refers to a promoter that can be regulated by positive or negative control. Factors that can regulate an inducible promoter include, but are not limited to, chemical agents (e.g., the metallothionein promoter or a hormone inducible promoter), temperature, and light.

As used herein, “operably linked” means that expression of a gene or a transgene is under the control of a promoter or control element with which it is spatially connected. A promoter can be positioned 5′ (upstream) or 3′ (downstream) of a gene under its control. The distance between the promoter and a gene can be approximately the same as the distance between that promoter and the gene it controls in the gene from which the promoter is derived. As is known in the art, variation in this distance can be accommodated without loss of promoter function.

As used herein, an “enhancer” such as a transcription or transcriptional enhancer refers to regulatory DNA segment that is typically found in multicellular eukaryotes. An enhancer can strongly stimulate (“enhance”) the transcription of a linked transcription unit, i.e., it acts in cis. An enhancer can activate transcription over very long distances of many thousand base pairs, and from a position upstream or downstream of the site of transcription initiation. An enhancers can have a modular structure by being composed of multiple binding sites for transcriptional activator proteins. Many enhancers control gene expression in a cell type-specific fashion. Several remote enhancers can control the expression of a singular gene while a singular enhance can stimulate the transcription of one or more genes.

As used herein, “expression cassette” or “transgene cassette” can refer to a distinct component of vector DNA comprising a transgene and one or more regulatory sequences to be expressed by a transfected cell. Generally, an expression cassette or transgene cassette can comprise a promoter sequence, an open reading frame (i.e., the transgene such as, for example, an HB-EGF), and a 3′ untranslated region (e.g., in eukaryotes a polyadenylation site).

As used herein, an “isolated” biological component (such as a nucleic acid molecule, protein, or virus) has been substantially separated or purified away from other biological components (e.g., other chromosomal and extra-chromosomal DNA and RNA, proteins and/or organelles). Nucleic acids, proteins, and/or viruses that have been “isolated” include nucleic acids, proteins, and viruses purified by standard purification methods. The term also embraces nucleic acids, proteins, and viruses prepared by recombinant expression in a host cell, as well as chemically synthesized nucleic acids or proteins. The term “isolated” (or purified) does not require absolute purity; rather, it is intended as a relative term. Thus, for example, an isolated or purified nucleic acid, protein, virus, or other active compound is one that is isolated in whole or in part from associated nucleic acids, proteins, and other contaminants. In an aspect, the term “substantially purified” refers to a nucleic acid, protein, virus or other active compound that has been isolated from a cell, cell culture medium, or other crude preparation and subjected to fractionation to remove various components of the initial preparation, such as proteins, cellular debris, and other components.

“Sequence identity” and “sequence similarity” can be determined by alignment of two peptide or two nucleotide sequences using global or local alignment algorithms. Sequences may then be referred to as “substantially identical” or “essentially similar” when they are optimally aligned. For example, sequence similarity or identity can be determined by searching against databases such as FASTA, BLAST, etc., but hits should be retrieved and aligned pairwise to compare sequence identity. Two proteins or two protein domains, or two nucleic acid sequences can have “substantial sequence identity” if the percentage sequence identity is at least 70%, 75%, 80%, 85%, 90%, 95%, 98%, 99% or more, preferably 90%, 95%, 98%, 99% or more. Such sequences are also referred to as “variants” herein, e.g., other variants of glycogen branching enzymes and amylases. It should be understood that sequence with substantial sequence identity do not necessarily have the same length and may differ in length. For example, sequences that have the same nucleotide sequence but of which one has additional nucleotides on the 3′- and/or 5′-side are 100% identical.

Disclosed are the components to be used to prepare one or more of the disclosed nucleic acids, the disclosed vectors, the disclosed compositions, and/or the disclosed pharmaceutical formulations used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

### B. Compositions

### 1. Heparin-Binding Epidermal Growth Factor

Disclosed herein are compositions comprising HB-EGF. In an aspect, HB-EGF can comprise a recombinant HB-EGF. In an aspect, a disclosed HB-EGF can comprise epidermal growth factor a (HB-EGFa) or recombinant heparin binding epidermal growth factor a (rHB-EGFa). In an aspect, a disclosed HB-EGF can comprise heparin binding epidermal growth factor b (HB-EGFb) or recombinant heparin binding epidermal growth factor b (rHB-EGFb). In an aspect, a disclosed HB-EGF can comprise recombinant human HB-EGF. In an aspect. HB-EGF can comprise the sequence set forth in SEQ ID NO:35, SEQ ID NO:36, SEQ ID NO:37, or SEQ ID NO:55 or a fragment thereof. In an aspect, HB-EGF can comprise a sequence having about 50%, about 55%, about 60%, about 65%, about 70%, about 75%, about 80%, about 85%, about 90%, about 95%, or more than 95% identity to the sequence set forth in SEQ ID NO:35, SEQ ID NO:36, SEQ ID NO:37, or SEQ ID NO:55 or a fragment thereof. In an aspect. HB-EGF can treat a spinal cord injury, can stimulate regeneration of injured and/or damaged spinal cord tissue, can promote glial cell proliferation in injured and/or damaged spinal cord tissue, can promote axonal tract regeneration in injured and/or damaged spinal cord tissue, can trigger neurite outgrowth in injured and/or damaged spinal cord tissue, can trigger neuron formation in injured and/or damaged spinal cord tissue, can improve spinal cord function in a subject in need thereof, or any combination thereof. Spinal cord function can comprise sensory function, motor function, or a combination thereof. Spinal cord function can be assessed, examined, and/or measured by one or more methods known to the skilled person. For example, HB-EGF can improve a subject's ASIA score.

### 2. Isolated Nucleic Acid Molecules

Disclosed herein is an isolated nucleic acid molecule, comprising a nucleic acid sequence encoding a tissue regeneration enhancer element (TREE); an encoded polypeptide; and a promoter directing expression of the encoded polypeptide in damaged and/or injured spinal cord tissues. Disclosed herein is an isolated nucleic acid molecule, comprising a nucleic acid sequence encoding a tissue regeneration enhancer element (TREE); an encoded polypeptide; a promoter directing expression of the encoded polypeptide in damaged and/or injured spinal cord tissues; and a 3′ UTR noncoding region.

In an aspect, a disclosed isolated nucleic acid molecule can comprise a 3′ UTR noncoding region. In an aspect, a disclosed 3′ UTR noncoding region can stabilize the transcribed RNA message. In an aspect, a disclosed 3′ UTR noncoding region can comprise a polyadenylation (polyA) sequence and/or a structural element that stabilizes the transcribed RNA message. In an aspect, a disclosed isolated nucleic acid molecule can comprise inverted terminal repeats (for example, ITRs derived from a viral genome such as an AAV genome).

In an aspect, damaged and/or injured spinal cord tissues can comprise mammalian or non-mammalian spinal cord tissue. For example, in an aspect, mammalian spinal cord tissue can comprise human spinal cord tissue. For example, in an aspect, mammalian spinal cord tissue can comprise non-human spinal cord tissue.

In an aspect, spinal cord tissues can comprise neurons, neuroglia, or a combination thereof. In an aspect, neuroglia can comprise microglia and/or macroglia. In an aspect, neuroglia can comprise microglia, astrocytes, oligodendrocytes, ependymal cells, radial glia, Schwann cells, satellite cells, or any combination thereof.

In an aspect of a disclosed isolated nucleic acid molecule, a disclosed promoter can comprise a minimal promoter. In an aspect, a disclosed minimal promoter can comprise a human minimal promoter or a murine minimal promoter. In an aspect, a disclosed minimal promoter can comprise little or no basal activity in mammalian or non-mammalian spinal cord tissues.

In an aspect, a disclosed promoter can comprise a Hsp70 promoter or a fragment thereof. In an aspect, a disclosed Hsp70 promoter can be found at danRer10 coord chr8:4,740.922-4,742,463 in NCBI sequence ID no. FP017299.16. In an aspect, a disclosed zebrafish Hsp70 promoter can comprise a sequence having about 70%, about 75%, about 80%, about 85%, about 90%, about 95%, or more than 95% identity to the sequence at danRer10 coord chr8:4,740.922-4,742.463 in NCBI sequence ID no. FP017299.16. In an aspect, a disclosed Hsp70 promoter can comprise the sequence set forth in SEQ ID NO:38 or a fragment thereof, or a sequence having about 50%, about 55%, about 60%, about 65%, about 70%, about 75%, about 80%, about 85%, about 90%, about 95%, or more than 95% identity to the sequence set forth in SEQ ID NO:38 or a fragment thereof. In an aspect, a disclosed Hsp70 promoter can comprise the sequence set forth in SEQ ID NO:39 or a fragment thereof, or a sequence having about 50%, about 55%, about 60%, about 65%, about 70%, about 75%, about 80%, about 85%, about 90%, about 95%, or more than 95% identity to the sequence set forth in SEQ ID NO:39 or a fragment thereof. Hsp70 promoters as well as the sequences for Hsp70 promoters are known to the art.

In an aspect, a disclosed promoter can comprise a H-sp68 promoter or a fragment thereof. In an aspect, a disclosed Hsp68 promoter can be found at mm10 coord chr17:34,971,928-34,972,798 in NCBI sequence ID no. CU457784.5. In an aspect, a disclosed murine Hsp68 promoter can comprise a sequence having about 70%, about 75%, about 80%, about 85%, about 90%, about 95%, or more than 95% identity to the sequence at/mm10 coord chr17:34,971,928-34,972,798 in NCBI sequence ID no. CU457784.5. Hsp68 promoters as well as the sequences for Hsp68 promoters are known to the art.

In an aspect, a disclosed promoter can comprise a cfos promoter or a fragment thereof. In an aspect, a disclosed promoter can comprise a murine cfos promoter or a fragment thereof. In an aspect, a disclosed murine cfos promoter can be found at mm10 coord chr12:85,473,820-85,473,917 in NCBI sequence ID no. AF332140.1. In an aspect, a disclosed murine cfos promoter can comprise a sequence having about 70%, about 75%, about 80%, about 85%, about 90%, about 95%, or more than 95% identity to the sequence at mm10 coord chr12:85,473,820-85,473,917 in NCBI sequence ID no. AF332140.1. In an aspect. NCBI sequence ID no. AF332140.1 can comprise the sequence set forth in SEQ ID NO:40. cfos promoters as well as the sequences for cfos promoters are known to the art.

In an aspect, a disclosed promoter can comprise an AAV e1b promoter. In an aspect, a disclosed AAV e1B promoter can comprise the sequence set forth in SEQ ID NO:41 or a fragment thereof, or a sequence having about 70%, about 75%, about 80%, about 85%, about 90%, about 95%, or more than 95% identity to the sequence set forth in SEQ ID NO:41 or a fragment thereof. In an aspect, a disclosed AAV e1B promoter can comprise the sequence set forth in GenBank Accession No. KU664676.1 or a fragment thereof, or a sequence having about 50%, about 55%, about 60%, about 65%, about 70%, about 75%, about 80%, about 85%, about 90%, about 95%, or more than 95% identity to the sequence set forth in GenBank Accession No. KU664676.1 or a fragment thereof. AAV e1B promoters as well as the sequences for e1B promoters are known to the art.

In an aspect, a disclosed promoter can comprise a CMV promoter/enhancer, a Thy1 promoter, a GFAP promoter, or a FoxJ1 promoter, all of which are known in the art.

In an aspect, a disclosed CMV promoter/enhancer can comprise the sequence set forth in SEQ ID NO:42 or a fragment thereof, or a sequence having about 70%, about 75%, about 80%, about 85%, about 90%, about 95%, or more than 95% identity to the sequence set forth in SEQ ID NO:42 or a fragment thereof. In an aspect, a disclosed CMV promoter can comprise the sequence set forth in GenBank Accession No. AF105229.1 or a fragment thereof, or a sequence having about 50%, about 55%, about 60%, about 65%, about 70%, about 75%, about 80%, about 85%, about 90%, about 95%, or more than 95% identity to the sequence set forth in GenBank Accession No. AF105229.1 or a fragment thereof. CMV promoters and/or enhancers as well as the sequences for CMV promoters and/or enhancers promoters are known to the art (see. e.g., U.S. Pat. Nos. 5,168,062, 5,385,839, and 6,218,140).

In an aspect, a disclosed Thy1 promoter can comprise the sequence set forth in SEQ ID NO:43 or a fragment thereof, or a sequence having about 50%, about 55%, about 60%, about 65%, about 70%, about 75%, about 80%, about 85%, about 90%, about 95%, or more than 95% identity to the sequence set forth in SEQ ID NO:43 or a fragment thereof. In an aspect, a disclosed CMV promoter can comprise the sequence set forth in GenBank Accession No. JN959674.1 or a fragment thereof, or a sequence having about 50%, about 55%, about 60%, about 65%, about 70%, about 75%, about 80%, about 85%, about 90%, about 95%, or more than 95% identity to the sequence set forth in GenBank Accession No. JN959674.1 or a fragment thereof. Thy1 promoters as well as the sequences for Thy1 promoters are known to the art.

In an aspect, a disclosed GFAP promoter can comprises the sequence set forth in SEQ ID NO:45 or a fragment thereof, or a sequence having about 70%, about 75%, about 80%, about 85%, about 90%, about 95%, or more than 95% identity to the sequence set forth in SEQ ID NO:45 or a fragment thereof. In an aspect, a disclosed GFAP promoter can comprises the sequence set forth in SEQ ID NO:46 or a fragment thereof, or a sequence having about 70%, about 75%, about 80%, about 85%, about 90%, about 95%, or more than 95% identity to the sequence set forth in SEQ ID NO:46 or a fragment thereof. In an aspect, a disclosed GFAP promoter can comprise the sequence set forth in GenBank Accession No. AY279974.1 (mouse) or Accession No. NG_008401.1 (human) or a fragment thereof, or a sequence having about 50%, about 55%, about 60%, about 65%, about 70%, about 75%, about 80%, about 85%, about 90%, about 95%, or more than 95% identity to the sequence set forth in GenBank Accession No. AY279974.1 (mouse) or Accession No. NG_008401.1 (human) or a fragment thereof. GFAP promoters as well as the sequences for GFAP promoters are known to the art.

In an aspect, a disclosed FoxJ1 promoter can comprise the sequence set forth in SEQ JD NO:47, SEQ ID NO:48, or SEQ ID NO:49 or a fragment thereof, or a sequence having about 50%, about 55%, about 60%, about 65%, about 70%, about 75%, about 80%, about 85%, about 90%, about 95%, or more than 95% identity to the sequence set forth in SEQ ID NO:47, SEQ ID NO:48, or SEQ ID NO:49 or a fragment thereof. In an aspect, a disclosed FoxJ1 promoter can comprise the sequence set forth in GenBank Accession No. JN959674.1, GenBank Accession No. NG_13345.1, or GenBank Accession No. NM 008240.3 or a fragment thereof, or a sequence having about 50%, about 55%, about 60%, about 65%, about 70%, about 75%, about 80%, about 85%, about 90%, about 95%, or more than 95% identity to the sequence set forth in GenBank Accession No. JN959674.1, GenBank Accession No. NG_13345.1, or GenBank Accession No. NM_008240.3 or a fragment thereof. Foxj1 promoters as well as the sequences for Foxj1 promoters are known to the art.

In an aspect, a disclosed encoded polypeptide can have a pro-regenerative activity. In an aspect, a disclosed encoded polypeptide can promote glial cell proliferation, can promote axonal tract regeneration, can trigger neurite outgrowth, can trigger neuron formation, can stimulate regeneration of injured and/or damage spinal cord tissue, or any combination thereof. In an aspect, a disclosed encoded polypeptide can improve spinal cord function. In an aspect, spinal cord function can comprise sensory function and/or motor function.

In an aspect, a disclosed encoded polypeptide can be a transcription factor, a modified transcription factor, or a recombinant transcription factor. In an aspect, a disclosed transcription factor can promote glial cell proliferation, can promote axonal tract regeneration, can trigger neurite outgrowth, can trigger neuron formation, can stimulate regeneration of injured and/or damage spinal cord tissue, or any combination thereof. In an aspect, a disclosed transcription factor can improve spinal cord function. In an aspect, spinal cord function can comprise sensory function and/or motor function.

In an aspect, a disclosed encoded polypeptide can comprise a secreted factor, a modified secreted factor, or a recombinant secreted factor. In an aspect, a disclosed secreted factor can promote glial cell proliferation, can promote axonal tract regeneration, can trigger neurite outgrowth, can trigger neuron formation, can stimulate regeneration of injured and/or damage spinal cord tissue, or any combination thereof. In an aspect, a disclosed secreted factor can improve spinal cord function. In an aspect, spinal cord function can comprise sensory function and/or motor function.

In an aspect, a disclosed HB-EGF can promote glial cell proliferation, can promote axonal tract regeneration, can trigger neurite outgrowth, can trigger neuron formation, can stimulate regeneration of injured and/or damage spinal cord tissue, or any combination thereof. In an aspect, a disclosed HB-EGF can improve spinal cord function. In an aspect, spinal cord function can comprise sensory function and/or motor function.

In an aspect, a disclosed TREE can control the ability of a disclosed promoter to direct expression of the encoded polypeptide in the injured and/or damaged spinal cord tissue. In an aspect, a disclosed TREE can activate expression of a disclosed encoded polypeptide in the injured and/or damaged spinal cord tissue. In an aspect, a disclosed TREE can alleviate expression of a disclosed encoded polypeptide after regeneration concludes in the injured and/or damaged spinal cord tissue.

In an aspect, a disclosed TREE can control the ability of a disclosed promoter to direct expression of HB-EGF in the injured and/or damaged spinal cord tissue. In an aspect, a disclosed TREE can activate expression of HB-EGF in the injured and/or damaged spinal cord tissue. In an aspect, a disclosed TREE can alleviate expression of HB-EGF after regeneration concludes in the injured and/or damaged spinal cord tissue. In an aspect, a disclosed TREE can control the ability of a disclosed promoter to direct expression of BH-EGF in the injured and/or damaged spinal cord tissue.

In an aspect, a disclosed TREE can activate expression of a disclosed endogenous gene in the injured and/or damaged spinal cord tissue. In an aspect, a disclosed TREE can maintain expression of a disclosed endogenous gene during regeneration in the injured and/or damaged spinal cord tissue. In an aspect, a disclosed TREE can alleviate expression of a disclosed endogenous gene after regeneration concludes in the injured and/or damaged spinal cord tissue.

In an aspect, a disclosed TREE can control the ability of a disclosed promoter to direct expression of a reporter gene. In an aspect, a disclosed TREE can activate expression of a disclosed reporter gene in the injured and/or damaged spinal cord tissue. In an aspect, a disclosed TREE can maintain expression of a disclosed reporter gene during regeneration in the injured and/or damaged spinal cord tissue. In an aspect, a disclosed TREE can alleviate expression of a disclosed reporter gene after regeneration concludes in the injured and/or damaged spinal cord tissue.

In an aspect, a disclosed TREE can comprise a zebrafish TREE or a mammalian TREE.

In an aspect, a disclosed TREE can comprise hb-egfa-linked enhancer (hb-egfa-EN). In an aspect, a disclosed hb-egfa-EN can comprise the sequence set forth in SEQ ID NO:34 or a fragment thereof or a sequence having about 50%, about 55%, about 60%, about 65%, about 70%, about 75%, about 80%, about 85%, about 90%, about 95%, or more than 95% identity to the sequence set forth in SEQ ID NO:34 or a fragment thereof. In an aspect, a disclosed hb-egfa can comprise the hb-egfa sequence set forth in Gene ID: 797938 or a fragment thereof, or a sequence having about 50%, about 55%, about 60%, about 65%, about 70%, about 75%, about 80%, about 85%, about 90%, about 95%, or more than 95% identity to the sequence set forth in Gene ID: 797938 or a fragment thereof. In an aspect, a disclosed hb-egfa can be a zebrafish hb-egfa.

In an aspect, a disclosed hb-egfa can comprise the hb-egfa sequence set forth in Gene ID: 15200 or a fragment thereof, or a sequence having about 50%, about 55%, about 60%, about 65%, about 70%, about 75%, about 80%, about 85%, about 90%, about 95%, or more than 95% identity to the sequence set forth in Gene ID: 15200 or a fragment thereof. In an aspect, a disclosed hb-egf can be a mouse hb-egf.

In an aspect, a disclosed hb-egfa can comprise the hb-egfa sequence set forth in Gene ID: 1839 or a fragment thereof, or a sequence having about 50%, about 55%, about 60%, about 65%, about 70%, about 75%, about 80%, about 85%, about 90%, about 95%, or more than 95% identity to the sequence set forth in Gene ID: 1839 or a fragment thereof. In an aspect, a disclosed hb-egf can be a human hb-egfa.

52) In an aspect, a disclosed encoded polypeptide can comprise heparin binding epidermal growth factor (HB-EGF) or recombinant heparin binding epidermal growth factor (rHB-EGF). In an aspect, a disclosed human HB-EGF can comprise the sequence set forth in Accession No. Q99075.1 or a fragment thereof, or a sequence having about 50%, about 55%, about 60%, about 65%, about 70%, about 75%, about 80%, about 85%, about 90%, about 95%, or more than 95% identity to the sequence set forth in Accession No. Q99075.1 or a fragment thereof. In an aspect, a disclosed encoded polypeptide can comprise heparin binding epidermal growth factor a (HB-EGFa) or recombinant heparin binding epidermal growth factor a (rHB-EGFa). In an aspect, a disclosed encoded polypeptide can comprise heparin binding epidermal growth factor b (HB-EGFb) or recombinant heparin binding epidermal growth factor b (rHB-EGFb).

In an aspect. HB-EGF can comprise the sequence set forth in SEQ ID NO:35, SEQ ID NO:36, SEQ ID NO:37 or a fragment thereof. In an aspect. HB-EGF can comprise a sequence having about 50%, about 55%, about 60%, about 65%, about 70%, about 75%, about 80%, about 85%, about 90%, about 95%, or more than 95% identity to the sequence set forth in SEQ ID NO:35, SEQ ID NO:36, SEQ ID NO:37 or a fragment thereof.

54) In an aspect, expression of a disclosed encoded polypeptide can promote glial cell proliferation, can promote axonal tract regeneration, can trigger neurite outgrowth, can trigger neuron formation, can stimulate regeneration of spinal cord tissue, or any combination thereof.

In an aspect, expression of a disclosed polypeptide can improve spinal cord function. Spinal cord function can comprise sensory function, motor function, or a combination thereof. Spinal cord function can be assessed, examined, and/or measured by one or more methods known to the skilled person. For example, expression of a disclosed encoded polypeptide can improve an ASIA score.

In an aspect, expression of HB-EGF or rHB-EGF can promote glial cell proliferation, can promote axonal tract regeneration, can trigger neurite outgrowth, can trigger neuron formation, can stimulate regeneration of spinal cord tissue, or any combination thereof. In an aspect, expression of HB-EGF or rHB-EG can improve spinal cord function. Spinal cord function can comprise sensory function, motor function, or a combination thereof. Spinal cord function can be assessed, examined, and/or measured by one or more methods known to the skilled person. For example, expression of a disclosed encoded polypeptide can improve an ASIA score.

In an aspect, a disclosed isolated nucleic acid molecule can comprise a reporter transgene. Reporter genes are known to the art. In an aspect, a disclosed reporter gene can comprise green fluorescent protein or mCherry.

In an aspect, a disclosed isolated nucleic acid molecule can be flanked by inverted terminal repeats such as, for example, ITRs derived from the adeno-associated viral (AAV) genome.

In an aspect, a disclosed isolated nucleic acid molecule can be packaged in an AAV capsid or AAV particle or can be packaged in an recombinant AAV capsid or a recombinant AAV particle. In an aspect, a disclosed isolated nucleic acid can comprise be packaged in an CC47 AAV capsid or a CC47 AAV particle or can be packaged in a recombinant CC47 AAV capsid or a recombinant CC47 AAV particle.

In an aspect, a disclosed isolated nucleic acid molecule can be packaged in a viral vector or a recombinant viral vector. In an aspect, a disclosed viral vector can be an AAV vector or a recombinant AAV vector, or can be a lentiviral vector or a recombinant lentiviral vector.

In an aspect, a disclosed isolated nucleic acid molecule can comprise a coding sequence that is less than about 4.5 kilobases.

Disclosed herein are plasmids that comprise a disclosed isolated nucleic acid molecule, a disclosed transgene, and/or a disclosed transgene cassette. Plasmids are known to the art and described in the Examples provided herein. For example, disclosed herein are plasmids comprising a hb-egfaEN-cfos:EGFP construct and a cfos:EGFP zebrafish construct.

63) Disclosed herein is a transgene cassette comprising a disclosed isolated nucleic acid molecule comprising a nucleic acid sequence encoding a tissue regeneration enhancer element (TREE); an encoded polypeptide; and a promoter directing expression of the encoded polypeptide in damaged and/or injured spinal cord tissues.

Disclosed herein is a transgene cassette comprising isolated nucleic acid molecule, comprising a nucleic acid sequence encoding a tissue regeneration enhancer element (TREE); an encoded polypeptide; a promoter directing expression of the encoded polypeptide in damaged and/or injured spinal cord tissues; and a 3′ UTR noncoding region.

Disclosed herein are transgenic animals comprising a disclosed transgene cassette. In an aspect, a disclosed transgenic animal can be used for identification and/or validation on a putative TREE. In an aspect, a disclosed transgenic animal can comprise a mouse or a zebrafish.

Disclosed herein are cells comprising a disclosed isolated nucleic acid molecule, a disclosed vector, a disclosed plasmid, and/or a disclosed gene cassette or transgene cassette. Host cells are known to the art.

### 3. Vectors

Disclosed herein is a vector comprising a disclosed isolated nucleic molecule.

Disclosed herein is a vector comprising an isolated nucleic acid molecule, comprising a nucleic acid sequence encoding a tissue regeneration enhancer element (TREE); an encoded polypeptide; and a promoter directing expression of the encoded polypeptide in damaged and/or injured spinal cord tissues. Disclosed herein is a vector comprising an isolated nucleic acid molecule, comprising a nucleic acid sequence encoding a tissue regeneration enhancer element (TREE); an encoded polypeptide; a promoter directing expression of the encoded polypeptide in damaged and/or injured spinal cord tissues; and a 3′ UTR noncoding region.

In an aspect, a disclosed encoded polypeptide can comprise HB-EGF or rHB-EGF.

In an aspect, a disclosed vector can comprise a viral vector or a non-viral vector. In an aspect, a disclosed non-viral vector can be a polymer-based vector, a peptide-based vector, a lipid nanoparticle, a solid lipid nanoparticle, or a cationic lipid based vector. In an aspect, a disclosed viral vector can be an adenovirus vector, an adeno-associated virus (AAV) vector, a herpes simplex virus vector, a retrovirus vector, a lentivirus vector, and alphavirus vector, a flavivirus vector, a rhabdovirus vector, a measles virus vector, a Newcastle disease viral vector, a poxvirus vector, or a picornavirus vector. In an aspect, a disclosed viral vector can be an lentiviral vector or a recombinant lentiviral vector.

In an aspect, a disclosed viral vector can be an AAV vector or a recombinant AAV vector (rAAV). In an aspect, a disclosed AAV vector can comprise AAV1, AAV2, AAV3 (including 3a and 3b), AAV4, AAV5, AAV6, AAV7, AAV8, AAVrh8, AAV9, AAV10, AAVrh10, AAV11, AAV12, AAV13, AAVrh39, AAVrh43, or AAVcy.7. In an aspect, a disclosed AAV vector can comprise bovine AAV, caprine AAV, canine AAV, equine AAV, ovine AAV, avian AAV, primate AAV, or non-primate AAV. In an aspect, a disclosed AAV vector can comprise AAV-DJ, AAV-HAE1, AAV-HAE2, AAVM41, AAV-1829, AAV2 Y/F, AAV2 T/V, AAV2i8, AAV2.5, AAV9.45, AAV9.61, AAV-B1, AAV-AS, AAV9.45A-String (e.g., AAV9.45-AS), AAV9.45Angiopep, AAV9.47-Angiopep, AAV9.47-AS, AAV-PHP.B. AAV-PHP.eB, AAV-PHP.S, AAV-F, AAVcc.47, or AAVcc.81.

In an aspect, a disclosed AAV vector can comprise a tissue-specific promoter operably linked to disclosed encoded polypeptide, a disclosed gene cassette, or a disclosed isolated nucleic acid molecule.

In an aspect, a disclosed vector can comprise one or more CRISPR-based epigenome editing tools. In an aspect, a disclosed vector can comprise the sequence for one or more gRNAs. gRNAs are known to the art. In an aspect, a disclosed gRNA can target an endogenous gene in injured and/or damaged spinal cord tissue. In an aspect, a disclosed vector can comprise a promoter operably linked to the one or more gRNAs. In an aspect, a disclosed promoter operably linked to the one or more gRNAs can comprise a ubiquitous promoter, a constitutive promoter, or a tissue specific promoter. In an aspect, a disclosed promoter can comprise a U6 promoter.

Disclosed herein is a vector comprising a disclosed gene or transgene cassette. In an aspect, a disclosed gene or transgene cassette can comprise a disclosed isolated nucleic acid molecule.

Disclosed herein is an AAV or an rAAV vector comprising a nucleic acid sequence encoding a tissue regeneration enhancer element (TREE); an encoded polypeptide; and a promoter directing expression of the encoded polypeptide in damaged and/or injured spinal cord tissues.

Disclosed herein is an AAV or an rAAV vector comprising a nucleic acid sequence encoding a tissue regeneration enhancer element (TREE); an encoded polypeptide; a promoter directing expression of the encoded polypeptide in damaged and/or injured spinal cord tissues; and a 3′ UTR noncoding region.

Disclosed herein is an AAV or an rAAV vector comprising a disclosed plasmid such as, for example, a hb-egfaEN-cfos:EGFP plasmid construct and a cfos:EGFP zebrafish plasmid construct.

Disclosed herein is an AAV or an rAAV vector comprising a disclosed isolated nucleic acid molecule, a disclosed transgene, and/or a disclosed gene or transgene cassette.

Disclosed herein is an AAV or an rAAV vector comprising a disclosed gene or transgene cassette.

Disclosed herein is AAV or an rAAV vector comprising a gene or transgene cassette comprising a disclosed isolated nucleic acid molecule comprising a nucleic acid sequence encoding a tissue regeneration enhancer element (TREE); an encoded polypeptide; and a promoter directing expression of the encoded polypeptide in damaged and/or injured spinal cord tissues.

Disclosed herein is AAV or an rAAV vector comprising a transgene cassette comprising isolated nucleic acid molecule, comprising a nucleic acid sequence encoding a tissue regeneration enhancer element (TREE); an encoded polypeptide; a promoter directing expression of the encoded polypeptide in damaged and/or injured spinal cord tissues; and a 3′ UTR noncoding region.

Disclosed herein is vector comprising a nucleic acid sequence encoding a tissue regeneration enhancer element (TREE); and a promoter directing expression of a endogenous polypeptide in damaged and/or injured spinal cord tissues.

Disclosed herein is a vector comprising a nucleic acid sequence encoding a tissue regeneration enhancer element (TREE); a promoter directing expression of a endogenous polypeptide in damaged and/or injured spinal cord tissues; and a 3′ UTR noncoding region.

Disclosed herein is a vector comprising a disclosed plasmid such as, for example, a hb-egfaEN-cfos:EGFP plasmid construct and a cfos:EGFP zebrafish plasmid construct.

Disclosed herein is a vector comprising a disclosed isolated nucleic acid molecule, a disclosed transgene, and/or a disclosed transgene cassette.

Disclosed herein is a vector comprising a disclosed gene or transgene cassette.

Disclosed herein is a vector comprising a gene or transgene cassette comprising a disclosed isolated nucleic acid molecule comprising a nucleic acid sequence encoding a tissue regeneration enhancer element (TREE); an encoded polypeptide; and a promoter directing expression of the encoded polypeptide in damaged and/or injured spinal cord tissues.

Disclosed herein is a vector comprising a gene or transgene cassette comprising isolated nucleic acid molecule, comprising a nucleic acid sequence encoding a tissue regeneration enhancer element (TREE); an encoded polypeptide; a promoter directing expression of the encoded polypeptide in damaged and/or injured spinal cord tissues; and a 3′ UTR noncoding region.

### 4. Pharmaceutical Formulations

Disclosed herein is a pharmaceutical formulation comprising a disclosed isolated nucleic acid molecule and a pharmaceutically acceptable carrier.

Disclosed herein is a pharmaceutical formulation comprising an isolated nucleic acid molecule, comprising a nucleic acid sequence encoding a tissue regeneration enhancer element (TREE); an encoded polypeptide; and a promoter directing expression of the encoded polypeptide in damaged and/or injured spinal cord tissues, and a pharmaceutically acceptable carrier.

Disclosed herein is a pharmaceutical formulation comprising a nucleic acid sequence encoding a tissue regeneration enhancer element (TREE); an encoded polypeptide; a promoter directing expression of the encoded polypeptide in damaged and/or injured spinal cord tissues; and a 3′ UTR noncoding region, and a pharmaceutically acceptable carrier.

Disclosed herein is a pharmaceutical formulation comprising a disclosed vector and a pharmaceutically acceptable carrier.

Disclosed herein is a pharmaceutical formulation comprising a therapeutically effective amount of a disclosed HB-EGF. Disclosed herein is a pharmaceutical formulation comprising a therapeutically effective amount of a disclosed recombinant HB-EGF. Disclosed herein is a pharmaceutical formulation comprising a therapeutically effective amount of a disclosed epidermal growth factor a (HB-EGFa) or a recombinant heparin binding epidermal growth factor a (rHB-EGFa). Disclosed herein is a pharmaceutical formulation comprising a therapeutically effective amount of a heparin binding epidermal growth factor b (HB-EGFb) or a recombinant heparin binding epidermal growth factor b (rHB-EGFb). Disclosed herein is a pharmaceutical formulation comprising a therapeutically effective amount of recombinant human HB-EGF. Disclosed herein is a pharmaceutical formulation comprising a therapeutically effective amount of a disclosed HB-EGF. Disclosed herein is a pharmaceutical formulation comprising a therapeutically effective amount of a disclosed HB-EGF and a pharmaceutically acceptable carrier. In an aspect, a disclosed HB-EGF can comprise a recombinant HB-EGF. In an aspect, a disclosed HB-EGF can comprise epidermal growth factor a (HB-EGFa) or recombinant heparin binding epidermal growth factor a (rHB-EGFa). In an aspect, a disclosed HB-EGF can comprise a disclosed heparin binding epidermal growth factor b (HB-EGFb) or a recombinant heparin binding epidermal growth factor b (rHB-EGFb). In an aspect, a disclosed HB-EGF can comprise recombinant human HB-EGF.

A disclosed pharmaceutically acceptable carrier can refer to a sterile aqueous or nonaqueous solution, a dispersion, a suspension, an emulsion, or any combination thereof, as well as a sterile powder for reconstitution into a sterile injectable solution, dispersion, suspension, emulsion, or any combination thereof just prior to use. Examples of suitable aqueous and nonaqueous carriers, diluents, solvents, or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. In an aspect, a pharmaceutical carrier employed can be a solid, liquid, or gas. In an aspect, examples of solid carriers can include lactose, terra alba, sucrose, tale, gelatin, agar, pectin, acacia, magnesium stearate, and stearic acid. In an aspect, examples of liquid carriers can include sugar syrup, peanut oil, olive oil, and water. In an aspect, examples of gaseous carriers can include carbon dioxide and nitrogen. In preparing a disclosed composition for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions that are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

### 5. Kits

Disclosed herein is a kit comprising one or more components and/or reagents for use in a disclosed method of treating a spinal cord injury. Disclosed herein is a kit comprising one or more components and/or reagents for use in a disclosed method of stimulating regeneration of injured and/or damaged spinal cord tissue. Disclosed herein is a kit comprising one or more components and/or reagents for use in a disclosed method of promoting glial cell proliferation in injured and/or damaged spinal cord tissue. Disclosed herein is a kit comprising one or more components and/or reagents for use in a disclosed method of promoting axonal tract regeneration in injured and/or damaged spinal cord tissue. Disclosed herein is a kit comprising one or more components and/or reagents for use in a disclosed method of comprising triggering neurite outgrowth in injured and/or damaged spinal cord tissue. Disclosed herein is a kit comprising one or more components and/or reagents for use in a disclosed method of triggering neuron formation in injured and/or damaged spinal cord tissue. Disclosed herein is a kit comprising one or more components and/or reagents for use in a disclosed method of comprising improving spinal cord function in a subject in need thereof. Disclosed herein is a kit comprising one or more components and/or reagents for use in a disclosed methods of generating a disclosed viral vector such as, for example, a disclosed lentiviral vector or a disclosed AAV vector. Disclosed herein is a kit comprising one or more components and/or reagents for use in a disclosed method of identifying one or more putative TREEs. In an aspect, a disclosed kit can comprise the components and/or reagents necessary to perform one or more steps of a disclosed methods, such as, for example, obtaining a first population of cells, isolating the nuclei from the first population of cells, analyzing chromatin structure and function of the isolated nuclei to obtain a chromatin profile for the first population of cells, obtaining a second population of cells, isolating the nuclei from the second population of cells, analyzing chromatin structure and function of the isolated nuclei to obtain a chromatin profile for the second population of cells and comparing the chromatin profiles between the two populations of cells to identify one or more putative TREEs.

In an aspect, a disclosed kit can comprise one or more disclosed isolated nucleic acid molecules, disclosed vectors, disclosed pharmaceutical formulations, disclosed expression cassettes, disclosed plasmids, or any combination thereof. In an aspect, a disclosed kit can comprise a disclosed HB-EGF or disclosed recombinant HB-EGF or a pharmaceutical formulation comprising a disclosed HB-EGF or a disclosed recombinant HB-EGF.

In an aspect, a disclosed kit can comprise one or more restriction enzymes, fixative agents, digestion agents, primers, polymerases, ligases, or any combination thereof. In an aspect, a disclosed kit can comprise at least two components and/or reagents constituting the kit. Together, the components and/or reagents constitute a functional unit for a given purpose (such as, for example, a method of treating stressed, damaged, and/or injured tissues). Individual member components may be physically packaged together or separately. For example, a kit comprising an instruction for using the kit may or may not physically include the instruction with other individual member components and/or reagents. Instead, the instruction can be supplied as a separate member component and/or reagent, either in a paper form or an electronic form which may be supplied on computer readable memory device or downloaded from an internet website, or as recorded presentation. In an aspect, a kit for use in a disclosed method can comprise one or more containers holding a disclosed component and/or reagent and a label or package insert with instructions for use. In an aspect, suitable containers include, for example, bottles, vials, syringes, blister pack, etc. The containers can be formed from a variety of materials such as glass or plastic. The container can hold, for example, a disclosed component and/or reagent and can have a sterile access port (for example the container may be an intravenous solution bag or a vial having a stopper pierceable by a hypodermic injection needle). The label or package insert can indicate that a disclosed component and/or reagent can be used in a disclosed method. In an aspect, a disclosed kit can comprise additional components and/or reagents necessary for administration such as, for example, other buffers, polymerases, primers, chemical reagents, diluents, filters, needles, and syringes.

### C. Methods

### 1. Methods of Treating a Spinal Cord Injury

Disclosed herein is a method of treating a spinal cord injury, the method comprising administering to a subject in need thereof a disclosed isolated nucleic acid molecule, a disclosed vector, a disclosed pharmaceutical formulation, or any combination thereof. Disclosed herein is a method of treating a spinal cord injury, the method comprising stimulating regeneration of injured and/or damaged spinal cord tissue in a subject in need thereof by administering to a subject a disclosed isolated nucleic acid molecule, a disclosed vector, a disclosed pharmaceutical formulation, or any combination thereof. Disclosed herein is a method of treating a spinal cord injury, the method comprising: promoting glial cell proliferation in injured and/or damaged spinal cord tissue in a subject by administering to a subject a disclosed isolated nucleic acid molecule, a disclosed vector, a disclosed pharmaceutical formulation, or any combination thereof. Disclosed herein is a method of treating a spinal cord injury, the method comprising promoting axonal tract regeneration in injured and/or damaged spinal cord tissue in a subject in need thereof by administering to a subject a disclosed isolated nucleic acid molecule, a disclosed vector, a disclosed pharmaceutical formulation, or any combination thereof. Disclosed herein is a method of treating a spinal cord injury, the method comprising triggering neurite outgrowth in injured and/or damaged spinal cord tissue in a subject in need thereof by administering to the subject a disclosed isolated nucleic acid molecule, a disclosed vector, a disclosed pharmaceutical formulation, or any combination thereof. Disclosed herein method of treating a spinal cord injury, the method comprising triggering neuron formation in injured and/or damaged spinal cord tissue in a subject in need thereof by administering to a subject a disclosed isolated nucleic acid molecule, a disclosed vector, a disclosed pharmaceutical formulation, or any combination thereof. Disclosed herein is a method of treating a spinal cord injury, the method comprising improving spinal cord function in a subject in need thereof by administering to a subject a disclosed isolated nucleic acid molecule, a disclosed vector, a disclosed pharmaceutical formulation, or any combination thereof.

Disclosed herein is a method of treating a spinal cord injury, the method comprising administering to a subject in need thereof a disclosed isolated nucleic acid molecule, a disclosed vector, a disclosed pharmaceutical formulation, or any combination thereof, and administering a therapeutically effective amount of HB-EGF. Disclosed herein is a method of treating a spinal cord injury, the method comprising stimulating regeneration of injured and/or damaged spinal cord tissue in a subject in need thereof by administering to a subject a disclosed isolated nucleic acid molecule, a disclosed vector, a disclosed pharmaceutical formulation, or any combination thereof, and administering a therapeutically effective amount of HB-EGF. Disclosed herein is a method of treating a spinal cord injury, the method comprising: promoting glial cell proliferation in injured and/or damaged spinal cord tissue in a subject by administering to a subject a disclosed isolated nucleic acid molecule, a disclosed vector, a disclosed pharmaceutical formulation, or any combination thereof, and administering a therapeutically effective amount of HB-EGF. Disclosed herein is a method of treating a spinal cord injury, the method comprising promoting axonal tract regeneration in injured and/or damaged spinal cord tissue in a subject in need thereof by administering to a subject a disclosed isolated nucleic acid molecule, a disclosed vector, a disclosed pharmaceutical formulation, or any combination thereof, and administering a therapeutically effective amount of HB-EGF. Disclosed herein is a method of treating a spinal cord injury, the method comprising triggering neurite outgrowth in injured and/or damaged spinal cord tissue in a subject in need thereof by administering to the subject a disclosed isolated nucleic acid molecule, a disclosed vector, a disclosed pharmaceutical formulation, or any combination thereof, and administering a therapeutically effective amount of HB-EGF. Disclosed herein method of treating a spinal cord injury, the method comprising triggering neuron formation in injured and/or damaged spinal cord tissue in a subject in need thereof by administering to a subject a disclosed isolated nucleic acid molecule, a disclosed vector, a disclosed pharmaceutical formulation, or any combination thereof, and administering a therapeutically effective amount of HB-EGF. Disclosed herein is a method of treating a spinal cord injury, the method comprising improving spinal cord function in a subject in need thereof by administering to a subject a disclosed isolated nucleic acid molecule, a disclosed vector, a disclosed pharmaceutical formulation, or any combination thereof, and administering a therapeutically effective amount of HB-EGF.

Disclosed herein is a method of treating a spinal cord injury, the method comprising administering to a subject in need thereof a therapeutically effective amount of HB-EGF or a pharmaceutical formulation comprising a therapeutically effective amount of HB-EGF. Disclosed herein is a method of treating a spinal cord injury, the method comprising stimulating regeneration of injured and/or damaged spinal cord tissue in a subject in need thereof by administering to a subject a therapeutically effective amount of HB-EGF. Disclosed herein is a method of treating a spinal cord injury, the method comprising: promoting glial cell proliferation in injured and/or damaged spinal cord tissue in a subject by administering to a subject a therapeutically effective amount of HB-EGF or a pharmaceutical formulation comprising a therapeutically effective amount of HB-EGF. Disclosed herein is a method of treating a spinal cord injury, the method comprising promoting axonal tract regeneration in injured and/or damaged spinal cord tissue in a subject in need thereof by administering to a subject a therapeutically effective amount of HB-EGF or a pharmaceutical formulation comprising a therapeutically effective amount of HB-EGF. Disclosed herein is a method of treating a spinal cord injury, the method comprising triggering neurite outgrowth in injured and/or damaged spinal cord tissue in a subject in need thereof by administering to the subject a therapeutically effective amount of HB-EGF or a pharmaceutical formulation comprising a therapeutically effective amount of HB-EGF. Disclosed herein is a method of treating a spinal cord injury, the method comprising triggering neuron formation in injured and/or damaged spinal cord tissue in a subject in need thereof by administering to a subject a therapeutically effective amount of HB-EGF or a pharmaceutical formulation comprising a therapeutically effective amount of HB-EGF. Disclosed herein is a method of treating a spinal cord injury, the method comprising improving spinal cord function in a subject in need thereof by administering to a subject a therapeutically effective amount of HB-EGF or a pharmaceutical formulation comprising a therapeutically effective amount of HB-EGF.

In an aspect, a disclosed HB-EGF can comprise a recombinant HB-EGF. In an aspect, a disclosed HB-EGF can comprise epidermal growth factor a (HB-EGFa) or recombinant heparin binding epidermal growth factor a (rHB-EGFa). In an aspect, a disclosed HB-EGF can comprise heparin binding epidermal growth factor b (HB-EGFb) or recombinant heparin binding epidermal growth factor b (rHB-EGFb). In an aspect, a disclosed HB-EGF can comprise recombinant human HB-EGF.

In an aspect, a disclosed method of treating a spinal cord injury can comprise stimulating regeneration of injured and/or damaged spinal cord tissue, promoting glial cell proliferation in injured and/or damaged spinal cord tissue, promoting axonal tract regeneration in injured and/or damaged spinal cord tissue, triggering neurite outgrowth in injured and/or damaged spinal cord tissue, triggering neuron formation in injured and/or damaged spinal cord tissue, improving spinal cord function in a subject in need thereof, or any combination thereof.

In an aspect, improving spinal cord function can comprise improving sensory function and/or motor function. In an aspect, improving sensory function and/or motor function can comprise transient improvements. In an aspect, improving sensory function and/or motor function can comprise sustained improvements. In an aspect, improvements can be sustained for at least 2 months, at least 3 months, at least 4 months, at least 6 months, at least 1 year, at least 18 months, at least 2 years, or at least 3 years, or more than 3 years.

In an aspect, a disclosed method can comprise reducing inflammation in the injured and/or damaged spinal cord tissue. In an aspect, a disclosed method can comprise reducing scar tissue in the injured and/or damaged spinal cord tissue.

In an aspect, a disclosed method can comprise applying a disclosed hydrogel to the injured and/or damaged spinal cord tissue. In an aspect, a disclosed hydrogel can comprise one or more therapeutic agents. In an aspect, the one or more additional disclosed therapeutic agents can comprise agents that promote glial cell proliferation, promote axonal tract regeneration, trigger neurite outgrowth, trigger neuron formation, stimulate regeneration of spinal cord tissue, or any combination thereof. In an aspect, the one or more additional disclosed therapeutic agents can comprise methylprednisolone or can comprise one or more corticosteroids. In an aspect, a disclosed hydrogel can comprise heparin binding epidermal growth factor (HB-EGF) or recombinant heparin binding epidermal growth factor (rHB-EGF). In an aspect, a disclosed hydrogel can comprise heparin binding epidermal growth factor a (HB-EGFa) or recombinant heparin binding epidermal growth factor a (rHB-EGFa). In an aspect, a disclosed hydrogel can comprise heparin binding epidermal growth factor b (HB-EGFb) or recombinant heparin binding epidermal growth factor b (rHB-EGFb). In an aspect, a disclosed hydrogel can comprise recombinant human HB-EGF.

In an aspect, a disclosed method can comprise applying HB-EGF to, about, or near injured and/or damaged spinal cord tissue. In an aspect, a disclosed HB-EGF can comprise a disclosed recombinant HB-EGF. Applying HB-EGF to, about, or near injured and/or damaged spinal cord tissue can comprise any means known to the art to apply a composition.

In an aspect of a disclosed method, a disclosed isolated nucleic acid molecule, a disclosed vector, or a disclosed pharmaceutical formulation can be systemically or directly administered to the subject, or can be intravenously, subcutaneously, or intramuscularly administered to the subject, or can be directly administered to the injured and/or damaged spinal cord tissue.

In an aspect of a disclosed method, a disclosed HB-EGF or a disclosed pharmaceutical formulation comprising a disclosed HB-EGF can be systemically or directly administered to the subject, or can be intravenously, subcutaneously, or intramuscularly administered to the subject, or can be directly administered to the injured and/or damaged spinal cord tissue.

In an aspect of a disclosed method, a disclosed promoter can direct the expression of the encoded polypeptide in the subject's injured and/or damaged spinal cord tissue. In an aspect, spinal cord tissue can comprise neurons, neuroglia, or a combination thereof. In an aspect, neuroglia can comprise microglia and/or macroglia. In an aspect, neuroglia can comprise microglia, astrocytes, oligodendrocytes, ependymal cells, radial glia, Schwann cells, satellite cells, or any combination thereof.

In an aspect, a disclosed method can further comprise repeating the administering step. For example, in an aspect, a disclosed method can comprise administering one or more times a disclosed isolated nucleic acid molecule, a disclosed vector, or a disclosed pharmaceutical formulation.

In an aspect, a disclosed method can comprise administering one or more times a therapeutically effective amount of HB-EGF or a pharmaceutical formulation comprising a disclosed HB-EGF or a disclosed pharmaceutical formulation comprising a disclosed HB-EGF.

In an aspect, a disclosed method can comprise administering to the subject one or more additional therapeutic agents. Therapeutic agents are known to the art. In an aspect, therapeutic agents can comprise agents that promote glial cell proliferation, promote axonal tract regeneration, trigger neurite outgrowth, trigger neuron formation, stimulate regeneration of spinal cord tissue, or any combination thereof. In an aspect, a therapeutic agent can comprise methylprednisolone or can comprise one or more corticosteroids. In an aspect, a therapeutic agent can comprise any agent that is capable of providing a local or systemic biological, physiological, or therapeutic effect in the biological system to which it is applied. For example, the a therapeutic agent can act to control infection or inflammation, enhance cell growth and tissue regeneration, control tumor growth, act as an analgesic, promote anti-cell attachment, and enhance bone growth, among other functions. Therapeutic agents as well as the specifics of the administration of therapeutic agents (i.e., dosing amount and schedule, administration route, etc.) are known the art. As used herein, the recitation of a biologically active agent inherently encompasses the pharmaceutically acceptable salts thereof.

In an aspect, a disclosed method can further comprise monitoring the subject for adverse effects. In an aspect, in the absence of adverse effects, the method can further comprise continuing to treat the subject. In an aspect, in the presence of adverse effects, the method can further comprise modifying the treating step. In an aspect of a disclosed method, methods and techniques to monitor a subject can comprise qualitative (or subjective) means as well as quantitative (or objective) means. In an aspect, qualitative means (or subjective means) can comprise a subject's own perspective. For example, a subject can report how he/she is feeling, whether he/she has experienced improvements and/or setbacks, whether he/she has experienced an amelioration or an intensification of one or more symptoms, or a combination thereof. In an aspect, quantitative means (or objective means) can comprise methods and techniques that include, but are not limited to, the following: (i) fluid analysis (e.g., tests of a subject's fluids including but not limited to aqueous humor and vitreous humor, bile, blood, blood serum, breast milk, cerebrospinal fluid, cerumen (earwax), digestive fluids, endolymph and perilymph, female ejaculate, gastric juice, mucus (including nasal drainage and phlegm), peritoneal fluid, pleural fluid, saliva, sebum (skin oil), semen, sweat, synovial fluid, tears, vaginal secretion, vomit, and urine), (ii) imaging (e.g., ordinary x-rays, ultrasonography, radioisotope (nuclear) scanning, computed tomography (CT), magnetic resonance imaging (MRI), positron emission tomography (PET), and angiography), (iii) endoscopy (e.g., laryngoscopy, bronchoscopy, esophagoscopy, gastroscopy, GI endoscopy, coloscopy, cystoscopy, hysteroscopy, arthroscopy, laparoscopy, mediastinoscopy, and thoracoscopy), (iv) analysis of organ activity (e.g., electrocardiography (ECG), electroencephalography (EEG), and pulse oximetry), (v) biopsy (e.g., removal of tissue samples for microscopic evaluation), and (vi) genetic testing.

In an aspect, a disclosed method can further comprise generating a disclosed viral or non-viral vector. In an aspect, generating a disclosed viral vector can comprise generating an AAV vector (such as, for example, an cc47 AAV vector).

In an aspect, a disclosed method can further comprise preparing a disclosed hydrogel.

In an aspect, a disclosed method of treating a spinal cord injury can comprise spatiotemporally targeted tissue regeneration. In an aspect, a disclosed method of treating a spinal cord injury can be used in a platform for spatiotemporally targeted tissue regeneration.

2. Methods of Stimulating Regeneration of Injured and/or Damaged Spinal Cord Tissue

Disclosed herein is a method of stimulating regeneration of injured and/or damaged spinal cord tissue, the method comprising administering to a subject in need thereof a disclosed isolated nucleic acid molecule, a disclosed vector, a disclosed pharmaceutical formulation, or any combination thereof.

Disclosed herein is a method of stimulating regeneration of injured and/or damaged spinal cord tissue, the method comprising administering to a subject in need thereof a disclosed isolated nucleic acid molecule, a disclosed vector, a disclosed pharmaceutical formulation, or any combination thereof, and administering a therapeutically effective amount of HB-EGF.

Disclosed herein is a method of stimulating regeneration of injured and/or damaged spinal cord tissue, the method comprising administering to a subject in need thereof a disclosed HB-EGF. Disclosed herein is a method of stimulating regeneration of injured and/or damaged spinal cord tissue, the method comprising administering to a subject in need thereof a therapeutically effective amount of HB-EGF or a pharmaceutical formulation comprising a therapeutically effective amount of HB-EGF.

In an aspect, a disclosed HB-EGF can comprise a recombinant HB-EGF. In an aspect, a disclosed HB-EGF can comprise epidermal growth factor a (HB-EGFa) or recombinant heparin binding epidermal growth factor a (rHB-EGFa). In an aspect, a disclosed HB-EGF can comprise heparin binding epidermal growth factor b (HB-EGFb) or recombinant heparin binding epidermal growth factor b (rHB-EGFb). In an aspect, a disclosed HB-EGF can comprise recombinant human HB-EGF.

In an aspect, a disclosed method of stimulating regeneration of injured and/or damaged spinal cord tissue can comprise treating a spinal cord injury, promoting glial cell proliferation in injured and/or damaged spinal cord tissue, promoting axonal tract regeneration in injured and/or damaged spinal cord tissue, triggering neurite outgrowth in injured and/or damaged spinal cord tissue, triggering neuron formation in injured and/or damaged spinal cord tissue, improving spinal cord function in a subject in need thereof, or any combination thereof.

In an aspect, improving spinal cord function can comprise improving sensory function and/or motor function. In an aspect, improving sensory function and/or motor function can comprise transient improvements. In an aspect, improving sensory function and/or motor function can comprise sustained improvements. In an aspect, improvements can be sustained for at least 2 months, at least 3 months, at least 4 months, at least 6 months, at least 1 year, at least 18 months, at least 2 years, or at least 3 years, or more than 3 years.

In an aspect, a disclosed method can comprise reducing inflammation in the injured and/or damaged spinal cord tissue. In an aspect, a disclosed method can comprise reducing scar tissue in the injured and/or damaged spinal cord tissue.

In an aspect, a disclosed method can comprise applying a disclosed hydrogel to the injured and/or damaged spinal cord tissue. In an aspect, a disclosed hydrogel can comprise one or more therapeutic agents. In an aspect, the one or more additional disclosed therapeutic agents can comprise agents that promote glial cell proliferation, promote axonal tract regeneration, trigger neurite outgrowth, trigger neuron formation, stimulate regeneration of spinal cord tissue, or any combination thereof. In an aspect, the one or more additional disclosed therapeutic agents can comprise methylprednisolone or can comprise one or more corticosteroids. In an aspect, a disclosed hydrogel can comprise heparin binding epidermal growth factor (HB-EGF) or recombinant heparin binding epidermal growth factor (rHB-EGF). In an aspect, a disclosed hydrogel can comprise heparin binding epidermal growth factor a (HB-EGFa) or recombinant heparin binding epidermal growth factor a (rHB-EGFa). In an aspect, a disclosed hydrogel can comprise heparin binding epidermal growth factor b (HB-EGFb) or recombinant heparin binding epidermal growth factor b (rHB-EGFb). In an aspect, a disclosed hydrogel can comprise recombinant human HB-EGF.

In an aspect, a disclosed method can comprise applying HB-EGF to, about, or near injured and/or damaged spinal cord tissue. In an aspect, a disclosed HB-EGF can comprise a disclosed recombinant HB-EGF Applying HB-EGF to, about, or near injured and/or damaged spinal cord tissue can comprise any means known to the art to apply a composition.

In an aspect of a disclosed method, a disclosed isolated nucleic acid molecule, a disclosed vector, or a disclosed pharmaceutical formulation can be systemically or directly administered to the subject, or can be intravenously, subcutaneously, or intramuscularly administered to the subject, or can be directly administered to the injured and/or damaged spinal cord tissue.

In an aspect of a disclosed method, a disclosed HB-EGF or a disclosed pharmaceutical formulation comprising a disclosed HB-EGF can be systemically or directly administered to the subject, or can be intravenously, subcutaneously, or intramuscularly administered to the subject, or can be directly administered to the injured and/or damaged spinal cord tissue.

In an aspect of a disclosed method, a disclosed promoter can direct the expression of the encoded polypeptide in the subject's injured and/or damaged spinal cord tissue. In an aspect, spinal cord tissue can comprise neurons, neuroglia, or a combination thereof. In an aspect, neuroglia can comprise microglia and/or macroglia. In an aspect, neuroglia can comprise microglia, astrocytes, oligodendrocytes, ependymal cells, radial glia, Schwann cells, satellite cells, or any combination thereof.

In an aspect, a disclosed method can comprise repeating the administering step. For example, in an aspect, a disclosed method can comprise administering one or more times a disclosed isolated nucleic acid molecule, a disclosed vector, or a disclosed pharmaceutical formulation. In an aspect, a disclosed method can comprise administering one or more times a therapeutically effective amount of HB-EGF or a pharmaceutical formulation comprising a disclosed HB-EGF or a disclosed pharmaceutical formulation comprising a disclosed HB-EGF.

In an aspect, a disclosed method can comprise administering to the subject one or more additional therapeutic agents. Therapeutic agents are known to the art. In an aspect, therapeutic agents can comprise agents that promote glial cell proliferation, promote axonal tract regeneration, trigger neurite outgrowth, trigger neuron formation, stimulate regeneration of spinal cord tissue, or any combination thereof. In an aspect, a therapeutic agent can comprise methylprednisolone or can comprise one or more corticosteroids. In an aspect, a therapeutic agent can comprise any agent that is capable of providing a local or systemic biological, physiological, or therapeutic effect in the biological system to which it is applied. For example, the a therapeutic agent can act to control infection or inflammation, enhance cell growth and tissue regeneration, control tumor growth, act as an analgesic, promote anti-cell attachment, and enhance bone growth, among other functions. Therapeutic agents as well as the specifics of the administration of therapeutic agents (i.e., dosing amount and schedule, administration route, etc.) are known the art. As used herein, the recitation of a biologically active agent inherently encompasses the pharmaceutically acceptable salts thereof.

In an aspect, a disclosed method can comprise monitoring the subject for adverse effects. In an aspect, in the absence of adverse effects, the method can further comprise continuing to treat the subject. In an aspect, in the presence of adverse effects, the method can further comprise modifying the treating step. In an aspect of a disclosed method, methods and techniques to monitor a subject can comprise qualitative (or subjective) means as well as quantitative (or objective) means. Such methods and techniques are known to the art and discussed supra.

In an aspect, a disclosed method can comprise generating a disclosed viral or non-viral vector. In an aspect, generating a disclosed viral vector can comprise generating an AAV vector (such as, for example, an cc47 AAV vector).

In an aspect, a disclosed method can comprise preparing a disclosed hydrogel.

In an aspect, a disclosed method of stimulating regeneration can comprise spatiotemporally targeted tissue regeneration. In an aspect, a disclosed method of stimulating regeneration can be used in a platform for spatiotemporally targeted tissue regeneration.

3. Methods of Promoting Glial Cell Proliferation of Injured and/or Damaged Spinal Cord Tissue

Disclosed herein is a method of promoting glial cell proliferation in injured and/or damaged spinal cord tissue, the method comprising administering to a subject in need thereof a disclosed isolated nucleic acid molecule, a disclosed vector, a disclosed pharmaceutical formulation, or any combination thereof.

Disclosed herein is a method of promoting glial cell proliferation in injured and/or damaged spinal cord tissue, the method comprising administering to a subject in need thereof a disclosed isolated nucleic acid molecule, a disclosed vector, a disclosed pharmaceutical formulation, or any combination thereof and administering a therapeutically effective amount of HB-EGF.

Disclosed herein is a method of promoting glial cell proliferation in injured and/or damaged spinal cord tissue, the method comprising administering to a subject in need thereof a disclosed HB-EGF. Disclosed herein is a method of promoting glial cell proliferation in injured and/or damaged spinal cord tissue, the method comprising administering to a subject in need thereof a therapeutically effective amount of HB-EGF or a pharmaceutical formulation comprising a therapeutically effective amount of HB-EGF.

In an aspect, a disclosed HB-EGF can comprise a recombinant HB-EGF. In an aspect, a disclosed HB-EGF can comprise epidermal growth factor a (HB-EGFa) or recombinant heparin binding epidermal growth factor a (r-HB-EGFa). In an aspect, a disclosed HB-EGF can comprise heparin binding epidermal growth factor b (HB-EGFb) or recombinant heparin binding epidermal growth factor b (rHB-EGFb). In an aspect, a disclosed HB-EGF can comprise recombinant human HB-EGF.

In an aspect, a disclosed method of promoting glial cell proliferation in injured and/or damaged spinal cord tissue can comprise treating a spinal cord injury, stimulating regeneration of injured and/or damaged spinal cord tissue, promoting axonal tract regeneration in injured and/or damaged spinal cord tissue, triggering neurite outgrowth in injured and/or damaged spinal cord tissue, triggering neuron formation in injured and/or damaged spinal cord tissue, improving spinal cord function in a subject in need thereof, or any combination thereof.

In an aspect, improving spinal cord function can comprise improving sensory function and/or motor function. In an aspect, improving sensory function and/or motor function can comprise transient improvements. In an aspect, improving sensory function and/or motor function can comprise sustained improvements. In an aspect, improvements can be sustained for at least 2 months, at least 3 months, at least 4 months, at least 6 months, at least 1 year, at least 18 months, at least 2 years, or at least 3 years, or more than 3 years.

In an aspect, a disclosed method can comprise reducing inflammation in the injured and/or damaged spinal cord tissue. In an aspect, a disclosed method can comprise reducing scar tissue in the injured and/or damaged spinal cord tissue.

In an aspect, a disclosed method can comprise applying a disclosed hydrogel to the injured and/or damaged spinal cord tissue. In an aspect, a disclosed hydrogel can comprise one or more therapeutic agents. In an aspect, the one or more additional disclosed therapeutic agents can comprise agents that promote glial cell proliferation, promote axonal tract regeneration, trigger neurite outgrowth, trigger neuron formation, stimulate regeneration of spinal cord tissue, or any combination thereof. In an aspect, the one or more additional disclosed therapeutic agents can comprise methylprednisolone or can comprise one or more corticosteroids. In an aspect, a disclosed hydrogel can comprise heparin binding epidermal growth factor (HB-EGF) or recombinant heparin binding epidermal growth factor (rHB-EGF). In an aspect, a disclosed hydrogel can comprise heparin binding epidermal growth factor a (HB-EGFa) or recombinant heparin binding epidermal growth factor a (rHB-EGFa). In an aspect, a disclosed hydrogel can comprise heparin binding epidermal growth factor b (HB-EGFb) or recombinant heparin binding epidermal growth factor b (rHB-EGFb). In an aspect, a disclosed hydrogel can comprise recombinant human HB-EGF.

In an aspect of a disclosed method, a disclosed isolated nucleic acid molecule, a disclosed vector, or a disclosed pharmaceutical formulation can be systemically or directly administered to the subject, or can be intravenously, subcutaneously, or intramuscularly administered to the subject, or can be directly administered to the injured and/or damaged spinal cord tissue.

In an aspect of a disclosed method, a disclosed HB-EGF or a disclosed pharmaceutical formulation comprising a disclosed HB-EGF can be systemically or directly administered to the subject, or can be intravenously, subcutaneously, or intramuscularly administered to the subject, or can be directly administered to the injured and/or damaged spinal cord tissue.

In an aspect of a disclosed method, a disclosed promoter can direct the expression of the encoded polypeptide in the subject's injured and/or damaged spinal cord tissue. In an aspect, spinal cord tissue can comprise neurons, neuroglia, or a combination thereof. In an aspect, neuroglia can comprise microglia and/or macroglia. In an aspect, neuroglia can comprise microglia, astrocytes, oligodendrocytes, ependymal cells, radial glia, Schwann cells, satellite cells, or any combination thereof.

In an aspect, a disclosed method can comprise repeating the administering step. For example, in an aspect, a disclosed method can comprise administering one or more times a disclosed isolated nucleic acid molecule, a disclosed vector, or a disclosed pharmaceutical formulation. In an aspect, a disclosed method can comprise administering one or more times a therapeutically effective amount of HB-EGF or a pharmaceutical formulation comprising a disclosed HB-EGF or a disclosed pharmaceutical formulation comprising a disclosed HB-EGF.

In an aspect, a disclosed method can comprise administering to the subject one or more additional therapeutic agents. Therapeutic agents are known to the art. In an aspect, therapeutic agents can comprise agents that promote glial cell proliferation, promote axonal tract regeneration, trigger neurite outgrowth, trigger neuron formation, stimulate regeneration of spinal cord tissue, or any combination thereof. In an aspect, a therapeutic agent can comprise methylprednisolone or can comprise one or more corticosteroids. In an aspect, a therapeutic agent can comprise any agent that is capable of providing a local or systemic biological, physiological, or therapeutic effect in the biological system to which it is applied. For example, the a therapeutic agent can act to control infection or inflammation, enhance cell growth and tissue regeneration, control tumor growth, act as an analgesic, promote anti-cell attachment, and enhance bone growth, among other functions. Therapeutic agents as well as the specifics of the administration of therapeutic agents (i.e., dosing amount and schedule, administration route, etc.) are known the art. As used herein, the recitation of a biologically active agent inherently encompasses the pharmaceutically acceptable salts thereof.

In an aspect, a disclosed method can comprise monitoring the subject for adverse effects. In an aspect, in the absence of adverse effects, the method can further comprise continuing to treat the subject. In an aspect, in the presence of adverse effects, the method can further comprise modifying the treating step. In an aspect of a disclosed method, methods and techniques to monitor a subject can comprise qualitative (or subjective) means as well as quantitative (or objective) means. Such methods and techniques are known to the art and discussed supra.

In an aspect, a disclosed method can comprise generating a disclosed viral or non-viral vector. In an aspect, generating a disclosed viral vector can comprise generating an AAV vector (such as, for example, an cc47 AAV vector).

In an aspect, a disclosed method can comprise preparing a disclosed hydrogel.

In an aspect, a disclosed method of promoting glial cell proliferation in injured and/or damaged spinal cord tissue can comprise spatiotemporally targeted tissue regeneration. In an aspect, a disclosed method of promoting glial cell proliferation in injured and/or damaged spinal cord tissue can be used in a platform for spatiotemporally targeted tissue regeneration.

4. Methods of Promoting Axonal Tract Regeneration in Injured and/or Damaged Spinal Cord Tissue

Disclosed herein is a method of promoting axonal tract regeneration in injured and/or damaged spinal cord tissue, the method comprising administering to a subject in need thereof a disclosed isolated nucleic acid molecule, a disclosed vector, a disclosed pharmaceutical formulation, or any combination thereof.

Disclosed herein is a method of promoting axonal tract regeneration in injured and/or damaged spinal cord tissue, the method comprising administering to a subject in need thereof a disclosed isolated nucleic acid molecule, a disclosed vector, a disclosed pharmaceutical formulation, or any combination thereof, and administering a therapeutically effective amount of HB-EGF.

Disclosed herein is a method of promoting axonal tract regeneration in injured and/or damaged spinal cord tissue, the method comprising administering to a subject in need thereof a disclosed HB-EGF. Disclosed herein is a method of promoting axonal tract regeneration in injured and/or damaged spinal cord tissue, the method comprising administering to a subject in need thereof a therapeutically effective amount of HB-EGF or a pharmaceutical formulation comprising a therapeutically effective amount of HB-EGF.

In an aspect, a disclosed HB-EGF can comprise a recombinant HB-EGF. In an aspect, a disclosed HB-EGF can comprise epidermal growth factor a (HB-EGFa) or recombinant heparin binding epidermal growth factor a (rHB-EGFa). In an aspect, a disclosed HB-EGF can comprise heparin binding epidermal growth factor b (HB-EGFb) or recombinant heparin binding epidermal growth factor b (rHB-EGFb). In an aspect, a disclosed HB-EGF can comprise recombinant human HB-EGF.

In an aspect, a disclosed method of promoting axonal tract regeneration in injured and/or damaged spinal cord tissue can comprise treating a spinal cord injury, stimulating regeneration of injured and/or damaged spinal cord tissue, promoting glial cell proliferation in injured and/or damaged spinal cord tissue, triggering neurite outgrowth in injured and/or damaged spinal cord tissue, triggering neuron formation in injured and/or damaged spinal cord tissue, improving spinal cord function in a subject in need thereof, or any combination thereof.

In an aspect, improving spinal cord function can comprise improving sensory function and/or motor function. In an aspect, improving sensory function and/or motor function can comprise transient improvements. In an aspect, improving sensory function and/or motor function can comprise sustained improvements. In an aspect, improvements can be sustained for at least 2 months, at least 3 months, at least 4 months, at least 6 months, at least 1 year, at least 18 months, at least 2 years, or at least 3 years, or more than 3 years.

In an aspect, a disclosed method can comprise reducing inflammation in the injured and/or damaged spinal cord tissue. In an aspect, a disclosed method can comprise reducing scar tissue in the injured and/or damaged spinal cord tissue.

In an aspect, a disclosed method can comprise applying a disclosed hydrogel to the injured and/or damaged spinal cord tissue. In an aspect, a disclosed hydrogel can comprise one or more therapeutic agents. In an aspect, the one or more additional disclosed therapeutic agents can comprise agents that promote glial cell proliferation, promote axonal tract regeneration, trigger neurite outgrowth, trigger neuron formation, stimulate regeneration of spinal cord tissue, or any combination thereof. In an aspect, the one or more additional disclosed therapeutic agents can comprise methylprednisolone or can comprise one or more corticosteroids. In an aspect, a disclosed hydrogel can comprise heparin binding epidermal growth factor (HB-EGF) or recombinant heparin binding epidermal growth factor (rHB-EGF). In an aspect, a disclosed hydrogel can comprise heparin binding epidermal growth factor a (HB-EGFa) or recombinant heparin binding epidermal growth factor a (rHB-EGFa). In an aspect, a disclosed hydrogel can comprise heparin binding epidermal growth factor b (HB-EGFb) or recombinant heparin binding epidermal growth factor b (rHB-EGFb). In an aspect, a disclosed hydrogel can comprise recombinant human HB-EGF.

In an aspect of a disclosed method, a disclosed isolated nucleic acid molecule, a disclosed vector, or a disclosed pharmaceutical formulation can be systemically or directly administered to the subject, or can be intravenously, subcutaneously, or intramuscularly administered to the subject, or can be directly administered to the injured and/or damaged spinal cord tissue.

In an aspect of a disclosed method, a disclosed HB-EGF or a disclosed pharmaceutical formulation comprising a disclosed HB-EGF can be systemically or directly administered to the subject, or can be intravenously, subcutaneously, or intramuscularly administered to the subject, or can be directly administered to the injured and/or damaged spinal cord tissue.

In an aspect, a disclosed method can comprise applying HB-EGF to, about, or near injured and/or damaged spinal cord tissue. In an aspect, a disclosed HB-EGF can comprise a disclosed recombinant HB-EGF Applying HB-EGF to, about, or near injured and/or damaged spinal cord tissue can comprise any means known to the art to apply a composition.

In an aspect of a disclosed method, a disclosed promoter can direct the expression of the encoded polypeptide in the subject's injured and/or damaged spinal cord tissue. In an aspect, spinal cord tissue can comprise neurons, neuroglia, or a combination thereof. In an aspect, neuroglia can comprise microglia and/or macroglia. In an aspect, neuroglia can comprise microglia, astrocytes, oligodendrocytes, ependymal cells, radial glia, Schwann cells, satellite cells, or any combination thereof.

In an aspect, a disclosed method can comprise repeating the administering step. For example, in an aspect, a disclosed method can comprise administering one or more times a disclosed isolated nucleic acid molecule, a disclosed vector, or a disclosed pharmaceutical formulation. In an aspect, a disclosed method can comprise administering one or more times a therapeutically effective amount of HB-EGF or a pharmaceutical formulation comprising a disclosed HB-EGF or a disclosed pharmaceutical formulation comprising a disclosed HB-EGF.

In an aspect, a disclosed method can comprise administering to the subject one or more additional therapeutic agents. Therapeutic agents are known to the art. In an aspect, therapeutic agents can comprise agents that promote glial cell proliferation, promote axonal tract regeneration, trigger neurite outgrowth, trigger neuron formation, stimulate regeneration of spinal cord tissue, or any combination thereof. In an aspect, a therapeutic agent can comprise methylprednisolone or can comprise one or more corticosteroids. In an aspect, a therapeutic agent can comprise any agent that is capable of providing a local or systemic biological, physiological, or therapeutic effect in the biological system to which it is applied. For example, the a therapeutic agent can act to control infection or inflammation, enhance cell growth and tissue regeneration, control tumor growth, act as an analgesic, promote anti-cell attachment, and enhance bone growth, among other functions. Therapeutic agents as well as the specifics of the administration of therapeutic agents (i.e., dosing amount and schedule, administration route, etc.) are known the art. As used herein, the recitation of a biologically active agent inherently encompasses the pharmaceutically acceptable salts thereof.

In an aspect, a disclosed method can comprise monitoring the subject for adverse effects. In an aspect, in the absence of adverse effects, the method can further comprise continuing to treat the subject. In an aspect, in the presence of adverse effects, the method can further comprise modifying the treating step. In an aspect of a disclosed method, methods and techniques to monitor a subject can comprise qualitative (or subjective) means as well as quantitative (or objective) means. In an aspect, qualitative means (or subjective means) can comprise a subject's own perspective. For example, a subject can report how he/she is feeling, whether he/she has experienced improvements and/or setbacks, whether he/she has experienced an amelioration or an intensification of one or more symptoms, or a combination thereof. In an aspect, quantitative means (or objective means) can comprise methods and techniques that include, but are not limited to, the following: (i) fluid analysis (e.g., tests of a subject's fluids including but not limited to aqueous humor and vitreous humor, bile, blood, blood serum, breast milk, cerebrospinal fluid, cerumen (earwax), digestive fluids, endolymph and perilymph, female ejaculate, gastric juice, mucus (including nasal drainage and phlegm), peritoneal fluid, pleural fluid, saliva, sebum (skin oil), semen, sweat, synovial fluid, tears, vaginal secretion, vomit, and urine), (ii) imaging (e.g., ordinary x-rays, ultrasonography, radioisotope (nuclear) scanning, computed tomography (CT), magnetic resonance imaging (MRI), positron emission tomography (PET), and angiography), (iii) endoscopy (e.g., laryngoscopy, bronchoscopy, esophagoscopy, gastroscopy, GI endoscopy, coloscopy, cystoscopy, hysteroscopy, arthroscopy, laparoscopy, mediastinoscopy, and thoracoscopy), (iv) analysis of organ activity (e.g., electrocardiography (ECG), electroencephalography (EEG), and pulse oximetry), (v) biopsy (e.g., removal of tissue samples for microscopic evaluation), and (vi) genetic testing.

In an aspect, a disclosed method can comprise generating a disclosed viral or non-viral vector. In an aspect, generating a disclosed viral vector can comprise generating an AAV vector (such as, for example, an cc47 AAV vector).

In an aspect, a disclosed method can comprise preparing a disclosed hydrogel.

In an aspect, a disclosed method of promoting axonal tract regeneration in injured and/or damaged spinal cord tissue can comprise spatiotemporally targeted tissue regeneration.

In an aspect, a disclosed method of promoting axonal tract regeneration in injured and/or damaged spinal cord tissue can be used in a platform for spatiotemporally targeted tissue regeneration.

5. Methods of Triggering Neurite Outgrowth and/or Triggering Neuron Formation in Injured and/or Damaged Spinal Cord Tissue

Disclosed herein is a method of triggering neurite outgrowth and/or triggering neuron formation in injured and/or damaged spinal cord tissue, the method comprising administering to a subject in need thereof a disclosed isolated nucleic acid molecule, a disclosed vector, a disclosed pharmaceutical formulation, or any combination thereof.

Disclosed herein is a method of triggering neurite outgrowth and/or triggering neuron formation in injured and/or damaged spinal cord tissue, the method comprising administering to a subject in need thereof a disclosed isolated nucleic acid molecule, a disclosed vector, a disclosed pharmaceutical formulation, or any combination thereof and administering a therapeutically effective amount of HB-EGF.

Disclosed herein is a method of triggering neurite outgrowth and/or triggering neuron formation in injured and/or damaged spinal cord tissue, the method comprising administering to a subject in need thereof a disclosed HB-EGF. Disclosed herein is a method of triggering neurite outgrowth and/or triggering neuron formation in injured and/or damaged spinal cord tissue, the method comprising administering to a subject in need thereof a therapeutically effective amount of HB-EGF or a pharmaceutical formulation comprising a therapeutically effective amount of HB-EGF.

In an aspect, a disclosed HB-EGF can comprise a recombinant HB-EGF. In an aspect, a disclosed HB-EGF can comprise epidermal growth factor a (HB-EGFa) or recombinant heparin binding epidermal growth factor a (rHB-EGFa). In an aspect, a disclosed HB-EGF can comprise heparin binding epidermal growth factor b (HB-EGFb) or recombinant heparin binding epidermal growth factor b (rHB-EGFb). In an aspect, a disclosed HB-EGF can comprise recombinant human HB-EGF.

In an aspect, a disclosed method of triggering neurite outgrowth and/or triggering neuron formation in injured and/or damaged spinal cord tissue can comprise treating a spinal cord injury, stimulating regeneration of injured and/or damaged spinal cord tissue, promoting glial cell proliferation in injured and/or damaged spinal cord tissue, promoting axonal tract regeneration in injured and/or damaged spinal cord tissue, improving spinal cord function in a subject in need thereof, or any combination thereof.

In an aspect, improving spinal cord function can comprise improving sensory function and/or motor function. In an aspect, improving sensory function and/or motor function can comprise transient improvements. In an aspect, improving sensory function and/or motor function can comprise sustained improvements. In an aspect, improvements can be sustained for at least 2 months, at least 3 months, at least 4 months, at least 6 months, at least 1 year, at least 18 months, at least 2 years, or at least 3 years.

In an aspect, a disclosed method can comprise reducing inflammation in the injured and/or damaged spinal cord tissue. In an aspect, a disclosed method can comprise reducing scar tissue in the injured and/or damaged spinal cord tissue.

In an aspect, a disclosed method can comprise applying a disclosed hydrogel to the injured and/or damaged spinal cord tissue. In an aspect, a disclosed hydrogel can comprise one or more therapeutic agents. In an aspect, the one or more additional disclosed therapeutic agents can comprise agents that promote glial cell proliferation, promote axonal tract regeneration, trigger neurite outgrowth, trigger neuron formation, stimulate regeneration of spinal cord tissue, or any combination thereof. In an aspect, the one or more additional disclosed therapeutic agents can comprise methylprednisolone or can comprise one or more corticosteroids. In an aspect, a disclosed hydrogel can comprise heparin binding epidermal growth factor (HB-EGF) or recombinant heparin binding epidermal growth factor (rHB-EGF). In an aspect, a disclosed hydrogel can comprise heparin binding epidermal growth factor a (HB-EGFa) or recombinant heparin binding epidermal growth factor a (rHB-EGFa). In an aspect, a disclosed hydrogel can comprise heparin binding epidermal growth factor b (HB-EGFb) or recombinant heparin binding epidermal growth factor b (rHB-EGFb). In an aspect, a disclosed hydrogel can comprise recombinant human HB-EGF.

In an aspect, a disclosed method can comprise applying HB-EGF to, about, or near injured and/or damaged spinal cord tissue. In an aspect, a disclosed HB-EGF can comprise a disclosed recombinant HB-EGF Applying HB-EGF to, about, or near injured and/or damaged spinal cord tissue can comprise any means known to the art to apply a composition.

In an aspect of a disclosed method, a disclosed HB-EGF or a disclosed pharmaceutical formulation comprising a disclosed HB-EGF can be systemically or directly administered to the subject, or can be intravenously, subcutaneously, or intramuscularly administered to the subject, or can be directly administered to the injured and/or damaged spinal cord tissue.

In an aspect of a disclosed method, a disclosed isolated nucleic acid molecule, a disclosed vector, or a disclosed pharmaceutical formulation can be systemically or directly administered to the subject, or can be intravenously, subcutaneously, or intramuscularly administered to the subject, or can be directly administered to the injured and/or damaged spinal cord tissue. In an aspect of a disclosed method, a disclosed promoter can direct the expression of the encoded polypeptide in the subject's injured and/or damaged spinal cord tissue. In an aspect, spinal cord tissue can comprise neurons, neuroglia, or a combination thereof. In an aspect, neuroglia can comprise microglia and/or macroglia. In an aspect, neuroglia can comprise microglia, astrocytes, oligodendrocytes, ependymal cells, radial glia, Schwann cells, satellite cells, or any combination thereof.

In an aspect, a disclosed method can comprise repeating the administering step. For example, in an aspect, a disclosed method can comprise administering one or more times a disclosed isolated nucleic acid molecule, a disclosed vector, or a disclosed pharmaceutical formulation. In an aspect, a disclosed method can comprise administering one or more times a therapeutically effective amount of HB-EGF or a pharmaceutical formulation comprising a disclosed HB-EGF or a disclosed pharmaceutical formulation comprising a disclosed HB-EGF.

In an aspect, a disclosed method can comprise administering to the subject one or more additional therapeutic agents. Therapeutic agents are known to the art. In an aspect, therapeutic agents can comprise agents that promote glial cell proliferation, promote axonal tract regeneration, trigger neurite outgrowth, trigger neuron formation, stimulate regeneration of spinal cord tissue, or any combination thereof. In an aspect, a therapeutic agent can comprise methylprednisolone or can comprise one or more corticosteroids. In an aspect, a therapeutic agent can comprise any agent that is capable of providing a local or systemic biological, physiological, or therapeutic effect in the biological system to which it is applied. For example, the a therapeutic agent can act to control infection or inflammation, enhance cell growth and tissue regeneration, control tumor growth, act as an analgesic, promote anti-cell attachment, and enhance bone growth, among other functions. Therapeutic agents as well as the specifics of the administration of therapeutic agents (i.e., dosing amount and schedule, administration route, etc.) are known the art. As used herein, the recitation of a biologically active agent inherently encompasses the pharmaceutically acceptable salts thereof.

In an aspect, a disclosed method can comprise monitoring the subject for adverse effects. In an aspect, in the absence of adverse effects, the method can further comprise continuing to treat the subject. In an aspect, in the presence of adverse effects, the method can further comprise modifying the treating step. In an aspect of a disclosed method, methods and techniques to monitor a subject can comprise qualitative (or subjective) means as well as quantitative (or objective) means. Such methods and techniques are known to the art and discussed supra.

In an aspect, a disclosed method can comprise generating a disclosed viral or non-viral vector. In an aspect, generating a disclosed viral vector can comprise generating an AAV vector (such as, for example, an cc47 AAV vector).

In an aspect, a disclosed method can comprise preparing a disclosed hydrogel.

In an aspect, a disclosed method of triggering neurite outgrowth and/or triggering neuron formation in injured and/or damaged spinal cord tissue can comprise spatiotemporally targeted tissue regeneration. In an aspect, a disclosed method of triggering neurite outgrowth and/or triggering neuron formation in injured and/or damaged spinal cord tissue can be used in a platform for spatiotemporally targeted tissue regeneration.

### 6. Methods of Improving Spinal Cord Function

Disclosed herein is a method of improving spinal cord function, the method comprising administering to a subject in need thereof a disclosed isolated nucleic acid molecule, a disclosed vector, a disclosed pharmaceutical formulation, or any combination thereof.

Disclosed herein is a method of improving spinal cord function, the method comprising administering to a subject in need thereof a disclosed isolated nucleic acid molecule, a disclosed vector, a disclosed pharmaceutical formulation, or any combination thereof, and administering a therapeutically effective amount of HB-EGF.

Disclosed herein is a method of improving spinal cord function, the method comprising administering to a subject in need thereof a disclosed HB-EGF. Disclosed herein is a method of improving spinal cord function, the method comprising administering to a subject in need thereof a therapeutically effective amount of HB-EGF or a pharmaceutical formulation comprising a therapeutically effective amount of HB-EGF.

In an aspect, a disclosed HB-EGF can comprise a recombinant HB-EGF. In an aspect, a disclosed HB-EGF can comprise epidermal growth factor a (HB-EGFa) or recombinant heparin binding epidermal growth factor a (rHB-EGFa). In an aspect, a disclosed HB-EGF can comprise heparin binding epidermal growth factor b (HB-EGFb) or recombinant heparin binding epidermal growth factor b (rHB-EGFb). In an aspect, a disclosed HB-EGF can comprise recombinant human HB-EGF.

In an aspect, a disclosed method of improving spinal cord function can comprise triggering neurite outgrowth and/or triggering neuron formation in injured and/or damaged spinal cord tissue, stimulating regeneration of injured and/or damaged spinal cord tissue, promoting glial cell proliferation in injured and/or damaged spinal cord tissue, promoting axonal tract regeneration in injured and/or damaged spinal cord tissue, improving spinal cord function in a subject in need thereof, or any combination thereof.

In an aspect, improving spinal cord function can comprise improving sensory function and/or motor function. In an aspect, improving sensory function and/or motor function can comprise transient improvements. In an aspect, improving sensory function and/or motor function can comprise sustained improvements. In an aspect, improvements can be sustained for at least 2 months, at least 3 months, at least 4 months, at least 6 months, at least 1 year, at least 18 months, at least 2 years, or at least 3 years.

In an aspect, a disclosed method can comprise reducing inflammation in the injured and/or damaged spinal cord tissue. In an aspect, a disclosed method can comprise reducing scar tissue in the injured and/or damaged spinal cord tissue.

In an aspect, a disclosed method can comprise applying a disclosed hydrogel to the injured and/or damaged spinal cord tissue. In an aspect, a disclosed hydrogel can comprise one or more therapeutic agents. In an aspect, the one or more additional disclosed therapeutic agents can comprise agents that promote glial cell proliferation, promote axonal tract regeneration, trigger neurite outgrowth, trigger neuron formation, stimulate regeneration of spinal cord tissue, or any combination thereof. In an aspect, the one or more additional disclosed therapeutic agents can comprise methylprednisolone or can comprise one or more corticosteroids. In an aspect, a disclosed hydrogel can comprise heparin binding epidermal growth factor (HB-EGF) or recombinant heparin binding epidermal growth factor (rHB-EGF). In an aspect, a disclosed hydrogel can comprise heparin binding epidermal growth factor a (HB-EGFa) or recombinant heparin binding epidermal growth factor a (rHB-EGFa). In an aspect, a disclosed hydrogel can comprise heparin binding epidermal growth factor b (HB-EGFb) or recombinant heparin binding epidermal growth factor b (r-B-EGFb). In an aspect, a disclosed hydrogel can comprise recombinant human HB-EGF.

In an aspect of a disclosed method, a disclosed isolated nucleic acid molecule, a disclosed vector, or a disclosed pharmaceutical formulation can be systemically or directly administered to the subject, or can be intravenously, subcutaneously, or intramuscularly administered to the subject, or can be directly administered to the injured and/or damaged spinal cord tissue. In an aspect of a disclosed method, a disclosed HB-EGF or a disclosed pharmaceutical formulation comprising a disclosed HB-EGF can be systemically or directly administered to the subject, or can be intravenously, subcutaneously, or intramuscularly administered to the subject, or can be directly administered to the injured and/or damaged spinal cord tissue.

In an aspect of a disclosed method, a disclosed promoter can direct the expression of the encoded polypeptide in the subject's injured and/or damaged spinal cord tissue. In an aspect, spinal cord tissue can comprise neurons, neuroglia, or a combination thereof. In an aspect, neuroglia can comprise microglia and/or macroglia. In an aspect, neuroglia can comprise microglia, astrocytes, oligodendrocytes, ependymal cells, radial glia, Schwann cells, satellite cells, or any combination thereof. In an aspect of a disclosed method, a disclosed HB-EGF or a disclosed pharmaceutical formulation comprising a disclosed HB-EGF can be systemically or directly administered to the subject, or can be intravenously, subcutaneously, or intramuscularly administered to the subject, or can be directly administered to the injured and/or damaged spinal cord tissue.

In an aspect, a disclosed method can comprise repeating the administering step. For example, in an aspect, a disclosed method can comprise administering one or more times a disclosed isolated nucleic acid molecule, a disclosed vector, or a disclosed pharmaceutical formulation. In an aspect, a disclosed method can comprise administering one or more times a therapeutically effective amount of HB-EGF or a pharmaceutical formulation comprising a disclosed HB-EGF or a disclosed pharmaceutical formulation comprising a disclosed HB-EGF.

In an aspect, a disclosed method can comprise administering to the subject one or more additional therapeutic agents. Therapeutic agents are known to the art. In an aspect, therapeutic agents can comprise agents that promote glial cell proliferation, promote axonal tract regeneration, trigger neurite outgrowth, trigger neuron formation, stimulate regeneration of spinal cord tissue, or any combination thereof. In an aspect, a therapeutic agent can comprise methylprednisolone or can comprise one or more corticosteroids. In an aspect, a therapeutic agent can comprise any agent that is capable of providing a local or systemic biological, physiological, or therapeutic effect in the biological system to which it is applied. For example, the a therapeutic agent can act to control infection or inflammation, enhance cell growth and tissue regeneration, control tumor growth, act as an analgesic, promote anti-cell attachment, and enhance bone growth, among other functions. Therapeutic agents as well as the specifics of the administration of therapeutic agents (i.e., dosing amount and schedule, administration route, etc.) are known the art. As used herein, the recitation of a biologically active agent inherently encompasses the pharmaceutically acceptable salts thereof.

In an aspect, a disclosed method can comprise monitoring the subject for adverse effects. In an aspect, in the absence of adverse effects, the method can further comprise continuing to treat the subject. In an aspect, in the presence of adverse effects, the method can further comprise modifying the treating step.

In an aspect of a disclosed method, methods and techniques to monitor a subject can comprise qualitative (or subjective) means as well as quantitative (or objective) means. Such methods and techniques are known to the art and discussed supra.

In an aspect, a disclosed method can comprise generating a disclosed viral or non-viral vector. In an aspect, generating a disclosed viral vector can comprise generating an AAV vector (such as, for example, an cc47 AAV vector).

In an aspect, a disclosed method can comprise preparing a disclosed hydrogel.

In an aspect, a disclosed method of improving spinal cord function can comprise spatiotemporally targeted tissue regeneration. In an aspect, a disclosed method of improving spinal cord function can be used in a platform for spatiotemporally targeted tissue regeneration.

### 7. Methods of Generating Vectors

Disclosed herein is a method of generating a disclosed non-viral vector or a disclosed viral vector Methods of generating non-viral and viral vectors are known to the art and are disclosed in the Examples provided herein. Disclosed herein is a method of generating an AAV vector, the method comprising: employing triple-plasmid transfection protocol. In an aspect in a disclosed method of generating an AAV vector, employing a triple-plasmid transfection protocol can comprise a capsid-specific helper plasmid, an adenoviral helper plasmid, and pTR-Enhancer-HSP68-GFP plasmids. In an aspect, a disclosed capsid-specific helper plasmid can comprise AAV2 Rep and AAVcc47 Cap genes. In an aspect, a disclosed pTR-Enhancer-HSP68-GFP plasmids can comprise differing enhance elements.

### 8. Methods of Generating Hydrogels

Disclosed herein is a method of generating a disclosed hydrogel. Methods of generating hydrogels are known to the art and are disclosed in the Examples provided herein. Disclosed herein is a method of generating a hydrogel, the method comprising synthesizing a UPy-bearing linker, synthesizing HA-UPy; synthesizing FITC-conjugated HA-UPy; oxidizing HA-UPy; adding one or more therapeutic agents to the oxidized HA-UPy-DA; and obtaining the hydrogel through dissolution. In an aspect, generation a disclosed hydrogel can comprise In an aspect, a disclosed HB-EGF can comprise a recombinant HB-EGF. In an aspect, a disclosed HB-EGF can comprise epidermal growth factor a (HB-EGFa) or recombinant heparin binding epidermal growth factor a (rHB-EGFa). In an aspect, a disclosed HB-EGF can comprise heparin binding epidermal growth factor b (HB-EGFb) or recombinant heparin binding epidermal growth factor b (rHB-EGFb). In an aspect, a disclosed HB-EGF can comprise recombinant human H B-EGF.

### 9. Methods of Identifying Putative TREEs

Disclosed herein is a method of identifying one or more putative TREEs, the method comprising isolating the nuclei from a first population of spinal cord cells and a second population of spinal cord cells; analyzing chromatin structure and function of the isolated nuclei to obtain a chromatin profile for the first population of spinal cord cells and for the second population of spinal cord cells; and comparing the chromatin profiles of the two populations of spinal cord cell to identify one or more putative TREEs.

In an aspect, a disclosed first population of spinal cord cells has sustained an injury, damage, and/or an insult, and wherein a disclosed second population of spinal cord cells has not sustained an injury, damage, and/or an insult, or vice versa. In an aspect, a disclosed first population of spinal cord cells has sustained an injury, damage, and/or an insult and is regenerating, and wherein a disclosed second population of spinal cord cells has not sustained an injury, damage, and/or an insult, or vice versa. In an aspect, a disclosed first population of spinal cord cells has sustained an injury, damage, and/or an insult and is regenerating, and a disclosed second population of spinal cord cells has sustained an injury, damage, and/or an insult and is not regenerating, or vice versa. In an aspect, a disclosed first population of spinal cord cells can be obtained from a subject having sustained an injury, damage, and/or an insult, and wherein a disclosed second population of spinal cord cells can be obtained from a subject not having sustained an injury, damage, and/or an insult, or vice versa. In an aspect, a disclosed first population of spinal cord cells can be obtained from a subject having sustained an injury, damage, and/or an insult and is regenerating, and wherein a disclosed second population of spinal cord cells can be obtained from a subject not having sustained an injury, damage, and/or an insult, or vice versa. In an aspect, a disclosed first population of spinal cord cells can be obtained from a subject having sustained an injury, damage, and/or an insult and is regenerating, and wherein a disclosed second population of spinal cord cells can be obtained from a subject having sustained an injury, damage, and/or an insult and is not regenerating, or vice versa. In an aspect, a disclosed first population of spinal cord cells can comprise a compilation and/or aggregate of cells. In an aspect, a disclosed second population of spinal cord cells can comprise a compilation and/or aggregate of cells. In an aspect, a disclosed compilation and/or aggregate of cells can comprise spinal cord cells from one or more subjects. In an aspect, cells can be mammalian spinal cord cells, human spinal cord cells, porcine or mouse spinal cord cells, and/or zebrafish spinal cord cells. In an aspect, one or both subjects can comprise a mammal, a human, a pig, a mouse, or a zebrafish.

In an aspect, injury, damage, and/or an insult can comprise a contusion injury, a compression injury, a transection injury, or any combination thereof. In an aspect, injury, damage, and/or an insult can comprise a disease such as, for example, a degenerative disease. In an aspect, a disease can comprise transverse myelopathy, Brown-Séquard syndrome, central cord syndrome, anterior cord syndrome, and conus medullaris syndrome. In an aspect, injury, damage, and/or an insult can comprise a vascular injury, damage, and/or insult.

In an aspect, one or more putative TREEs can be incorporated into a vector or an isolated nucleic acid molecule.

In an aspect, a disclosed method can comprise validating a putative TREE. In an aspect, validating a putative TREE can comprise generating a transgenic zebrafish and assessing the ability of the putative TREE to drive expression of a report gene in damaged and/or injured tissue.

Disclosed herein is a method of identifying one or more TREEs, the method comprising obtaining a first population of spinal cord cells; isolating the nuclei from the first population of spinal cord cells; analyzing chromatin structure and function of the isolated nuclei to obtain a chromatin profile for the first population of spinal cord cells; obtaining a second population of spinal cord cells; isolating the nuclei from the second population of spinal cord cells; analyzing chromatin structure and function of the isolated nuclei to obtain a chromatin profile for the second population of spinal cord cells; and comparing the chromatin profiles between the two populations of spinal cord cells to identify one or more putative TREEs.

In an aspect, a disclosed first population of spinal cord cells has sustained an injury, damage, and/or an insult, and wherein a disclosed second population of spinal cord cells has not sustained an injury, damage, and/or an insult, or vice versa. In an aspect, a disclosed first population of spinal cord cells has sustained an injury, damage, and/or an insult and is regenerating, and wherein a disclosed second population of spinal cord cells has not sustained an injury, damage, and/or an insult, or vice versa. In an aspect, a disclosed first population of spinal cord cells has sustained an injury, damage, and/or an insult and is regenerating, and a disclosed second population of spinal cord cells has sustained an injury, damage, and/or an insult and is not regenerating, or vice versa. In an aspect, a disclosed first population of spinal cord cells can be obtained from a subject having sustained an injury, damage, and/or an insult, and wherein a disclosed second population of spinal cord cells can be obtained from a subject not having sustained an injury, damage, and/or an insult, or vice versa, in an aspect, a disclosed first population of spinal cord cells can be obtained from a subject having sustained an injury, damage, and/or an insult and is regenerating, and wherein a disclosed second population of spinal cord cells can be obtained from a subject not having sustained an injury, damage, and/or an insult, or vice versa. In an aspect, a disclosed first population of spinal cord cells can be obtained from a subject having sustained an injury, damage, and/or an insult and is regenerating, and wherein a disclosed second population of spinal cord cells can be obtained from a subject having sustained an injury, damage, and/or an insult and is not regenerating, or vice versa.

In an aspect, a disclosed first population of spinal cord cells can comprise a compilation and/or aggregate of cells. In an aspect, a disclosed second population of spinal cord cells can comprise a compilation and/or aggregate of cells. In an aspect, a disclosed compilation and/or aggregate of cells can comprise spinal cord cells from one or more subjects. In an aspect, cells can be mammalian spinal cord cells, human spinal cord cells, porcine or mouse spinal cord cells, and/or zebrafish spinal cord cells. In an aspect, one or both subjects can comprise a mammal, a human, a pig, a mouse, or a zebrafish.

In an aspect, injury, damage, and/or an insult can comprise a contusion injury, a compression injury, a transection injury, or any combination thereof.

In an aspect, injury, damage, and/or an insult can comprise a disease such as, for example, a degenerative disease. In an aspect, a disease can comprise transverse myelopathy, Brown-Séquard syndrome, central cord syndrome, anterior cord syndrome, and conus medullaris syndrome. In an aspect, injury, damage, and/or an insult can comprise a vascular injury, damage, and/or insult.

In an aspect, one or more putative TREEs can be incorporated into a vector or an isolated nucleic acid molecule.

In an aspect, a disclosed method can comprise validating a putative TREE. In an aspect, validating a putative TREE can comprise generating a transgenic zebrafish and assessing the ability of the putative TREE to drive expression of a report gene in damaged and/or injured tissue.

In an aspect, a disclosed method of identifying putative TREEs can be used in a platform for spatiotemporally targeted tissue regeneration.

### EXAMPLES

Whereas axon growth across damaged spinal cord tissue is minimal in adult mammals, zebrafish regenerate a bridge of glia and axons and recover locomotor ability. Here, a heparin binding epidermal growth factor (hb-egf) was identified to be as a secreted factor gene preferentially induced in caudal regions of spinal cord injury sites in zebrafish. Zebrafish deficient in the hb-egfa isoform had defects in ependymal cell proliferation, bridge formation, and axon regeneration, disrupting recovery from paralyzing injuries. Whereas broad transgenic hb-egfa overexpression disrupted axon regeneration and functional recovery, local delivery of human HB-EGF in engineered hydrogels accelerated these events. Epigenetic profiling for tissue regeneration enhancer elements (TREEs) revealed sequences distal to hb-egfa sufficient to activate gene expression in caudal injury sites, where its production instructs axon regeneration across the wound site. This TREE directed gene expression in spinal cord injuries in neonatal mice and increased axon regeneration in neonates as a delivery vehicle when engineered with a human HB-EGF transgene. These results identified Hb-egf as an instructive and therapeutic factor for spinal cord repair.

### A. Materials and Methods

### 1. Zebrafish

Wild-type, mutant, or transgenic male and female zebrafish of the Ekkwill (EK) strain were used for all experiments. Ages of larvae used were between 3 days and 6 days post-fertilization (dpf). Adult animals were between 3 months and 12 months of age and measured ˜2 cm in length. To minimize differences in regeneration and recovery due to different manipulations, clutchmates were used as controls for all experiments. Experiments with zebrafish were approved by the Institutional Animal Care and Use Committee (IACUC) at Duke University.

### 2. Generation of Transgenic and Mutant Lines

a. Generation of GFAP:H2B-mCherry Zebrafish

H2A-mCherry sequence (allele^{pd367}) was obtained from the pSKS-ubiq-H2AmCherry plasmid by PCR using the following primers: Kozak-H2A-mCherry-F-Primer: 5′-gccaccATGGCAGGTGGAAAAGCAGG-3′ (SEQ ID NO:01) and H2A-mCherry-polyA-Rev-Primer: 5′-GATACATTGATGAGTTTGGACAAACCAC-3′ (SEQ ID NO:02). This PCR fragment was A-tailed using Taq Polymerase and TA cloned into a pCRK/GW/TOPO vector (Invitrogen, Cat. N. K2500-20). A multi-gateway reaction was performed with p5e-gfap, p3-pA, and a destination vector containing ISce1 sites flanking the clonase cassette. Plasmids were digested using 1-SceI enzyme for 30 minutes at 37° C. before injection into Casper strain embryos at the one-cell stage.

b. Generation of hb-egfa:EGFP Zebrafish

To generate the hb-egfa:EGFP_BAC construct, the first exon of the hb-egfa gene in the BAC clone CH73-26113 (containing ˜66 kb upstream and 31 kb downstream the hb-egfa gene) was replaced with the EGFP-SV40 polyA cassette using Red/ET recombineering technology (Gene Bridges). The 5′ and 3′ homologous arms for recombination were a 50 bp fragment upstream and downstream the first exon of hb-egfa and were included in PCR primers to flank the EGFP-SV40 polyA cassette. The same technology was used to insert a I-SceI site in the final BAC construct, which was purified with Nucleobond BAC 100 kit (Clontech) and co-injected with I-Sce 1 into one-cell-stage zebrafish embryos. A stable transgenic line showing EGFP fluorescence was selected.

c. Generation of hb-egfb^{egfp} Zebrafish

Transgenic fish were generated using TALEN-directed knock-in (Bedell V M, et al. (2012) Nature. 491:114-118) and PhiC31 mediated recombination (Hu G. et al. (2011) Dev. Dyn. 204:2101-2107). Briefly, a pair of obligated heterodimeric TALENs targeting hbegh ATG region (5′-TCAGTCAGACCGACTA-3′ (SEQ ID NO:03) and 5′-TTTCTTGGGATAGTCCAA-3′ (SEQ ID NO:04)) were assembled with standard golden gate assembly (Cermak T, et al. (2011) Nucleic Acids Res. 39:e82; Dahlem T J, et al. (2012) PLoS Genet. 8:e1002861) and in vitro transcribed with mMESSAGE mMACHINE SP6 Kit (Life Technologies).

A 5′ phosphorylated single stranded oligo (ssOligo) was synthesized from Integrated DNA Technologies (IDT) and used as a homology-directed repair (HDR) template (5′-CCTTTTCTTTGGGATAGTCCAAGACACCCCCAACTGAGAGAACTCAAAGGTTACCCC AGTTGGGGTCTGACTGAACCTCCCTGCCTCCAGCGCCGTC-3′)(SEQ ID NO:05). hbegfb TALEN mRNAs and ssOligo were co-injected into one-cell stage zebrafish embryos and stable lines were screened by PCR. Next, stable hbegfb^{attP} F1 zebrafish were inter crossed and F2 embryos were injected with PhiC31o mRNA, FLPase mRNA as well as a donor plasmid pERBF-EGFP containing attB, GFP-SV40 polyA, and two FRT sites flanking the vector sequences. Finally, stable hb-egfb:EGFP zebrafish were isolated based on GFP expression and sequenced to ensure correct integration.

d. Generation of hb-egfaKO and kb-egfbKO Zebrafish.

hb-egf and hb-egfb mutants were generated using the CRISPR/Cas9 technology. The target sequences were 5′-TGGCCACGTTCATATTTAAGCGG-3′ (SEQ ID NO:06) and 5′-AGCCCTTGCTGTGGTAGCTGTCG-3′ (SEQ ID NO:07) for hb-egfa and 5′-CCACCAAACCCAAACATCCGTCG-3′ (SEQ ID NO:08) and 5′-CCACAGCGCTGGCGGTCATAGCA-3′ (SEQ ID NO:09) for hb-egfb and led to deletion of a 1512 bp and 2375 bp fragment, respectively. Injected embryos were raised to adulthood and screened using the following primers:

| Primer Name | Primer Sequence | SEQ ID NO: |
|---|---|---|
| Tg(hb-egfaKO) Fw1 | GCAGGTAACCATACCAGGGATAAAAGG | SEQ ID NO: 10 |
| Tg(hb-egfaKO) Rev1 | GGTAAAGACGAAAAGACGCAAGACTG | SEQ ID NO: 11 |
| Tg(hb-egfaKO) Rev2 | CAGGAGGAGGCCAATGATGG | SEQ ID NO: 12 |
| Tg(hb-egfbKO) Fw1 | GCACTGACATCACTCTTGCTCAAC | SEQ ID NO: 13 |
| Tg(hb-egfbKO) Rev1 | CCATGAATGCAGAAATCTTTGTATTCCTCC | SEQ ID NO: 14 |
| Tg(hb-egfbKO) Rev2 | GCCTCGAGTTTGACCTTTTCTTCG | SEQ ID NO: 15 |

e. Generation of hsp70:shb-egfa-P2A-TBFP Zebrafish

hb-egfa cDNA was amplified from the BAC clone CH73-26113 using the following primers: hb-egfa_Fw 5′-ATGAACTTTTTAACAGTCTT-3′ (SEQ ID NO:16) and hb-egfa . . . Rev 5′ CAGAGAGAAATCGTGACATC-3′ (SEQ ID NO:17). Primers were linked to homology arms for Gibson assembly and inserted into an hasp70-2A-TBFP vector using Gateway LR Clonase II Enzyme mix (ThermoFisher Cat #11791020). The final plasmid was co-injected into one-cell stage wild-type embryos with I-SceI. Multiple founders were isolated, propagated, and screened for germline transmission of the transgene selecting for TBFP expression after heat shock. A single line was chosen for maintenance.

f. Generation of hb-egfaEN-cfos:EGFP and cfos:EGFP Zebrafish

hb-egfaEN was PCR-amplified from genomic DNA of 3 dpf EK zebrafish embryos using primers 5′-ACACGTTTCCTCTAGTCCCAG-3′ (SEQ ID NO:18) and 5′-GGTTTTACTGTGCTCAAATTGC-3′ (SEQ ID NO:19). The amplified sequence was inserted into pCR8-GW-topoTA (invitrogen K2500-20) to generate pEntry vectors that were subsequently recombined with PMP6, a Gateway vector containing LR recombination sites upstream of the 95 bp minimal mouse cfos promoter driving EGFP (Fivaz J. et al. (2000) Gene. 255:185-194). The hb-egfaEN-cfos:EGFP construct was injected into fertilized zebrafish embryos along with I-Sce1 meganuclease, using standard transgenesis techniques. F1 embryos were genotyped to check for transgene insertion and transmission with the following primers: EGFP Fw 5′-ATGGTGAGCAAGGGCGAG-3′ (SEQ ID NO:20) and EGFP Rev 5′-CTTGTACAGCTCGTCCATGC-3′ (SEQ ID NO:21). Three stable lines were established. Animals PCR-positive for the transgene were used for experiments. As a negative control, generated control lines only carrying a cfos:EGFP construct were generated.

### 3. Spinal Cord Injuries in Zebrafish

For larval spinal cord transection, 3 dpf larvae were anesthetized using egg water containing 20× tricaine methanesulfonate (MS222; Sigma-Aldrich), placed in the well of an injection agarose tray and the spinal cord was transected dorsal to the anal pore using a 30 G needle (Wehner D. et al. (2017) Nat. Commun. 8:126). Transection was visually confirmed by visualizing a complete gap between rostral and caudal spinal cord stumps, using experimental lines crossed with transgenics to visualize the spinal cord by fluorescence.

For adult spinal cord injuries, zebrafish were anesthetized using 0.75% 2-phenoxyethanol in fish water. Fine scissors were used to make a small incision and expose the vertebral column by pushing the muscle tissue aside. Then, the vertebral column was transected halfway between the dorsal fin and the operculum. Complete transection was visually confirmed at the time of surgery.

### 4. RNA- and ATAC-Sequencing

All tissue samples were generated from adult zebrafish spinal cord regions collected 2 mm rostral and 2 mm caudal to the lesion site. Sham-operated clutchmate animals were used as controls, collecting the same portion of spinal cord as in injured experimental animals. All samples were prepared in triplicate for each time point.

For ATAC-seq, spinal cords from 60 male and female zebrafish at equal ratios were digested into a single cell suspension using 0.25% trypsin-EDTA; dissociated cells were processed for FACS sorting using an Astrios sorter, to collect live and single cells. 50,000 cells were processed for ATAC-seq library preparation as previously described (Thompson J D, et al. (2020) Development. 147(14):dev191262), and sequencing was performed at the Duke Center for Genomic and Computational Biology on the Illumina HiSeq 4000 platform, with over 40 million 150 bp paired-end reads obtained for each library. Sequences were aligned to the zebrafish genome (danRer10) using Bowtie2 v 2.2.5 (Langmead B, et al. (2012) Nat. Methods. 9(4):357-359). The mapped reads were filtered by samtools (v 1.3.1, with parameter -q 30) (Li H, et al. (2009) Bioformatics. 25(16):2078-2079) and duplicates were removed by picard (v 1.91). Peak calls were determined using MACS2 (v 2.1.0, with parameter -f BAM -g 1.5e9 -q 0.05 --nomodel -shift 37 --extsize 73) (Zhang Y, et al. (2008) Genome Biol 9(9):R137), and csaw (v 1.20.0, cutoff P value<0.05) (Lun A T, et al. (2016) Nucleic Acids Res. 44(5):e45). DiffBind (v 2.14.0) was used to call differential accessible sites. Filter conditions were p value<0.05 and fold change >1.2.

For RNA-seq, RNA was extracted using TRI reagent (Sigma), and genomic DNA was eliminated using the RNA Clean & Concentrator Kit (Zymo Research, R1013). Library preparation and sequencing was performed at the Duke Center for Genomic and Computational Biology using an Illumina HiSeq 4000, with over 40 million 50-bp single-end reads obtained for each library. As for ATAC-seq, reads were aligned to the zebrafish genome (danRer10) using Tophat2 (v 2.1.1)(Kim D, et al. (2013) Genome Biol. 14(4):R36). The mapped reads were filtered by samtools (v 1.3.1, with parameter -q 30 (Li H. et al. (2009) Bioformatics. 25(16):2078-2079) and counted by htseq-count (v 0.6.0) (Anders S, et al. (2015) Bioinformatics. 31(2):166-169). Differential analyses were performed by Bioconductor package DESeq2 (v1.26.0) (Love M I, et al. (2014) Genome Biol. 15(12):550).

ATAC-Seq peaks were paired to RNA-Seq differential expression data by annotated nearest gene symbols by ChIPpeakAnno (v3.20.1) (Zhu L J, et al. (2010) BMC Bioinformatics. 11:237). A conservation test was performed using the DNA sequence alignment visualization online tool mVista (Mayor C, et al. (2000) Bioinformatics. 16(11):1046-1047) and a circle plot was generated using circus (v 0.69-8) (Kryzwinski M, et al. (2009) Genome Res. 19(9):1639-1645). Analyses of TF binding sites (Sox2) in the promoter region of hb-egfa were performed using motifmatchr (v 1.12.0) (Schep A N, et al. (2017) Nat. Methods. 14(10): 975-978 with motifs downloaded from JASPAR (v2018) (Sandelin A. et al (2004) Nucleic Acid Res. 32(D91-94).

The tracks and heatmaps of ATAC-seq and RNA-seq signals were plotted by Bioconductor package trackViewer (v 1.22.1) (Ou J, et al. (2019) Nat. Methods. 16(6):453-454) and ComplexHeatmap (v 2.2.0) (Gu Z, et al. (2016) Bioinformatic. 32(18):2847-2849).

5. RNA Isolation and qRT-PCR

Spinal cords were homogenized in Trizol, and RNA was extracted using the standard Trizol protocol. Genomic DNA removed using RNA clean and Concentrator Kit (Zymo Research, R1013). cDNA synthesis was performed using Transcriptor First Strand cDNA Synthesis Kit (Roche. 04897030001) and qPCR run was performed with LightCycler480 SYBR Green 1 Master (Roche, 04707516001). All gene expression values were normalized to beta-actin levels. Primers used were the following:

| Primer Name | Primer Sequence | SEQ ID NO: |
|---|---|---|
| hb-egfa Fw | 5′-GGGCCCTCATGCATATGTGA-3′ | SEQ ID NO: 22 |
| hb-egfa Rev | 5′-AGAATTTCCACACGGTCGCC-3′ | SEQ ID NO: 23 |
| hb-egfb Fw | 5′-AGCGGGATTTACGCACTCAT-3′ | SEQ ID NO: 24 |
| hb-egfb Rev | 5′-GCATGCAGAATATCTTAATGCCA-3′ | SEQ ID NO: 25 |
| sox2 Fw | 5′-GGGCACGGGGAACACCAACT-3′ | SEQ ID NO: 26 |
| sox2 Rev | 5′-TGGTCGCTTCTCGCTCTCGG-3′ | SEQ ID NO: 27 |
| actin Fw | 5′-GACAACGGCTCCGGTATG-3 | SEQ ID NO: 28 |
| actin Rev | 5′-CATGCCAACCATCACTCC-3′ | SEQ ID NO: 29 |

### 6. Western Blotting

Zebrafish spinal cords were homogenized in RIPA buffer containing Proteinase and Phosphatase inhibitor (Thermo Fisher Scientific, 78442). Samples were denatured at 95° C. for 5 min. Quantified and tissue lysates were analyzed on Mini-Protein tetra cell (Bio-Rad) using sodium dodecyl sulfate polyacrylamide gel electrophoresis (SDS-PAGE) in Tris/glycine/SDS buffer. After electrophoresis proteins were transferred to a PVDF membrane using the Mini-Protein tetra cell in Tris/glycine buffer (v/v). Membranes were blocked for 1 hr at room temperature using 3% milk in Tris-buffered saline and Tween-20 (TBST), then were incubated with primary anti-Sox2 antibody (Abcam, ab97959, 1:500) and anti-GAPDH (Proteintech 60004-1-1G, 1:500) overnight at 4° C. Membranes were incubated with appropriate HRP-conjugated secondary antibodies (Thermo Fisher Scientific), washed in TBST and developed with Pierce ECL western blotting substrate. Western blot signals were quantified as previously described (Davarinejad H. (2017)).

### 7. Edu, Biocytin, and Human Recombinant (HR) HB-EGF Treatment

For EdU incorporation, zebrafish were injected intraperitoneally with 10 μL of 10 mM 5-ethynyl-2′-deoxyuridine (EdU, Molecular Probes, A10055), and tissue was collected 24 hrs post-treatment. For biocytin treatment used for anterograde axon tracing, adult fish were anaesthetized using 0.75% 2-phenoxyethanol in fish water. Scissors were used to make a small incision on the dorsal side of the skin and to transect the spinal cord 2 mm rostral to the original spinal cord transection site. A biocytin-soaked gelfoam gelatin sponge was applied at a new injury site (Gelfoam, Pfizer. 09-0315-08; Alexa Fluor 594 Biocytin, Thermofisher Scientific A12922). The incision was closed and glued using Vetbond tissue adhesive material (Santa Cruz Biotechnology, sc-361931). Tissue was collected 24 hrs after biocytin application.

For HR-HBEGF treatment, HR-HBEGF protein (R&D Systems, 259-HE-250) was conjugated with hydrogel (see below) and 3 μL of solution was locally injected just rostral to the original spinal cord transection site with an Hamilton syringe.

### 8. Generation of Synthetic Biomaterials for Local HR-HB-EGF Delivery

a. Synthesis of UPy-Bearing Linker

Briefly, 2-amino-4-hydroxy-6-methylpyrimidine (10 g. 0.08 mol) was suspended in excess 1,6-diisocyanatohexane (107 g, 0.64 mol). The mixture was stirred at 100° C. overnight under argon atmosphere. The product termed as 1-(6-isocyanatohexyl)-3-(6-methyl-4-oxo-1,4-dihydropyrimidin-2-yl)urea was then precipitated in n-hexane, filtered and dried in vacuum. 1-(6-Isocyanatohexyl)-3-(6-methyl-4-oxo-1,4-dihydropyrimidin-2-yl)urea (5 g, 0.017 mol) was mixed with N-boc-1,6-hexanediamine (5.5 g, 0.025 mol) in anhydrous dichloromethane (DCM, ˜75 mL) and kept at 50° C. overnight to obtain tert-butyl(6-(3-(6-(3-(6-methyl-4-oxo-1,4-dihydropyrimidin-2-yl)ureido)hexyl) ureido)hexyl)carbamate, which was precipitated in chilled diethyl ether, filtered, and dried in vacuum.

Next, tert-butyl(6-(3-(6-(3-(6-methyl-4-oxo-1,4-dihydropyrimidin-2-yl)ureido)hexyl) ureido)hexyl)carbamate (5 g) was dispersed in dichloromethane (90 mL) and trifluoroacetic acid (TFA, 10 mL) was added to the suspension. The mixture was stirred vigorously for about 6 hr at room temperature.

After the reaction, the DCM and TFA were removed completely using a rotary evaporator. The solid residue was then dissolved in minimum amount of DCM and precipitated in excess ice-cold acetone to obtain 1-(6-(3-(6-aminohexyl)ureido)hexyl)-3-(6-methyl-4-oxo-1,4-dihydropyrimidin-2-yl)urea-trifluoroacetic acid which was filtered, washed repeatedly with acetone and finally dried in vacuum. 1-(6-(3-(6-Aminohexyl)ureido)hexyl)-3-(6-methyl-4-oxo-1,4-dihydropyrimidin-2-yl)urea-trifluoroacetic acid was next treated with amberlite IRA 400 chloride ion exchange resin in a DMSO:water (1:1) mixture at room temperature for about 2 hrs. Next, the resin was filtered off and the solution of the UPy-bearing linker 1-(6-(3-(6-aminohexyl)ureido)hexyl)-3-(6-methyl-4-oxo-1,4-dihydropyrimidin-2-yl)urea·HCl was directly used to conjugate with hyaluronic acid (HA).

b. Synthesis of HA-UPy

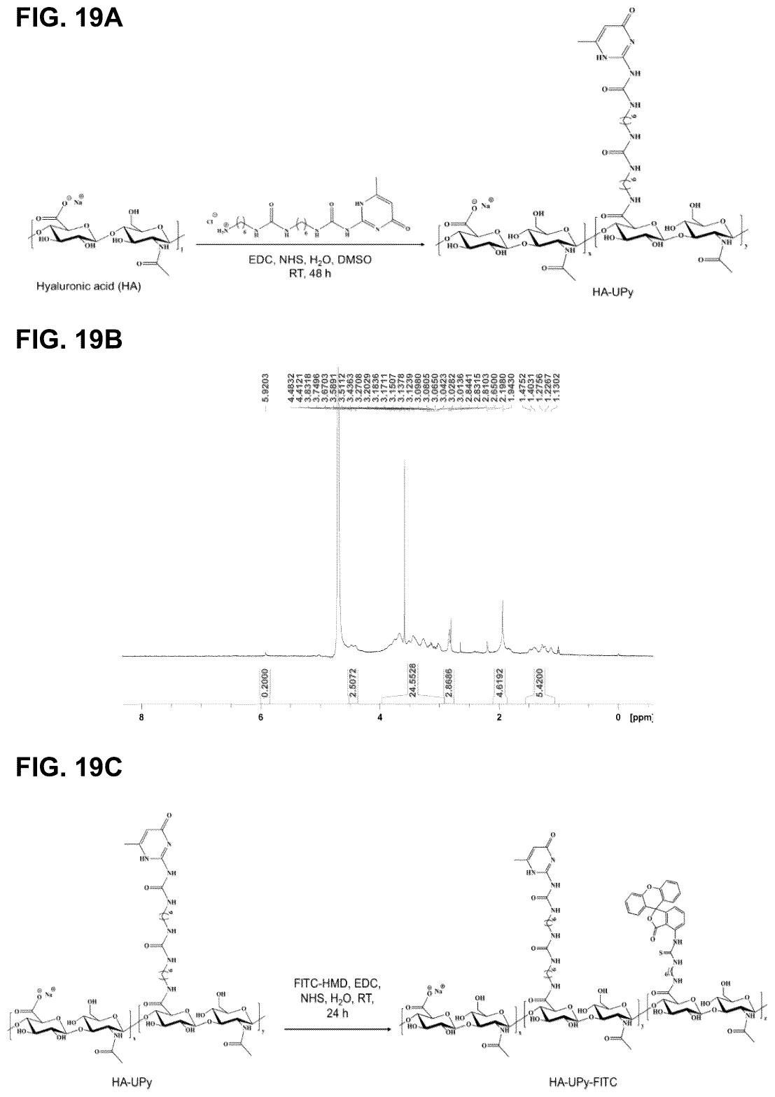

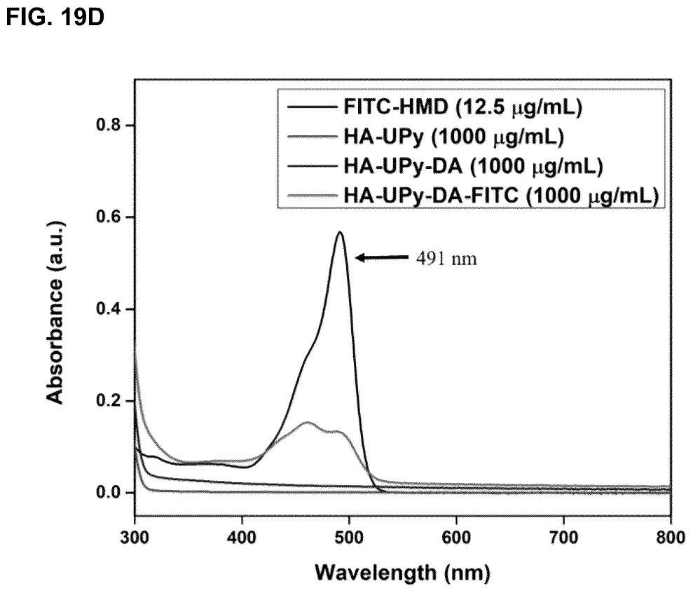

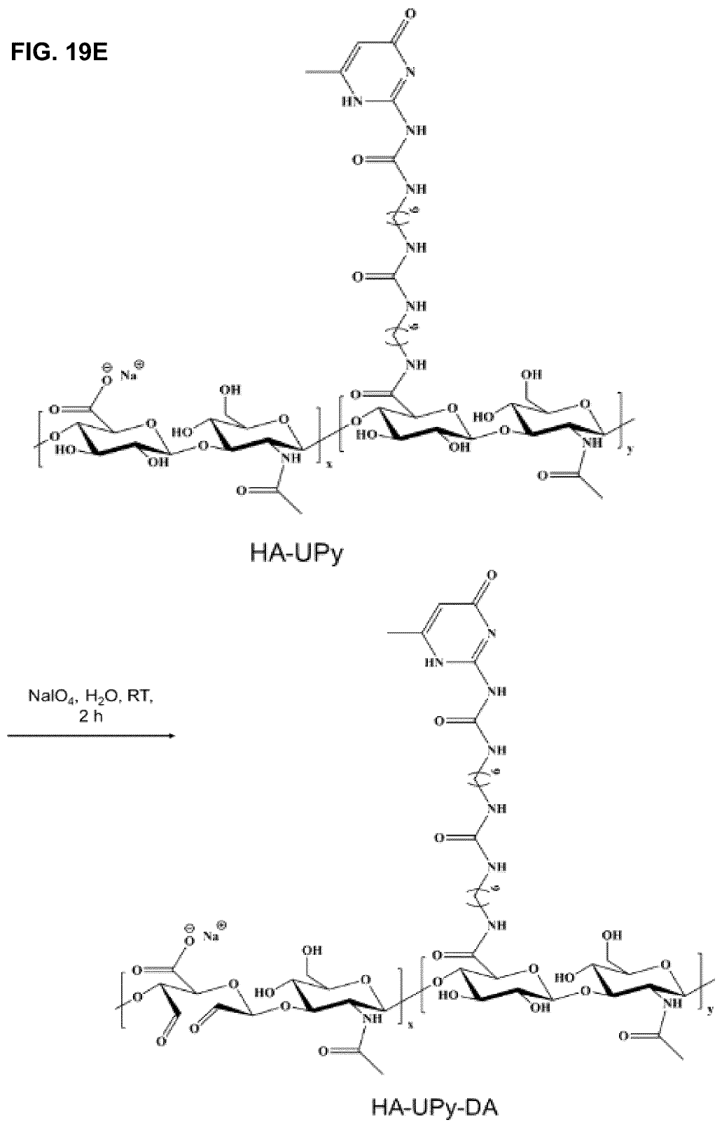

HA-UPy was synthesized by coupling the UPy-bearing linker, 1-(6-(3-(6-aminohexyl)ureido)hexyl)-3-(6-methyl-4-oxo-1,4-dihydropyrimidin-2-yl)urea·HCl, with sodium hyaluronate (HA, Mol. Wt. 200 kDa) via the amide coupling reaction using 1-ethyl-3-(3-dimethylaminopropyl)carbodiimide (EDC)/N-hydroxysuccinimide (NHS) chemistry (FIG. 19A). Briefly, HA was dissolved in a mixture of deionized water and DMSO (1:1) at ˜5 mg/mL. To the solution, EDC and NHS (each 1 equivalent with respect to the carboxylic acid groups of HA) were added at 15 min intervals. Next, UPy-bearing linker (1 equivalent with respect to the carboxylic acid groups of HA) was added and stirred at room temperature for ˜48 hrs. The resulting HA-UPy was purified via dialysis against water and lyophilized. The extent of UPy conjugation was quantified via 1HNMR spectroscopy and found to be 20±2% (FIG. 19B).

c. Synthesis of FITC Conjugated HA-Upy (HA-Upy-FITC)

Fluorescein isothiocyanate (FITC) was reacted with a hexamethylenediamine (HMD) to obtain a FITC conjugated linker (FITC-HMD). Briefly, HMD was dissolved in methanol (1.5 mmol HMD in 50 mL methanol). Triethylamine (0.5 mL) was added to the HMD solution. FITC (117 mg) dissolved in a mixture of 10 mL methanol and 0.1 mL triethylamine was added dropwise to the HMD solution for ˜30 min. The mixture was stirred for 1 hr at room temperature and then kept overnight at room temperature in dark.

The resultant red-colored solid was filtered using Whatman filter paper and washed with 10 mL methanol. The solid fluoresceinthiocarbamyl hexamethylenediamine (FITC-HMD) was dried in vacuum oven overnight. FITC-HMD was next conjugated to HA-UPy via EDC/NHS amide coupling reaction (FIG. 19C). Briefly, HA-UPy (400 mg) was dissolved in DI water to obtain a concentration of 5 mg/mL. EDC (113 mg) and NHS (69 mg) were added to the polymer solution at 15-min intervals. FITC-HMD (29 mg) dissolved in 10 mL water was added to the mixture, and the reaction was continued for 24 hrs at room temperature.

The reaction mixture was dialyzed (3.5 kDa membrane) against water for 4 days and lyophilized. FITC conjugation was confirmed via ¹HNMR spectroscopy as the spectrum showed the presence of aromatic protons (at 6.7-7.9 ppm) from the olefinic/aromatic protons of FITC. The presence of FITC in HA-UPy was further confirmed by the UV-visible spectroscopy as the UV-visible spectra showed typical FITC absorption at ˜480-500 nm (FIG. 19D).

### 9. Oxidation of HA-UPy

HA-UPy-DA was synthesized via sodium periodate oxidation of the sugar unit of HA-UPy or HA-UPy-FITC (FIG. 19E). Briefly, the polymers were dissolved in deionized water at 5 mg/mL. Sodium periodate (NaIO4, 1 equivalent with respect to the sugar ring of HA-UPy or HA-UPy-FITC) was dissolved in 5 mL of water and added slowly into the polymer solutions. After stirring for about 2 hrs at room temperature, the reaction mixture was quenched with excess of ethylene glycol (10 equivalent with respect to NaIO₄) for about 30 min. Next, the reaction mixtures were dialyzed extensively with water for 4 days. The solutions were then freeze-dried to obtain oxidized HA-UPy. The degree of oxidation was determined via ¹HNMR spectroscopy.

### 10. Quantification of Aldehyde Content in HA-UPy-DA

The amount of dialdehyde content in HA-UPy-DA was determined by reacting tert-butyl carbazate (t-BC) followed by reduction with sodium cyanoborohydride (NaBH₃CN). HA-UPy-DA was dissolved in pure water at a concentration of 5 mg/mL. A 10-fold molar excess of t-BC solution was added to the mixture and was stirred for about 1 hr at room temperature. NaBH₃CN (10-fold molar excess) was then added and reacted for about 24 hrs. The reaction mixture was purified by dialysis in a 2 kDa molecular-weight cut off dialysis bag and lyophilized. The aldehyde content was determined by ¹HNMR and the degree of oxidation was calculated by comparing the signal of tert-butyl groups (1.38 ppm, 9H) to that of acetamide methyl group in hyaluronic acid (1.9 ppm, 3H). The DA content in the oxidized polymer was found to be 9±1%.

### 11. Hydrogelation and Growth Factor Loading

The growth factor was first dissolved (at 0.5 mg/mL) in 1×PBS (pH 7.4) containing 0.1% bovine serum albumin (BSA). The buffered solution of the growth factor was next added to HA-UPy-DA to obtain 3.5 wt % polymer concentration. A soft hydrogel with growth factor was achieved upon complete dissolution of the polymer in the buffer. The gel was transferred to a Hamilton syringe for syringe application.

### 12. Histological Analysis in Zebrafish

Spinal cords were fixed with 4% paraformaldehyde. PFA-fixed tissues were post-fixed in 4% paraformaldehyde, rinsed in phosphate buffer, then cryoprotected in 30% sucrose. Samples were embedded in optimal cutting temperature compound (OCT) (Tissue-Tek) and frozen in a dry ice. 20 μm longitudinal or 16 μm transversal cryosections were used for histology. For immunohistochemistry, tissue sections were rehydrated in PBS, permeabilized in PBS containing 0.2% Triton X-100, and incubated with blocking buffer (5% goat serum in PBS-Tween) for 1 hr at room temperature. Sections were incubated overnight with primary antibodies diluted in blocking agent, washed in PBS, and treated for 1 hr in secondary antibodies and DAPI (Thermo Fisher Scientific, D3571, 1:5000).

Following washes, sections were mounted in in VECTASHIELD® Antifade Mounting Medium (Vector Laboratories H-1000-10). Primary antibodies used for fish experiments were: rabbit anti-GFP (Life Technologies, A 11122), chicken anti-GFP (Aves Labs, GFP-1020, 1:500), mouse anti-GFAP (ZIRC, Zrf1, 1:1000), rabbit anti-GFAP (Sigma, G9269, 1:200), rabbit anti-Sox2 (Abcam, ab97959, 1:200), mouse anti-HuC/D (Invitrogen, A-21271, 1:100), mouse anti-acetylated-at-tubulin (Sigma, T6793, 1:1000), rabbit anti-dsRed (Clontech. 632496, 1:200. Secondary antibodies (Life Technologies, 1:200) used in this study were highly cross-absorbed Alexa Fluor 488/546/594 goat anti-rabbit, anti-mouse or anti-chicken antibodies. EdU staining was performed using 20 μM Alexa Fluor 594 azide (Molecular Probes, A10270). All confocal images were acquired with either a Zeiss LSM 700 or LSM 880 microscope.

In situ hybridization was performed on cryosections of paraformaldehyde-fixed spinal cord as previously described (Mokalled M H, et al. (2016) Science. 354(6312):630-634), using an Intavis in situ robot. To generate probels, target sequences were placed upstream of a T7 promoter and gBlock fragments were ordered at IDT. Probes were generated using T7 RNA polymerase (M0251, New England BioLab). Signals were visualized by immunoassay using an anti-DIG-AP (alkaline phosphatase) antibody (11093274910, Sigma-Aldrich) and subsequent catalytic color reaction with NBT (nitroblue tetrazolium) (11383213001, Sigma-Aldrich)/BCIP (5-bromo-4-chloro-3-indolyl-phosphate)(11383221001, Sigma-Aldrich). Sections were imaged using a Leica DM6000 compound microscope.

### 13. Swim Capacity Assays

Swim capacity was measured by exercising fish in groups of 8-12 in a 5 L swim tunnel respirometer device (Loligo, SW 100605L, 120V/60 Hz) as previously described (Mokalled M H, et al. (2016) Science. 354(6312):630-634). Fish were acclimated for 20 minutes at a fixed low water current in the enclosed tunnel, then water current velocity was increased every two minutes and fish swam against the current until they reached exhaustion. For each animal reaching exhaustion, swim time and current velocity at exhaustion were recorded.

### 14. Handling of Mice

All experimental procedures with mice were performed in compliance with animal protocols reviewed and approved by the Duke IACUC. Wild-type male and female C57BL/6 mice were purchased from The Jackson Laboratory and used for all experiments. For neonatal experiments, injuries were performed at postnatal day 3 (P3). For experiments with adults, 3 month-old mice were used.

### 15. Spinal Cord Injuries in Mice

Neonatal spinal cord crush injury was performed as previously described (Li Y, et al. (2020) Nature. 587(7835):613-618). Briefly, mice at postnatal day 3 (P3) were anaesthetized by hypothermia on an ice bed. A laminectomy was performed at thoracic level (T9-T10) to completely expose the spinal cord. The spinal cord was crushed for 2 sec using forceps. After visually confirming establishment of the injury, muscle and skin were sutured in layers with 6-0 absorbable sutures. Mice were warmed until awake and placed into a cage containing bedding from their original cage for at least 30 min before the mother was returned. In case of bladder dysfunction, bladder expression was performed daily. Sham-operated pups underwent the same procedure involving laminectomy without spinal cord crush.

Adult crush injury was performed similarly to as described before (Li Y, et al. (2020) Nature. 587(7835):613-618). In brief, mice were anesthetized with ketamine-xylazine and given antibiotics (gentocin, 1 mg/kg). A midline incision was made over the thoracic vertebrae, followed by removal of the T9-T10 lamina to expose the spinal cord. The spinal cord was fully crushed for 2 see with forceps. Muscles were then sealed with 6-0 absorbable sutures and the skin was closed with wound clips. 2 mL of sterile saline was administered subcutaneously (sq) to prevent de-hydration. During recovery, mice were kept on a heating pad and received antibiotic agents (1 mg/kg gentocin) and saline daily for five days. Manual bladder expression was performed twice per day until tissue harvest. Sham-operated mice underwent laminectomy without spinal cord crush and received all post-operative cares as injured mice.

### 16. Histological Analyses in Mice

Mice were given a lethal dose of anesthesia and were transcardially perfused with PBS followed by 4% paraformaldehyde (PFA). PFA-fixed tissues were post-fixed in 4% paraformaldehyde, rinsed in phosphate buffer, then cryoprotected in 30% sucrose. Samples were embedded in OCT and frozen in dry ice. Longitudinal sections were cut on a cryostat at 20-μm thickness and stored at −20° C. until processed. Before staining, sections were warmed to room temperature, permeabilized using Triton X-100, treated with a blocking agent, and incubated over night at 4 degrees with primary antibodies.

Following washes, sections were incubated washed in PBS and mounted in VECTASHIELD® Antifade Mounting Medium (Vector Laboratories 11-1000-10). Primary antibodies used for mouse immunofluorescence anti-EGFP (rabbit, A11122, Life Technologies), anti-EGFP (chicken, GFP-1020, Aves Labs), mouse anti-GFAP (Sigma, G3893, 1:500), rabbit anti-Sox2 (Abcam, ab97959, 1:200), mouse anti-HuC/D (Invitrogen, A-21271, 1:100), rabbit anti-5-HT (Immunostar, 20079, 1:5,000), rabbit anti Ki67 (Abcam, ab15580, 1:200), rabbit anti-fibronectin (Sigma. F3648, 1:200), rat anti-CD68 (Bio-Rad, MCA1957, 1:600), F4/8 (Biorad, MCA497R). Secondary antibodies (Life Technologies, 1:200) used in this study were highly cross-absorbed Alexa Fluor 4885461594 donkey or goat anti-rabbit, anti-mouse, anti-rat or anti-chicken antibodies. Confocal images were acquired with Zeiss LSM 700 or Zeiss LSM 880 microscopes.

### 17. Virus Production and Titers

A triple-plasmid transfection protocol was used to produce recombinant AAV vectors in suspension HEK293s. Specifically, the transfected plasmids include a capsid-specific helper plasmid (containing AAV2 Rep and AAVcc47 Cap genes), the adenoviral helper plasmid pXX680, and pTR-Enhancer-HSP68-GFP plasmids (encoding different enhancer elements), flanked by inverted terminal repeats (TRs) derived from the AAV2 genome. Culture media was harvested 6 days post transfection and cells were pelleted via centrifugation (1000 g×15 min) and discarded. Viral particles were precipitated from the culture media overnight at 4° C. with polyethylene glycol (PEG; final concentration of 12%). Media was subsequently centrifuged at 3,000 g×1 hr and discarded. The PEG pellet was resuspended in formulation buffer (1×PBS with 1 mM MgCl and 0.001% puronic F-68) and treated with DNase at 37° C. for 1 hr. Viral vectors were purified using iodixanol density gradient ultracentrifugation.

Vectors were subsequently subjected to buffer exchange using Pierce Protein PES centrifugation columns (100,000 MWCO, Thermo Scientific, catalog no. 88524). Following purification, viral genome titers were determined via quantitative PCR using a Roche Lightcycler 480 (Roche Applied Sciences). Quantitative PCR primers were designed to specifically recognize the AAV2 inverted terminal repeats (forward, 5′-AACATGCTACGCAGAGAGGGAGTGG-3′ (SEQ ID NO:30); reverse, 5′-CATGAGACAAGGAACCCCTAGTGATGGAG-3′ (SEQ ID NO:31)) (Integrated DNA Technologies). 10¹¹ virus particles were injected into adult neonatal and adult mice, respectively, by temporal and tail vein injection.

### 18. Data Analyses and Statistics

Quantification of cells co-expressing EGFP and GFAP was performed using a Zeiss LSM 700 microscope software, by calculating a Mander's overlap coefficient (MOC) for GFAP/EGFP in the acquired images. MOC was calculated for GFAP/GFAP as a control, and the normalized MOC was used to estimate the percent of GFAP⁺EGFP⁺ signal relative to GFAP.

Glial bridge diameter was calculated using ImageJ software, measuring 3-5 sections per fish with the thickest bridge. Measurements at the lesion site were normalized on diameter of spinal cord caudal to the lesion.

Biocytin-labeled axons were quantified using the “threshold” and “particle analysis” tools in the ImageJ software. 3-5 sections per fish proximal and rostral to the lesion core were analyzed. Axon growth was normalized to biocytin labeling rostral to the lesion for each fish.

qPCRs were performed 3 times, 3 technical replicates were run each time. Fold changes were calculated with the 2-ΔΔCT method.

For violin plots, solid lines indicate the group median; dotted lines indicate the 25^{th} and 75^{th} quartiles. All samples are shown in each violin plot.

Statistical tests were performed using Prism software. Student's t-test with Welch's correction where appropriate was used when comparing two groups. Where three or more groups were compared, one-way ANOVA with appropriate corrections for multiple comparisons was used. For swim analyses Mann-Whitney tests were performed at each time point. Sample sizes, statistical tests, and P values are indicated in the figures or the legends.

### B. Specific Examples

### Example 1

### HB-EGF Paralogues were Induced and Required for Regeneration after Spinal Cord Injury

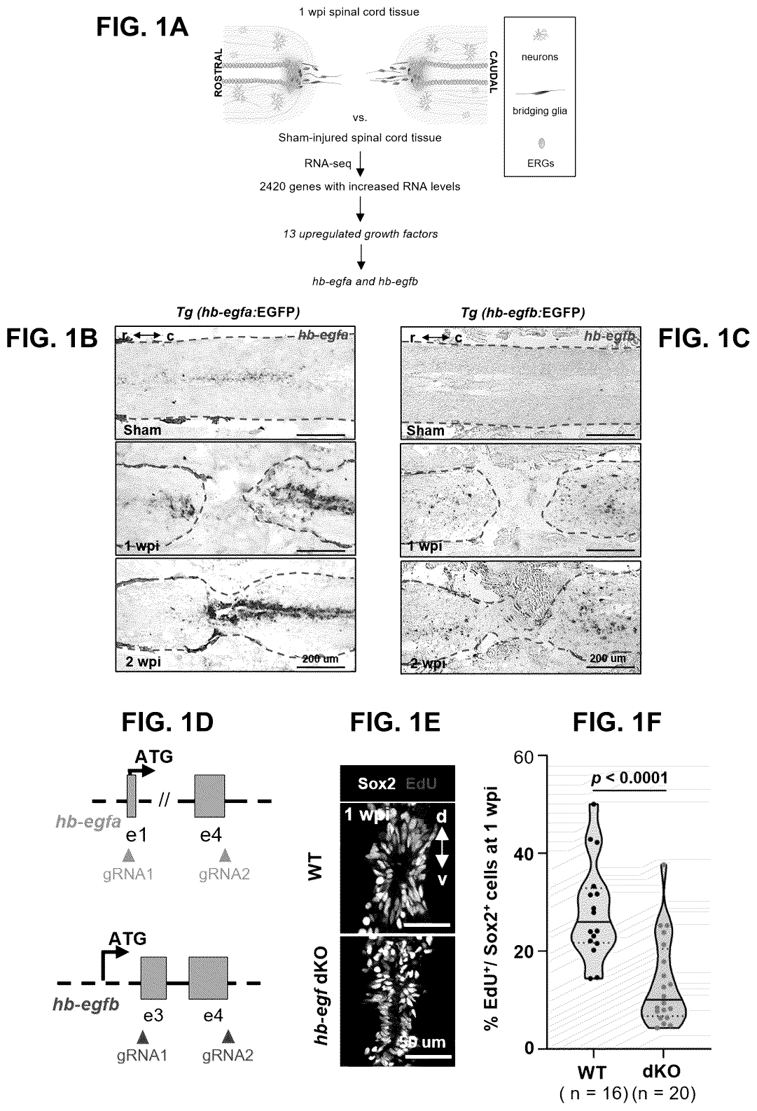

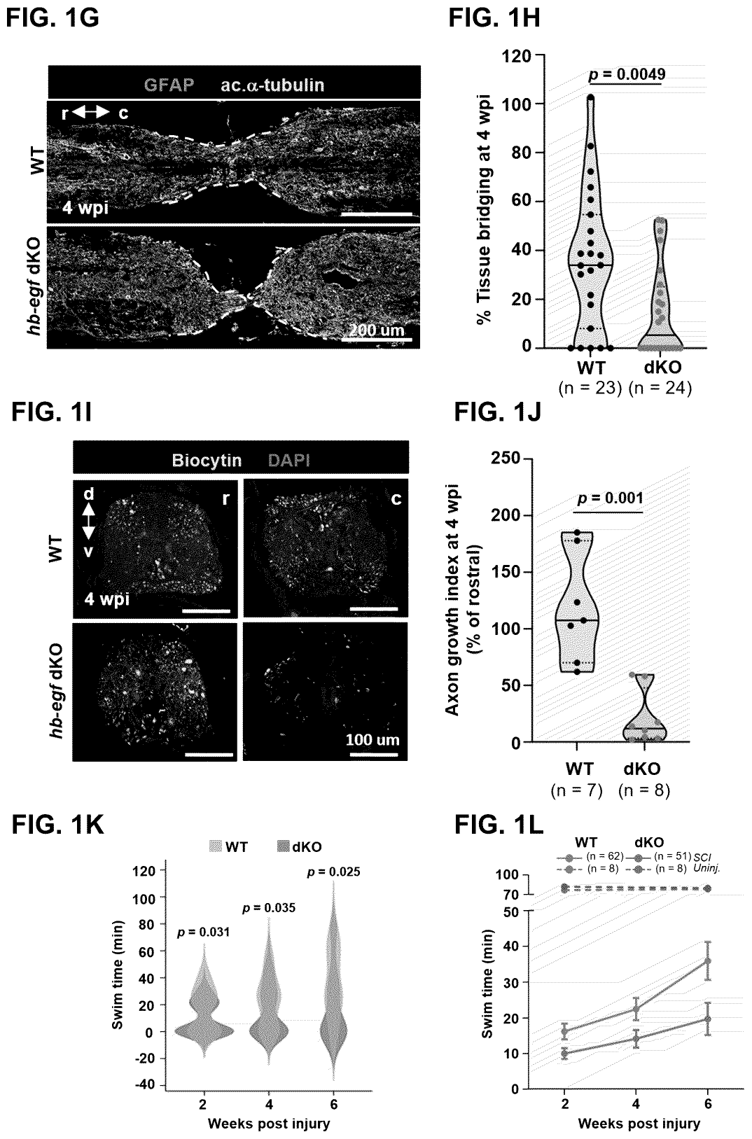

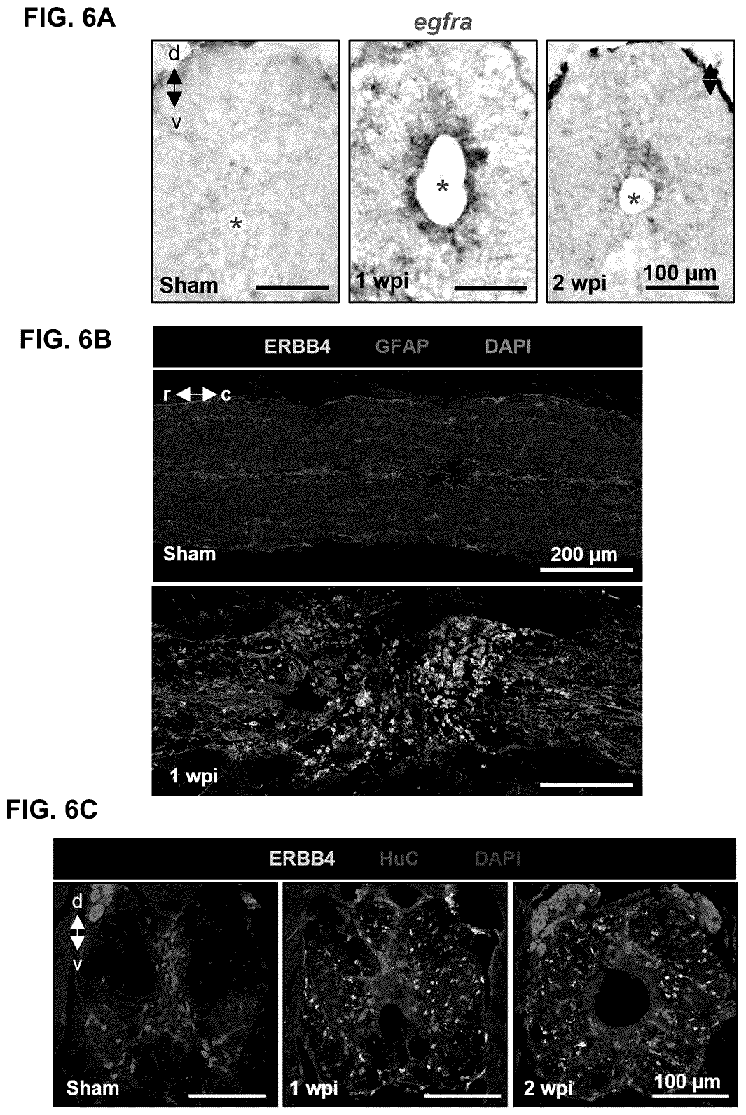

In an expression profiling experiment for genes induced during zebrafish spinal cord regeneration at one week post injury (wpi), both hb-egf paralogs hb-egfa and hb-egfb displayed sharp increases in RNA levels (FIG. 1A-FIG. 1C, Table 1). Hb-egf is a secreted glycoprotein that was previously implicated in Müller glial cell dedifferentiation during zebrafish retina regeneration, in experiments using antisense oligonucleotides (Want J, et al. (2012) Dev Cell. 22(2):334-347). HB-EGF has also been reported to stimulate mammalian neurogenesis and neurite outgrowth and to affect astrocyte morphology and proliferation in vitro (Jin K, et al. (2002) J Neurosci. 22(13):5365-5373; Puschmann T B, et al. (2014) J Neurochem. 128(6):878-889; Zhou Y, et al. (2010) Neurosignals. 18(3):141-151). Intracerebral HB-EGF administration also induced proliferation of neuronal precursors after cerebral ischemia in rats (Jin K, et al. (2002) J Neurosci. 22(13):5365-5373). Using in situ hybridization (ISH), hb-egfa transcripts expressed at low levels in ependymal cells of uninjured spinal cord were visualized, and then induced strongly in these cells in severed cord ends, as well as in other cells surrounding the central canal (FIG. 1B). hb-egfb expression was undetectable in uninjured spinal cord but could be detected sparsely in cells throughout the injured cord at one and 2 wpi (FIG. 1C). Expression of Erb-B2 Receptor Tyrosine Kinase 4 (ERBB4) and epidermal growth factor receptor (EGFR), which bind Hb-egf ligands, were undetectable in uninjured sections (FIG. 6A-FIG. 6C). By one wpi, egfra receptor transcripts were detectable in cells lining the central canal (FIG. 6A), and Erbb4 protein was localized at the lesion site and throughout white and gray matter at one and 2 wpi (FIG. 6B and FIG. 6C).

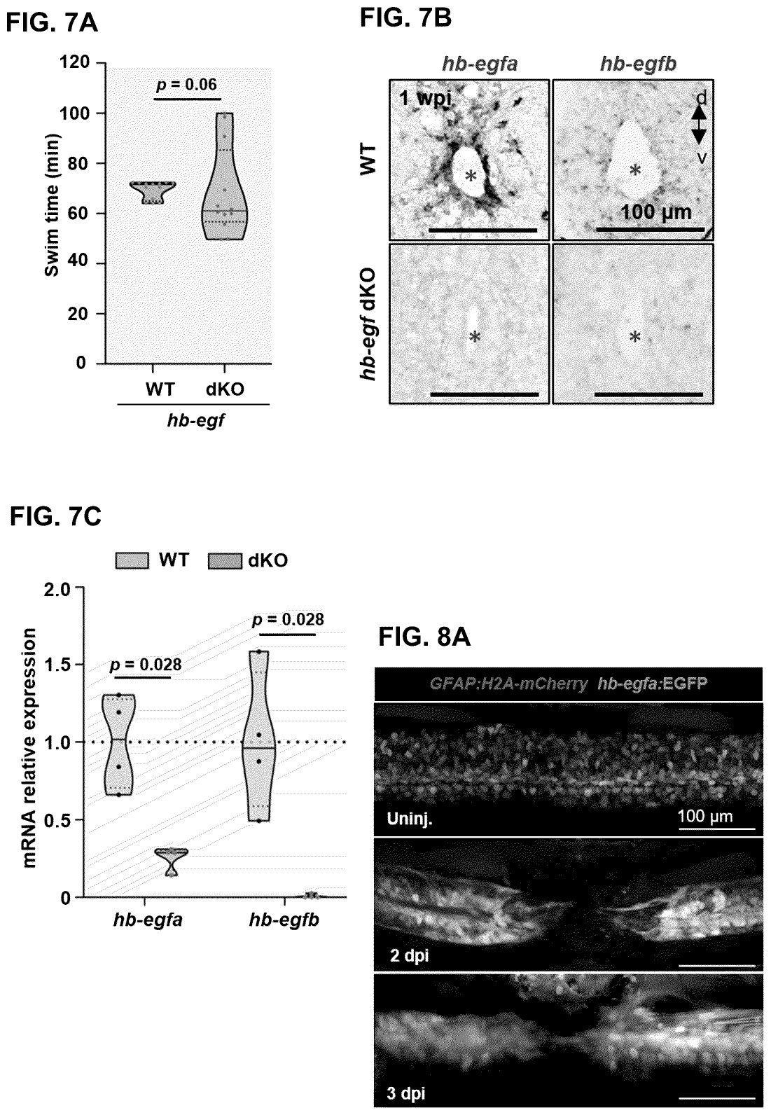

To test requirements for hb-egf gene function during spinal cord regeneration, sequences contained between exon 1 and 4 of hb-egfa (allele hb-egfa^{pd360}, referred to as hb-egfaKO) were removed and sequenced contained between exons 3 and 4 of hb-egfb (allele hb-egfb^{pd361}, referred to as hb-egfbKO) were removed using CRISPR/Cas9 methods (FIG. 1D). Animals with mutations in both hb-egf paralogues (hb-egdKO) were immediately generated and analyzed first due to an expectation of compensatory effects by gene paralogs (El-Brolosy M A, et al. (2019) Nature. 568(7751):193-197). hb-egfdKO animals showed no detectable hb-egf messages and are viable to adulthood with grossly normal swim capacity (FIG. 7A-FIG. 7C).

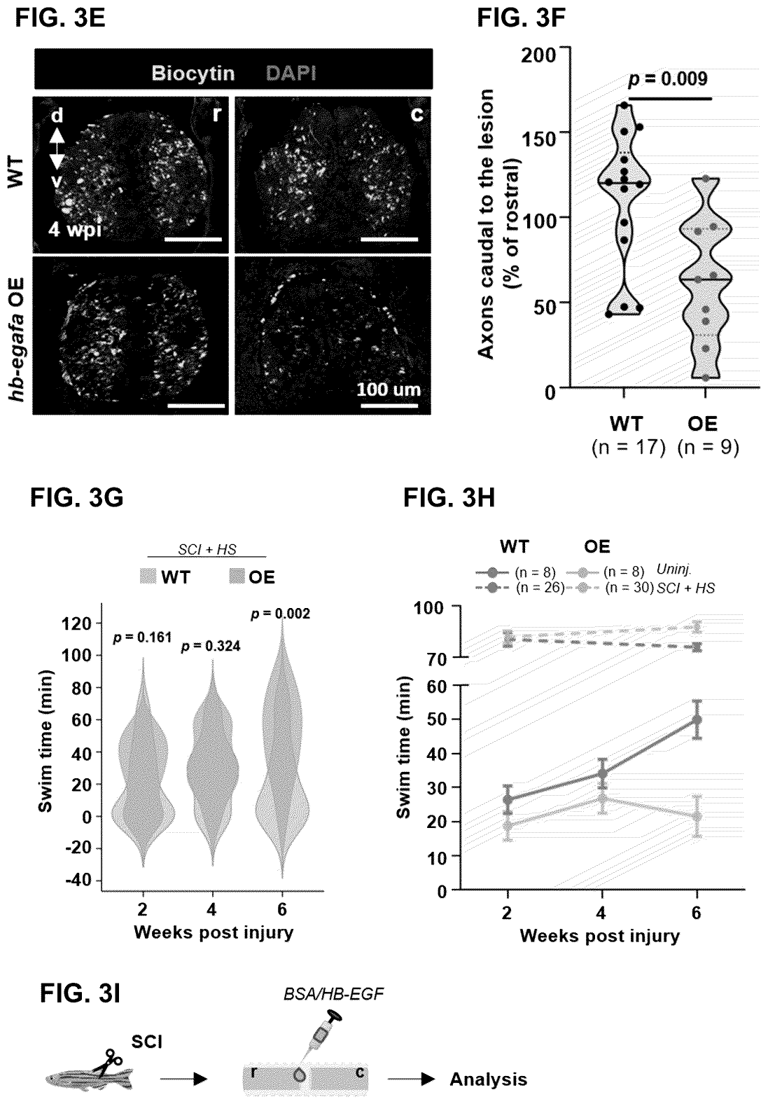

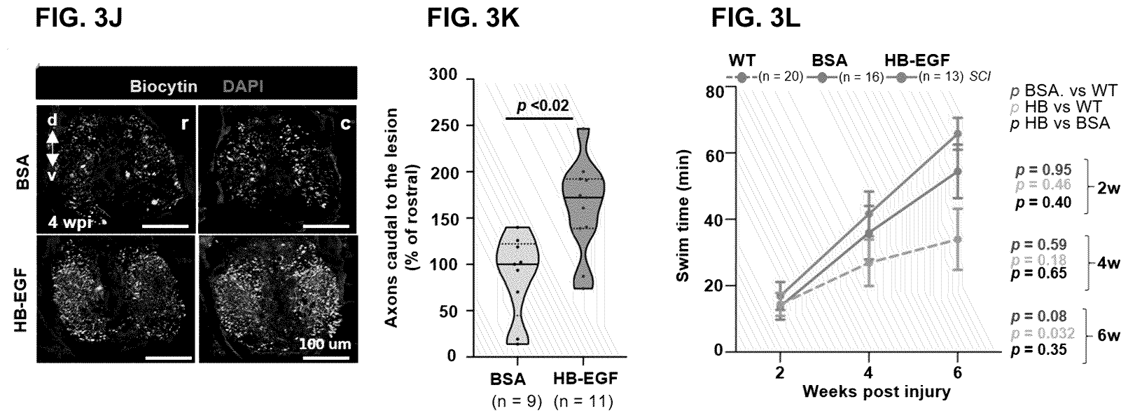

To evaluate effects of hb-egf mutations on spinal cord regeneration, transection injuries and a panel of histological analyses were then performed. First, 5-ethynyl-2′-deoxyuridine (EdU) incorporation assays were performed, which indicated a ˜48% reduction in cycling of Sox2⁺ ERGs in hh-egfdKO cords as compared to wild-type clutchmates at one wpi (FIG. 1E-FIG. 1F). Next, to measure the extent of tissue bridging. hb-egfdKO and wild-type spinal cord at 4 wpi was stained for the glial marker GFAP and the axonal marker acetylated α-tubulin. The diameter of bridges was reduced by ˜40% in hb-egfdKO animals (FIG. 1G-FIG. 1H, Table 1, and Table 2). Table 1 shows representative spinal cord RNA-seq profiling at 1 wpi, while the complete data set resides with Applicant. Table 2 shows representative merged ATAC-seq peaks and RNA-seq genes, while the complete data set resides with Applicant. To quantify axon regeneration, a biocytin-soaked gelfoam sponge was applied rostral to the injury site at 4 wpi and assessed axon labeling caudal to the lesion. hb-egfdKO zebrafish displayed a ˜80% decrease in axon density compared to controls (FIG. 1I-FIG. 1J). Finally, the ability of these animals to swim against increasing velocities of water current was examined, and hb-egfdKO animals after spinal cord injury had significantly reduced swim capacity (FIG. 3K-FIG. 3L). At 4 wpi, differences between hb-egfdKO and wild-type animals in swim behavior were observed, just by viewing animals in their standard aquarium setting. Thus, Hb-egf factors were required for normal ependymal cell proliferation, tissue bridging, axon regeneration, and functional recovery after a paralyzing injury.

### Example 2

### HB-EGFA was Required for Spinal Cord Regeneration

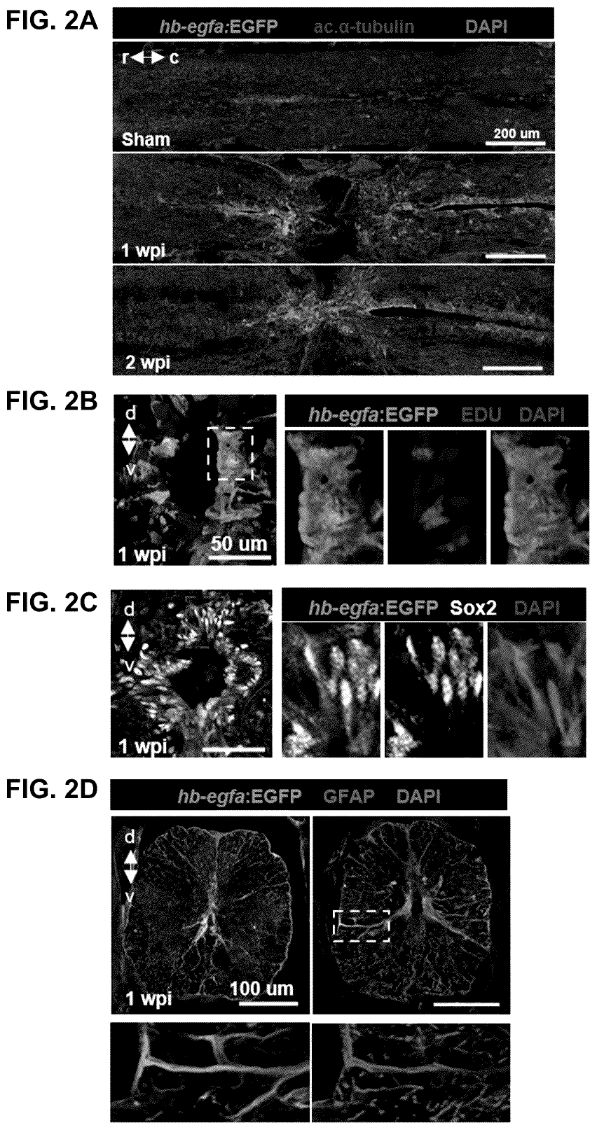

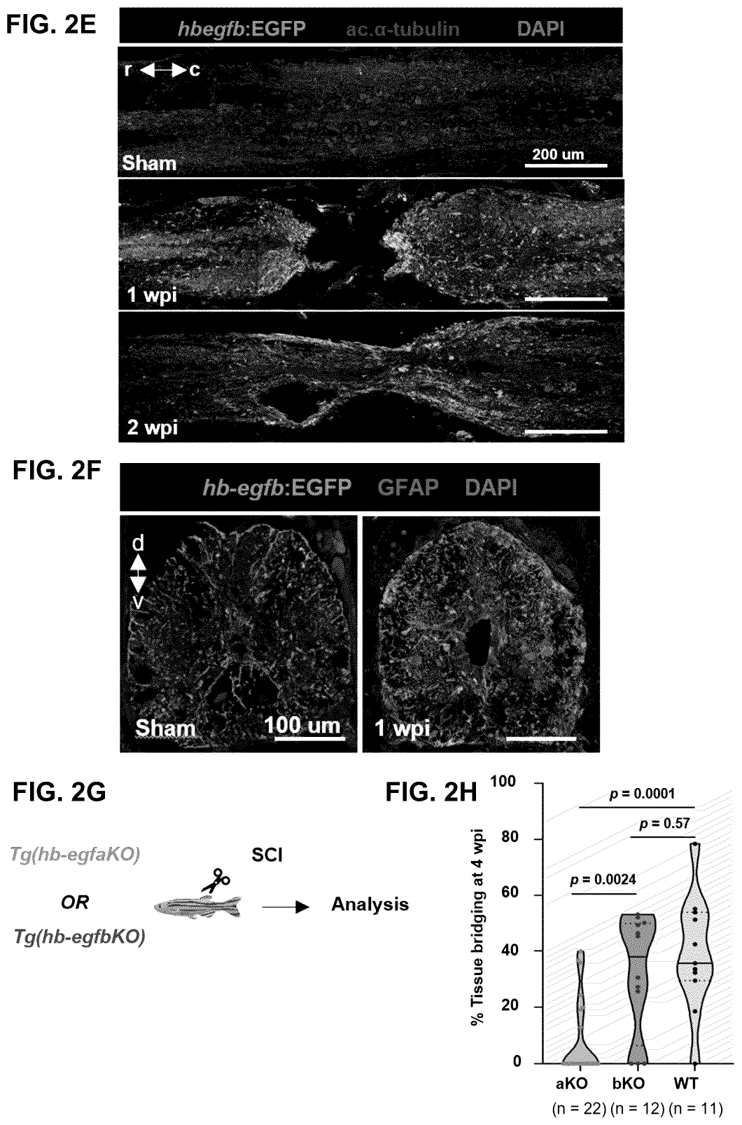

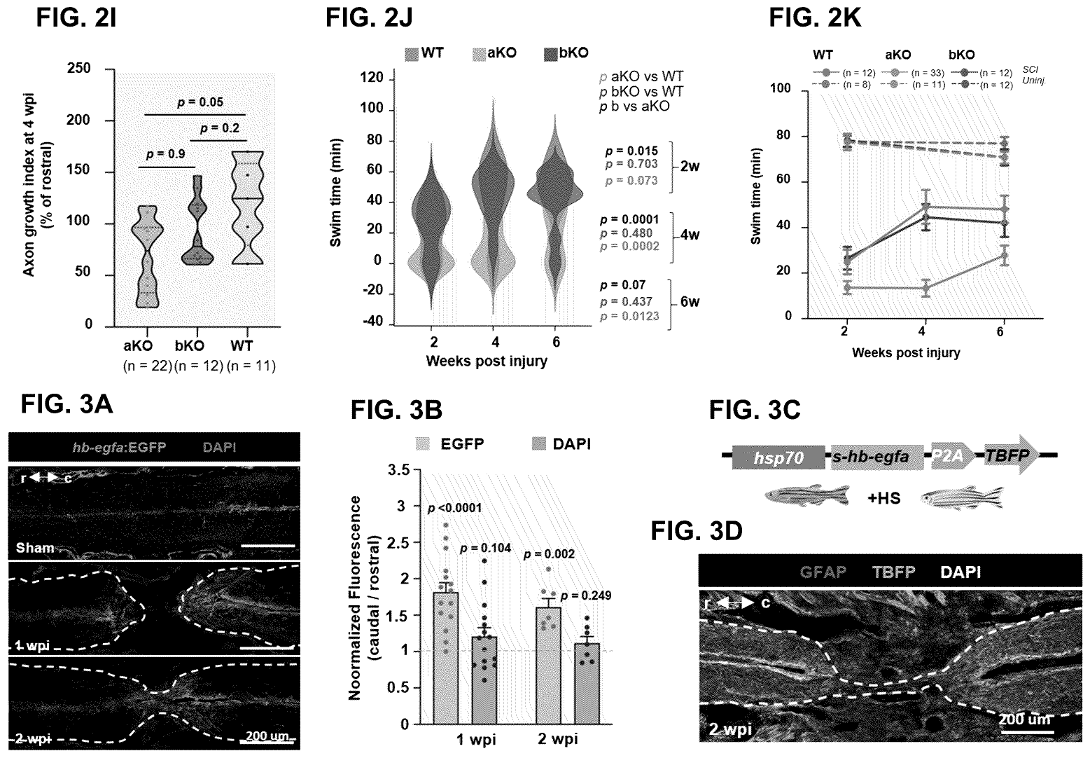

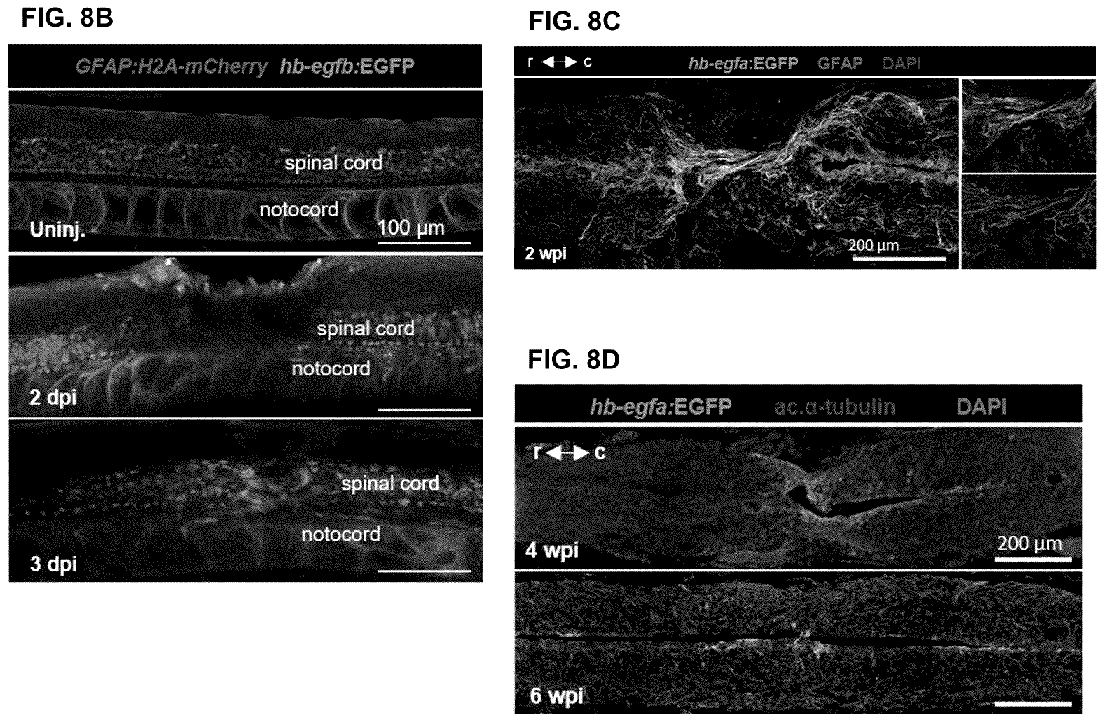

To better visualize cell types expressing hb-egf paralogs and to determine which gene (or genes) had required functions, new reporter lines and individual mutants representing each paralog were assessed (FIG. 2A-FIG. 2K, FIG. 8A-FIG. 8D). Sham-injured transgenic hh-egfa:EGFP fish (allele hb-egfa:EGFP^{pd362}) generated with a large BAC sequence had little or no detectable EGFP expression at larval or adult stages (FIG. 8A, FIG. 2A). Upon spinal cord transection, hb-egfa-directed EGFP was induced by 1 wpi in the central canal, mimicking hb-egfa ISH patterns.

Fluorescence largely co-localized with Sox2 and GFAP, markers of ERGs and glia (FIG. 1A. FIG. 1C, and FIG. 1D). hb-egfa-directed fluorescence also marked bridging glia at 2 wpi (FIG. 8C), an observation in agreement with recent single-cells RNA-seq datasets of regenerating spinal cord, reporting hb-egfa amongst the top 5 candidates in a subpopulation of bridging glia (Klatt Shaw, et al. (2021) Dev Cell. 56(6):613-626). Many cells marked by hb-egfa:EGFP showed indicators of cell proliferation at one wpi in EdU incorporation assays (FIG. 2B). hb-egfa:EGFP largely faded by 6 wpi, a late timepoint in regeneration (FIG. 8D). The cellular localization of the hb-egfb was localized using anew EGFP knock-in allele (allele hb-egfb^{egfb pd363}). EGFP signal was negligible in larvae or uninjured adult hb-egfb^{egfp} spinal cord while diffuse EGFP fluorescence was observed at one and 2 wpi (FIG. 2E, FIG. 8B). As indicated by ISH, hb-egfb^{egfp}-directed fluorescence was not well-represented in ependymal cells of the central canal or in tissue bridges composed of axons and glia (FIG. 2E-FIG. 2F).

To assess which paralog (or paralogs) has prominent functions in spinal cord regeneration, regeneration assays in hb-egfa and hb-egfb single mutants were performed (FIG. 2G). hb-egfbKO displayed similar tissue bridging and axon regeneration as wild-types after spinal cord injury. By contrast, hb-egfaKO animals had ˜80% and ˜57% reductions in tissue bridging and axon regeneration compared to wild-types, respectively (FIG. 2H-FIG. 2I). Furthermore, swim performance after spinal cord injury was reduced in hh-egfaKO animals compared to controls, and swim behavior was noticeably different from wild-types at 6 wpi in aquaria. Swim capacity in hb-egfbKO mutants was comparable to wild-types (FIG. 2J-FIG. 2K). Thus, the hb-egfa gene product was required for spinal cord regeneration.

### Example 3

### HB-EGFA Polarized Expression and had Instructive Effect on Axon Regeneration

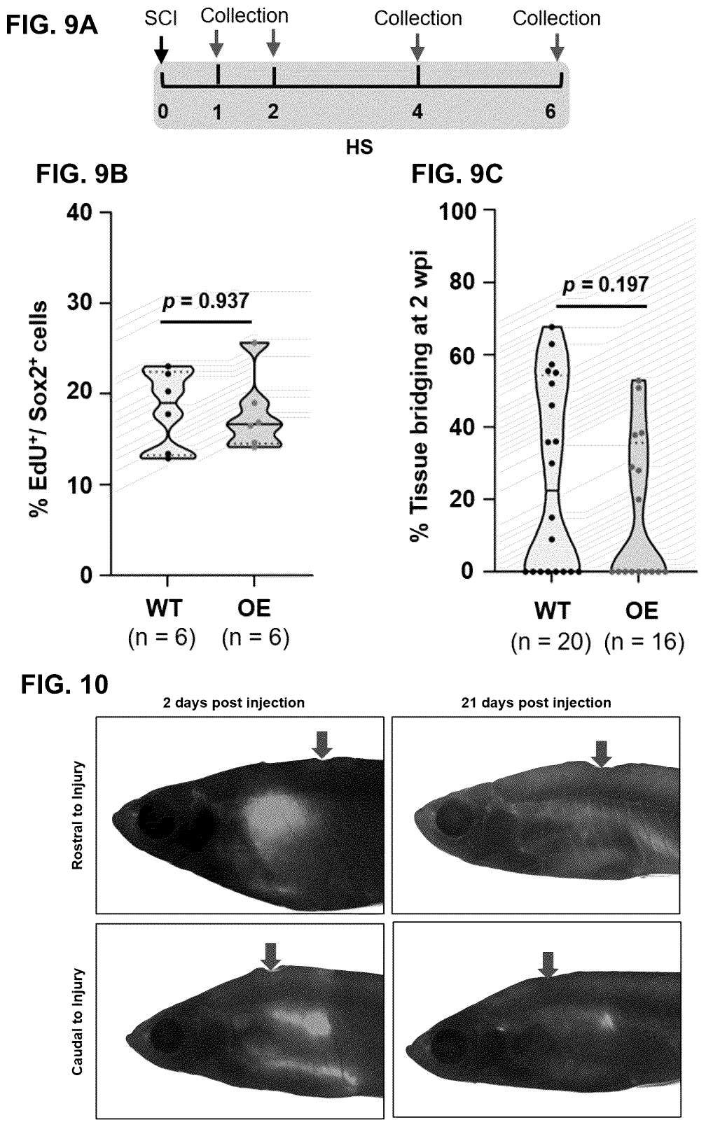

Interestingly, the assessment of hb-egfa mRNA or hb-egfa:EGFP expression in longitudinal sections of regenerating spinal cord indicated a polarized distribution, with greater signals in the caudal versus rostral ends (FIG. 1B and FIG. 3A). hb-egfa:EGFP fluorescence in both ends was quantified, and while DAPI signal was comparable, EGFP signal was ˜81% and ˜61% enriched in caudal stumps vs. rostral at one and 2 wpi, respectively. This type of quantified difference has not been reported for models of zebrafish spinal cord regeneration. To attempt to disrupt this polarized expression, transgenic fish expressing a soluble form of hb-egfa under the control of a heat-inducible promoter (hsp70:hb-egfa-2A-TBFP, allele hb-egfa^{pd364}, referred to as hb-egfaOE) was generated (FIG. 3C). Daily heat shocks led to whole-animal overexpression visualized by TBFP fluorescence, disrupting Hb-egfa rostro-caudal distribution (FIG. 3D and FIG. 9A). Animals were generated under this heat shock regimen and examined features of spinal cord regeneration. Ependymal cell cycling identified at one wpi was unaffected in these animals (FIG. 9B), and tissue bridging at 2 wpi was only mildly reduced if at all (FIG. 9C). By contrast, daily, animal-wide induction of h-egfa caused a ˜62% decrease in axon regeneration at 4 wpi, and recovery of swim capacity was grossly impaired (FIG. 3E-FIG. 3H). These results indicate that higher levels of Hb-egf per se do not improve regeneration and appear instead to have a disruptive effect on axon regeneration.

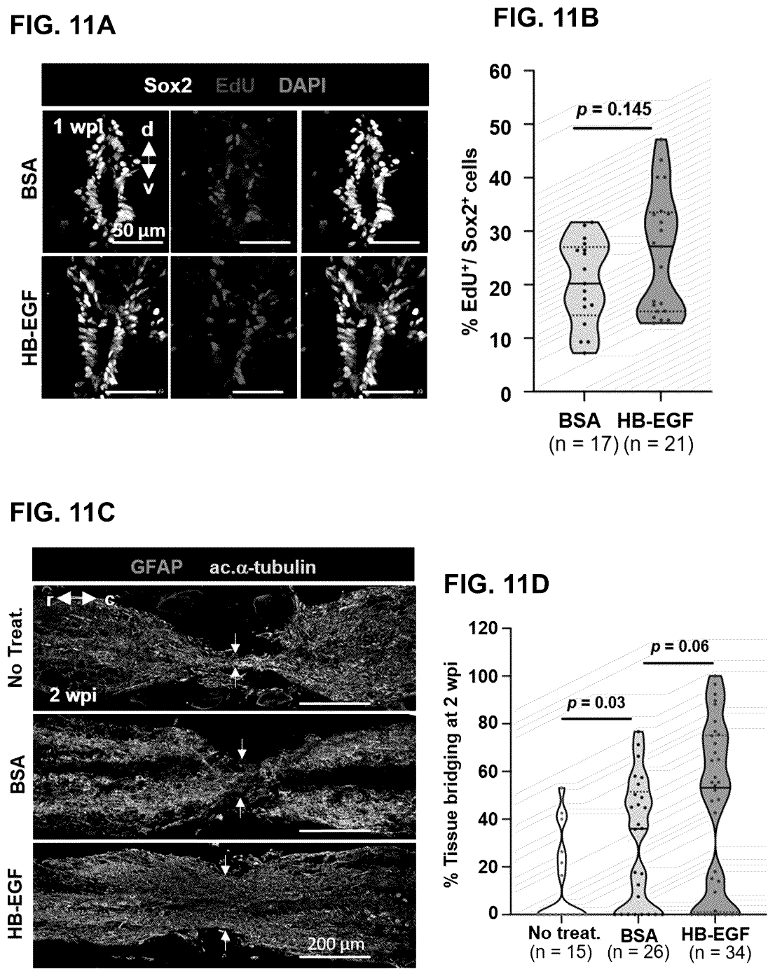

To assess effects of targeted Hb-egf augmentation to the lesion site alone, a slow-release strategy was tested using biomaterial depots of synthetic hydrogels made of hyaluronic acid (Gilpin A, et al. (2021) Adv Healthc Mater. 3:e2100777). The dynamics of compound release from these hydrogels was assessed by injecting a FITC-loaded version at the spinal cord lesion site and following diffusion of the dye over time. FITC gradually diffused from the injection site and faded by 21 days post injection (FIG. 10). To supplement Hb-Egf at injury sites, animals were injured and hydrogels containing either BSA or human recombinant HB-EGF (HR-HB-EGF) peptide were immediately applied (FIG. 3I). These two groups showed similar ependymal cell proliferation at one wpi (FIG. 11A-FIG. 11B). Interestingly, BSA-loaded hydrogel increased the average diameters of tissue bridges, likely by promoting extracellular cell remodeling as previously shown in mice (Hong L T A, et al. (2017) Nat Commun. 8(1):533. HR-HB-EGF administration increased this slightly further (FIG. 11C-FIG. 11D). More impressively, localized treatment with HR-HB-EGF caused a ˜-61% increase in axon regeneration at 4 wpi relative to vehicle (FIG. 3J-FIG. 3K). This was accompanied by a mild improvement in swim capacity, which was enhanced by the vehicle hydrogel itself compared to untreated animals, and even more so by HR-HB-EGF delivery (FIG. 3L). Together, the gain-of-function experiments indicated that Hb-egf has instructive effects on spinal cord regeneration; that is, it can modulate spinal cord regeneration when expressed ectopically. Based on its naturally polarized source in caudal cord ends, its inhibitory effects when enhanced animal wide, and its stimulatory effects particularly on axon regeneration—when locally enhanced, the regulation of hb-egfa expression preferentially to the caudal stump was important for its required activity.

### Example 4

### An Enhancer Near HB-EGFA was Sufficient to Direct Polarized Gene Expression in Spinal Cord Injuries

Emerging evidence indicates that regeneration programs are orchestrated by gene regulatory elements named “tissue regeneration enhancer elements” or TREEs in an earlier study (Kang J, et al. (2016) Nature. 532(7598):201-2006). These enhancers possess all necessary sequences to direct gene expression specifically or preferentially upon injury to the damaged area, maintain expression during regeneration, and then shut down expression as regeneration concludes. Such enhancers have been implied from profiling or validated by transgenesis in many regeneration contexts (Gehrke A R, et al. (2019) Science. 363; Goldman J A, et al. (2017) Dev Cell. 40:392-404 e395; Guenther C A, et al. (2015) Bone. 77:31-41; Harris R E, et al. (2016) Elife. 5; Harris R E, et al. (2020) Elife. 9; Thompson J D, et al. (2020) Development. 147; Vizcaya-Molina E, et al. (2018) Genome Res. 28:1852-1866; Wang W, et al. (2020) Science. 369).

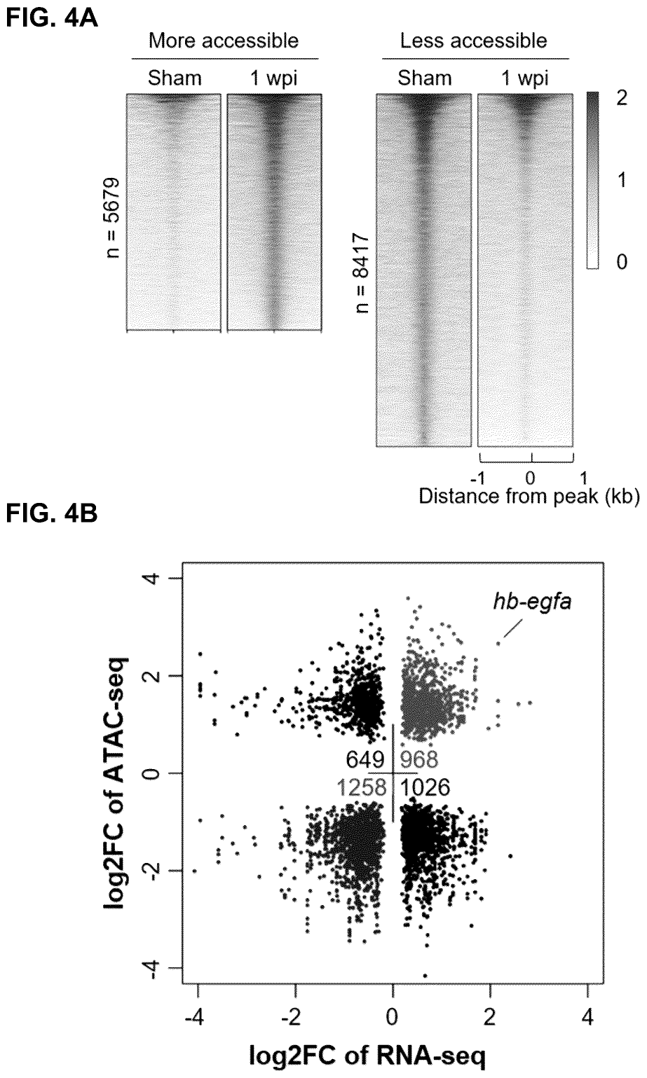

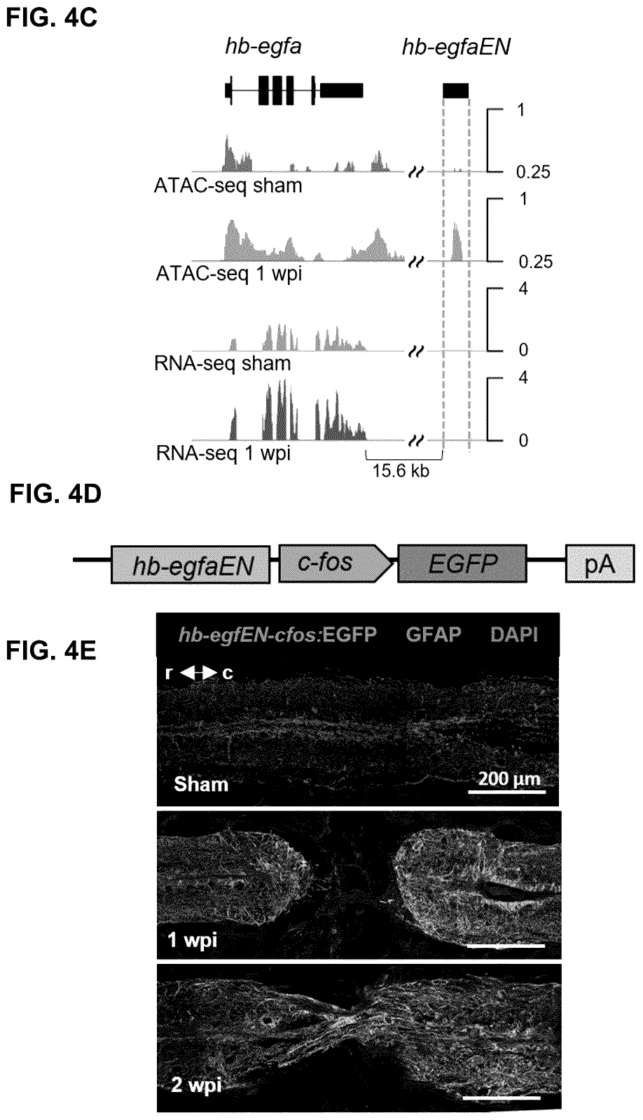

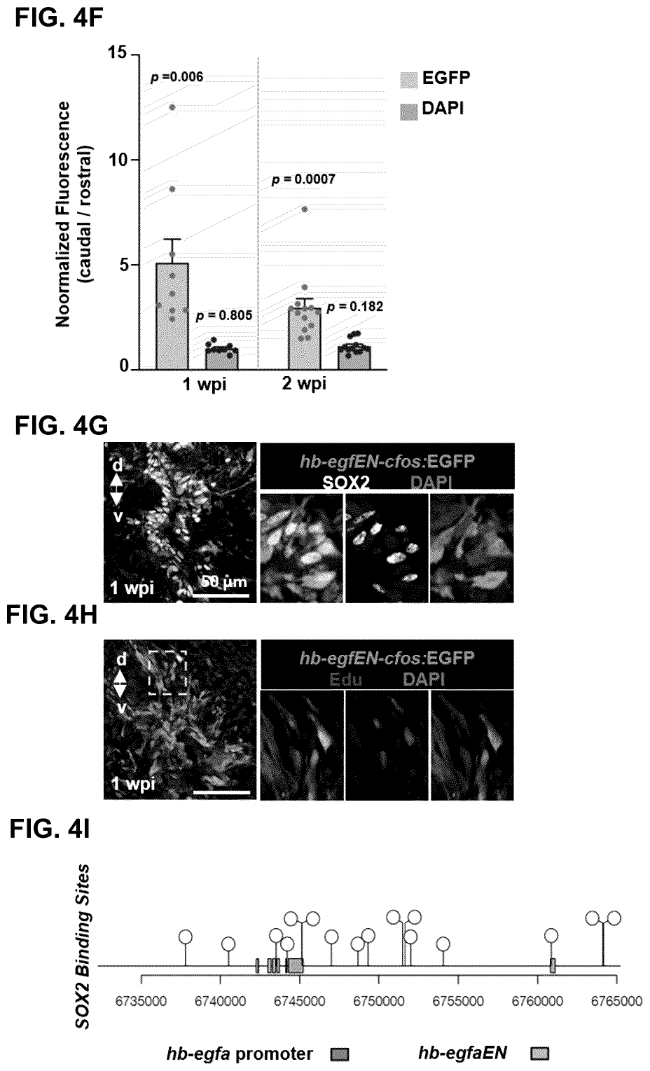

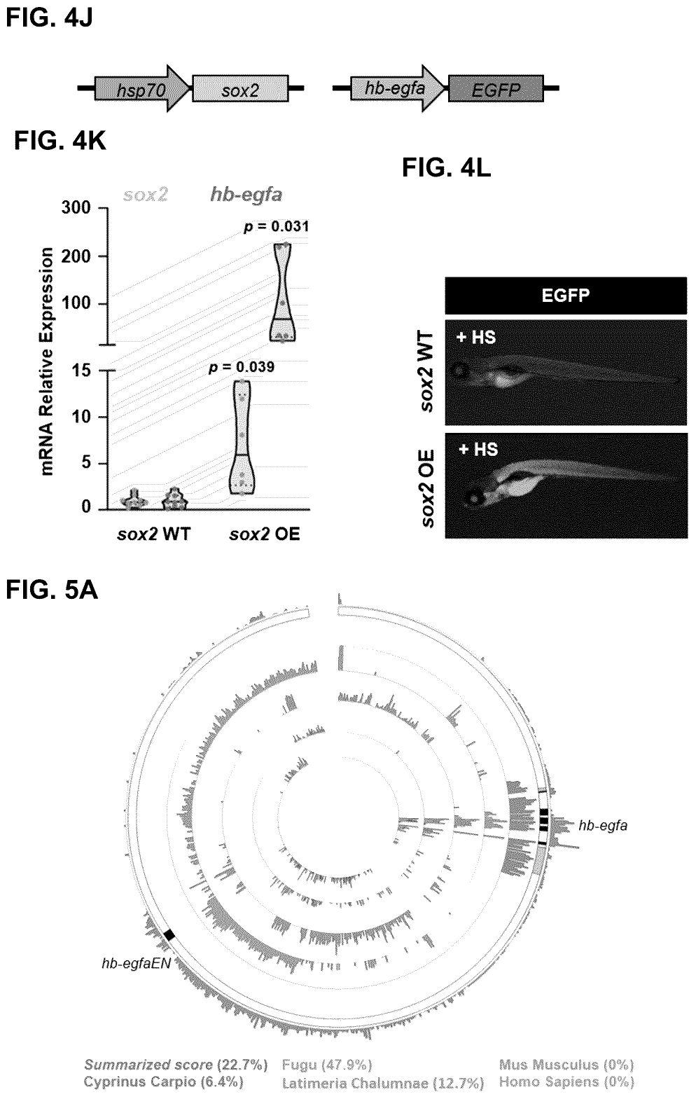

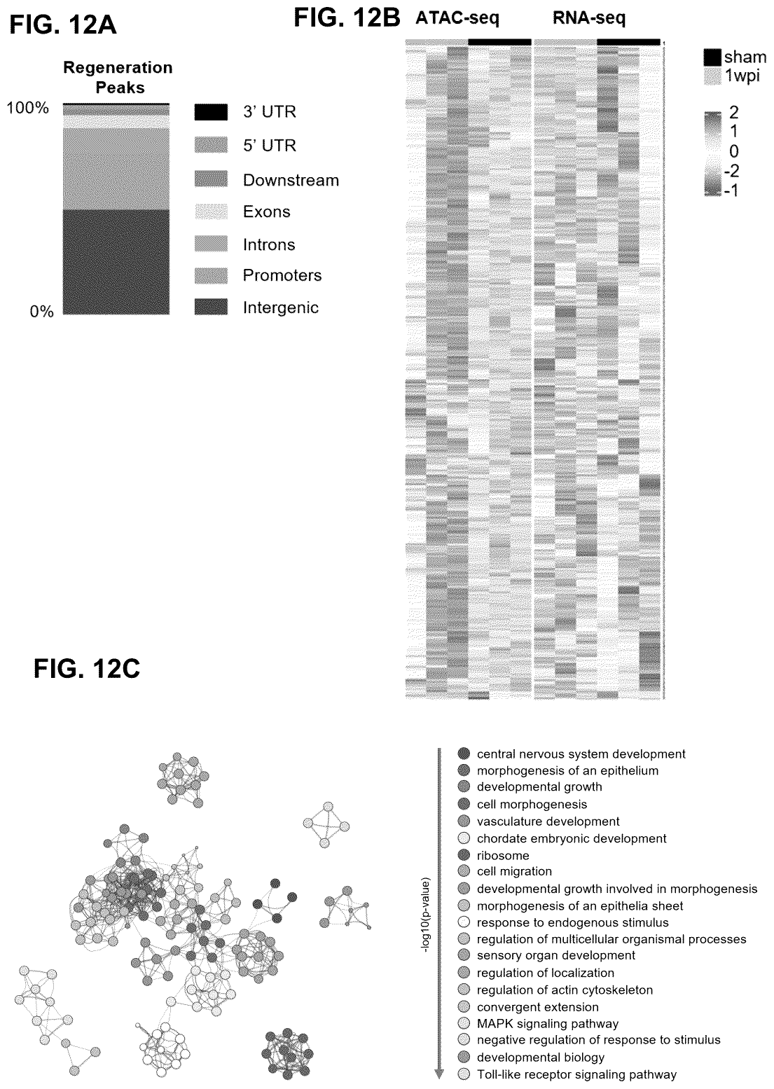

To identify regulatory TREEs relevant to spinal cord regeneration, an Assay for Transposase-Accessible Chromatin using sequencing (ATAC-seq), a genome-wide assay for chromatin accessibility (Buenrostro J D, et al. (2015) Curr Protoc Mol Biol. 109:21.29.1-21.29.9), was employed. Chromatin accessibility profiles were obtained from sham injured spinal cord tissue as well as tissue at one wpi (FIG. 4A-FIG. 4L and FIG. 12A-FIG. 12C). A summary of the analysis of these profiles is presented in FIG. 12A-FIG. 12C. From this assay. 5679 regions with increased chromatin accessibility and 8417 regions with reduced accessibility were identified (p<0.05) (FIG. 4A). ATAC-seq and RNA-seq datasets were integrated, finding 968 regions with significantly increased accessibility bioinformatically assigned to genes with increased RNA levels, indicative of candidate TREEs (FIG. 4B; Table 2 and Table 3). Interestingly, a 310 bp region located ˜18 kb downstream of the hb-egfa start site was identified. This 310 bp region with increased accessibility at one wpi as compared to uninjured spinal cords, referred to hereafter as hb-egfa-linked enhancer (hb-egfaEN) (i.e., DNA range=chr14:6760806-6761115) (FIG. 4C).

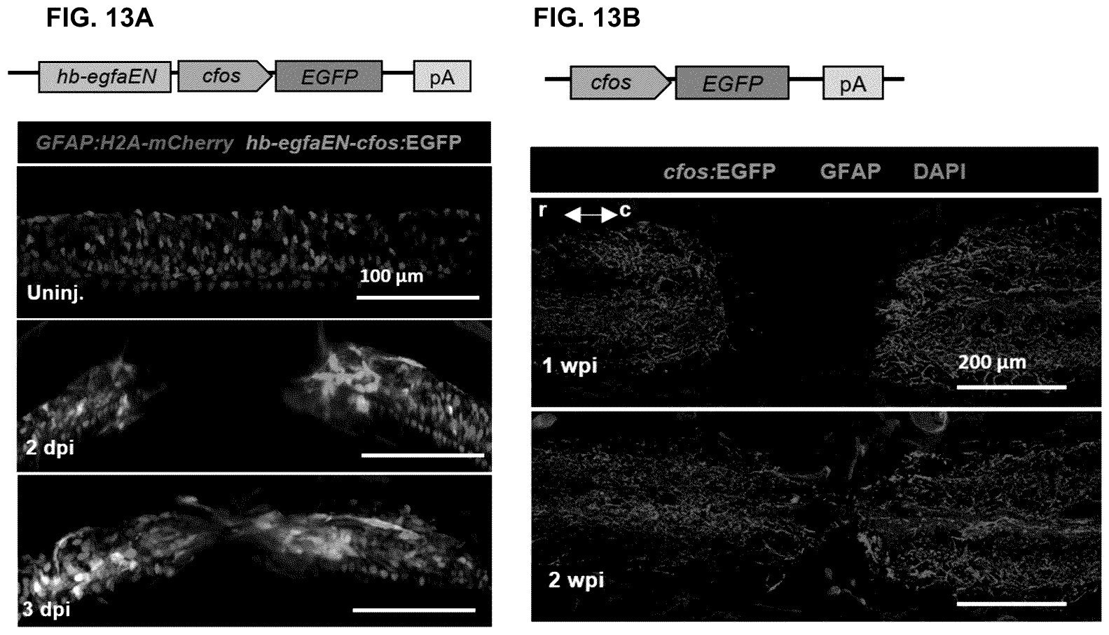

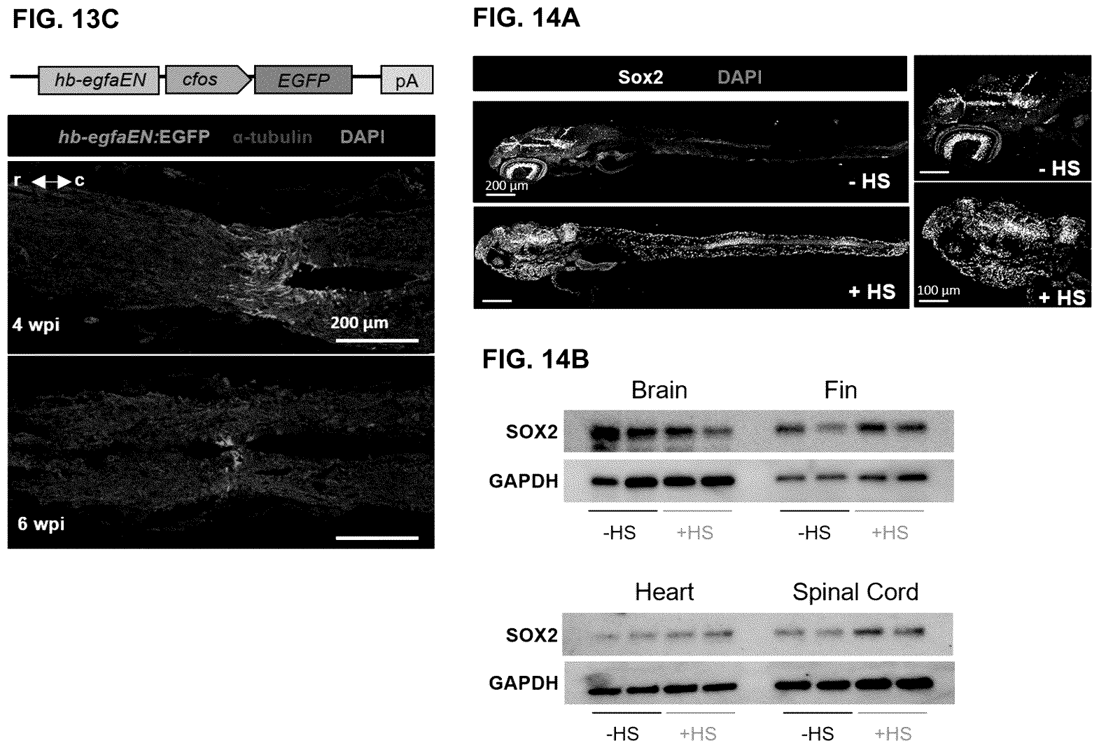

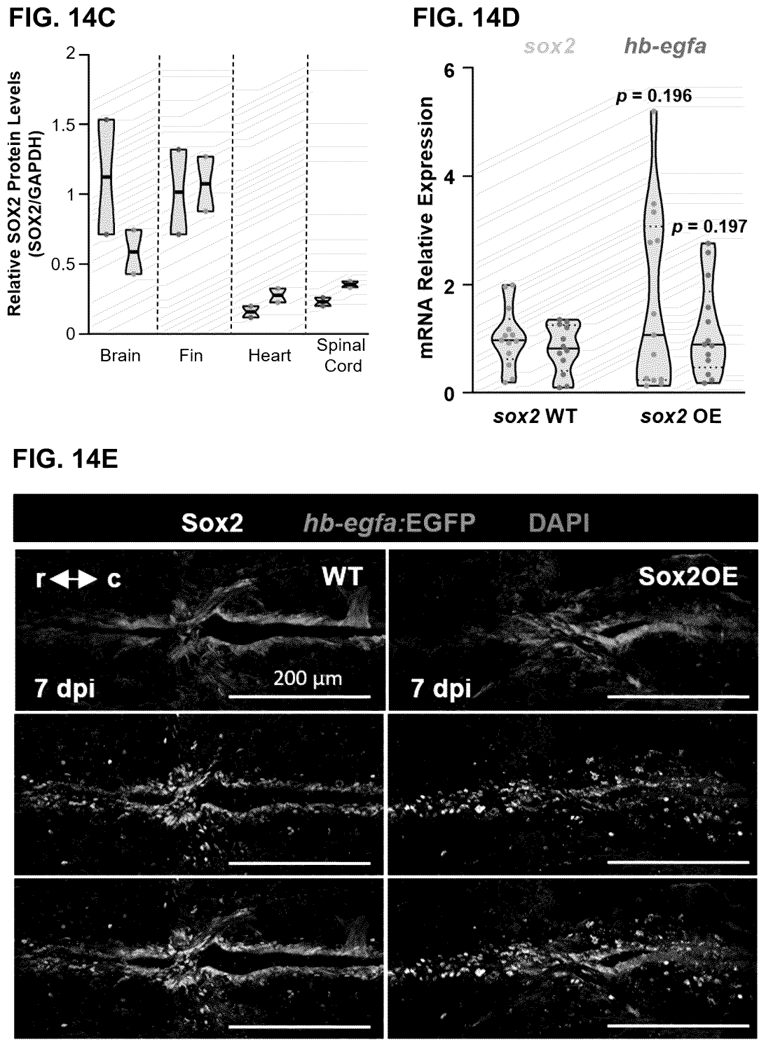

To test whether hb-egfaEN can direct injury induced gene expression after spinal cord injury, a region encompassing it upstream of the minimal promoter c-fos was fused to an EGFP reporter gene and stable transgenic lines (hb-egfaEN-cfos:EGFP, allele hb-egfaEN^{pd365}) were established (FIG. 4D). Transection injuries were performed and EGFP fluorescence was assessed (FIG. 4E). While spinal cord fluorescence was not detected in uninjured or injured cfos:EGFP control transgenics (allele cfos:EGFP^{pd366}) or in uninjured hb-egfaEN-cfos:EGFP animals (FIG. 13B, FIG. 4E), hb-egfaEN directed EGFP expression was detected at spinal cord injury sites in both larval and adult zebrafish (each of 3 stable lines generated) (FIG. 4E, FIG. 13A, and FIG. 13C). hb-egfaEN-cfos:EGFP had similar spatiotemporal dynamics after spinal cord injury as those in hb-egfa:EGFP BAC transgenics (FIG. 4E-FIG. 4F, FIG. 13A, FIG. 13C). Cells activating hb-egfaEN lined the central canal at one wpi (FIG. 4E), and often showed EdU incorporation activity and expression of Sox2 (FIG. 4G-FIG. 411). Most notably, hb-egfaEN-cfos:EGFP had highly polarized localization in central canal and glial tissue comprising the caudal site of the lesion, with 410% and 190% greater fluorescence in caudal stumps at one 1 wpi and 2 wpi, respectively (FIG. 4E-FIG. 4F). To implicate transcription factors that regulate hb-egfa expression, Homer2 software was used to predict TF binding motifs surrounding and within the hb-egfa gene region. Among the most enriched sites were those predicted to be recognized by Sox2, known to be key for ERG activation and division upon injury (Ogai K, et al. (2014) Neurosci Res. 88:84-87) (FIG. 4I, Table 4). Sox2 has been reported to activate HB-EGF transcription in in vitro cancer models (Xiao W, et al. (2020) Mol Ther Oncolytics. 17:118-129), and colocalizes with many hb-egfa:EGFP-expressing cells during spinal cord regeneration as mentioned earlier (FIG. 2C). To test if hb-egfa is a direct or indirect target of Sox2, published transgenic fish enabling heat shock-inducible expression of sox2 were used (Gou Y, et al. (2018) Dev Biol. 435:84-95). sox2 inducibility was silenced in adults, as commonly seen with lines not originally filtered for adult functionality, but was potent in larvae (FIG. 14A-FIG. 14E). A single daily heat-shock from 3 to 6 dpf was sufficient to induce a 7-fold increase in sox2 mRNA levels and a 106-fold increase in hb-egfa RNA levels. Ectopic sox2 expression also elevated hb-egfa:EGFP fluorescence throughout BAC transgenic larvae (FIG. 14J-FIG. 141). These data identified a TREE linked to hb-egfa that is sufficient to direct context-dependent gene expression, and is likely to contribute at least in part to polarized hb-egfa expression during spinal cord regeneration.

### Example 5

### Zebrafish HB-EGFEN Regulatory Sequences Directed Injury-Responsive Gene Expression in Neonatal Mouse Spinal Cord

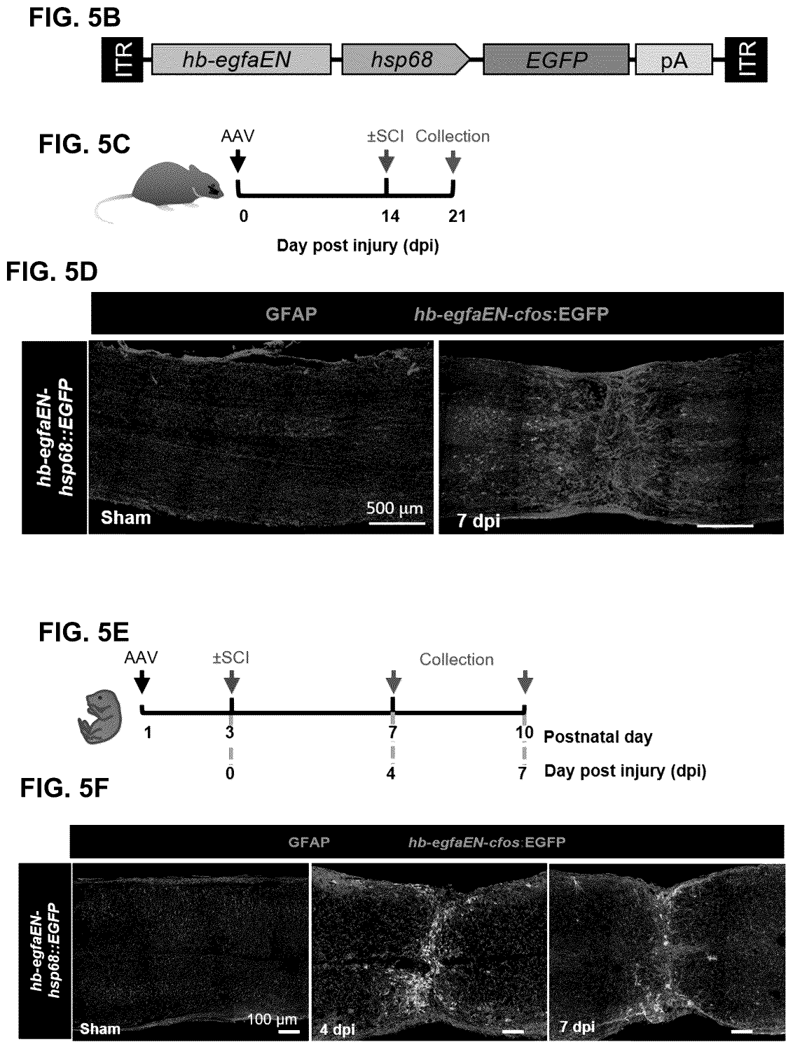

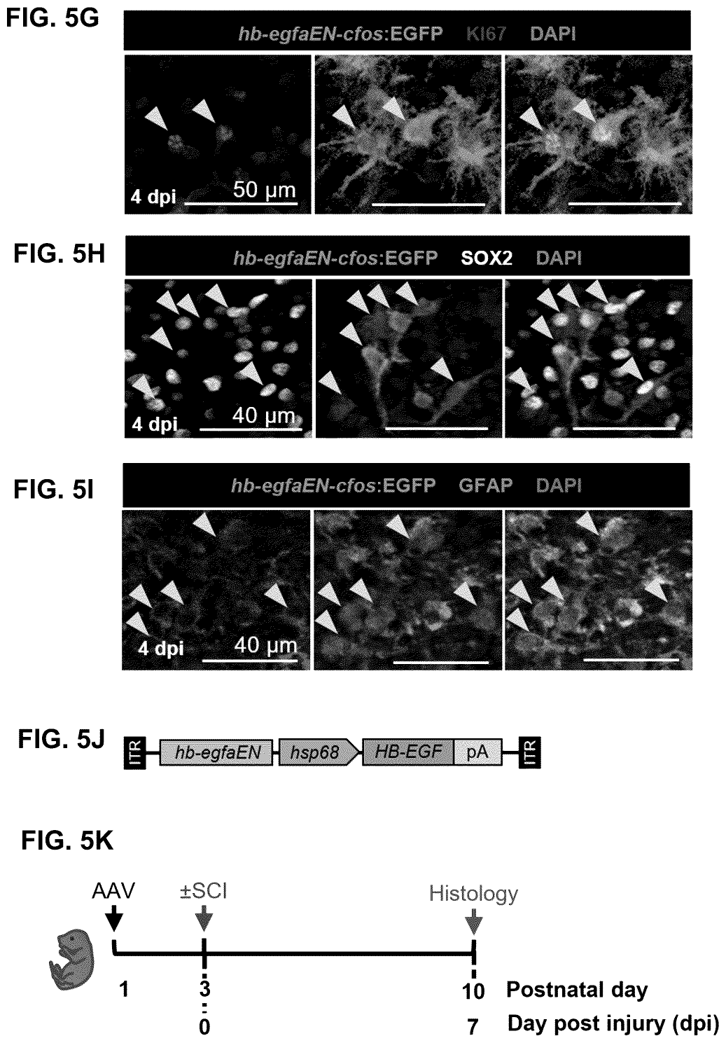

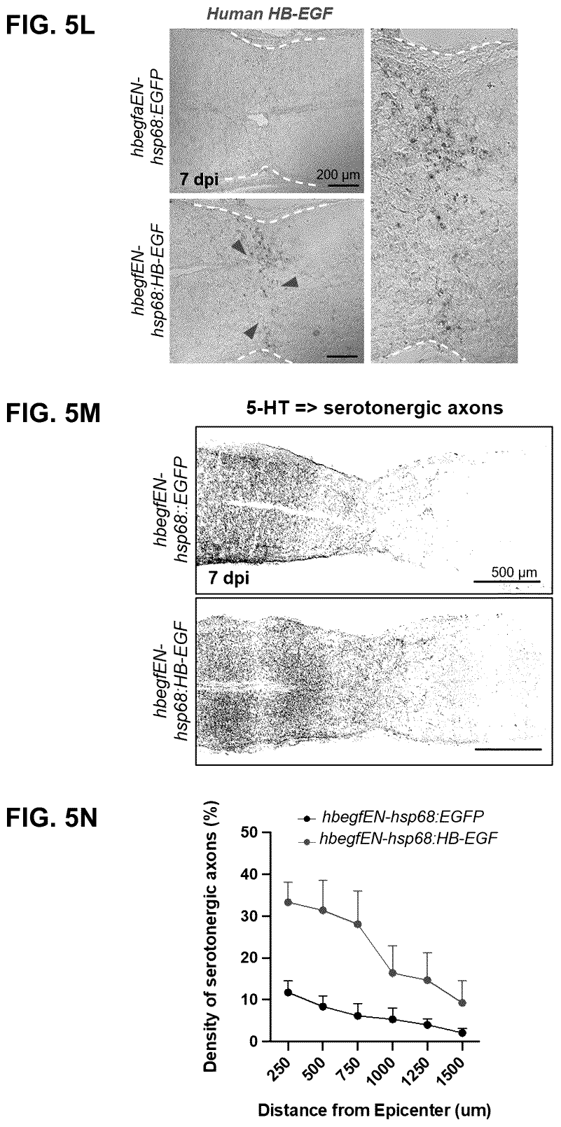

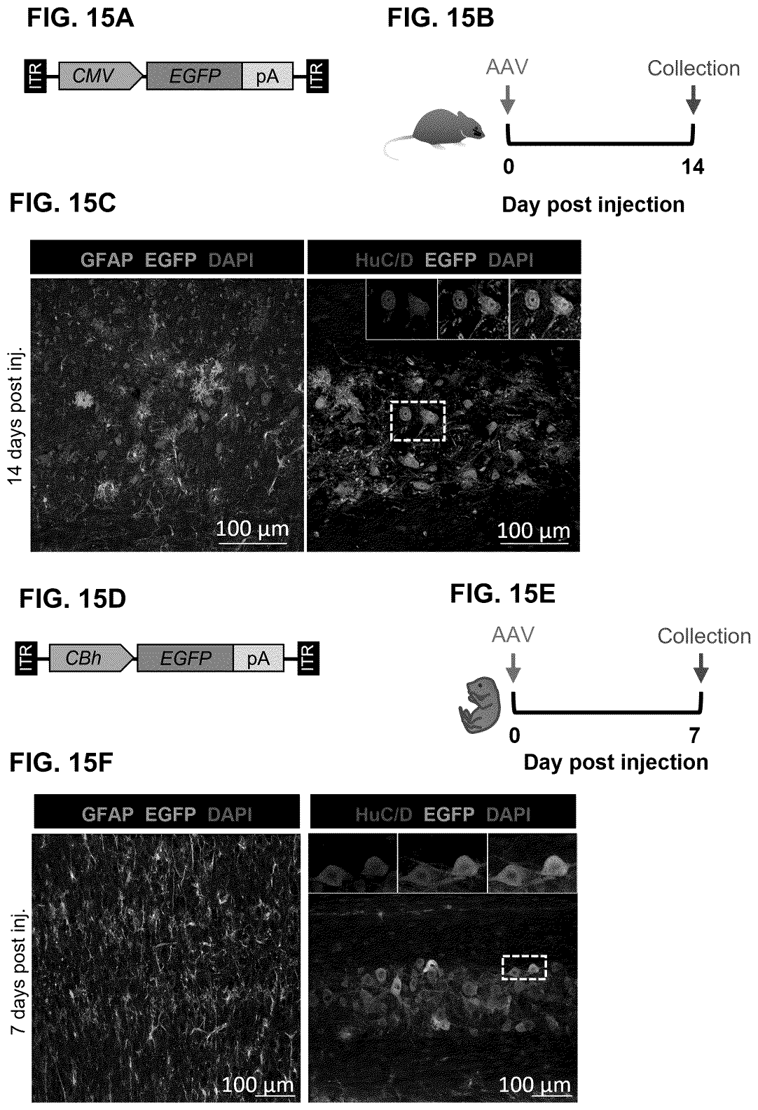

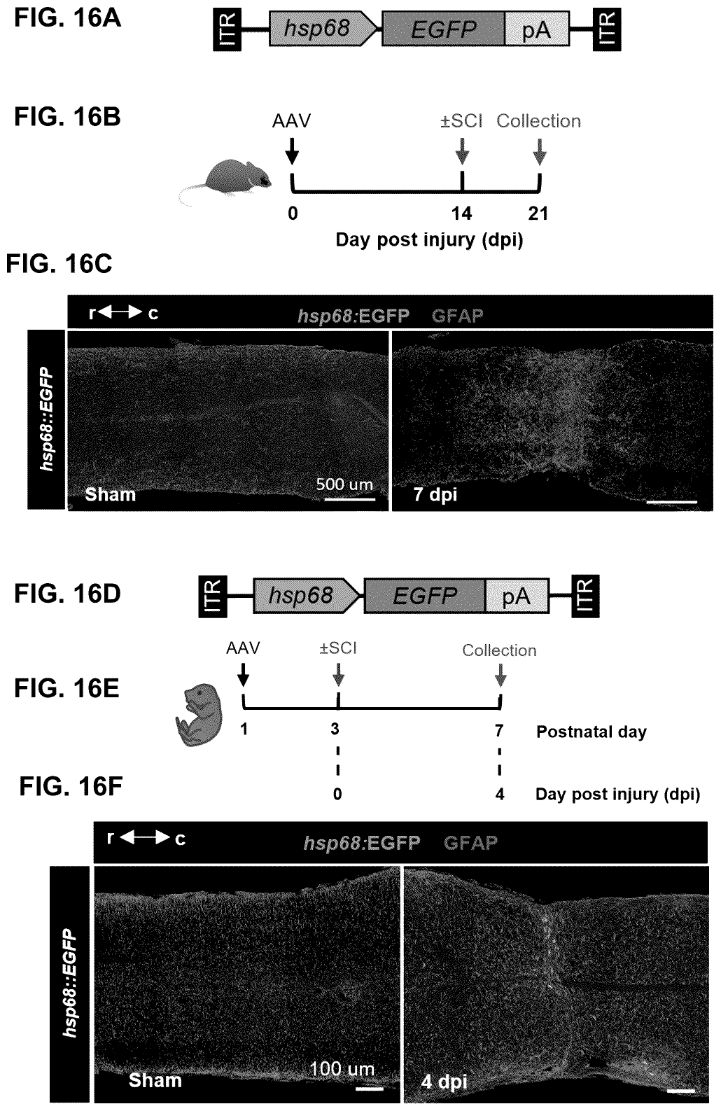

Several vertebrate genomes for sequence conservation of hb-egfEN, finding significant sequence identify only in Fugu (FIG. 5A). To test for functional conservation of hb-egfaEN in mammals, an adeno-associated viral vector (AAV) containing hb-egfaEN upstream of a murine minimal hsp68 promoter and an EGFP reporter gene was generated (FIG. 5B). To maximize infection of spinal cord tissue, the CC47 capsid was used to effectively transduce many or most spinal cord cell types when delivered systemically by tail vein injection to adult mice (FIG. 15A-FIG. 15C). To assess whether zebrafish hb-egfaEN can direct similar injury-induced expression in mice, 3-month-old adult mice were injected with CC47 carrying an hh-egfEN-hsp68:EGFP construct. Then, thoracic crush injuries (T9-T10 level) were performed 2 weeks after injection, and EGFP fluorescence was assessed at one wpi (FIG. 5C). Mice injected with CC47 carrying hsp68:EGFP sequences alone were used as controls (FIG. 16A). Little or no EGFP expression was observed in mice treated with either AAV preparation in sham or crush injuries, at the injury site or elsewhere (FIG. 5D, FIG. 16A-FIG. 16C).

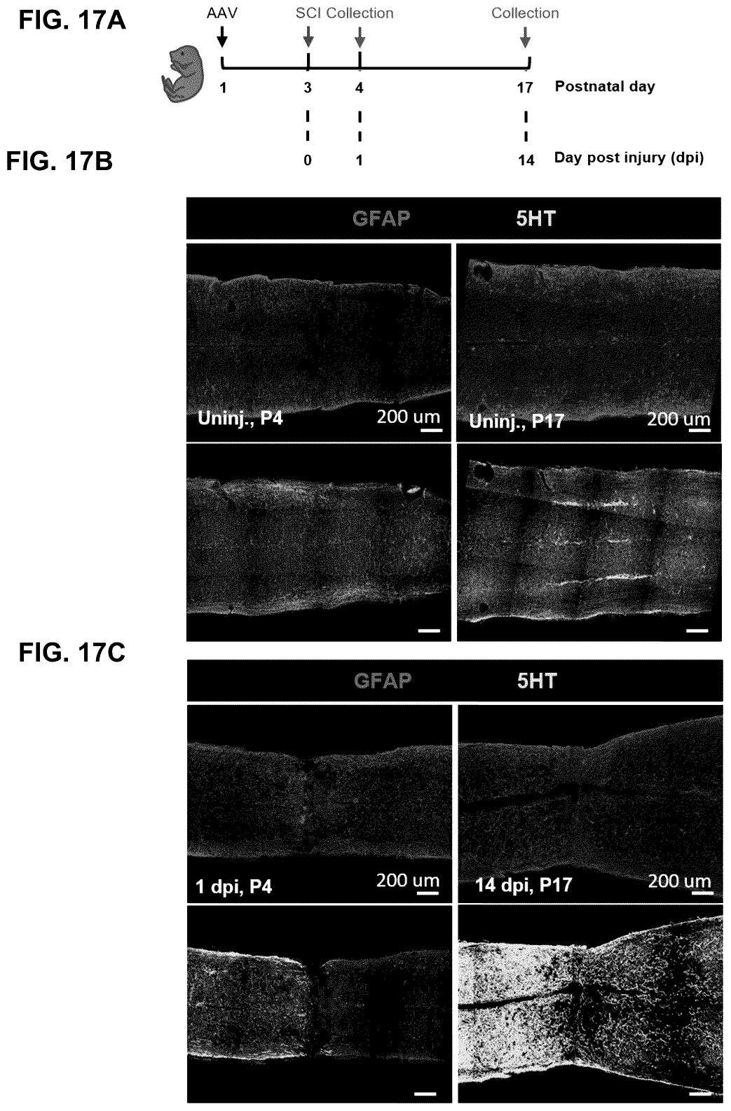

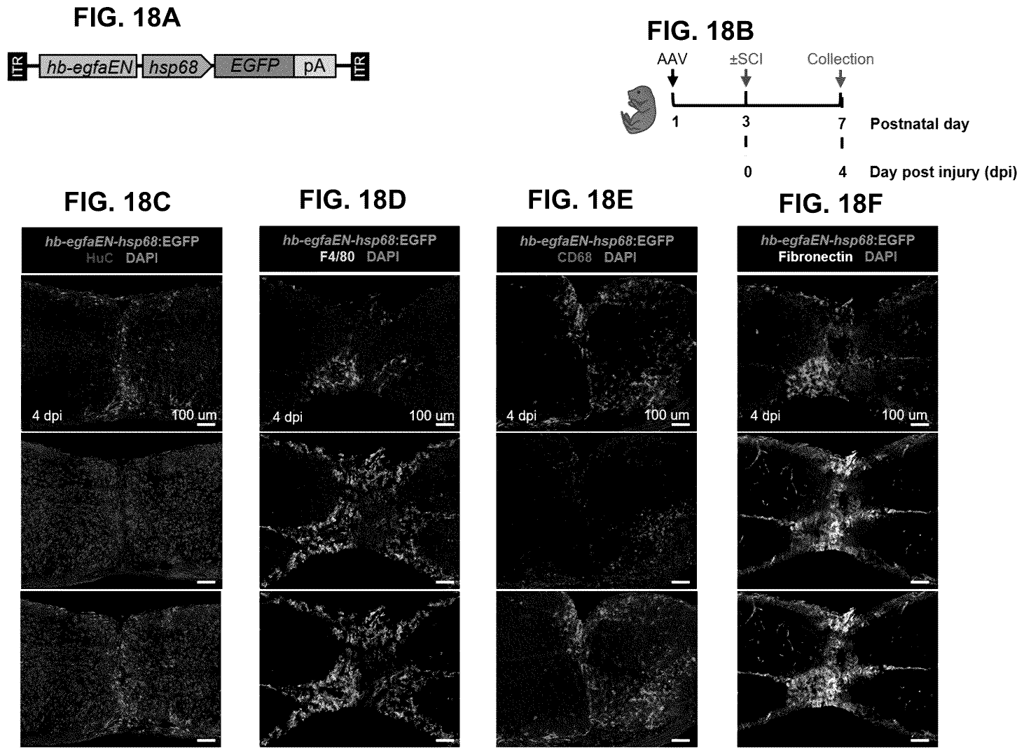

One explanation for these findings is that hb-egfaEN sequences had changed during evolution so as to not be recognizable by mammalian transcriptional machinery. Another possibility is that gene regulatory factors that bind and are guided by these zebrafish sequences are not present or active after injury in adult murine spinal cord tissue. To address this, these same vectors were administered to neonatal mice, which were recently shown to possess some capacity to regenerate axons across a major spinal cord injury (Li Y. et al. (2020) Nature. 587(7835):613-618). First, a spinal cord crush injury was performed in P3 pups and the extent of axon growth was assessed across lesions at one and 14 dpi. Indeed, the serotonergic axons distal to the injury were overserved to show modest GFAP accumulation at the lesion site by 14 dpi, indicating similar regenerative capacity to that reported (FIG. 17A-FIG. 17C). To assess hb-egfEN activity in neonates, the CC47 vector carrying hb-egfEN-hsp68:EGFP⁺ or control hsp68-EGFP sequences, which effectively transduces spinal cord cell types in neonates (FIG. 15D-FIG. 15F), was systematically administered. The virus was introduced at PI, animals were subjected to crush injuries (T9-T10 level) at P3, and tissue was analyzed at two timepoints after injury (FIG. 5E, FIG. 16A-FIG. 16F). hsp68:EGFP directed occasional but minimal EGFP expression in sham or crush injuries, similar to experiments in adults (FIG. 16D-FIG. 16F). Strikingly, neonatal mice infected with hb-egfEN-hsp68:EGFP expressed EGFP in a tight, injury localized domain at 4 and 7 dpi (FIG. 5E-FIG. 5F). hb-egfEN:EGFP⁺ cells lined the lesion site and expressed markers characteristic of zebrafish ERGs like Sox2 and GFAP at 1 wpi, with many also positive for the proliferation marker Ki67 (FIG. 5G-FIG. 5I). Markers of neurons (HuC, FIG. 18A), macrophages (F4/80, FIG. 18B) and microglia (CC68), FIG. 18C) did not show major overlap with EGFP⁺ cells at 4 dpi. A subset of cells expressed the marker fibronectin (FIG. 18F). Based on these findings, the zebrafish TREE hb-egfaEN and its injury-targeting abilities can be recognized by murine transcription complexes. However, key components of this recognition, which could be chromatin features, DNA binding proteins, or other factors, are present only at early life stages and become inaccessible with age.

### Example 6

### hB-EGFEN-Directed HB-EGF Expression Improved Axon Regeneration in Neonatal Mouse Spinal Cord Injuries

To test whether HB-EGF has instructive effects in mammals, a strategy harnessing hb-egfaEN was used to augment HB-EGF in the lesion sites of neonatal mice. An AAV was generated having the zebrafish hb-egfaEN upstream of an hsp68 minimal promoter and a gene cassette encoding a constitutively secreted form of human HB-EGF (CC47 hbegfaEN-hsp68:HB-EGF) (FIG. 5J). Following systemic administration of this virus or hbegfaEN-hsp68:EGFP control at P1 and thoracic crush injuries at P3 as above, the spinal cord tissue was histologically examined at P10 (FIG. 5K). Expression of human HB-EGF mRNA was concentrated at the lesion sites in mice pre-treated systemically with experimental virus, indicating targeting of the payload as expected (FIG. 5L). To determine whether this increase in localized HB-EGF had any impact on neonatal spinal cord regeneration, the density of serotonergic axons caudal to the crush site was examined at 7 dpi, as a percentage of densities rostral to the lesion. These data revealed increases in serotonergic axons crossing the lesion in hbegfaEN-hsp68-HB-EGF mice compared to controls, measured at several points within a region 1 mm from the trauma (FIG. 5M-FIG. 5N). Thus, by use of an enhancer sequence important for localizing Hb-egfa during innate regeneration of the zebrafish spinal cord, these experiments spatiotemporally augment HB-EGF to the extent that it can enhance axon regeneration after spinal cord injury in mice.

TABLE 1
Representative Spinal cord RNA-seq Profiling at 1 Week Post Injury

| | | log2 | | | | | | | | | | | | |
|---|---|---|---|---|---|---|---|---|---|---|---|---|---|---|
| | | Fold | | log2 | | | | | counts - | | | counts - | | |
| Ensemble | | Δ W/O | Base | Fold | | | | | injured | | | uninjured | | |
| (ENSDARG) | gene | Shrink | Mean | Change | lfcSE | stat | pvalue | padj | rep1 | rep2 | rep3 | rep1 | rep2 | rep3 |
| 29615 | zgc: 77056 | −0.439 | 805.494 | −0.406 | 0.147 | −2.764 | 0.006 | 0.026 | 847 | 708 | 664 | 652 | 921 | 1014 |
| 45797 | si: ch211- | −0.373 | 230.203 | −0.348 | 0.138 | −2.516 | 0.012 | 0.044 | 249 | 212 | 191 | 208 | 248 | 265 |
| | 68a17.7 | | | | | | | | | | | | | |
| 58940 | si: ch211- | 0.938 | 243.115 | 0.703 | 0.240 | 2.932 | 0.003 | 0.018 | 374 | 396 | 273 | 208 | 107 | 140 |
| | 248c11.2 | | | | | | | | | | | | | |
| 34457 | si: ch211- | 0.990 | 183.743 | 0.819 | 0.209 | 3.924 | 0.000 | 0.001 | 271 | 313 | 212 | 133 | 106 | 98 |
| | 163121.7 | | | | | | | | | | | | | |
| 94809 | ms4a17a.11 | 1.016 | 1302.882 | 0.857 | 0.201 | 4.260 | 0.000 | 0.000 | 2161 | 1934 | 1585 | 1008 | 638 | 708 |
| 07018 | ms4a17a.6 | 0.775 | 189.288 | 0.678 | 0.183 | 3.708 | 0.000 | 0.002 | 295 | 284 | 202 | 144 | 109 | 132 |
| 90552 | si: dkey- | 0.692 | 3193.207 | 0.617 | 0.172 | 3.586 | 0.000 | 0.003 | 4578 | 4646 | 3605 | 2672 | 1849 | 2175 |
| | 7j14.6 | | | | | | | | | | | | | |
| 05989 | rg11 | 0.713 | 1910.928 | 0.686 | 0.107 | 6.436 | 0.000 | 0.000 | 2864 | 2728 | 2140 | 1386 | 1241 | 1367 |
| 94809 | ms4a17a.11 | 1.016 | 1302.882 | 0.850 | 0.201 | 4.260 | 0.000 | 0.000 | 2161 | 1934 | 1585 | 1008 | 638 | 708 |
| 07018 | masda17a.6 | 0.775 | 189.288 | 0.678 | 0.183 | 3.708 | 0.000 | 0.002 | 295 | 284 | 202 | 144 | 109 | 132 |
| 90552 | si: dkey- | 0.692 | 3193.207 | 0.617 | 0.172 | 3.586 | 0.000 | 0.003 | 4578 | 4646 | 3605 | 2672 | 1849 | 2175 |
| | 7j14.6 | | | | | | | | | | | | | |
| 37142 | zgc: 153146 | 0.661 | 120.901 | 0.571 | 0.190 | 3.004 | 0.003 | 0.015 | 190 | 172 | 123 | 92 | 75 | 92 |
| 100439 | zgc: 153383 | 0.774 | 1007.282 | 0.723 | 0.136 | 5.306 | 0.000 | 0.000 | 1453 | 1342 | 1305 | 719 | 644 | 685 |
| 79636 | fam177a1 | 0.473 | 1421.361 | 0.459 | 0.095 | 4.849 | 0.000 | 0.000 | 1928 | 1908 | 1532 | 1096 | 1073 | 1117 |

| | | | | | | | | | mmusculus_homo- |
|---|---|---|---|---|---|---|---|---|---|
| | fpkm.injured | | | fpkm.uninjured | | | | mmusculus_homo- | log_associat- |
| ensembl | rep1 | rep2 | rep3 | rep1 | rep2 | rep3 | entriz_id | log_ensembl_gene | ed_gene_name |
| 29615 | 7.8780292 | 6.9929467 | 8.0429133 | 8.57531 | 11.3132 | 11.1637 | 327040 | ENSMUSG00000032551 | 1110059G10Rik |
| 45797 | 4.9599878 | 4.4844575 | 4.9547959 | 5.85885 | 6.52415 | 6.24834 | 100190888 | ENSMUSG00000063320 | 1190007I07Rik |
| 58940 | 3.9558204 | 4.4478786 | 3.7604407 | 3.11097 | 1.49465 | 1.75279 | 557793 | ENSMUSG00000026831 | 1700007K13Rik |
| 34457 | 3.6561235 | 4.4842383 | 3.7247623 | 2.5373 | 1.88864 | 1.565 | 567726 | ENSMUSG00000027886 | 1700013F07Rik |
| 94809 | 5.9575594 | 5.6619088 | 5.690547 | 3.92954 | 2.32287 | 2.31039 | 550363 | ENSMUSG00000024729 | 1700017D01Rik |
| 7018 | 1.4082353 | 1.4396746 | 1.2557863 | 0.97204 | 0.68718 | 0.74587 | 100009643 | ENSMUSG00000024729 | 1700017D01Rik |
| 90552 | 12.033944 | 12.968931 | 12.34095 | 9.932 | 6.41889 | 6.76751 | 556585 | ENSMUSG00000024729 | 1700017D01Rik |
| 5989 | 1.7075571 | 1.727187 | 1.6616024 | 1.16851 | 0.97716 | 0.96474 | 402933 | ENSMUSG00000029620 | 1700018F24Rik |
| 94809 | 5.9575594 | 5.6619088 | 5.690547 | 3.92954 | 2.32287 | 2.31039 | 550363 | ENSMUSG00000024728 | 1700025F22Rik |
| 7018 | 1.4082353 | 1.4396746 | 1.2557863 | 0.97204 | 0.68718 | 0.74587 | 100009643 | ENSMUSG00000024728 | 1700025F22Rik |
| 90552 | 12.033944 | 12.968931 | 12.34095 | 9.932 | 6.41889 | 6.76751 | 556585 | ENSMUSG00000024728 | 1700025F22Rik |
| 37142 | 0.337649 | 0.3245885 | 0.2846611 | 0.23119 | 0.17602 | 0.19353 | 751702 | ENSMUSG00000071103 | 1700029J07Rik |
| 100439 | 2.6340511 | 2.5834727 | 3.0809186 | 1.84313 | 1.54183 | 1.4699 | 751751 | ENSMUSG00000094103 | 1700047I7Rik2 |
| 79636 | 9.4936844 | 9.9769757 | 9.8242106 | 7.63144 | 6.9778 | 6.51056 | 100126028 | ENSMUSG00000094103 | 1700047I17Rik2 |

TABLE 2
Representative Merged ATAC-seq Peaks and RNA-seq Genes

| | | log2 | | | | | | | | | | | | | | |
|---|---|---|---|---|---|---|---|---|---|---|---|---|---|---|---|---|
| | | Fold | | | | | | | | | | | | | | |
| | | Change | log2 | | | | | | | | | | | | | ensembl_{—} |
| | | w/o | Fold | p | p | base | fpkem.uninjured | | | fpkm.injured | | | | | | gene_id |
| | gene | Shrink | Change | value | adj | Mean | rep1 | rep2 | rep3 | rep1 | rep2 | rep3 | Fold | p.value | FDR | (ENSDARG) |
| 554 | chmp4c | −4.96 | −0.46 | 0.00 | 0.01 | 24.72 | 0.81 | 0.14 | 0.03 | 0.00 | 0.02 | 0.01 | −1.75 | 0.01 | 1.00 | 7418 |
| 3103 | si: dkeyp- | −4.92 | −0.26 | 0.01 | 0.03 | 391.40 | 45.62 | 0.23 | 1.80 | 0.10 | 1.06 | 0.41 | 0.89 | 0.04 | 0.45 | 92498 |
| | 46h3.2 | | | | | | | | | | | | | | | |
| 3830 | zgc: 171446 | −4.81 | −0.27 | 0.01 | 0.03 | 615.08 | 12.54 | 0.08 | 0.52 | 0.03 | 0.29 | 0.14 | −1.33 | 0.01 | 1.00 | 116005 |
| 3831 | zgc: 171446 | −4.81 | −0.27 | 0.01 | 0.03 | 615.08 | 12.54 | 0.08 | 0.52 | 0.03 | 0.29 | 0.14 | −1.29 | 0.03 | 0.39 | 116005 |
| 3890 | zp211 | −4.67 | −0.25 | 0.01 | 0.04 | 342.58 | 35.96 | 0.29 | 1.62 | 0.03 | 0.81 | 0.64 | −1.26 | 0.04 | 1.00 | 55415 |
| 3891 | zp211 | −4.67 | −0.25 | 0.01 | 0.04 | 342.58 | 35.96 | 0.29 | 1.62 | 0.03 | 0.81 | 0.64 | −1.24 | 0.02 | 1.00 | 55415 |
| 3892 | zp211 | −4.67 | −0.25 | 0.01 | 0.04 | 342.58 | 35.96 | 0.29 | 1.62 | 0.03 | 0.81 | 0.64 | −2.15 | 0.05 | 1.00 | 55415 |
| 3834 | zgc: 171776 | −4.64 | −0.25 | 0.01 | 0.04 | 286.69 | 28.07 | 0.28 | 1.31 | 0.03 | 0.8 | 0.27 | −0.96 | 0.04 | 1.00 | 32156 |
| 3835 | zgc: 171776 | −4.64 | −0.25 | 0.01 | 0.04 | 286.69 | 28.07 | 0.28 | 1.31 | 0.03 | 0.89 | 0.27 | 1.12 | 0.01 | 0.29 | 32156 |
| 3836 | zgc: 171776 | −4.64 | −0.25 | 0.01 | 0.04 | 286.69 | 28.07 | 0.28 | 1.31 | 0.03 | 0.89 | 0.27 | −1.09 | 0.04 | 1.00 | 32156 |
| 3837 | zgc: 171776 | −4.64 | −0.25 | 0.01 | 0.04 | 286.69 | 28.07 | 0.28 | 1.31 | 0.03 | 0.89 | 0.27 | −1.90 | 0.03 | 1.00 | 32156 |
| 3838 | zgc: 171776 | −4.64 | −0.25 | 0.01 | 0.04 | 286.69 | 28.07 | 0.28 | 1.31 | 0.03 | 0.89 | 0.27 | −1.98 | 0.02 | 1.00 | 32156 |
| 335 | btg4 | −4.62 | −0.28 | 0.01 | 0.03 | 178.72 | 19.81 | 0.13 | 0.96 | 0.07 | 0.43 | 0.35 | −1.62 | 0.04 | 1.00 | 35171 |
| 336 | btg4 | −4.62 | −0.28 | 0.01 | 0.03 | 178.72 | 19.81 | 0.13 | 0.96 | 0.07 | 0.43 | 0.35 | −1.24 | 0.03 | 1.00 | 35171 |
| 3022 | sich211- | −4.45 | −0.29 | 0.01 | 0.03 | 196.97 | 35.33 | 0.26 | 2.03 | 0.17 | 0.94 | 0.61 | −1.15 | 0.03 | 1.00 | 116290 |
| | 250c5.16 | | | | | | | | | | | | | | | |
| 675 | crp2 | −4.39 | −0.38 | 0.01 | 0.04 | 7.01 | 1.02 | 0.07 | 0.15 | 0.00 | 0.03 | 0.03 | −1.36 | 0.04 | 1.00 | 56462 |
| 3092 | si: dkey- | −4.08 | −0.31 | 0.01 | 0.04 | 239.01 | 3.52 | 0.04 | 0.22 | 0.03 | 0.13 | 0.06 | −2.01 | 0.02 | 1.00 | 8835 |
| | 46g23.5 | | | | | | | | | | | | | | | |

| | | | | distance | uninj | | | injured | | | | |
|---|---|---|---|---|---|---|---|---|---|---|---|---|
| gene | seqnames | start | end | to Feature | rep 1 | rep2 | rep3 | rep1 | rep2 | rep3 | quadrant | col |
| chmp4c | chr24 | 10935864 | 10936264 | −128.00 | 7.46 | 10.19 | 14.07 | 2.33 | 3.02 | 4.08 | III | blue |
| si: dkeyp- | chr19 | 27957851 | 27958700 | 46334.00 | 15.00 | 15.00 | 21.00 | 48.00 | 66.00 | 50.00 | II | black |
| [symbol] | | | | | | | | | | | | |
| zgc: 171446 | chr3 | 3320258 | 3320658 | 310958.00 | 26.85 | 33.47 | 20.56 | 4.67 | 16.89 | 10.61 | III | blue |
| zgc: 171446 | chr3 | 3320251 | 3320650 | 310966.00 | 23.00 | 22.00 | 21.00 | 9.00 | 32.00 | 12.00 | III | blue |
| zp211 | chr9 | 35831034 | 35831434 | −6760.00 | 29.84 | 16.01 | 14.07 | 6.42 | 13.27 | 5.72 | III | blue |
| zp211 | chr9 | 35893764 | 35894164 | −69490.00 | 7.46 | 11.64 | 2.16 | 1.17 | 3.62 | 0.00 | III | blue |
| zgc: 171776 | chr9 | 8183031 | 8183431 | − [symbol] | 20.89 | 18.92 | 22.72 | 9.92 | 13.27 | 8.98 | III | blue |
| zgc: 171776 | chr9 | 8089901 | 8090050 | − [symbol] | 9.00 | 9.00 | 7.00 | 53.00 | 29.00 | 27.00 | II | black |
| zgc: 171776 | chr9 | 8264757 | 8265531 | − [symbol] | 13.43 | 8.73 | 22.72 | 5.25 | 6.03 | 9.80 | III | blue |
| zgc: 171776 | chr9 | 8343389 | 8343789 | −74836.00 | 10.44 | 11.64 | 3.25 | 2.33 | 3.62 | 0.82 | III | blue |
| zgc: 171776 | chr9 | 8403981 | 8404381 | −14244.00 | 8.95 | 10.19 | 5.41 | 1.73 | 1.21 | 3.27 | III | blue |
| btg4 | chr5 | 57307110 | 57307510 | 52916.00 | 14.92 | 7.28 | 7.57 | 5.84 | 0.60 | 3.27 | III | blue |
| btg4 | chr5 | 57344867 | 57345660 | 90870.00 | 17.90 | 13.10 | 14.07 | 9.34 | 4.83 | 4.90 | III | blue |
| si: ch211- | chr22 | 25730486 | 25730886 | −3443.00 | 16.41 | 17.46 | 18.39 | 7.59 | 10.25 | 5.72 | III | blue |
| [symbol] | | | | | | | | | | | | |
| crp2 | chr24 | 38183951 | 38184351 | 11332.00 | 13,43 | 8.73 | 12.98 | 5.25 | 6.03 | 2.45 | III | blue |
| si: dkey- | chr3 | 3739443 | 3739843 | −73801.00 | 11.93 | 4.37 | 12.98 | 5.25 | 1.21 | 0.82 | III | blue |
| [symbol] | | | | | | | | | | | | |
| zglp1 | chr6 | 188965 | 189365 | 54070.00 | 7.46 | 13.10 | 5.41 | 18.09 | 40.41 | 31.84 | II | black |

[symbol]

indicates data missing or illegible when filed

TABLE 3
Representative GO Analyses of ATAC-Peaks Assigned to Differentially Expressed Genes at 1 WPI

| | | #Gene | | | | | | | | | Best | Best | |
|---|---|---|---|---|---|---|---|---|---|---|---|---|---|
| | | In GO | #Gene | #Total | | | | | | | Enrich- | Log | canon- |
| #COUT_{—} | #Gene | and Hit | In Hit | Gene In | % In | _In | _LogP_{—} | MEMBER_{—} | | | ment | P In | ical |
| 001 | In GO | List | List | Library | GO | GO | My List | My List | PATTERN | RANK | In Group | Group | Name |
| 117 | 700 | 117 | 1397 | 18430 | 8.375 | | −15.723 | 1 | M1 | 1 | 2.205 | −15.723 | GO: |
| | | | | | | | | | | | | | 0007417 |
| 22 | 139 | 22 | 1397 | 18430 | 1.575 | | −3.116 | 1 | M1 | 1 | 2.205 | −15.723 | GO: |
| | | | | | | | | | | | | | 0030900 |
| 79 | 501 | 79 | 1397 | 18430 | 5.655 | | −9.489 | 1 | M1 | 1 | 2.205 | −15.723 | GO: |
| | | | | | | | | | | | | | 0007420 |
| 81 | 527 | 81 | 1397 | 18430 | 5.798 | | −9.169 | 1 | M1 | 1 | 2.205 | −15.723 | GO: |
| | | | | | | | | | | | | | 0060322 |
| 49 | 267 | 49 | 1397 | 18430 | 3.508 | | −8.236 | 1 | M1 | 1 | 2.421 | −12.277 | GO: |
| | | | | | | | | | | | | | 0007369 |
| 85 | 493 | 85 | 1397 | 18430 | 6.084 | | −12.277 | 1 | M1 | 1 | 2.421 | −12.277 | GO: |
| | | | | | | | | | | | | | 0002009 |
| 93 | 584 | 93 | 1397 | 18430 | 6.657 | | −11.326 | 1 | M1 | 1 | 2.421 | −12.277 | GO: |
| | | | | | | | | | | | | | 0048729 |
| 111 | 744 | 111 | 1397 | 18430 | 7.946 | | −11.544 | 1 | M1 | 1 | 2.421 | −12.277 | GO: |
| | | | | | | | | | | | | | 0048598 |
| 36 | 260 | 36 | 1397 | 18430 | 2.577 | | −3.494 | 1 | M1 | 1 | 2.421 | −12.277 | GO: |
| | | | | | | | | | | | | | 0060562 |
| 85 | 621 | 85 | 1397 | 18430 | 6.084 | | −7.180 | 1 | M1 | 1 | 2.421 | −12.277 | GO: |
| | | | | | | | | | | | | | 0048568 |
| 56 | 437 | 56 | 1397 | 18430 | 4.009 | | −4.123 | 1 | M1 | 1 | 2.421 | −12.277 | GO: |
| | | | | | | | | | | | | | 0048562 |
| 28 | 134 | 28 | 1397 | 18430 | 2.004 | | −6.143 | 1 | M1 | 1 | 2.757 | −11.158 | GO: |
| | | | | | | | | | | | | | 0042246 |
| 17 | 83 | 17 | 1397 | 18430 | 1.217 | | −3.864 | 1 | MI | 1 | 2.757 | −11.158 | GO: |
| | | | | | | | | | | | | | 0031101 |
| 68 | 371 | 68 | 1397 | 18430 | 4.868 | | −11.158 | 1 | M1 | 1 | 2.757 | −11.158 | GO: |
| | | | | | | | | | | | | | 0048589 |
| 36 | 198 | 36 | 1397 | 18430 | 2.577 | | −6.126 | 1 | M1 | 1 | 2.757 | −11.158 | GO: |
| | | | | | | | | | | | | | 0031099 |
| 73 | 417 | 73 | 1397 | 18430 | 5.225 | | −10.941 | 1 | M1 | 1 | 2.757 | −11.158 | GO: |
| | | | | | | | | | | | | | 0040007 |
| 37 | 205 | 37 | 1397 | 18430 | 2.649 | | −6.196 | 1 | M1 | 1 | 3.694 | −6.196 | R-DRE- |
| | | | | | | | | | | | | | 1266738 |

| | | | | | | First In | First In | |
|---|---|---|---|---|---|---|---|---|
| | Category | | | | Evidence | Group By | Group By | |
| Category | ID | CLUSTER_LABEL | Description | Enrichment | Cutoff | Enrichment | LogP | Gene |
| GO Biological | 19 | central nervous | central nervous | 2.205041415 | 0 | 1 | 1 | GO: 0007417 |
| Processes | | system development | system development | | | | | |
| GO Biological | 19 | | forebrain | 2.088030363 | 0 | 0 | 0 | GO: 0030900 |
| Processes | | | development | | | | | |
| GO Biological | 19 | | brain development | 2.080263239 | 0 | 0 | 0 | GO: 0007420 |
| Processes | | | | | | | | |
| GO Biologieat | 19 | | head development | 2.02769828 | 0 | 0 | 0 | GO: 0060322 |
| Processes | | | | | | | | |
| GO Biological | 19 | | gastrulation | 2.421105687 | 0 | 0 | 0 | GO: 0007369 |
| Processes | | | | | | | | |
| GO Biological | 19 | morphogenesis of | morphogenesis of | 2.27457853 | 0 | 1 | 1 | GO: 0002009 |
| Processes | | an epithelium | an epithelium | | | | | |
| GO Biological | 19 | | tissue | 2.10086928 | 0 | 0 | 0 | GO: 0048729 |
| Processes | | | morphogenesis | | | | | |
| GO Biological | 19 | | embryonic | 1.968244164 | 0 | 0 | 0 | GO: 0048598 |
| Processes | | | morphogenesis | | | | | |
| GO Biological | 19 | | epithelial tube | 1.826661527 | 0 | 0 | 0 | GO: 0060562 |
| Processes | | | morphogenesis | | | | | |
| GO Biological | 19 | | embryonic organ | 1.805744308 | 0 | 0 | 0 | GO: 0048568 |
| Processes | | | development | | | | | |
| GO Biological | 19 | | embryonic organ | 1.690579191 | 0 | 0 | 0 | GO: 0048562 |
| Processes | | | morphogenesis | | | | | |
| GO Biological | 19 | | tissue | 2.756653383 | 0 | 0 | 0 | GO: 0042246 |
| Processes | | | regeneration | | | | | |
| GO Biological | 19 | | fin | 2.702089676 | 0 | 0 | 0 | GO: 0031101 |
| Processes | | | regeneration | | | | | |
| GO Biological | 19 | developmental | developmental | 2.418042513 | 0 | 1 | 1 | GO: 0048589 |
| Processes | | growth | growth | | | | | |
| GO Biological | 19 | | regeneration | 2.39864645 | 0 | 0 | 0 | GO: 0031099 |
| Processes | | | | | | | | |
| GO Biological | 19 | | growth | 2.309488129 | 0 | 0 | 0 | GO: 0040007 |
| Processes | | | | | | | | |
| Reactome Gene Sets | 6 | developmental | developmental | 2.381095379 | 0 | 1 | 1 | R-DRE-1266738 |
| | | biology | biology | | | | | |

| | | | | | | | | | STDV | |
|---|---|---|---|---|---|---|---|---|---|---|
| | | | Log_q_{—} | | | | | shared | % In | |
| Gene | id | Log(q-value) | value_{—} | LogP | name | PARENT_GO | selected | name | GO | Z-score |
| GO: 0007417 | 128 | −11.663 | | −15.723 | 128 | 19_GO: 0032502 | FALSE | 128 | 0.741 | 9.309 |
| | | | | | | developmental | | | | |
| | | | | | | process | | | | |
| GO: 0030900 | 3 | −1.515 | | −3.116 | 3 | 19_GO: 0032502 | FALSE | 3 | 0.333 | 3.688 |
| | | | | | | developmental | | | | |
| | | | | | | process | | | | |
| GO: 0007420 | 1 | −6.582 | | −9.489 | 1 | 19_GO: 0032502 | FALSE | 1 | 0.618 | 7.021 |
| | | | | | | developmental | | | | |
| | | | | | | process | | | | |
| GO: 0060322 | 2 | −6.340 | | −9.169 | 2 | 19_GO: 0032502 | FALSE | 2 | 0.625 | 6.855 |
| | | | | | | developmental | | | | |
| | | | | | | process | | | | |
| GO: 0007369 | 6 | −5.623 | | −8.236 | 6 | 19_GO: 0032502 | FALSE | 6 | 0.492 | 6.699 |
| | | | | | | developmental | | | | |
| | | | | | | process | | | | |
| GO: 0002009 | 134 | −8.518 | | −12.277 | 134 | 19_GO: 0032502 | FALSE | 134 | 0.640 | 8.215 |
| | | | | | | developmental | | | | |
| | | | | | | process | | | | |
| GO: 0048729 | 5 | −7.867 | | −11.326 | 5 | 19_GO: 0032502 | FALSE | 5 | 0.667 | 7.742 |
| | | | | | | developmental | | | | |
| | | | | | | process | | | | |
| GO: 0048598 | 4 | −7.960 | | −11.544 | 4 | 19_GO: 0032502 | FALSE | 4 | 0.724 | 7.721 |
| | | | | | | developmental | | | | |
| | | | | | | process | | | | |
| GO: 0060562 | 9 | −1.792 | | −3.494 | 9 | 19_GO: 0032502 | FALSE | 9 | 0.424 | 3.845 |
| | | | | | | developmental | | | | |
| | | | | | | process | | | | |
| GO: 0048568 | 7 | −4.743 | | −7.180 | 7 | 19_GO: 0032502 | FALSE | 7 | 0.640 | 5.850 |
| | | | | | | developmental | | | | |
| | | | | | | process | | | | |
| GO: 0048562 | 8 | −2.253 | | −4.123 | 8 | 19_GO: 0032502 | FALSE | 8 | 0.525 | 4.184 |
| | | | | | | developmental | | | | |
| | | | | | | process | | | | |
| GO: 0042246 | 11 | −3.909 | | −6.143 | 11 | 19_GO: 0040007 | FALSE | 11 | 0.375 | 5.845 |
| | | | | | | growth | | | | |
| GO: 0031101 | 13 | −2.071 | | −3.864 | 13 | 19_GO: 0040007 | FALSE | 13 | 0.293 | 4.451 |
| | | | | | | growth | | | | |
| GO: 0048589 | 131 | −7.797 | | −11.158 | 131 | 19_GO: 0040007 | FALSE | 131 | 0.576 | 7.902 |
| | | | | | | growth | | | | |
| GO: 0031099 | 12 | −3.902 | | −6.126 | 12 | 19_GO: 0032502 | FALSE | 12 | 0.424 | 5.667 |
| | | | | | | developmental | | | | |
| | | | | | | process | | | | |
| GO: 0040007 | 10 | −7.659 | | −10.941 | 10 | 19_GO: 0040007 | FALSE | 10 | 0.595 | 7.746 |
| | | | | | | growth | | | | |
| R-DRE-1266738 | 121 | −3.955 | | −6.196 | 121 | | FALSE | 121 | 0.430 | 5.695 |

TABLE 4
Representative Predicted Transcription Factor Binding Motifs in hb-egfaEN

| | | Motif | GeneID | | | | | |
|---|---|---|---|---|---|---|---|---|
| TF ID | Name | ID | (ENSDARG) | Family | Sequence | From-To | Direction | Score |
| T208502_2.00 | pdx1 | M10787_ | 2779 | Homeo- | GAAGCAATTAAG | 298-309 | R | 17.694 |
| T208527_2.00 | boxb6a | 2.00 | 10630 | domain | (SEQ ID | 298-309 | R | 17.694 |
| T208534_2.00 | boxb5a | | 13057 | | NO: 33) | 298-309 | R | 17.694 |
| T208536_2.00 | boxb4a | | 13533 | | | 298-309 | R | 17.694 |
| T208573_2.00 | hoxb6b | | 26513 | | | 298-309 | R | 17.694 |
| T208576_2.00 | hoxb3a | | 29263 | | | 298-309 | R | 17.694 |
| T208628_2.00 | hoxb8b | | 54025 | | | 298-309 | R | 17.694 |
| T208629_2.00 | hoxb5b | | 54030 | | | 298-309 | R | 17.694 |
| T208640_2.00 | hoxb8a | | 56027 | | | 298-309 | R | 17.694 |
| T208641_2.00 | hoxb7a | | 56030 | | | 298-309 | R | 17.694 |
| T208660_2.00 | hoxd4a | | 59276 | | | 298-309 | R | 17.694 |
| T208661_2.00 | hoxd3a | | 59280 | | | 298-309 | R | 17.694 |
| T208679_2.00 | hoxc4a | | 70338 | | | 298-309 | R | 17.694 |
| T208681_2.00 | hoxc5a | | 70340 | | | 298-309 | R | 17.694 |
| T208682_2.00 | hoxc6a | | 70343 | | | 298-309 | R | 17.694 |
| T208683_2.00 | hoxc8a | | 70346 | | | 298-309 | R | 17.694 |
| T208736_2.00 | hoxc6b | | 101954 | | | 298-309 | R | 17.694 |
| T208738_2.00 | hoxa5a | | 102501 | | | 298-309 | R | 17.694 |
| T208744_2.00 | hoxa4a | | 103862 | | | 298-309 | R | 17.694 |

### SUMMARY OF EXPERIMENTAL RESULTS

Here, Hb-egfa was identified as an instructive factor for axon regeneration during functional recovery of zebrafish from a paralyzing spinal cord injury. A major crush or transection injury to a longitudinal structure like spinal cord presents a patterning challenge, to stitch back two severed ends and reestablish connections of a complex bundle of wires that grow unidirectionally. It may be expected that certain bridging and trophic signals for these events are preferentially localized with a bias toward the rostral or caudal end, while others will have no intended bias. These studies indicate that Hb-egfa has a reproducible bias for expression toward the caudal side of injuries, where its activity is required for the successful crossing of regenerating axons. Gross disruption of this bias by whole-animal overexpression impedes regeneration, versus the pro-regenerative effects of a more precise Hb-egf supplementation. Localization of stimuli or inhibitory factors in this model is fundamentally analogous to placement of mitogenic triggers in the wound epidermis of regenerating fins or limbs of fish and salamanders, or in the border zone of regenerating zebrafish hearts. Spinal cord regeneration, based on these observations, likely involves some capacity to maintain, or quickly recall and recognize, positional information that distinguishes rostral from caudal tissue domains. This property resembles positional memory, which has been studied for decades in regenerating appendages (REF).

Regeneration programs are controlled in part by TREEs. The evidence provided herein that presence of a key TREE, hb-egfEN, was a likely mechanism by which hb-egf expression is biased to caudal tissue during spinal cord regeneration. Within this short DNA sequence are presumably all motifs necessary for triggering gene expression in certain cells and at a defined time window. The hb-egfaEN contains transcription factor binding sites to distinguish rostral from caudal position. It is unclear, but of interest to clarify, whether hb-egfa gene activation is upstream in this cascade, or instead is regulated through transduction of signaling initiated by upstream factors with a biased presence.

It does not escape our attention that hb-egfaEN TREE recognition is associated with permissive, pro-regenerative environments of zebrafish and neonatal mice, but not with the scarring profile of adult mice. This stage-dependent, cross-species recognition invites speculation about broader concepts in regenerative capacity. The potential for regeneration is unlikely to be defined on its own by the ability to read and follow the instructions of a panel of key TREEs. However, it is clear from past studies that regeneration invokes a context-dependent chromatin environment, and comparisons of these environments between cell types in neonatal and adult spinal cord tissues, including their ability to recognize other TREEs, are likely to provide additional perspective.

Finally, human HB-EGF has stimulatory effects on regeneration of axons across a spinal cord crush injury in neonatal mice. It will be important to identify a more precise developmental window of these effects, as well as the influence of dose and delivery method on the extent of axon regeneration. In these experiments, although the experimental viral vector was delivered systemically, inclusion of hb-egfaEN in viral sequences was sufficient to focus HB-EGF expression to the injury site.

There are many components of this delivery system that can be optimized with the intent to alter dynamics and dose, and reduce off-target expression, including choice of AAV serotype, choice of TREE or modified TREE, modifications to payload, and use of multiple enhancers or gene cassettes per vector. With such optimization, TREE-based delivery systems have potential applications in precision interventions to enhance regenerative responses.

1. An isolated nucleic acid molecule, comprising:
a nucleic acid sequence encoding
a tissue regeneration enhancer element (TREE);
an encoded polypeptide; and
a promoter directing expression of the encoded polypeptide in damaged and/or injured spinal cord tissues.

2. The isolated nucleic acid molecule of claim 1, further comprising a 3′ UTR noncoding region, inverted terminal repeats, or a combination thereof.

3.-7. (canceled)

8. The isolated nucleic acid molecule of claim 1, wherein the promoter comprises a Hsp68 promoter or a fragment thereof or a cfos promoter or a fragment thereof.

9.-10. (canceled)

11. The isolated nucleic acid molecule of claim 1, wherein the TREE comprises hb-egfa-linked enhancer (hb-egfa-EN).

12. The isolated nucleic acid molecule of claim 11, wherein hb-egfa-EN comprises the sequence set forth in SEQ ID NO:34 or a fragment thereof or a sequence having about 50%, about 55%, about 60%, about 65%, about 70%, about 75%, about 80%, about 85%, about 90%, about 95%, or more than 95% identity to the sequence set forth in SEQ ID NO:34 or a fragment thereof.

13. The isolated nucleic acid molecule of claim 1, wherein the encoded polypeptide comprises heparin binding epidermal growth factor (HB-EGF), recombinant heparin binding epidermal growth factor (rHB-EGF), heparin binding epidermal growth factor a (HB-EGFa) or recombinant heparin binding epidermal growth factor a (rHB-EGFa).

14.-17. (canceled)

18. The isolated nucleic acid molecule of claim 13, wherein HB-EGF, rHB-EGF, HB-EGFa or rHB-EGFa improves spinal cord function.

19. (canceled)

20. A vector, comprising an isolated nucleic molecule of claim 1.

21. The vector of claim 20, wherein the vector comprises a viral vector.

22. The vector of claim 21, wherein the viral vector comprises an AAV vector or a recombinant AAV vector (rAAV).

23.-26. (canceled)

27. A method of treating a spinal cord injury, the method comprising:
administering to a subject in need thereof the vector of claim 20.

28. The method of claim 27, wherein treating a spinal cord injury comprises stimulating regeneration of injured and/or damaged spinal cord tissue, promoting glial cell proliferation in injured and/or damaged spinal cord tissue, promoting axonal tract regeneration in injured and/or damaged spinal cord tissue, triggering neurite outgrowth in injured and/or damaged spinal cord tissue, triggering neuron formation in injured and/or damaged spinal cord tissue, improving spinal cord function in a subject in need thereof, or any combination thereof.

29. The method of claim 28, wherein improving spinal cord function comprises improving sensory function and/or motor function.

30. The method of claim 27, further comprising reducing inflammation in the injured and/or damaged spinal cord tissue, reducing scar tissue in the injured and/or damaged spinal cord tissue, or a combination thereof.

31. The method of claim 27, further comprising administering to the subject one or more additional therapeutic agents.

32. The method of claim 31, wherein the one or more additional therapeutic agents comprise agents that promote glial cell proliferation, promote axonal tract regeneration, trigger neurite outgrowth, trigger neuron formation, stimulate regeneration of spinal cord tissue, or any combination thereof.

33.-39. (canceled)

40. The method of claim 27, further comprising repeating the administering of the composition, repeating the administering of the vector, repeating the administering of one or more therapeutic agents, or a combination thereof.

41. The method of claim 27, further comprising monitoring the subject for adverse effects.

42. The method of claim 41, wherein in the absence of adverse effects, the method further comprises continuing to treat the subject.

43. The method of claim 41, wherein in the presence of adverse effects, the method further comprises modifying the treating step.

44.-51. (canceled)
